SEQUENCE LISTING (131 sequences)

SEQ ID NO: 1 - DNA, 127130 bp, Homo sapiens
  exon (2001)...(2150): Exon 0
cctgtctctggtgtgccctggcccccgacttggaggcctcctgggccaggccaagaccttccccggcagcgatggtctccagccacactcaactgccctgaagggacatttcctgcttattcccttgcccggctgtgtcctccacccggaaggcctgtgccttcttcgcctgcatgtcctaccctgaggaggctcccttggtctttcatcgctctccctatgggtcttcacgccttcccgaaccaccgcgcccaagcaggagcacgttctcggccctcttcacagggcgctcctcctcacaggggtgcccgggatttttattctgtgccttcctggtggctcctacaagtctggaagggcaggaggcgcatctcactcctctgggtcccctcccctagcgcctggcgggagcccaggctgcatttgtggaattcatgactttttctctcctgctcaagctgaacacattgctggctcctgctcgggtggagcccggctaattagagtgaggggctccccgtagggcgaaggggtgcgctgtcagatgtggcattcccgttttacggagacacacggtgtcttacacgccagggagaggtctgagacgcaaagagccgtcgagcgggctgcgggattgcttcgctgtcacctccgcctgcagccacccttccgcacgcacttgtgtgtgcacccaggccaacatggaaggcgccatcctaacttctgccgtgagcaggtgggagggaagagagacgagaggtattccattggttgtctgggaaaatgaattgcaccttcccctcccttgcggaggatcaacttttcccaccccctcgggtgggcactcgcatcctggggccggagcctgaacccgggagccaaggggccccagttccagggacgtgaagctgagcgtacagcgggcgctcccagacactggggaaagtgctttacgatgtcccgagtccctccagtctcgccagcggggcgagcgtgagggtgccccgaccgaccagcggccccgggtgcagggtggcgggcccggcggcgcgcgtccccctccccctcctggcggcccgcacgtgtcgcccgcgccgcgcccccacgggttacgcgcgggtcccgcagcgccgcggccgagccgggctgcccggcccgcggacacagcgccggccgccgcatcccgtgcggggccgcggcgcgatgctgcgctggaatgaggaagcgcggcggcgaggggagggcccgggcgcggtgcgcgcgggggtggcggcggcgcgccgagcgggcccggcgcgggcgagcgggctgcagccggcggcggcgccagcaggtacggcccgcacccgccgccgccccggcggcctttgggggctgagccggagcccggcgcgattgcaaagttttcgtgcgcggcccctctggcccggagttgcggctgagacgcgcgccgcgcgagccgggggactcggcgacggggcggggacgggacgacgcaccctctccgtgtcccgctctgcgcccttctgcgcgccccgctccctgtaccggagcagcgatccgggaggcggccgagaggtgcgcgcggggccgagccggctgcggggcaggtcgagcagggaccgccagcgtgcgtcaccccaaagtttgcggggtggcagggcgcgcgctctggccacccgccgctctgggcggcagcaggtggcaacgcaagggcgcggcgggggcggccggcgcggagggggccaggtacgcggcccgcgggcggcgctgtgcgcgcggggcagccggtcggccgggagcgcgaaagcctggtctgagccggctgggggcggggagtgtggcggagaaatggggaacaatgcgagtgagcaacttcaggaagtcattgtgaaagaaagctgggaagagctccgcggccaagttagcaggacactctaacaagtgactgcgcggcccgcgcccggggcggtgactgcggcaagccccctgggtccccgcgcggcgcatcccagcctgggcgggacgctcggccgcggcgaggcgggcaagcctggcagggcagagggagccccggctccgaggttgctcttcgcacccgaggatcagtcttggccccaaagcgcgacgcacaaatccacgtgagtgttttcaaattgaatttcaataggaaaacttggggtaactggtgaatttaaaaaaaaaaaaacacagtaaagaaaagcggtaaggttggtagaccctggtgtcgctcaggtccgcctctcttttctgaggacagtgagagagttcacttctgtcaagcgtctgttgctctgcactgtgccagcaggtgcaggaccaggccgacatgggacacttctgagcagccccgctgtcaccaggagaggagttctagctcccaaccatatttaaatttatgtagacctacatatacccacggaagtcagcctttataaagtcgtgtgtaaagagttttccttatatttgagccgggagctttctttttatactataaatatgatgagatcgagtctgaacttaatttctgcaagagaggaattatcccggctttgaaaagttagtccttttgctgaccgcaggtttgacgctcaagtcaccaaaccttctcaggaaaacccttagtaatattaaggcatcaggttacttgcggttatatttgaaatgtattttaaatatttgtcaagcatcgctgctgatgcctaaggaacctcgtgagggcttgtttttccttctaatttggaggcatctaatgaccgaaaaccgtagcgattccatagggtctgaccaggcacagctttcaaatgcagcttccctctctctagggactgcagcccacccagactgaatttcaatgcggtgcgctttgcttaggttacccactcacaatttcccactgcgccgcaggcagtatatttcagctttgagataccttgttttaaaattccagacaaaatggtgttgaggaaatgtctccttactagtcccatcaacttctgttaaaagaggaaaatttatggaatttgaaaatactgcgtatgatatttaaactttcatagacattcaaatgcttttaaggccaggttcaatttggttatgagtcgaggggtgggggggacccacatagaaatgtcctgggtcctcttgagtttatttctttgtttgaagatgtttgttcaatgagttttattgtactcatcttttatatggaattttaaaaagtaacaatttcagtattatttatattagaatgtgtcagaattatttccgtgacaaatcagatcatttgggctatggcttaaaatgtacacgaggcaaatattcatgacaagaagattcaccttcttacgctggcatcttgtaaaatgcagaacaagttaaagaaataatgtgtacacatacaaataatgatgtcacattaaaaatactacactattcttgcttgatggaatgtatctgatttccaatttcaccatgaacatatttcatacattttttacatgaaaaaaaacgtgactcttaagtctcacagtcaatcagagctggtgaccagaacattttattgaactaaatggtcatgttttcttccccttttgtttcacggtgagagttgaaggaaggagtttagaaactctccagtacttgtttaattcatcagtgttctaattagagtggtacctcttggaaaactacacacccccctaatgcagaaacatcatagcaataatcacccactctcagggtctccaggagaccacaagggctgcagataaaagtctggatgtgttaggtttgaccctttcgaagagttttacacaggctcctaaagagaagatcagctgtggccgtttgtagccatttcctttgtcgaaaaactaagatcgcagtgaatgtattagccaagaggtctaaagccctgttgtactgcaggccactgtcttccttgtttgactagagacttggagtttgagaacagtggttctttggtttggatacattttttgttcttgatttggatgtgtgtgtttcatgcgtggttaatatagcatattttcaatataaatgtcaaaaattttgaaataggaaagaactctctatatattaatgtacttatacacacacttcaagattatgcatttattaacagatacatgaaataaattccatgtgcatatgcacatatgcacacagagcgtgcacacacacagcatgcacacagcgtggagtgagaggcatggggcagtgtggaagagttttaacatcaaacagacctgaaatgagtattaaaggccccctttatttttaaacttttactaaaacaagatggatttccctatgttatataatggtgaattttaggcataaataacgttttttgagtgttgcataattgtacgtattaatgtaatgtaactgtggttaacgaagaattcatcaaggatatcactgttttgtggcattttttttttcctcctctaatctttggacttgtgaaataatttcactatgaaataaatgttggttcttgtcatattctaagggagattgatgtaagtggctccactccagcttacagaaggtaaaccacgacctttttgcgttctctgaaaacgcttgtcttccgatgcctctgtttctaagactgacaagcactctgggggcactgtgacgcctgcttctagcggcagagttgctgcagctcctgtcctggctgtgaacattgttctctctctggtgtctctatgttcataactacagagacttcagctctattccatttcatatttgtgctgaataatcattccattttatgggagaaaacacaagatgtaaaagcaacaagtgacccatcctttgaagcttacaagaagagaaacattaatctatttcacgtcttgaaaacagatcagttttattttgctcaaaaagggcacatgtacatttttgatctaggtcttagaaacgtagagtttcagaggatcagcattatacacactgtcacacacacacacacttaaaattcagatgaggaacaagataggaatgaggttttgttagggacgcagagcacctaaaaccaaaggatatcgacagtaacaaagctgtttttactgtagtgctgactgaacactcatgctggtgtcttcatgtggaccatggctttcttgtatttctttgcagtttaataaatgacttcatatctcaggttacctttccacatctcctggaatatatgtttatgtccttaaagtttcagtgtcgtcactttagtagctttagtttgagtttttaaatgtttggtaatattccaacaaatattttttaagacattatgaaaccttatgaagtgccatatattacaagtgagataaaacagcaagcaaaagaaggtttgcagaaggtttttaagtggcgaagtgcgggcctgcccattttggtgtctccttggtggttactcctgagaagggcctggaggaagagcaactgaggcctaatctacaggcaactgccaaattgtttcagttgacgtttttccctctcatgtttgactataataaataggtagttgccagtggagccttcagccaaccacctggtaataaactgttaaaaatggtgcaaaccctaggtcacaggtgtgggggccatttgtcttgcctgttaacaggcctggccttaattcttttctcccatggccatttctgcctttggggaactcacaattcctgttgactaaaagagcacccttttccaccacaagcctgacaaatcagacgtccacataatttctgaactcgttttggttaggacaggaagcacaggctcccttcctgtctgtgttttcctaagagaaaacggtcttccctccttttttgcatatttggcaagtggttccacctttctctgcaccctggtggagtgtgaaggcagcagaggaaccttttggaggaggaagaggacacagaggccctgtagccaggcaccaagatccctcccaggtggctgggtctgaggggaactccgagcagccctaggtcctcaaagtctggatttgtgtggaaaaggcagctctcacttggccttggcgaggcctcggttggttggtgagtgccacacggtttctttgtgtgcttgcatggattggaatagccattgtgttcttccgtcttccctgctggtgtttccacagtgggtggcctgagcccagagcagctccccatatccctgtgcaggccacctgtctcgggtgatggagagcatcattatgctccgtctgaacgctctgctttcggatggccccatgctccacctcctgatagctcgtggcgcggggccacggcttaacgaatggctgaaaatgggtcctaattagtggaaaagtgctttcttcatattttctcactcgagtgtgcagtgattcatttttcttctgcaatcagctcactgctaaagtaaatctgactctcttcccgccattgcacaccaaaagttaactctaatgggtaggaggttaggtttgttgagagagcaatgcagtaaaaagaggggatccaatgtggtcttgtctgtctggtcttcctttcttcgttttttcctcccttgtcttctctgtcattcccttccctccatttgccttgcctttcctgtccttcccttcccttccttcccctctttctttctataattggtggggggtttgcacagactgccaaaacactaagaactgtgtaaagtgtttttgaatggccttacacatattgaagtagatttttatgctccatttttgagatcacacactaaaatctatacctttaaagcattttctgttagtttgaaactatttgaaaatgaacaatgtggtttagattagagtcctgttctgaagctaggagttccactatgaatattgatttatcagtttttgacaaatttttgttgttataccagattttcactggcaaacctagagcaaataaaattccacataagatacttccctagacctaatgggaaaaatgtttaatttagagtctttaggagaaatgagaatgaggaattgaccttttgtaagcttacttctgaggcactctgaagtgtgttccagtgcttttaatggaaactagagagagccagcaaccccctagtgtgagccccacttttaaccggaaaaagtgaccttttcctcctcctttgtgctgagttttgcgtagggcagaaaattaagctgatattcaaagagattcactgcaaaaacatattgataaatcgtatattctatttcattaaattaaaaccatactgctaattatctcaggttgttaaacataaggcaattaattatcattttaaaagttggtaggaagttgtgagtacttttgcagtatgagtgttttcccgctttagtatgaggttgtgtatgtttgcttgaatttacagaattttcactttaagagcagacaatgttttgttaaagaaatgaaatttgctaaaaaggagcatgtaaagtgaaacattaaaaataaataatttcaacttacttaagagctgcagaaaaatctgattgctgtgtttaaaatgaattttcccacatttcgctctcttatggacaggagcattttctgtcaggttataaataaagacatgcccattttttgtacccccacaaatgaggaagttgtaagctctctgaggttttactgatgagccccctccccctgggtttgcatgaagagatcataggccacaaataaaggactacaaaatggggtctaaactatcctggtggggcctgatacccacgtttcgcatggaccttacgatgtgatgaatggttttggcatgagtgtcttaagaatgcttccagattcaggttacaggacagccagcgctgagctccctattgcagaacaaagtaggaatctagaactttcttgctaacaggatccagctaaaacaccaagttagattcttaaatgatgttcttttctgtcattatttgattgttgtcagtagcagtaattgttaccaagccattgatgcttctattcttccctttgcccttctgagacacagctcattttgacttcagtggaacccctcgaaggtggggtgatgagcaaggtgaattttcaaagtaaagctactaagagaccaaactacaatttaaggaacctgatttttgaatcaaattccatatactgtgggtatagttcaacatagattaatttcttatagttattatgaaaaaaatctcatcttgatgatagctgataattttgtgggtgtcgtaaacaaaacagaggtcagaattcagtcccttggggaaaatttccaattagtaggaaaccaagtggcctaccttagtttgaagacacccatcaggatgtctgcaccttttcatcctctctggaggaaagactaaatacccattattgtatataggtcaggccaaagcagccttttatattgcaaggaataagaggtaaatagatatatgtgcaacaatgaatcccctaatgtgtttactctagaacacatgttctttctgtatttatatgtagattttgtagatcttgtcttaccacctgctaatggtagatactgtatctaaataagttgaggaaaatttatagtacctaggaatgtgtcctcagtgggccaatcaatcaatcatgacttcaggttatttttaataaatatacacgtatgggttcataaacaatgggatgttcttgtgaagatctaaataattttacttctttgggactaaataaaatatagcttttgccaaataaactcacacaagcacttattttaatagaagtcaaatggctttgcagaaacttcagttttacaggtgcattgtttgaaatgttacgggtatacaagtggatttctctattatgtacagtgttaagtttgagtttcaaaatgtccacctgaaatgatttacttgtacgttaagataatttaactgctaagaaggcaagataaagcattctttgtgacaccatatggccttgctgagggaaaaacttactgttataagtttgtgtttatctctctttttaaaaaaaaatgaagaaaaaaacgtttaaaataatgggaacacagcagttcctggggtcctctgtctctttatcttattatagtaaattaccaaaaaaataatgacctggggcatgtctgtgtggacccttcttttggaggcagtttctgtgttttgtaaagctgtaggttctattttcattgcacttcatattgctgcacagctcctgaccatgcatgaaggtcctctgaaatcggtaagagggcagaagaaaatgattctaaacttagatttttttaacttaagtgatgaagtgtgaaacgccatttatatttgaggaagctacctaggaagtggctcatgtcgatggcccaaatcagaagagggcctgtaaaagcttctatcaattttgactgtgtatgcttctaccatggcggctcaataaacagcagtattagtttaagagtggatggtacagtagtatagacgggaagcctctcctctccgtgtgaaccgtgcacccctatgagagggtagagacaatacaatatgcctgtaacgtcaggacagacagtcatggccagcttgaactccagccctgggcttcttgcagcaacaaacgtgaacacagaggactgtctccaactccactttctctatttttaaaacaactttttgaatacagtatctgccatcttttcttatacctcactttgaaacaggtggctccactgtggcatttaaaatgttctgtttcttttccctctgtatcaaatacctctttaccaagaaaacattcaaacagcatagtttttaactgtattttgaaaggtttccttagttccctttgacccttcctcttttgcatatcagttcctggccataaaaataaaaaatgctaggacagaattgcacatctgagctgatttgccctcaaaaagtttcacagtggaacaaaccgcaggaggagttttctgtggctcagttaaatgtcgggggagggtggtgtgaaagccaaattggattcctgctttcctgtttaaatcttgtttttcattgttatttgcaccagcaatactctgtggaataatcatgaaaatgtgtagattggcagctaatttttgaaaaatgaaaagaatcagaaatgaaataagagtgctcggaagtttttatgttctctcaacctgttttgtcaaattgttacgaaaacctataaggtctctttgactagatacaaagactttgcatattgccttagctttctcttgaagcatttccttttttaaaatacagtgtaattcacagtgatatgatagatttgcaaaagtaaaatctaccagtctgaagatgaaaggacttgtctcttagcaggaataatgggttttattaaagaggtctgtgacctaaggcattttaaataaattacaggcttggtccctgtctctcccatgtatctactcccttcaatataagcatcattgagtatttaaggaaataaccccaaatgtaactctagtgtagcttcacttgtcagggaggaaaaagtaaatagcatacatttggccaaataaccagaactttactgtagaagttttatgatgaaatttgcctttagtgcagagtattacaaagatcatgtttagtttctagcagtatataagtagcatccatccttatctgtcatgcatttggagtgcgcgacccctgcactgggctgcaacattctgatgggcaagagtgctagagagaaagaggcatcaccatcagactgcacgggttcaagtgtcagctctgtggttgattagctgtgtgacctggggaaagctatttctcttagccttggttctctcatctataaaatggagataatgatgcagatgccttgggtttaattgggagagttaaagacacatttacatatttagcaagtaggtgttgaattctagctctacattggacactatgccaggtgctcaaataaacaagtggacaagacagacaacacccatggtcttatgaggcttaaccatttgcctcttcaatgccagaaacttagtaggttgattagataaagccagtgagtaccagtatccttttctttgcagccttttcctggcacactaaaaatactcagtacatatgaaatatcactggacaaagaatcccccttagagtaccaatggagaaggaaggcatttgcttaaaagcaaaccaacagaaagacattgtaaggcagttgtttaagtctcagagacctataatttttttcttttttcttttttttcatctcgctctgtcgcccaggctggagtgcagtggcacaatctcagctcactgcaagctccaccttccgggttcatgccattcttctgcctcagcctcccaagtagcagagactacaggtgcccgccaccacacctggctaattttttgtatttttagtggagacggggtttcgccgtgttagccaggatggtcttgatctcctgacctcatgatccgcctgcctcggcctcccaaagtgctgggattactggcatgagccaccacgcccggcaactacaattgttcttaaagcttgtagaattactgtgtgctaccaacagacaggctaattttgagtgaccctcagtactttgtacagttaatttggcacgctgtgtacttagtggctttttaacagctataaatttgggctgctagaaaagtagtaaagttgtgattcttgacaggcatctatctgcattttcatttttacttcatttgtctagactcagcttgtcagaattatggaagagactccttgtgtcagggcaagcactgtgaagagaggtattcactgtcagaaaagagaggggagctggaggcagctcagaggcctgagacccgcctccacaggagccccagcaggttcggtggagctctggccacactctcctttgggatgctgaagtcagaatgagttcacttcccagccagtcttgccaaggctcctcacctggaagcagcaactgcccagggctgttggatgtttctccccaggggacagccaggtcccagtcccacctcggtgtggaaggaggaaaggcagggtccaggaagctgtttcaggacaggcccaaggtcccccagggatgcctttcagggtcagcggaggctgtaaatcagcagggcccacacggcctggaagaggcccctgtgctgtcggcttgcccggcttgcccggctcctagtccggcttctgctcctcctttgtaaagttatggatatgctaatagtttccaactgagactaggaaagtaagtcctacttgacactgtttggtcagaaagagggagagaaaggagaaggacagagagagactgagagagagacagtctcagacaaagggagacggagggagggagggagagacagagaaagagatgggaggtaggtgtgggaggagggagagatgcagaaggcagaggaaagacagacagagatttagacctcccaagtcagtgagcagtccagagttggagtggagggtgcctggtggcttgtgactgcagactccactccccgctcctagaggcacagccatggacagcttctgtcacgttggccctgcacttatctctgcatctatttccccttgtgcaagattcagaactgcatgctccaaaaaaacaataaaagcattcatgttcataagaattgcacaggtaaaaggtagtttgctgatattgttgtattttttactatcgcttcttttagctcttgcctgaaattgtttgggtttcccaggcaaagtagaaaactgcggtacgtttctgtgaaataattattccttctggcatctccctttacagacctactgatcttgatttttcatttaggtgaaagtttgtgaaaacatgccattagcttgctttgtgattaactccttttactgaatgtgagctccttttaaattgaggccatatcaagcttaaattccatattttacccggcactctgcatttcttccatgtgggagaggaggggctcagtaagtgctttgtaaaatacacagccgaagtgatgcacgtgctaacaaaggagtgtgacaggacttaagtgcccttctagacacttcaggctcccctttgtaagctgtcttggaagaggccacatttcctttccctcaaacagtttctcattgtttgattattcttttagcctttctctggaagcaaagccacttttacgagaaagtcactgctttttcatctcaagagatgcaagtttggagtttggggaagttttcaggtgcccgtcaagtcatcctttatgatgtcagacgagtcaggccacagaattcacagggctcagtgcagaccgaaaacttgaggcctcttgttcagaaattattaaaaattttggtgaacatcaccccaagcaaagagatcccctaagcaccagcccccaagcaactgcactcataagcccatgaagccccctgctgtcagaaacaatgtggttgaaattgtgtatgcacttggaagtgagatggattgcaaaacacaggtctccatgctggggcaggagtggtgatagggcatggagtggaaatgtccagcaggcccacgtgcgaaaatgcagagctctctggctcttgcagacttggctgctgacaatagacgcgctccaggaaggtgctcgctgtggtgtgatctgctgcccacccctagctccctccaggagactggtgcggggactgtttgcaaatgactgcaaaagtaagaaggttcccacagagcagagcttgatttggggaccagccgagggcagtttgtcaggattccggcttgaaactgttctcacatctcaccgcctgaaaggacgagtgtgtccagaggacttagcattgatcacctctgtctccatgcagcaaactcagaggctcagcccgcattccactggaagggcgtttgccagtggtgttggttggaagagccttgactttgccttaggaaacatctttttttaagaattgaaaataacttgagtatgcaacagtagggcatttgttatataaattagttgactagtgtgtagccagtaaaatgatgatggtggtgtgtatttgttaaataaaaagatatgtgtggtattaaattaaaaaatattttaaaacaacatatttgtaatctgtttagtgtcctctttttgtaaaaagtacagaaataaatatacagaaaaaatagtagtcctaagtggtagaaattatgagcattttcttgcctttaaaaaaagttgtaaaagattgtatcatttatgtagcaaaaagttttaagtcagcattctaaaaatttcgtgttgttatagttgctgtgacaagatttaacttctgtatgcttcaccaatcaatacagaggtatttaagacccggtgtgtgataggccgcgctaaaatactatacacatcttcagaaaactagagaactaacttctaacttcctatattagtgtggcacggctgttacaaagatttttctcatttgagtctatcttgcttctttatcattgttttgacagtttcagaagaatcgtggcttttccccttttttacagtaaaggtacctgagactcttgacgtattgctttttggaaatgcttgtgctggtcacatgcttgcatctgggctagtgtgtctggcttccgtgtgctggtggatgcttactctgttttctgaaatactttttctgtacagtggccactagctgtactcctaagccacacacctaccttgaaaattcatgtcacttttagaaatagataaaagcccctcccatccagaaaaagtgactatcatgtatatcctcatcatgactaatactgatattcctgaaattgaaaatacatattccatatgtaccataaaaggtattaaagatatatggagtgatagatatattatatataacacttctaccctcacagttttcagcctaattgagagggtaagatccctgaatcatccatcagtttttcaggtctctgctgaaagcaggccacagctcagatccacacatctgaaccagagacagaggtggccaaaaataaaaagggggacagggggacaacctggtttagagtcaacaaatagactgcattttctggttagtgaaggagctctcctgaaagtcatataccagagcataaatgagcagatttccttgaggtcaccttctgctggccatagctttcttatctgtggagctgccagctgtcatccactttggggcacctgagactgccgagcggcaggccaggacccaagtgcgaaacacagaacacctttttgtttctactccactgatgctggggttctctccctggtgtttgtggctcgtagtacactctgtggaacattcactatggtcatcgaagggcagcatcttcccagttgtttctttcttttcttttttttttttaatttaaaccgatctgagaagccagccatctgtcagcaaaacaggaaggctcgggctctctcctgggctcgttttgctgccgtagtgagcgtcacttctccccgtgtaagagtgctggtgaaggctgaggcaagggcccagaaagattgagggacaaagacaggagcgcccgcattgcccatctgccaggctggaggtgtattcattattgatggaggtagtgcagttgctgctcagatatgcagccctgcctgggtaaatgagacattcttcagcaaattgcttcgttttttgattgctgattgtacgcgtgtcaccaagctgactcaaggttcatcgatgcatgctcagtaaattagaaagaacataactatggatcagccaagagaatgaattctgtgcctacaatgacccagggccatttaattttctgcttaattttgttgcagtcagtttgcattttgggttattatgcagtaggaaattaacaataaataacaaatttggtcctcctgtgcttgtaatgatatttttataaatctttgtaatgctgtttttaaaaggatcaaggtctgtgccagtctgatactccagcaagtatgtgaggaggaaaatgcattattcttgctagataaccttgttgttaaatagcataggggttctttatctctctctctttctcatatcttattagtatttttgctttaaactaaaatcccttcctctctttctcagataacctgaggaccatggatgctgatgagggtcaagacatgtcccaagtttcaggtgagaccttatgagatagctgtgtgggaagttcatgagaaaagcttccctggggccggaagtcacagtgcttggtatgctcatgggggaggaataggggctattctgcaaaagaaaagaccatgatggaatttgcctgagtgtttccttcacctgttacaaattatctcactttgagctgaacagaaagcctccaagatgaaattagttttactgttaaacttcaggaaaaaaaaacgggaagagttaaatacatttttgtactgttggaaggaaaaatggctgattggtttaaaacccaaacacatgccaatgatggtacttaaagagagagagagagagaagcttgaaaaacataattgttgggcacagtcatgactgtttgttcattaagcatggacacaacattgctcccctttgccatatatcttttcaagccgtattggatatagctcttctcatccaggagacccaggaagtggagaagtctgtagtaggaaaagcctaagggtaggtcacagactgtgaccatttggcagcactgagggtggacggcgagccagtccaacaaaaccgcacagttccccagtgcatggacataggaagacagctttctatctggccctgtatccagaggcgtcagccccagtagcagctttcatggactttggggttttcggtatttcatatttttgagcctcacagactcacagccagccccagaggctgacttatatttgagaaagttctcagtggcaccttgccttggctgagcgccctcgtgttttgaagtttctatgggattctacaagttggtgctcctgatgaagaccaggacctatgtgtggctgctcccctgcttggtggtttccctggggaaggtgcaggagaggatcttctgagttccatggaactggagatagatctgccaatcacaggcttccttctccaccactcctcagccgctctattcatgtttcagattttggacttaaactctcccaggtgcaaagaacaaacaaaaggctagcttatttttcttttagagtgaggcttcgtatttattacaatataattgccacattctttgtgtaattctcacatttatatcttaaatataattctcatgaatgagaattatataattctctttttgtatatcattgaatattttcacttaatttttaatttttttaatcgtcacaaaataattgtgtacatagacacaaaataattgggtacatagtgatgttgtgatatatacaatgtatagtaatcggatcaggtaaatcagcatattcatcatctcaaacatttatcgtttctttgtattaggaacattcgacatcttccttctagctatttgaaactatatattattgttgactacagtcatcctgcaatggtgtagaacactagaacttattcttcctacctagctgtaattttgtctcctttaacaaatctctccctatcttccactcccccgacctttccagcctctattagcctctgtcctactttctacttataatgatgacagcagcatttgttagtttccacatgtgagtgagaacatgtggctttttaacttttagaatgtggtattcaggcacttcatggtacagttggtaaaagtgaaaatgtgtccaaaagtttgtgattatctatataaacaaaaatggtataaatccaaatatcaattttgcattgaagaacttaccttagaggtatattctcacaagtgcacagagcatttaagcatttgttcactgcagcattgttatcagtattttaaaactatggtacatccatgtacttccacatacagctcttaaaaataaggaggatatgaatgaactagtatgaaaagaagtccaaatacatgtgaaagtgagaatagcatggttctggatggtatgcaaagtatgatctcgttcttttaaaagaaaataaattacatacacatacatattttctatatgcttgcccataacgtttaggaaaattcttgggtgatatttattaacctggacttcctcttggaagactgatggtagaaggaaggggacgagttagggaagaggaggagaaggaaaactttgcttttcatcttctaccttttagcattatttgaatttattttccttaagcgtttactttgtttcgtaaacaaaaaagcacaaaaacaaaaaacgagttaaatgggaaaaaaagcagtttagctctttatagcctctcatttggcttcgccagcctctcactgcagcctcagagagctggtctgggaaacactggtagatgaggactgtaatcctcactcatggaagaggatctcattcactgggtttgctgactgtgactagaagtgattagggtgtcaaaaaacccaagcatgttaaaaatttccagaggccaaaaagatgctttcattgttctgctcttcttttccttgtcgctttcactttgggtagcttctaaattggtattttgcatggtgcatttaaagaaaatgagacccctttggccaatgcaggagtctacactctgatattctagagtcaaagctgaatgctgacacctaggaattcatctctagaatgtttatataaggaatagcccctcagtattccgatctcgtatcttagtaacgaaactaacaaaagcctgattctcctctggtagttttcttgtctttaccataatacaaaataagtaatttgttctgcaccctgactgttcaaaggatagggtagctgggggcggggacaagaatggagaccttattacataagacttcctgaaaaaggaaactctgtttttgtttgaaatgatttggtctgaaatttagtttgtgtacacttaccaaagggattcctatttctaaaatactcatactgcttttgattcctgttaacctttgagcactctacgtaatgatgagagcacttaaagagtcatgtcacttttagtaaagaatcaaagaatactttttctacttcttcgagtttgatctctgcttctccagttaaaaccagtatttgtttttttcatttctaaagttggaagaaatgacagttagttatggcataaggatgtacatttaaccaaataggagttgacattcttggtaagaaatcttaccaagattatgttatagattataagaaatcttatcaagaatatgttcctaaatcatcctcttttcccataaaatattaaagtatcagcaatttcataggattcaacctaatgtatgcgaaatgctagataaacagataaatacttaatatctggctttttttcaaagcactgggttatttgttccttgagatttatcctaaatgtgggctataccctggtttacagtgtctcacagatgtgtagtagtagacactccataagtgtttactgacttgaatccacagggtactgagaaaatgctactgatagacttggaggagagcatatctaaagcaagctaccctttcctttagggcacgtctcactaattctttgggtaaagcgtatttttcttccttttgtgtttttggcagtctttccaaaaatacgtgttatacctatgcattattttttggtttggtttctaaagaaagagtcagccggtgggaaagtgaaggatgtgggaactgagagatctgcatcagcatcccacctctacctcccacgatgggacctgagacagttatttttgcctcctggaccactatagtatcatctgtaacaggagggacttgagccagttgatctctaaggttcctctggcacctgtgaccctaaatagatattggatattggtttaatgctatttgtagtgtgtttttttggggatatggaaaccagaagtttgtttccataaacataaacataaactgtatatatctaaaggatatggaaacctttagatatatataatctgcttacgtaaagaaggtttgtatatattgcagtgtcaatgggaatattttatcaagttaagcatagtaaatcacattgattaaatgctttgtatttaccaaacattacccaaagtgttttctcctttcaacctcacaaggacccacagaagaaaatacagttatcatttccaacctgcagggagctgagacacagagaatttaagcaactgaccggaagtccaacagggagtcagagattgctctggggtgtgatccccacttggaccctagagtggaagcttctccactactttatagagttgagattctatattttgagcttgtatttacccagagaattatatcctcttgggcaattgtgtataataaaacctcatgcatttaggagaggcgggatgacagaactttgttgagtgaattataatctacttgagaaattatttgcttacattttataagctaattataccatatctcatccagttttcccagaacacttctcataggtaatgctttatttgaaacataggccataggtaagttaagtgtaaatgtgtatttttataatttaaccagaagtttatttcatttttctaaataagtgaaattgtattgcatcttctaaattattctatttaaacacttgatgtcttgctgtctccgtctctgtgtgtttgcatgtcattgtacatgttcttaggaaaagtgtgggagcttgacgcaatatataccttatgtttctatgtgcatatagtttaccaaataataccataagtttacttagcatattagaatccatgcacattatttttattttatcttcaccgcaaccctgtgggatagaccaaaatcatgcttttcagcctcctttttccacttgaggaaaggagtcttaaaaaagggaccagtctcatgttcccattcgtcttacaactaattggtcaagccagaaagccagaactatgtcctgggtcactaactcctagtcactgtgtgttagtatttgagatgcctgttggcttgatttagtcatttattttttagtgttttataatccttgcatacttttacattttaaatggttaaccaggcaaattggtttaaaatcagtgcataaaaatactgtgcctatcatgatgggtttcatgaagtgataacttttcatcatggagatcctcagctgtcacagaagatgaggggccctgggtacagaggctcacgtgagggatgaaagtctcagcagcccggacttacactttggggcttttaggcaaatcagacaacctcttaagaactatcactgagttcaggcaaggcgagcttgaattaacacagggcccttggtgggcatgtgaatatatctcacttcactaccatccagttctgactctttacgagatgcccctgtacataccaagactgattttttattctcccttctccccatgtggtttcttctgcatagagagttcctattgatcagtctgacccatggtattttagaattgcgatccctactgtttcattattcctttttctcccccatgttgaaaaaaataaatgtcctgagatgcaagatcagggacactggagcactgacatttagttcagtgcaggaactgaaggcagatgtaattcttaagaagcgtacctgttattatgaaccatcctcaacaaattgtagtggatcttgttttctcatagatacagcagttaaattttttaataaaagtaactaagagttatttggatgtattttagcatgcactgagcggaaagtacgacatttcttcattgggtaagtcctgattctttatgatcctcacttggttccagggccccatgcatctaagggtgtctcagagcatcctgcagtgctccagcatgatcgcagggaaaagctataggaggaaaagagtcaataaagtttagtttctcaacctcccacctccaccccataataatgacagctggttaatcatgagacacgtgcacaccccacacgccctgtacatgtttactcattgggatagcatgtcaggccagaaggctccatggtcatttctatgaaggtactttagcaggtcttcaagaaggcaagtggcctgggtccctgcctccccaaattgcaagctccctgctttatgtaggagacctatgtgtatattacagttctgtgtaagattattttgttattcttacccccacacccaccccccaaccccccgctgccaccaaaaaaaaaaaaaaaaattcctctgacaaccttcataaagtcctgggagtttgaacaccattgctctaggaagtcatcttatacaaaaataagagttgtgaggtggttcatatacctcctgcgttctcctatttggagtttttccccatttatgaaagaggtgaaaacgctaagatatttagcaattgttactttaaacattttctatttataggccgggcgcagtggctcatgcctgtaatcccagcgcttgggaggccaaggcaggcagatcacgaggtcaggagatcgagaccatcttggctaacacggtgaaaccctgtctctactaaaaaaatacaaaaatttagttgggcatggtgggggatacctgtggtcccagctactcgggaggctgagacaggagaatggcttgaacctgggaggccgagcttgcagtgagccaagatcgcgccactgcactccagcctgggtgacagagcaagactctgtctcgaaaaaaaaaaaaaattctatttacagcagtgaaaatagtagcgacttaatgcacattgccaaggctttagcataacatgaacactttcactcaatgtctctctggccttttgtttttccttgggaaattcttataatcctgctccgtctttaactattcattttgtattggctatccaaatatacccaataatgctctttctgaaaatatgccaattgtggtaattacagctaagctggaatattaaattgtgatgtctgttttccagagaatgaagtagtattccccagagcataggcttggtgcctgtgcaggttctattttaaatattccaggaagggttgttttatatactgaggatgattttactggtcttgccagtcgtctgaaatgctggtattactcttgtggaaggtttattcaaacaaacaaggacatttcacacaatacctagtcatgtttttcagacattttaatgtttggttcatcatttgcacacactctcaaaaatctaggtttgtctatgtgttcatatcattttgcctgttgccagctcagtcagcaggcacactctcccaggctgttgctgttttgttagacttcttcaggaccttcatctaaaatggtcttccacacgtagctatactgcataagttcacatcatctgtttcttgcatgtgggttgtgtctcaactcaagtttaagttagatttggaagggcggaaactataggagttgcagcttcagtggagaaaagagcatttcctactagttatggcttcccaaggaaggttagattcctcagagtaggagtgattccccaatgctagaacctttggtcaaatataattctaatccagtcaaaataaatacaggtattctgtaaaacccgatttcattttgtaaatcctactttgtatagtataagcaatttttgtatttgtgtggattatattttattttcctatttcaaagagaagaatttgtattagcagactccctttgcatgcggagaggggatcattttcccagtaggcatggggttcccttccattccttgtccagtcttcttttccccactaagttaagtcaaactaagcagctggtaagatattccctggttcttgcaaagaaagtgagcagatggcagaatgtatagctctaagcagaatacctggtgtggtatcctcaaacacaaattgacaggagggtgtggtgtggcaagctcattgtgggggtaaattggaataagcttacagggggaagagttgacaaaagataggaagaaccttaaaaatatagatgccttttatgcagtgataaaatgtctagatatttatactgtggtgattattaggaatatgtgcaaagattggctattaggatgttcattacagtgttgtttaataattataaaaggacagaaagcaatgtggactcaaaaataggaaaagaatttaaataaatcctagtgtacccgttatacatgaaattatggaaatatgaccctgagcatggaaatatgtacatgagaatgtctaaaagctagttcattttgaaaaacaaaataatgtcacctcatattatttatagtatataaagatgattttaagagtggcagtgtctgggattataggtgattgtatttcttcccttttgcacatctatgttctctcatttgtattgtgtggggagaagtgactttttttataaaaagaaaaaggtatatgcatcccagcagagaagcactggctccacccagtacctgcctcctcatgccaccctctcaagccaaaagccgggggaagcccaggcaccttgaccatgaccgcccgagactcacacttcttctttctcatcagggaaggaaagcccccctgtaagcgatactccagatgagggcgatgagcccatgccgatccccgaggacctctccaccacctcgggaggacagcaaagctccaagagtgacagagtcgtgggtaagtgggtcaccagcggcctctgtgcctgtgaaacctttatctctttgtatttttccaagacagtgatgaagggatgcaagtcattttatccattgtgttccctcaactggcatattaaagaaatatggcacaaagatcagcaggatgggggtcctctggtgtgtgggaggatggacactcacaggccagcatggccgtgagagccacacaccccgcaaaatgtccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagctttgattgggccgttactgtcttttgacctttcttgtttgcttgcccttttgtttctttcctcctttttccttccttcctgtctttcttcttccttccttcctttcttcctttttctttttaattttcctcctttctctttctcctttctttccttccttctttcctctttttttctttcctccctccctccctttctttcttccttcctttctttcttccctccttccctccccaccctccctcccttcccctcgtctcccttcccctcgtctcccttcccctcccctccctccctcccttcctcctttttctttccctttcctccttccttccttcctctcttcttttctccatccctttcttccttttaacatttgatcatgggtaccatttcatcaagattctgttacttaggtccaaggactagtgaccaaattgtttttcccaggagtaaacttttctacaagacatagagaaatgactgccagtgccatcaggcacaccatcctgtgtgataggtaacctaaaataatggtacagcggggagtaactggaagagtcacaactacacaaatctcttgcattttcctgatcattaaatgaattagggatttcctggtttgagatttgtggacaaggagaactacttctttgtacaagaggcttgtgccaggagaagacacgctgtggttacaatgttactgtctctagctatggtcatttcatgtctttaactgagtgtacctagagtgtaatacagtgcagcagtgatgggctgaaggcctaatgcaccagcacacatgcctggaagacctaacgttgagaatccgcatttttttcatcactcacatttattttgcatctccctgttttgattaccttcagagacccaagcacttgcttttgtatatttgagtctgggttttattttccttttagaaatagtccaaaatccttttagagcctagacctttgaataaagtgctaacttaacctgaggaatgctgtttttttgttcacaaataaggcttaattgtgaagtcgtgagaattgctttctgagctgacttataaatttacccacctctttttccccaaacccccaaaaaatcctccagaaggtgagcccagcttgttgtaaaagattttgaagcagaagtcctgggtttaagcttcagttctctgccttaccagaatcgggatgtgtggggcaggcctacctcctctttcttcatctgcaaaatacattgaatgaaatcatattttttcctaagtatatgacatgtaaagtggtatttcaaaaactcattaatctgcatgaccaggtcagaaaagacacaccttttgccttgtgtctagtgtgtgactgtcctcacatgtagatgacccaggccacatccgctgatgttgtcttcctttacttctttgtcccttccaatgactcctttatgaagtcaatcagcagtgtttctaagccaggttccttctcttcacacatccccaacccacagggcaagtcatccacattttgcctctctattttttgtgctttgggaaacacattttacaacttccaaactatttgcctaagttcatctgagcacatctagaattattccactcctctgcatcagtgctaaaagtacaatcatgtataagctgttatactcccgttattttgtacctcatgtgcaggtagattgcaagttccttgcaggcaggtactacatctacttgaattgcttcacgatcacataacacaatgcagtgttaaaagtagaaaagtaattgacatcttgactaacagaaacactaaattcagtgtattgtgatgatacctttttaagagataggctatagaaatagaaaatcatcttagttggaattgaaagtcaagaagcaacattttaagtaagagatgaaaatacttaaaagggaaaggagcagtgcttacacagatgaggggaaaaaataaaatgtatcatctaaaaatttaaatcaaatatattaggttggtgcaaaagtaactgcgatttttgccattaaaagtaatagcaaaaaccacggttacttttgcaccaacctaatagatgggaaaaataagaggaatgaatattttaagctttgctatataattaaaatattcttagaagtctggagtctgtgaaggtcacaccctctggtcttctcccagcccatagggtataaataatctgaattgacggcatccagggatctcagaaattattagtacatcccacagtgaattaccaccttactaaaatattcatgggtatatactatggatttgttttatcctatttagtcttaaaaactataaagaaatctgcaggcttattaacatattactcagaatcatattgtctccaaagcacaaactgaatcagttacaagatattggactagagatcatggcaaatcagaggtacataagacctagttccgttgtggagctaaacaaactgcagagacctaaagggaagccttgcaccacactctaggtttggagctcaggttttgagtggtgtcagcactccagaacacatgggatccccgggaggtggaaattgagccgtctttggagaatcagctaatgagacagatgcatgttaaatgtctgttgtggcccaggcactctgctaggcagaggggtgaaccagaagaatgagattcatggggccaaagaatttgccttctggtgtaagaaaagatggaggcagcttggcagaaagaaaaaaaaggtaaaagatagaaatgaaatacagaataatctatctcctcatcccacaagcaattctgcctggtttgtggctcacgcctgtaatcccagcactttgggaggccgaggcaggaggatcacttgaggtcaggagttcgagactagcctggaccacaaggtgaaacctcatctctactaaaaatgcaaaaaaatttagggccgggtgcagtggcagttctgcctggtttgaacatctggttcagttaaaatacttcatgaaaatatttagcttgatgcactgaaatccatcactgcttttgtgggatgcactgactgcagtctgactgttctcctagtggaaaggggagcaatagcttctgtctgcatgtgcatgaaacagatggaaatttagaagagattctagtgcagacaacttcatcccacccccgctgcccacagagctagccatgtgcacggtgtgtttgaacaatgttatgttgctgctgtgcacagagatccccagtctcataaaacatactggatggaaggctgtcgagagatcatcaggccccagtgcataagttcttcctaagcatccttaacaggtgatcacctcgcctattgagaaccctcaggaggcagcccacttacggaagcactgattgttacaaaagccctgaactaaatctgcttgtctctaaagccagccctgcagatgtctgcagtgtaactactttctcttctctcaagcagttcagcccttcaaatcattgacaacagctctacaaagccctctaaatagcccgttctcctggctaaagtgtgttggtttcctcagcatatgatgaaataaacaccatgatgcctttgccttgctttttcttgctggtctcactcttccctggcattcttcaatgtgcccaagtccttcctagaacattgcacccaaagggccttcctggtctttccagggaatgcagaatgaaaaagaaatcccatcccctgcctcgaaaggagctcttctgtgtgtaagtgtaccttccccctcagggcataatgggaaacagtttgccaaagctaactgtgtaatttcacatgactcactgccgttagtctgagaaagcatggcatattaaactcattttccatgcttcccaactttatttactattccatttctgaggggcagagagctgagcaggaataacaccttccttgctcctcctaatggggcacatgtgcacatataaaattgtgttcgatctttgcagccacgacccatgttaactgtcaactgagaactcatgagtcagtttctcaggaacagccctgaagacattatctcctgcatcttgtatttgtgcctttgatatttggagggaagggtaggatctggcaattatcgttatctcatttggttcagtgtatttcatttcagcctgttaagatcttttggaaatactgtatgtgctagttacagggacagtgataaataagacagcattcctgtccttgagatgctatttccacagctccatgagatgcctagttaaaaacagagccttttttggaaacagcctactaatctgttaaaataataatggaaaaagataaagttagcatacctgagctgcaagggctgcctcctggtctgatctctgataaggatcagatcctagagcctctgagatccctgtcctccctgtctgcacaagcactcgcagaaggagaaacagtttacggtggttcatcatgactttgaaccaagcttaaagtcaaagtcatactctttaaaccattggaaaccaagtttttgcgagttgcctaaagtgggcaaaaatcccacaatgcacctgagcacaagcaggaaaacatgccattgtctccccaggagccctccttgacttctactttactttatccattgacactagtcttataagtagctttgtctgtccaatttttaattgaatttcgtttttattttcgtgagtggaaatatcactttgaaagaaatcagtcctctcctgaaatccagaattcctggagctcagtttacatgtttgacgcgtgtcacatgattccacaagtcactcaagggcaggaggattaccatcgatacggaaagatttcttagaaagcttaagtgaaagggaaaccagggagaaagtgtgcttcgtgagaataggtatgcggacggctctttgtgacgctctgtgcaaccctcgtgtttgcattgaaccaagctgtgctgcagatggacacccatcccatttccccgactcataccagggaggcccactttgcaaggtacacagaggggaccacagagcagggggccatgcaggggaccaagggacattttagtgtaaccaaagtgtgaagtatgccctttatttcaaaagaataaagaaaatcacaggttttcccgctgatatgccagggacatttccaagagaattccttttttgagagaaatctcctttgatatcccatcagtcagccatactgcataattgttagatagtgaaagaaattcattttttaagtttgtcagaaaaataaattctttgaagtcttaaattgttgcctccggacatgacatatctggctcttccagaatccatgttagtcctagctgaggaagaaggagaaggagaggggcgtttgttgattattgattttgtaagatgccccacacgttggctattagcagaattctcacttctaaaaggaaaatgagtgtgagctatgttcaatgagaaacagttatttttgggacattctttgaggtaaaacacctccttaagatgctgcttccttattgctatgggaccaagattagagcaagaacatagtggttttcagaccctggacatcatccacagccgcagcagaggccctgcccacttgaacaatgagacaggccaccattttgtttcgagaatgagcaaagtgaaccaccatgccagatattgttaagtcagcaactcttctgaagatggacatagtaaaaaataaacaaacaaggcagcacttgagatggtcatggcagagcaatctcaacaagcgatttgttattttgcacagtgatgtacccactcattgaataaaatgcaccagaaccatgcactacagatgctaaggagagttgtccttcaacaaaggagataggccccactggccgtggggcagtttatgttatttggtccttgtcatgggcaggcatggccctttctatgcttcacagatgaggaaggtccctggcacaggtccagtctccagcatggcctagaggtggcaggtgctacttagcatcgccagcctcctgcttggtcatggggtcagccagtttataacacgaacggaggttaatgaactgatcttcccatcgcacaactggtatgaacccacatcttctgattataaatcttttgctctttaactcttagtcattaccactgtctagtgtaggcctgtgtgttcatggcctttgttgccaccaaaagatcaactattagctgaataacatactgagacatgttggtgttgtgttctaaatagcactagtaaactgttagggaaatctgactatatagctactatccagtctagtttttcttgcgaagtgtttgttgagtgtgtaatgaggagtaaggaaggtgaataagaaatggcctgagtctttataaaataagcaaggagagaaaacagttttgatgagaagcgcatggaatttttagaagataggacgtgtattatgtacctataagaatgggtaggattttagaagagatggatggggaaaaggaacagggagtgggaacaaaacgtggaccaaggaagagcaggtttagccatggaagcctcaccgccggctttggtttatcgtgggccaaggggacagaccctgtggggagggctggcagcagggaggtcttccagaagtctatcctgcaggcagtaacagccacccagtctataagctgagtgggcatgggggtgatggggaatgggtggggagttattggggtaacttaccccaaaatgatagctagctggaaccatttattctattgcattttatcaataaatcttataggaagtaccatcctagtgaaaaccctgtcacattgagggcattcatgcttatgttttaaaacatgttattgggtctatgaaaaataaggctgaaacctatgagcaccctccatgcaaagtttcagtcaagactttggaaacaagacagtgtcttactcactttataaattcattcagaaagccgtaggtttgaaaattccaaacttagatgtaagaagctctgagaaacacatgaaatcacccccacatcagtagagatgtctcagcagacatgggaagaggggcagcagggtgtagggaggtggggcagccccggggtgggccttgcaggctgggcttgaatcccatggccaccaccgtccgctgggaggcctggagccggctgcccactctctgaccagcacatgttgatgctgtatccttgaagggaccgtggtctgacatcctgtgatgcagacctgaatccagcacccacagggtctgcacattccctctttgaggtggagcccagctccagaggctggtccctgactctgtttctcaagaagcctgtacagatgttcccctcaccactgtttccagtcacctttggctttcacggtgcagatgctaagtttgattttcagagcccatctgggaatttagtgaactgaacaggtagcatttctgaacccacccataacccatgccctccccactgattttgaaagagagtttgctgcaggtgactttgcagctgggtagagaatcttggggcaggattccgaggcaggcagatgagtgaggataaattgggttctgacggcacgttacaccagtggactctaacgacgacctcacctcgtgcacagataattctgccttgtgcttaaccgttagaaatgtgtcactgaagtgtgaacatattatgctgttagatttcccatcatttctgttctttcattccctctcatttgcattggttactcataaatgtagatctttggtatgatttgtacaactgccgggtgtcaatctgtgaaagaaatagcagagcaagctgggctctggtagcgctttatccctgcgtgctggcttgcccgggttgactcagaggcagtctcacattcagctgcgctggggccaaggacccagggagccaagtgtgtttctgttttctgtatttagcaatttaagacctgcgtttaaatactagctatgcattctagcaaaagaggtttatattttaacacagtaactcttaattgtttaattcagttcgtgtgttacctctcggaataaaatagtggaagccaattaactatagacttcattagtttggatttaagatcaccaaaacatttaccacatctcaattgttcattacatgctctctcttttttaatgcagtttttataatatggggagtgggggtgtggatttaaccattatatttttatgatattgggagtagatttaaccattatgtaaattggattttttaattttaaaaagtcacttatcttgatgtaaaatcatgtcttagtaaccttgataaactaagttttgcatgattacaccttaaggttaaaacatatttcatttcatcatttcccagaaggggcactgaattcacccatttcctgtttttcatctcagaatgttctgtgtttcctccatatctactttcccggcagcaggaccctggaagcagtcacaccaacctcatttaccccacctgagatttgtgcgctttgaacatagttgcaatcaaatcaacaatatctttgctcagaaatggatatgtgaagtaaaatgtgctgccctgttcatgtatgaaatcattcagctagctggccagtgagcccttcattgcaacaaagatttttctagagcccctgcactatctggggctatgtcaggcctcacactcctgctcacacgtttggaggctaccttggccagtattaccttaatccagcatttaggggaaggagcatttcaagactaaattttctaaactgctcaagcctacctcattttatttcttgtgtattttaacacttttggatgaggactcttcctagaacctactaacaattcccccccgcccccatgccaagattctttaaagactttcttgaaaacgcttcagtcttttcttcttagctcaaaagtactatcctaaatactagctctggcattacagggagttaatttgtgggcacacacagtaaattataaaccccttaagcagagaagattgtattatcagtttattagttttcttattcattccttcagcacacatttctgttgcctccaatgcagcagagaaattgacttctacagtttccacaacagtccagatttcaactgtgtagtgctctttcagccagaaagtacacttgtgtttccctggccataggtcctgaacctcacttctgaaaagtcattgtgcatagagagctaatagctgtaccctaaatgatcctggctttgaattctcttatctgcttggatagtattatctgtctcttcctctgcattctaatttgctacttctaatctgctgggaattacaataagaaagaaccatttaatcatttttacaactgtgcctaaagagagtgtgtgtaagtgccgagagagtgtatgagggacttgcccatgagtaaatgcatgaattttaggtcaagggttttttgcttctcttttggttgattacctcagagatcagttttactttctttctcattcttgacctatcatcactagctgatatggatgatgtgtacaacttctgagtaagaataatgtcaatgggacgggatgggattggctggtgattctgttgatcttaaagtttatatattttaagtttagtgtttcagaatgagaccaaagcggtgacattttcaacctcttcggtctctcttcagttttttcattttaagtttttgttgtgcttctatcacttaaaggaagcctccaagttgaaatcaaatactaatgacatttttatctaatgtataaatgtgtttttattatttattaggaaatttattttacttggccctcagccatgacatatcatggcataatcactatcctaaatttgtatatcttatccttgcataaggtaagactttctatgaattacacatatttgtattttcctctcttacatattttaagaatttttttatactttgttttctgcaaatgaaatattgctcataagccaggtgatggctgtccacgcctcttttcccctgcttacccttgcttagatcttatggtagaatcttttcatagaagacacagaaagacatgaaagaaagagctggagaagcctgaggggctgcccagtgagtggtctgctaggatgctgtgccacagcccaggcacaggaggcagggagagcaatggggcccttcccttccaccaacattcagcagaatttgcagctccatgtttccaaagcttccagggcacttgcatttagagagagagagcaagcaagctgcctttcctcttcctcagttctgccagccacactcttgccatgatgagcagtttcagccaaaagctccttcccctgccctaacacctcctgcaaggcctgaggtctggaagccacctgcgcctgctggcccctcctttcttgtttctgcaatggatgttgtggccctgtgagggagaagagaaaaagaagttgccctcctccctctatcctcacctcctgccatgctgtcacccttataagagagaagggctaaccatccaggctaatcctccagtgatgcaggagaggacatcctggccggaagagtcagagcttccaggtgagctcaggtgggtcagcccccgaggctgtgaagagcccaggggccagtagatgccacttttgctccaggaagaatcttcaactgtgtcctttttattcaaggggctctcttttcagcgaatctctagatgtactagtcacaaacactcgcatttattaaaatgtatccatgatcccacaatccttttacatatttctctccaggagtatcacatttctgagggccctgtgcctgttttctgcaggaagttgctgttgtcccgggtccccctgcccccagcacctctgttacaagaagcagacccttcatgccacactgggacccagggaggccccaagccaggatgctgggatcttagtaaaggttggaatgatgtcagaacatagaggaggcagaattccccccatagcatcatcctggagggcgctgatttgtgtgctctgccaggttcatctgtgactcaggatttaaaagccccaggtgtggtgtcccttctgtgcctgcaaggtgcgtctttagcagttctccctggtgtggaaggatcagtggttcttgcagcctaggcaccctccacagcaagcccaacacaggtgctgtgagcagctggttcatgaacggtgatcctggggagaagaggaggatgaaaatggaaaaccagtgcaaaggtgtgtcatccagttggttactgctgtgggtgcccgggcttccatcccaccagagcccccactagccagggcagcctggcggaggtgagcatcattctgctttctggctgcacctgtgtggacagagcccgttccaaagcctcccaggcacagggcagacatcaagcaaggggcagagctggggcgagggctctgggtgcccccgttattgaacacaggcctgaagggagcctaagaggtggctggcaggagatactacaaggcagaaagacagggcagcagtctttgtactgtacgtttttgttttttaagagagaacaaaaagtcaaggttgagagatgagaaatactcttgccaaaaagagaacaccaaaatcctggagtcacgggtcttccattaccctctctagcatttctgcttgtcttttccctagtattggtgaagaatttttaaagtaaccaacccataccgttggttacaggccctgtggtcaccaggctgtctccccatgatggggatggagagtggttagtgcagaaacttagacctcccctccagcttgttgaatgccctgaagtttatctagaagggaagatactcaagcgtaggatttcagtgatgcatttggacagcatcagtactatgcttcagcgtcaaaaacgtcactttgggtaggaacaatacataatgcgtggcaatgccttttgtgacacctgttcaggagattcccataggaagcttctgaggcagagtcctcaggtgaggagggggacaggcctgggtcttgggggaaggtggagatgaccagcctcatgcctctctccccagtggcctcagtctccatcaggcaaatcttgagaagcctcctccattctgcaggcaaatgactgagatgtgtgagctctgcttcccaacaggcggaaattcacatggggaagggcacctgtgaatggccttctagaactatcaggaagttctggatttagtaccctgtgagagcagatggtcctgggtgcactcggatgatcctgcctagccagctggattgcagagtggtcatcattatcagtgagctattgaggggttgagagcagtcagtgtcatgtataaggatgggttggggtaccggccaacgcccgccttgaggcctggccgccggatgggaggcgagagcacaagagagtcctggctaggtgtgccccgctctgcagggccacaccatccactggaaaccctgtgcccactgggctgcgtgctaagaggctgtggccaatgctcttgtccaaattttactgaatctgcagtctctcttaattcactccaaagtttaatgtgttagcttgcgaataaataaataaataaataaataaataaataaatggaaagaaacagtcctcagaaagtcccagtcaaattttaattccaacagatattcagcagtttcctctaagaacaatgagagttctggggctagccagtgtttctcttggaaaataaggaagagggaaagcggtgcattcatttaaaaacctgccctagggaggcagcgttccctgtgaactccgaggccacagtgagcagagcaggctggaggcctcccggctgtcctgcccctcctgccacatgcctgtgaaaatgctagataaggactttttctcagcaacttccacgctccttcagtggggatgtctttgactcagagctctgccactggttatctccacgaacagaaaatgccacagatgggttaattcactgtgttgttctcattttccctcagtttcaggcttttctctccttgcctgttttcctcgcttaaaaaatgatgtgggggtccctaaacgcatctaccccgatagatttatgttttcttttccattagtccactttgcgtctcagccctaaaattaagtttttgattataatgtaaggaagttttaccatattttaactctggctttttaaatacaaaagaaaaataacagaatggccttctagaactatcaggaacttctggatctagtaccctgtggggacagatggtctggagccagcgcacttggatgatctctctcagccatctggaccatagagtgaacttgttactgtccctccaaggctgacaactatgagctcatactcttctagcagtttaatttagacccaagaaaggctgtgtgtgtgtgaatttgtgagagtgtgtgagtgtgtctttgtgcctgtgtttctgtgtgtgtctgtgtgtacatgtgtgtctgtggggtgtgtctgtgtgtgcacggctgtgtgtctgtgcgtgtgcatggctttgtctgtgtgtgtgcgcacgcactctgctaagctactcaatgcaattccttactcttacttccctttctgtcacttctccataattctttgtatttcggttgggctggtatctcgcggcggcttcctcttttcctgcattcctatatttcattatttgctcttgttcctcttctagggcttttacaatatagccaggaggatgtggaaacccagttacaagatgacacacaagcacagtgtcacaatcgctgtgcttgggccctacctcctggagaccagggtgggacggtgtctctggatatggagggaagggcggaagatcacggggtggcagaaggcccgactgtccggtcttctggaagctggggtctcgtggtcgctgggctggtggtgctctaaaacctagagtaaccgagagcaggatgactgcacccctgactgccgactcaccgggcttcggagggccatgggtgtgtgattccacttgtgatcctcctacttccccagcccaccctggtggtctcccacttctctctctctgcctgtggagtgttataaccgactccttcttctaggcatctccatcccaaccccagaccagttcccttaaaccacctcccaccacaggacccccacaagcagggacctctcacagttcctgttgcctggggacttcatctcagcttccaaggcctctggaaccaggccctgcatcctgggcatgactgtccccatgattctctgagccagccaccaccccaccacccccccaccgccaagtccactgctgaccctgctgtagccccccacctccacttgttgagattccctcttcctgcttctcattttgaatgtcacccttctgtgacttctcacaacccccctgccacccctccctccagttggcatggcttagtcttcctaagaacatgtgctgcttcccagtacccagggctgctgtgcaccgtggcacgtgagtttcggtgcatcttcctcctgctgtttataaattgcaacatccctggacagatcttggattttccctctataatttctctggagactccctcggggtttgcacacacatctgtggaatgcccaagttggagatggtcctccctgatgtgtagtgtgggctattcagagggcctcatttcataaactcattaacagctaggacgcgaagacttggagaagttagctcaggacacacggagtgggagggaggcaacgagttaagaaatgggctttggagtgcctcgaatttggcctccacttttcttttctactgtcttacctggtcttaggcaggtcgggtgaccatctggagtcggagtttctctcatttgtaaagcgggggtgatatttttctcacagggctgcttcaagggttaacaaagttaatgtctaggaaggacatagcatagcaccttcacctggttgcaagcccctggagtcacatggcaacagctgtggaaccaggtaaatgacttgacttacatgttacacgctcttagttgttcgatttgtaaatgggaagagtgtgtgacccaaattagtcgtttccagatatttcctggtaaatggttgttgaatcataaaactagaaagatgggaaagaaagggaggaaccccccttacttcgaagaagtgttgttgatgtgaaggacaggactttttgagacactctcatcctaagaaacagctgatatctactaggaaaaaacatgacatttgaagtttcttcctaagagatgtgaggttttgacagaagtgccaggaagccagaggtgtgtgagtagtgggccaatctcgcgtgaaggctgtgggacggggcaggagaggggcagccagcatttcctgagcgccagctaatggcggggacctggcacctttgctctgtgactctccagctgtatggtgacatgaggctggcgttttctttggtttcatacgtgaggaagctgaggcttggtgatgtcctgtgcatggctcggactccagagctactcagaggcagaggccgtctgtaaacaccttcaggtggttggaggcagggtgctgcagccaatgctgatcactcacaagccaaggctttattgtaagggctaaataaaataacatatgcctagcatttatacagcacggggctttttaggaaatagtgagacgaccatggagaggtggaggaagagagaagggaaagaagaaaaagaaaaaaaaagccttaaagagtttcttaagagaactatatattacaaagtccttggagttttttttccctggttagacataagtttacatgaaacgttaaacggcttaaagtagccacatctccttccttatgtcttctcacagccacttgtgagtttcttatcaagatacagaattatggccaagtgcggtagctcacacgtgtaatcccagcactttgagagactgaggtggaaggatcatgtgtgctcaggagtttcagaccaacctaggcaatatggtaaaaccctgtctctacaaaaaaatacaaaaattagctgggcatggtggcatattccaatagttccaggcactcgggaggctgagacaggaggattgcttgagcccaagaggtcaagactgcagtgagctgtgatcgagccactgcactacagcctgggccacagagcaagatcctgtctcaaaaaataaaataaaataaaataaattctgcaacaagtcacaattccttgctcagaaatctctacaggtgtgcttctttgttaagaaatggaggaaacatgaatttcatcctgacttctgagtcttgtagagaccagatctgactcctgcagccttctgcccacagagctggcaggcgggggaatgtgcgcagagtgaggaggagctgatctgacattccgaacagtggagactgctgcctatttggccgcaccactgtccttctgcatggaaaagtcacagtaaataatggactctctacctgtgagtcgattttactgagctgcctttttaaatatatgtgtagaaagggcgatctctgtgtgtcatttgcacatgtacatacacatgtacacacgtgcacacgtggtcactcatgtacacatgtgtgtgcaccagtgcaccagaccagatgcacccacccaattccacgctcctcagccccttgctttcctctgcagacagccagttcagctacagttatgccagattgcctgcactctgatctttaccatcaccagactctcagcttgagctcatgcctgagaaatccactttctgggaggaaggcagaagaaactgccaaggcaggactgaagacttgacctccacctgtagtggggtcaggtcaccagaggctggctgactccccaggatttctcagcagagtattacacaaataccccttccttaaagattagcaaccacttaaagacacaaagtgtgcaggactgtacccatggccgagagcggcgggattaagagggcaactgagttgttcttcccatccctcctcctctgtgctgagtcaccttctgcttggaattctgacagggacccgttgggcttctggaagggagggacagcagaggatccaccctctgtgtgtcctgggggagattacttatctctggcctccctgaagcagggcctgggcttctggaatctttgaggctgacaccctgccagccctggggatgagagggaatggccgtgtctgtctgcagagcctgaggaggagctgagcacagcctccagagttctcttcagttgatcgcttaggtggacaaaggccacagaaatggatttaaactcctcagccctttctttgcatgtttgttctcatttgaagtcagaagtgatatgtcctacacctcaagaacgtgtgaaatgcacatacaataaccccatttcaggaagccaagtccagcttaacagtcaaaacatttccttcagtctttagtccttcactttgccgaactcccttttacaccggcagcaacagtttaacctgttgcttctgtaagagtgtgctactgggaaaaccacatctaaaacacgtgtgcagttacatcagctagagcacatgctaaacagttgatcaaaggctcttgccttgtggcccacgctgcagacactctgacgactgccgagctccgcagccccatgtcgtcccttccgcactgcctgctgtgcctctcctctccatgtggcagggaacacagccagtcatcaccatgtggctctgcccggcgctgccccagcatgtcctgacagggcctagatatggaaaggtggctctccatgcacacaccccaagcccctcctgcccgccgtgtgacccacactcttatgggcagcccagttattttgtagcatttcccttccttatcattttggcccagtgatccagcacaaatctcccttattagaataaaatttggaatgacaaaattaaatttcatttttcacttatattgaggacctcacactcttcacccctgccacgatccctgacaagagccttcctatctaatcattgttcctccagccctcttagttttcttcagcctttcttgattgcctgaatgtccctttcccttctccttttaaagcatgaaccaagctttcttaccccgttctcattatcatttttgcattttcttctttgcatatgattctccttaaattataaaacttgggggtaatttctagaggtgccatcatagtgcttctgtctactcagtgtctttagaatcagcaaatatcatttttacaaaaaagtagtatttcttccaaaaaagagtaagcaagaaggttacaacactgggaaaatatccctaagcctgttcttcaacctgttgaatgttttcccctaaattgttatatggagatcctggacccggaagttggctgacatgaaacaggcctagcagggcagctgaggaaatgctccaacctcaggatccaggaagattgcacatggcaccaaaacaatcatttaaaagctaatcctggccaggcacagtggctcacgcctgtaatcccagcactttgggaggccgaggtgggctgatcacaaggtcaagagatcgagaccatcctgaccaacatggtgaaaccccatctctaccaaaagtacaaatattagctgggcatggtggtgcgcgcctgtagtcccagctacccgggaggctgaggcaggagaatcacttgaacccgggaggaggaggttgcagtgagccaagatcatgccactgcactccagcctggtgacagagcaagactccatctcaaaaaagaaaaaaaaagctaatcccataaaagaaccatcattttgaacctgctgtttcctttcttgtactctttagaagtacccatttctccctttctaagccataggtgtattaattggagctttttctatcttaattagttcactgtgaacaattaaaattgtgttaataaaacaaaaacaaaaataggactggtgcctagttgtactacatgaagagagaaagggccagacattggtttttcctaatcttctgtcaagttctccaaatcatctgctctggaaggtagctccaacagctgggatttgaagtaaagcatagtgactttggccatcactgacatgtcccatttgaagcaaccaaaactgtccctcgacctgacactcatccctgaaacaccatgagggtaagtgaggcgcttcggaaggtccactcaaccccattgccagatagagtaagtgtctgccaggggcatttggagctgaagggaagactgtacagactcacggtttgcaggcactgaaggcgttttcctgccttctttttcaccttcagtggacttgcaaagcactagccctattgtttttcccatctgggaaacatggtgaatgttggttggttgtagctaatcctatgggtcttgaggtctttgttgacaagaaggtagatgttatctttatctgcgtgtggctttctactaaaacatgagctacagggctctcttttttgttttaaagcatttttccataaggttcacccttactattgcttatctgaataatattacctgctgagaagtttattcattgctcaccagttgtagggagattttgacacaggactggaggatttttttctcatcgtaacagtgcagacccatggaaagcttggaagcagttgtgaccggataagagcaggttgaggatgataatcttagggcaatgagcaggttgattgagaggggtgcctgaaagcaaggtccccatgatgcaagcaaacaaactcacatgccagacggtggacaggaaacacaggcaggatgtggtgcaggctgggctgccttggctgggcaaaggcaggggcttcatggccatgctgggatgtgacgtggttggaaaacaataagaatggagacctctccaaagacctcagacataatttggccatgagaggcaagggtagaggcgagccccctgtgggttggaatgtatggaatccagatgaagcaccatcaacatgcatgggctacataggagagctgggtctgggggaaaagatgggatttgggtttgggtgtgcatgttccaggcatggagggaagaatgggcagaatgacgaggatcaagccctggtggagcccccagaccagggccctgggcatatttccacttgccttcttctgcaaacttttgtgtgcctgttgggtggaaggtactatgctggggccatccaggatccggagcagtgtaagacatcatggcctgcatggtgtgacccttgacatctgaaaagaacgagctggtggagtgggaaggagacaagtggcagcagagttgacatcatcaggactgactgcaggacaggcggtgaaacgagccactgaggagttacccagtgccctggggagcttagatgtgagacttacaccacccagcactccccagctctgtgtccctgggtcccactgaacttgattgtattttagttgcctcttttctaagaccggagcaacaataccttccttgagtgtgcatgtgcagtccatagatggcagccagtgttctcttcccatgagaagactctgagcctcctcactagagggtggtctaaaaacaaatggatccatccacagaaggtctgaaaggttttcatagaatcttgacttggtagcacttcaggatctaactcgtgctttccagaagtgtggcctgtggtgttctgtctggagtatttggtcctcccatccacctccaccttgctgtcctgtcctgtgactctcactagctcagtcaccgggtcaggaagccctgccatgaaatgctagatttggaggcattgccaaggtaccacaaaccatactcaaacaaggtcataatagactagtgatctcactgtggctaacaggttcatgtgatttaaagaatgacagcattttttctaaattttataaattttataaatttctcctaaatacaaatctgaatgtgtttacctttagacagattttcccagaaaattgtcagtgttctcgaatttgaaagtagatcagatctcctccctctagttttgaaaatctcaagtaggtttagcctctccccagagtgacaacggcacaagaagtttataattttatgcacagctaaatgcaaactgaaaagtgtggtttgtgggtatttcatttttcccataatgttcatcaatttggcaattcaaataggattcagaatccaaggtgttcgcagcattttatatgaaaatgtgcctgagagtttggggaatagaaattcttaccaaataagttaaatttgctcttgataagaatatattttgtaaaatgtagaaaactgagaaaaaaagtttgacacctgttttatctgcatccttttattaaacagtggcctgaatttactaaaggacagagaacagggttgagaggtaatcactgaataatagcttcgctaatcccacagtgactgcttcttatgacaagcaagagggtcaacttagaggcaggactgtcttcaggggcaaaagcaagtcagcacctacaaacctctccaagagagtgagaaaaagcaggttgtatggctcattttgcagcttgccagaagcaatgtgggaatattgtcagactgcacagacctggggccagctggctcctagagtgcgaggagctctcagtgactgctgtgcatactcacctgctctgtgactttgttcagtccctaccctggatgtgtggtgaagggtgccaggcttagctgctggaccagtgaggtccctgagggggcagggctaagaggtgtagcagtcgcacttgaccagtcattgattcactggttgggaatttgttcattgaggggacaccattactccccatgcctcacgtgcaggtgagtgtgtgtatatttgcattaacacacaaacacacttatgtgtgtccctgtatgtgcagctgcagtaccttacacatcttccagtgcctctcaagcatcataaaaacagcttccacaaaacctctactacccacctggcatccacagagctcccagtgattggctcccaaaagatactgaactttggcatggaacagagtgagtttccacatttgctgttgtacataattacctgtccagctagaatagtacacacttcttttactgagcccagaagtttatgtgcctgaacacggatggtacaggaacaagggtgagcgcttatcaattcagcattactctggagacatgaaacaaactaataggtacagctatattttgttttttaaacaattttgtgaaccacatatttgggacaaaaatcaacaagtaaatttgaattacactaggcaaaggactgattcttttgaaacgtgattgacattcaacgtctaactttccaaaagaagacatacatgtggcccacaatcatatgaaataaagttcatcatcattgatcattagagaaatgcaaatcaaaaccacaatgagataccatctcacaccagtcagaatggttattaataaaaagtcaaagaatgacagttgctggtgaggttgtagagaaaaaggaacgcttatacactgttgaggggattgtaaattagttcaaccatgtggaagacagtgtggcaattcctcaaagacctatataccatttgacccagcaatcccattactgcatatatacccaaaggaatatgagtcattctaccgtaagacacatgcacgcatttgtttgttgcagcactattcacgatagcaaaaacatggaatcaacctacatgcccatcaatgatagactggataaagaaaatgtggtacatataccccatggaatactatgcagccataagaaaaagaatgagatcatgccctttgcaggaacatggatggagctggaggccattatccttagcaaactaacccaggaacagaaaaccaagtgcagatgttctcacttttaagtgtgagctgaatgaggagaatacatgggcacatagaggggcctatcagagggtggagggtgggagaaggaagagtatcagaaaaaataacaggtagtagatttaataccggatgacaaaacaatctgtataacaaacccccatgacatgagtttacctatataacaaacccgcacatgtactcctgaacttaaaagttaaattaaaaaaaaattaatgtctaataatatattacagtattcttcatattcaatggcaatgtgtgaagtgggaagtgtcttgacagaattcggtgtttcaaggcttacactttgatgcccaagactgcacaaggctacattttctactggtgagacaaattccagacgcattgcattcagatctaatctcttagctccttaatcttcagggtactggtaaacatgaagacctccccagtgctgtagtcatcatgatatgtacagcaggtggctgagctctggatgtagactgcagggatatattaggaagttaattctcaaggcaagtcatcttcaagcaccatatcagcatgatcagcaatataagtagtatctcagtgctttgttgtttagtcagagttttgtactctatcacccattgtaatgttcctatttgcaaaaggtaatacataccctttaaaacatctttgctttttctcccattatcgagatgctagcagcttcataaagcagaataactaagggcaaacagattatataaagggttggagctcaatgaagacaacaagaacagcaaaggttattgtaaaactggctgcttgcaggccaacaagcacatccatatggaggcaatcagtttatgctacctctgtctgtttgatgggattcataatattgactttatccattagatttggactaccagggaataaaataagcagatggagagtaaggatttgctaggaaataattcagccagtcactttgaaagctgttcaagaaacagctttcaaagtgtctctcaaactatgtttgcccattatcccaataatttatttcccaataatttcatgggaaaagaaggaagttctgtggtcagataaatctggaaaacactggtttaagcaaagttcagtaggtctgcttccctgcaggtcacctcagagtctttactctgctaacctaggaactcatccaacaagtttaatttaacagctacactgtgtacgtcactttaacagtcactgagctgtgactcttgggggaaagattgtgcgtgtgtgtgtgtgtgtgtacacatgtgtgcacatgtgcagaatctaccaaatcttaagagaaaggaacatgctgggaaactgtcctgtgaaagagaatagaaacctgaagatttgaggcagtgatagcatttatgaaagcagcagataaggactaatcaccaaaaggggtagctcttttgttggttggggaaaacaggaatttttcccccacccaatgtgctgcattttctaattttctatgaacacttcctaagaaaaagctgaatgaagaacatttgcgatgcaatcagctcattaagaaacacgcacttttgtggagatacgtgctgtcccaggagatgctctgcgaggagccgagtgtttggactggagctgctgaatggtttctcacagttctagaatgtttggggctgcaccctctaagatgttgaacccatcagtaattgctccaaaccactttatgggatataatgctgtgagttgacacctgaggggattgtggtcctgttcatgagtaattacttttctgttgcctatagaagggccagcaatagcagatgagtagctgaacagtggttttgagtaataaaacgttcttttttaaaaaaaagtaatgctttctgttaaactctgactatactctctcctggtatcacaacccagctttctttttgccttctttattgcagttacatatggggctgatgactttagggatttccatgcaataattcccaaatctttctctcgtaagtatatgccttgcttctggaaaacaaaagcatgccttcatctcctatcatgtaaatatcgtacgtgcatgttccttcatcaacccccgagatacattaaatattcactgttctattcgttagacacctaccatatcatttttgggtatttataccataaagtgcaaaacgaaggtctaggcagttgtgcggtgtcctgggagcatggcagtggagtaacagtaagggttggagtcacagtagcactgatgggattgttacttcgcagatgctgctggacagctttgagattactcctgtaatcctttttattgagtgtgaggataaaatggccatattttctgtcaccaacaaagaactgaacatggtttcaagataatttttacaattttgtgggacaggagaaggagatatgctcttcatctgtggttctggagtattaatttcagaactgagaagagaaaattgcaggcctccaggccgttattctgtggtgcatcgctgctggagccaacgtactgttcctgcttggtgcccgcaggaccttctctgatctgagctgttaaaccaaaaacagatgtgaatttcatttttcttttttagatattgagctgataaatgataaatatatttgggtaacatcctcaaatgagtactttaaagaacagagagtgcttttgaaaatgtatagagagctgaatatttaaagccatacaagttgataatccccttaggacctgtattagtaaatcctctaatactataaagcaacatcagaaaacaggtcgcatttgttaacactgtgacaattataattataggctctaatttgaacagtgccagtaacagttaaagacaggtgtctgacatcctggttatcaaaaactatctggtgtctaaggaatttaacagtatcaaagtacatctttttgctcacaaactagattggctacttttccagcctatagaagttacagaaatctttcttttactgatattcctttgattccactttaaagcaatagcttgatacctactttttgagatgtatatgtatgtacttgtaaatgcttatatatgtgtatttgtttacacgtgagcttatatgaacatataaaacattatggtattggacagagaaatgatattgatttgaagaatcatattgttgaagtattttgagaagtcaaatgcacttgagagtaagctaatgcatcctaaaacatgtttctgtgaagtctgaggaaggtgtcagccagagcatatgttgaagcagacccttcagtgaagccttcagtctgtaagaatcgctgcatgtgaagatgtgttaatgttatgatagttacagtttttataaaagagatgatatacacttggatatgctttctgtctatatttatgcaaatgtgtccataaggtattggtgtctctctttctctcccactctccccagtgttggaattgtgactatcttctcacacaagcggctacttggtcttgatgccttcccccgcaaaacagcaaccaaactgttctgggccaatatcaccaccttgtggtcatgatgaagaattgccccctttgccctcaacacctcttttcttcttgaaaattaaaaacaacccctttcaccccctctactgtccttattccagtttgtgtccgtagttgctggaggaaagaaaatgcctattgctcttttttttattctctttatctgtttactcttctgcctttcttttctctccctgacttcattccatttaaaggccttaaattgcaaataacgaaaaaagtattttctaataatataccctagtgggacgaaaacaaccttctacattttaaatgatattaagttataataaatggttccatgaactttaaaacgcttaaaagttttataaatctcttttagaggcaacacaaaaatatctatatatatatcttctcttcacctcaaattttcatttaattaccgtgattcaagatatatcttaaatatttccatttcatttgatttttacttgctagtcaccagtgttgcaagaatatttctgtaccatttttcaagtaaaatattgagcatttcaagcttacatggcctaaactcacactagagtgactacttcggactatcttttcaatggaaaaatgcgttctgagtaccaaacatttactttttcagtaaatatgaagacacattctaaaaaggaaagggaaatgaagaggaagcttaattcattggtcaggtggggactaacagtgtacattatacctatgcattaaaaaatgtttaattatccatgagcacatacagtgcctggcacagattcaggcctcttggctcctcttggcatctgagttccttggattctccagattcttcaatccaaacttgtctatctaccaaagtccaccttccatacaggggtgtggggtctctcttagtactgttgggtgccaataacatagaagccctccaagataggtcaatgagattttaatttttcccaactttaaatagcatctgggaaggaaagcagcttttgactaggagtttgaaaacatccactcttcatatttattgtcacatttattgagtaataaaattgggctcttgtccattatctctcagtcaatgaaagagcccacggtgggcatgagccagagtgttccggagaaagagaggaccagtctccatttcatgttctatatttaatcagttcaattcagcaccttactgagtttgacatttttctaagtaatggggtggggggttacaaaatatttataactatctttcctatcttcagggagatgtaccatttcactgaagcaataccacagctaagcagttccagctccgagtaacagaggaaggagtgagcgatttaggaactcacagaagggcacggccaggcaactcgggccctggcaaatgttccatagagacagcatccatggggttgaaccaagcacaatgcctgcaaatcgggcaggcgggttcatggaattgattctagggcaggtatgatcagcaccatttaaggagtggcagttgttaagaaactgaagggatttctcatatggcattttcaacagaattcagcaggagccttgcatatcccttgggatgggttccggggtattatggagctgccaggaggtgctcagcttcagtaagtgccagtcattccttcccaacctcctccttcattagtggaagtatcagctggtgtcattgccttcttcaggaggatggatttcaaaatgaagggccaacaaaactcacattctcgcagagcctccgtcacaactgcatgtgttcactgccatcaacaggccaacacccacttgttcttttcttctaggtaaaggaaaaggtctgtgggacaaaggccctgaactcctcactccttgaatgggaggcatattggtggaggctgtcacacaactacaggtggcaggaacctgccatcagggttcccatcagcctccagggcttgcttctctgccctgtgtgtggacaccttccatcactggaaacccctctctcagaagaaccacttcttgccctcatctcccacttgcctctcctcggcaccttctccaccctcaggggccaagctgaggaagtgtgcatcctgtgccaccaaccttgctcgcaggaattagtttcagtccccagtgactgactcttccaaacctgcaccagcctatgctgcccttttgaaaggaggcgtgcgtacagcctggctggtacagaagattccagatttcagtgactacaggacgctgtcaccccttctgtttttcttttttctgtccacatctctgattgagcataccttcagggaaaaccccagaaacctttttcacacttacatgggaagccaagacactctcatcctgggcttgtgtttttaaaatctatattttaatttcacaggtccttacatttatgactaatagatttcagattttgagatacaatctgtagttgcaacttatgaagatagtttcaagccctgatttctgtcatctatgaaaacagtaagcaagttgcttgtattctcctcctagctggataagagacccccgttttcatggacagcctccttgggtggacagaagtctcctgatcttgtctatgtaaccagcacccactttgcatttttccgcaaagaaaagaggagcttaaccccatttacgggaaatccacgcactgcttgaatcttacgcgccccgtggtgacggtgttgcccactctgttcccttcccaccaaatcctgccagcacctggcagtcatccagtccttgtcttagacttcaccaacctttcccgtcataacatggcttagaagttgtccgaatagtgctagaagctttcagccctggaatgctcagcatctgccatctaacttccatttaaaacgttttgagcatttgctgatacaggttcctctgtcctttccccattattttcagttccttggccttgcccatagtgatctgtggatattgtctgcatactcttttttagaaacctagagcatcgcagtgtccttaattttcctcttcaaactctatatattcctgagctgatcatccctactgctgttcctacagaactgtgtgactaatcttctatatatgtccctacatctagccatctaggtggagatctaggtacctatctccctaaaattgtacttggtatccacattgacctcaggacagtatgtgataggctcttgtgccttttcgactaggagtttgaaaacatccactcttcatatttattgtcacatttgttgaataataaaattgggctcttgtccattatgtctcagtcagtgaaagagcccagggtgggcctgagccagagtgtttgtgtccctagttctcttttggttctgttacagctctcagtgacagtgtcattgaggctaacaaggacagagtactgctttcagccaccatttgtccaatgagtggctgatctccaggcctctggctttgagaacatctgtgatatctaaggcagcatccattgtgggctttcccccatgcttctgtttccttcctgtcacatagctttgcctcctcctgcaagcagcctgctgtagcagaaccggtgttcctgaagccagaaacccaaaggtcgtgtccaatgcttccctgctgttctgctccccacctgcaagcgcccacacactgatcaacagcacacgccatagagcatgccaaagaatcccagaaatactcattcttaatgatccagaagaacaagtgatagcttctggcaagttctaataggccatatgtgcccatgggaaagcaagagtcaattctctctagctggttgcctctgaatttatgaagtcaagccgcgtaggggaacatgccaagaggattatcccaatgcctgttctgcgtggtgtcctttttctagcctcggaagcactaggcctacaccttcgctatagccctcgtcttagcgcctgtatttgaaaaacttgccacaccaatcttaatttgctccattgtgttctaatctcattttaaaaaccacaaggtaaatgattaaaaataatcttagattaaggagtacacagatctttgcagcctcattgtgtttccagagcgggctagtgtagacactggtgaacaaggagggcctagagaattagctttctcccagaaagatgcacagcctctacctgagagtaccaattccagagaactcaatggtcattaagcaccattgcctcgacagccagccagcctcacttgcctgcctatctcctttattttccaagtatcttgtccctggcagtggggagaggttagcaggaggctgctcagatgctctcggtctctgatcttcaggatctgaaggggagagcatttgaagaatccccattgctggatttctcaggacaatcctgcataatgccaggatctgatggaggagacaggcagctctgttaatcctctggtgcatcctcacttctgtggtctccaagtccaccatgtcccagttaattcatttcattattcatctgtaagtcatttccctaaagagctaataagaaaacactggcagtacaatcagccctccatatccatgggttcagcctctggaaattcaacccactgtggcttgaaaatacagtattcaagagacatggaacctgtggatatagaaggcagatttttcgtatccataggttctatagggccattttagggacttgcacatctacagaccttcaggagtctcagaactgatgcactgcagataccaagagatgactgtaagtggttaggaatttggatgctggggccaggctgcctgcagttaccttccagctctgccacttgccagctatgtgaccttagcaagttgttcaacctctctgtgccttggcttcttcaactgtaaaataggataatgatagcacttcccttatggagtccttgtgaaggttaaatggcagagtacaattaatgtgctgtgtgcccagcgtgtggtattggggttaggtgaaagactgtactgggatcactgggggtgagcctccatgtgccatgccccaaccactatcctcagctaacttcttgtctgtcagtgttaggctggtgctataataatttccattttcatggatgaggaaatcaaggcacagagaagttacatgacttgcctaagatctcagtgcttttaaatagtgcagctaagattccagaccaggtatttttatttcagtgtctggactgtagatctctaaatcgagaggaactccctgaataaaataagcttggagtgctgttaactaagttggttatttaagacagttgttctcagtcctcagtgtacattagaatcttctggagggtatgttaaaacagactgctgggcccaccgagagttcctgagcctgcaactctgggttgagcctgagagtctgtgcttctagtaagttctcaggtgatgcttatgctgcttctcccaaattttgagaaccattgattgaaaatgttgatcaaaaattatgtggtctaggctgagcacagtggctcacaactgtaatctcaacactttgggaggccaagtcaggtggatcactgagctcaggagttcgagagcagcctggtcaacatgacaagacccatctctacaaaaaacaccaaaaaaaaatggctggatgtggtggtgcacacctgtagtcacaactactggggaggctgaggtgggaggatcacttgagcccgggagacagagcaagaccctgtctcaaaaaagaaacatttagtcttgattgtcatctatctcattgatcattttacttggcaaaatttacctacttactcttattagtctgtaaaaatggttattaaaatggtggctttcagtgtaactacaaattctcttagtcattatagtgttggattcaccaatgtatcatcagtcagtgtttctgaagtgtgtaaatgaaataactcctactttctttaggaagaaaaatattcaaatgacataattaccttgtgatgtgtgacttaaaggtaacagtaatgcagagtcaatagtggtcattgtattcaagacactaaaagttcacctcccaccccccccccccacccaccacccaacaaacacacaagcttctttccctttggaaaaaaaagctcttccagatacctacattcataaactatcccaattaacccttcagcaagtggaagaagtgtaagaaaggatacctcttctttagaacacagggtttgtttttatgttatttaagataaacaggaattcaaatggtcatgtaccaaagcaacacaaagaacttccggaaatctgaaagggaactgtggtcagaactctgagcatttttatgtttactgagttttgtcccaaagtttattaatgtttacatgccacaaggaaaggtagcatcacaataagagacgtttttcaggcttgataaccacttattaggtattttgccaaacaagttcacacatcctagagagctggattgtgtgacccagaacccaccctctagggcaaggtgcccatctgatgggtagggtgtaggagtaggcctcagaccactcctgacgtgaacctgcttaaagtgagggcccaattctaaagtgggaactatgtaaatacctttctagtgcattttcagataatcgccactgggcctatggatggagagggctggcagatctccctgtaaccccaggtgcatcccgaggcctgccaccgaagcccactcaaggctgaatgcacggcgagctcaggctgctctccccttggtatttgctaagaacttctgtttagtagctctccacacctatttgattgtctttttgctgctgtgttgttttgttgagttttttttttgcaatgacactgagtggcctcctgtattgtttctttcagccagtaatgttaaagtagagactcagagtgatgaagagaatgggcgtgcctgtgaaatgaatggggaagaatgtgcggaggatttacgaatgcttgatgcctcgggagagaaaatgaatggctcccacagggaccaaggcagctcggctttgtcgggagttggaggcattcgacttcctaacggaaaactaaagtgtgatatctgtgggatcatttgcatcgggcccaatgtgctcatggttcacaaaagaagccacactggtaaggcctggctcagtttttcctttagtggcctggagaaggtgcatggggtttgaaggaggaaagcatcctgtcttccttgtgttctgagcatgtttctaattgactggtagctcagttgttgcaagcgattggttccaagtggtaccgagtcatagagtccttgttctggtacagccttgtaaaggacttctcaacacgtaccaattccaccctataaataaaacaagggaaaagtgaacagcatcacatgagaggcttggcgagggctgctattatagtaacacatactaagtagtctcagctgagccctcagggtacgtgtgctgagtggtcaccctccacaaaacaaaaaatcctgatacaccaaaacttactcctcaaagtttccactgagaaaccatgagtaaaatgtgtgtttaaattgtatccaaactaacagggtttgatgtttagaaacaataatcaatgatggaatagcagcaaaatcgagttttcagaaagacctcagatgagctttcaaatggcttgccctctaacaggaaagactttgaatcagatgcttctattgccactggttatcagctcaaattcctaaagaacttcatcccaaataccctgtcttgctgaaaggtttactggaagtataagagaatgtcatgttctgtgtccagaaaggaaggaacacgggcaccctagtgtcagcgagttgtgctcaggctcacaggatgccccctcaccagaggcgtgggaacacggcgagccccagctggccgcgctctgccactgtttctaatagccggtcacgttgatggaagtgtcacagagttgtccaacagaactgtccagtcagaaaaccaccactcatggtgctggagtgtcttagaagtaaaatatgaataacacacacttatttaactataggcaggaggtttcttatgaattgaagagaaacttttctttgcgtgggaagctgttctaaagttgggtaaaacacaatagatccaccacctctagcagccactgatagctgaaacgtgaaacatagagaccctaagctatcactgcctctgagctggcattgttaggtcatcataaagctagcgtctcccactgcaaaacccaagaggaaaaaaatagttgaaaatcctattttaaaggcctagcagattctataagataccttgggaaaatgatgacgatgacttgaaatcagaccctcgtatgctgcttccgtgggggcgaacaaaaatatgttcatcaaaaattaagcagaaatgcagaaaattttgtgagccaaaaaagctgtgttatcaggaaatgcagatatttgtggggttgtaattttttatatttgaatcgggcggttttcaaaatgatctattccatttgtagtgtatctgaaaacctataaaaataagttgatatcaatagatatccatcttccataaaaattcaacttctaaaattaagcaaactttgctttttctaatggcccctttatcctcaaattacccactgaaatagacggatcacactcagccctaagtgaagcaagcgtgcatgagagtagtcccagcctcgcctttgtaatgaggtggaaattaacatgaaggtaggctaccctgtgatagacacttaacaggatactcggggacccatggtaatacatccctgataaggaatagacctcacaaatgaactacttgcctgttaattcatttaaagcctgactgtacagtgaaaattctcttaaataaatatttgataagtgaatcaaattctggcttactaaattgccaaaatataatgactgcctgccttgataaaaagaataattacatttaatgaataaacctgccaagtacagatatgccaggtggcacctgctgtttgctgctcacttctcccaacaaatagcagctaggtgccacctgcacaccaataagctgagtttttcacttacggaacaaataactttcagaagtcaattttatagtttctgccttgccctttgttaaaaaaatacacacacttgaagcaatgaactcatgaattgttttcatcatgcatttccacttgcctaaatataaagggtcccaggtagatacagaaatacctggtttggccaaacttggtttgaataactagacatgctagaaaaggttttcattttcctgggatctgagtggaatatgttagaaaaggcatgctttctgaattctctatgcttaaaacatttctagagcagtgcttctcaaacttgaaagagcatgtaaatcacctgagatctcgtgaacatacaactctgcttcagacctggggggaggcaggagagcaagcattcctaacaagctcccagcttgtggagcacagagccgcctcaggcagcagggcagtgcagctgagcagtgacagtgaacagggccattcagagggctctccacctggggcttaggtacgacgggaatccccactggacaggctaggacttgcactgtggccattgttcctcctcctgcccatggctgagtcagcttctcagtcccttccaggatacactgagaggattcaggggcggtctcgcctctgcccatatccccacgttggtgaagtaccactggcgccattttctaatcagctcatcggcaccagcacgtcacttcccctgttgtgcagctcagttttatatttctgactcgggtgtaatagtaacatcttccccaccaatgtctcagggttattgtgagaaccagagaggagagaggtaggaaggaagcaggaaatgcagggtgcaaatacaccacattattattctaaataatgggatttttaataacaaagaccaagaagattgtctgtgcctatctagttcccatctgtaagataaaaaggtgcagtccctgagcctcctaaaaacagcaccctaatggcactgctcctcccaggtatgattgcagtagacctacgtcagttcttgcagttcaaacacattttttcactttcttctgaaatgccctcaaattctatgacatagtatccatgaagacttccctttttacccagtgaagttactagagggggttcaaaccttcttacagagggttcctaaaatgggagatctgtggagtcattgggattaaaaattaaatttcaaattcatgacatcaatgtcctcatcccagttaaataaaagtaaatacaaccttgaagaaatttgacctactcagctgcaatagctgttgacctgcagaaggcaaatgatggaccccggccacgcaagaacacacatggcgtgggctgcgttcttgtgccccagctccatggacgtgtcttcttagctgtggacccctccaaaggaaactgcttgagtggagtgctctgtattgtcactcctgggaatgctctcttaacacccccatgccttgtgctatgtgttcataccagcataggcactcagaagcaaaaaaggtttccagtgaaatttgtttgcaggcaggtattaagacatcagacactgcaggatccaagtgaacccctgcccgcctgcctgcctgccctgctctgctctgcagaagggcatgtgtctgtagcaatctgccctttctgtctgcgatgggcagagaccctggctctgcctcctgttttccctgtagccctccagacctggggagagacatgggactccagcacctgcccagcacggaagggggtctttcccaggtgtaagtgtagggctgcattcactaatttagggtcataccacaaacactgttgtgtcaccaggatgttgtcacatgcttattgtgagcatttttgtgacatgagctttctcctgagaggccaccctgtccaaatgagagctgtagttggaggcacatccgtctcatgctgcctgggtctccctgcaccagctcactctacttttcctttttatccacttggagttaaatgcacacatttatgcacaagtgcacagaaggcccagaactgttcctaatagataattaaatgatctctctccagtggaaactctcctgggctacctggctgctagctgttttagcctcttcatccagtattgatttacatgcatcggtttgtaactcagacagtatagttagttagtttgtttcaccatcaccttccaacctgcttacgtttaaactccttctctctgcttagctaatacagatttctgtaaggatttggagaaataccagatacctcctggttagtatcaggacctctccattggcctataattgaacatcactgtaaattagtttcagcttttatagtaatgcatctaacccgccagcttaaaaaaaaagtgatgataatcatgaaattcaagtgtcaatatagacttttaaatctggtgtggatgttaatagtgccagatatgcttctgtgtaatcatttttgagaagtgctgtcttctttaccataatcacattaatcaatcgggaaatggttaaatagacattttaatacattgttttttcctgagaaaaaaagtgactccaggaaatatgttgttacaatatctcaacaccaggaattgtggtcacatagcattcttcttctaaataaagcatttgttttacaaaagagtttcatgtttacttaaacgtgacaaatgtatcactcttcgaagtgagagttaaatgaaatgttccctgatttagattccaagacctgtgactactcagtaggacaatagcatatttcttttactcagatacctgtttatccctttgtcagctccagaatggagacacaggtgctgccacacagaggagagggtcaggtgttttcttagtcacaaatgcagcagtctcacccctgaaagaattccttcataacaactaaattgcacctcttagaatacctgtacttaaaatataatttgtcttctatggatatagacaaataatagcttaaatgttttaggatttgtaaagaaagaaaaagctagaaatctcaatgcaactctgacttgtatgtcctgccataggaaggtttgggacttttctttggaagatgggaggcttccttcacagtttcttaacttctcattttattcaatctttctccatctctctctctctttctctcatcctcacgctctctgtctgtctctctcctgatttgtctgcttagggacagacaggtaatggagaagaaattgaaagggaaaatacagggaaaatgtaaatgaaaagaagttattataactgttataatgaagtgcctttcatttaaatataagaatgtaacgctgaaatgaacttgtgatcctgaaatgcatttttaatgagtttcctttttattttgctgcttcagtggcatattttaaagaccctttgaaaaaagccacattaaaaaaccccaccaaatgccacaacacgcaaaattggatattggtttattccaaaactgctgatcaggaagagtggctcttcatcacaaaatgttgcagtcactttatttaaatgagtttgaatttgcattgtgatgtgccattctgatttagggaaaaaaaattagattttcagatcaaattgacccagccagtgaagcgttaaggagctggcaggtttagtctgaaagcctcatgttctatcaaacctgcagccggtggaagtcaccaggcccccgtgggaaacaactttctcgtagcatcgtcctcatgtccccacgctgagtttagttctcaccagctctcctctctccgtcccaggagaacggcccttccagtgcaatcagtgcggggcctcattcacccagaagggcaacctgctccggcacatcaagctgcattccggggagaagcccttcaaatgccacctctgcaactacgcctgccgccggagggacgccctcactggccacctgaggacgcactccggtaggtcccctggatgcagtccggggctgtctgggtgtcccgggattcctccactctgcccgcctgggtcccggattgtgtccttgctggctaaccctgagtccctcccagctcgcagtcctgcatcgggtgtgagctgttgcttctttgacattgccccatcccccctccccatattctccttttcccactgcaccagggagattgggcgcaggacccccatgcacatacacacacagaagtctcaccttaggtagcatgtttcagaacaggcctcctcatcccgtttgtcaggctggtatggtttccctaaaggattttgcgggaaatgttttcaccaagtgtactgctaagacccaaaacgttttcaagtacaattctttctgttgttaagtcccgctttggagtgttttacacaggtgtaatggatagcttttttgcagagctggtagagaagggtgatttagggcgcacccacccagactgagccccgtgtggctctcacaccaaaaaccagccaaggccacagttacagaggccagtctggggctgttaccagattttagacagcagcctttctctttgaattagacagttaaagtacaacccacataatctggagtcttgacaagatcatcataggcataaacgctctatcattctcaaaacagtctcagcctgcaaagttcaaatccactaaagtttggttagatcctctgcctcctgagaaatggtcctgggtgtttcattatccagcagtcccataattctacagggcagaggaagagagggctcttggccggcctgtcatggatcatgtttgcctacagtgtggtctatacaacatgacatggcacaggtctccttcataccgtccagttggggatatttgctgtagcatactgcatgagacttcggaggcgaaaggttgatggcttttgtccttcccctcaaggagcttcctgccccagccagccaccagccagggcccttctccaagcagcagcctctctaagccggtgccctgggggatggcaatgcctcagaccagctactcctcacccaccctgggtagcaggataaggaggagcctccctcagggaggcagacgtgtgttctttgtgaaatatctgcagcggcccaggccatctcttcccaaatgtgatgcgtgtatttgatggttgagggttttagaggctgctcattgtgtccatctctttatcacacatttactgagcacccctgggtgcccattctgcacagaagacctcaggtgcacacaaggaaagcaccaatgtgttacaggaggcatgacagagtgtctgaggggacagtgggagcataaggaaggggacagtgggtcctggggtggaaagtcaggagagacttcccatagaagggaggggcttctgaagtacatgctggaaggcgagtgtctgaggtttgctttagagacattctaaactagtgtgtgagtcggcagtaatcccagagaggggtgcggcacccatgttggcaagaactcggtctcactcctggcaggtggtccctgcagtagttcacccagttggctggagacaaaaaggagaggagtcacaggggctggtgcatcacctcctctccatcctgacctccttcctggcggtgcacatggagaaggatccccacatgctcgccatcagaaaattgtcatgatttgggtgatttgacttcctaaaaacttccaagaaaggagctataccaagctgaggagttgcttgcccgagagggcgggcagatcagcaggggggccatggagtgggacacttgccttgttacagagggacagggagaacaggtggttcccctgagataggaagacagtagcatgacggtgagcatctttaaatgttcacatgggtgatgagcatgtatggtggtcccacctggagacaaaggtggtacctgggcctgataagcaaggcccatggaggcctcagctaagagaactgtgcagtctgtctgccatgctaaccagtggtctaggaagaagcttcacatctgttgtttctaaggagccatgtagacatagacacagttggcagcaatattgccttgggttagtgggaagcaccacttcccatgtcagtggaaaacacagacactcgttcttaggttgacaccaaccataggtcatcatcactcctagattactctgattgcaatggaatctctcatgcccatcagtccttaaaagcagagctgctctctttcctttcttgttgtggagttaggaacatgtagtggagtattttatgttattctggctccaggcctatgtatttcatttgaggcatgatggtttcctattctaagtaccactaagtattgagtagttataaaatggtggtagactttggactgtgtatttctattctggatccacggatgagcaagtggagaaaacccatcatggatgcagggctgctggggatctgtctgtccgaaaatgctatagagacacacactgctcttggggaaaaaaacagtgatgatttcttcaaaatgatttcactgaagtgaaataaggactttggccttgctctggcaaattcaaatgtggccacactctactccattgtggaatggtgactcccctgtcccatgaaaaaccaggatcaacatcacagctttctctagcaactggtggtggcctgaggtcttatgaactagcatgttagcagggatagagttagggaatttgtccgatgctgacagcaagagggtgctgggggagctggaggggtggagaagtgatctcccttcctgcctcatggcttcctttggaagttgcaagcttagaatttctttcccaaagaatattggactatgcttcaagagacacttgagttcagttgcttacagtgaacatacttcttatgttccagagttaaactcaatcatattttcagaaaaatagatattcagacatcatgtcatatttacaggaagctatttgatcatggaatttactgaagtgacagctttttcaggaaagggtatcgagtggttatctatatcctattaatttctaaaagcaatgctcaataaaatgtgcaattttaggcaatattctgtgtttcaacattatttccttatgttgggggtacatcactttatatagataaagaaagatccttttcattctctattaaatggtcgagaagtcaaattttggctttcatgttggtcttcttactgtagcttctgttcacctaagaaagagatttaaccaacataagctttggctaaaccattgtaccaaacatttctatttggatgctcttaacttttatacaatactttgaaatgtgttcaataaaacaacatgagaaagaagtagaagattctgctattaggttggaaattgatattccttacataagtgattaaagatactttattcagtttatcccttaaaatggtatctactgaagtccttaaacttaggcatgatattaattcctggctagtctttctttttcctaaaatatcactcaataaaatccaagaaggactaaaactcattatatagaactgcactgcccaatacagcagccactagccacatgcagtatttttaattataataagttaaaattaaacaaaattaaaacttcagccctcagtggcactagccacactttttgtgtccaacagctacatatggctggtggctgccatattggacagcactaatagagagcatttctatcatcataggaagttctgttgaacaaggctgatctggaagctacaccatcctaaagctctttggcaagaaagcagagcttttccataagctcagtttatgaacaatatattttgtattttctatgtatatgtatatgagaatacgtatacatagaaatatatgaaaatacatatacatataaatatacatatacataaaatagaaatacatatacatagaaaatacaaaatattttgtattacatatacatatgcatggaaatacaaaatattttgtattatataatgcaaatgtaagatatatttatattatgtaagaaaatacatttgtgcaataaaacgcttatttcattcaagcatatttgtccagcatggtttcttagagactgaagttacccagtttagcacctggaactgaattcactctggctgtgttagatgtagcaccagcgtcatgacagatccagcccacagcatagactgaggcctactctggaggcaaatctgcagacaatgtggccaacacatgggccttcctcacatctctgtccagtcgctggcatccacgttataacacagcagtggctcgttttctccagttaaaggctaggcttccgtgattgaagcaggagagaatttggtagtcagtaaagtagacaaactaagacaaaacaagagtcgcatgagttcttggtacaattaaagaaacccttggccaccaacgtttttaaattcaggaatttcaccaagtccgtaacagttttagctctcaagggtagaattttttttttaacttttttggtaataattgtattgcatgcattccccttacacagaaggctggcatttaattggggtcttgaactcaattgtgttttctgcagttggtaaacctcacaaatgtggatattgtggccgaagctataaacagcgaagctctttagaggaacataaagagcgctgccacaactacttggaaagcatgggccttccgggcacactgtacccaggtaagcgctgctgctcggaggccagcctggtgggctctccccccagcacggtggggaaggagggcgctctgcatgcagccttaggagcagagccttgggcctgcttcctgccggggctaggagggagggaagtttttggccaatagcatcagtttcaccagaagcacgttgtgcttcccagctttctaggtcctcatctgaccagagagagcttgattttaaaacccttcccacttccaatcgggagaaactcctaggatagcagtgaccttgaaagttttggggttgttttttggatgttggtgattttaaaacaacaacaaaaaaaacacctcaagtagtgatatttctttgtaaacaaaataaaatgtaaaatattgttttgaaacaattttttaacaaagttgatcaaataagaactttcaggctgtgattttaagcatcagttcagactgagggcagggtatccactcctgcatggagtgtgaggttgaagtttgttggaagcccctggtagttgataatattgaggttgttgaaggatgagaggaaaggtcgcttctttcttaggagctgtgatgctccactgtattgcataacgagatagcacttgattcagaccccagagcctgtagaataaatgatgtggagagagtattgaaaaaggctggatttatctgggggaatggcaatgttataaaaggttcttccctaattttttaagagataagcaatttataaagagatcaaataaaactaatctctttcaaaacagtattcatttagcactacccctggaaaaatcatcactagactgtgagtcgaaatttaacgaatggaggaaaatttattaagcctcattcacctttcggactttaacaaatggttcaaaagggaaaattcaccagagtaccctcagattattaatggcagatgagagcaggaaaaaagaatgttgagaatggctaaaaattgattccaattagtcttggcatggagaggcaacatctcacctggccagagccctgcagccagagtgctgtctcctctgccagtcagccagggatctggggcttacaggcgattcctccaggcctcaatcttctcacctgtgaaatgggaagataccattaaattgtcatttggcttctttcaaaatctcaggtctagaatggaaaggcattgaaggtgagtggaagagaagaaactggatgttaaaataataataatgtaacagttattaattcacatggccagaccccagggcagaagactaatggacgagaggatgattatgttcctttgaaataaaatgtcaacattcaaatattgtttttttctgtaaaaaataagtgaatgggtcttggaaggaagtgctcctaatgatgtagtttacatattaatgcttaataaaccattttatttactcaacaaatattcactgaggacttataatcaataataccaagccctattgaatacagtaatctaatatacacatagaattgaaaagacaaaatgaacaaaagaaacagacagtatatcatttctgtttcctccactgttttcattactaaagaaacaagctgcagtacacagccggctgagagctgcttccgggggagcaggagaagcagcactcatttcactgagacctgggtttgagttctctgtttttgaatgtgacccagagctcatcgcttaaatgcgactgataattcgtgcctggccaccctcatagcagcgctctgaaagaaacataaaggagtggatacacgtgtgagcaggagcctagacatgtgtcaggtacttacctaaacatggatgtttaggtcactgacatactgacaccatcaagctaattcttctctattggtctctatgtcagtaaggctacgtgccagtaatgtgtattaaatattaccctgtggtgagtatcaatttcaccctaaaatgttcatttattccctttggtggttaagttacttggactaccacagtgatgcctctatgtccccacactcaggaggtatgaaagcgtctctaggaaggagcagtggtctggtctcagtgtggcagcagacaaagccaggctcgcacttaattacattgaattatttctgtctggtatggcctcaaaaggtgcaaaatgatcataaatatctttcgacagtcacctttggtttttgcccttgccagcacctggcccttgtaggtgctgctggtttaaggaacttggagtattaaggggatccataaaaggactttgacagaagtcttcctgaactgtgcttagggccaggaaggcaggcaggggcttggtcatttcagctgtgctgtacccggatgagtggaaactgtgccttctgatgcatctctcctcctctgttcccctctaagaaccccttgtctgtgctcagatggatgagcaagtagtctcttctcccaccccaaaaacctccttagatggggtggattcagtaaaatcaaagcctcagttaggcaatataggaaaagatgaatttttgtgaatataattttctttagtaaaaggcttttggtcacctcttataatttctcaatgtctttatggaaaaaataagtttctcttgagcctttggtgcacaaacagagtcaccagcccttggaggtgcatgcatctaatgacagagcagcctgtgactcgcgagcatcaggctcacttgttccttgctgtgattgactctggggggtggaggcttttgggaggccctgcgccatggtgcagaggaagacccctgacctctgggagccctgggcaggctgtgggtgtggcttggcagagaaggatggacatgtcaggaagcatattagcttaatagatgaagtgtgtgaccagtgtaattgggttttatggatttatatttatcttgcatataaatcagggtgtggcctatatatgtttgcccatttaataaaaaacacatattccaaatgttaaaatataaaatataccagcaacaatagccaatttgaaatatatgttttcaaagtaacattcataatagcaatgaagacataaatacttagaaatgaagaaaaccagcaagccatagtgaggcaagaaagaaaacatgggagattggagaggtagagaggtctggatgccgtccatgggcctctgcccaaattagtgtatcatttcaacataaaactggcttgtaaattttactggaatctggtaaccatgtatcataaagtatctggatatatacatgagataattggcaactttttagaagaagaataatgtcaagtgatgtatgaaaatgtagctgaaatggatggaactctttgaagaatagacaaacaaatcaataatagagatggataccctactgaagaccccatgataaatcagcatgaaatatccgaggcaggaggaagcataaggcaggaacggtattggaagaattgatcaggcatttaagataatcagtttacattatcatctccttatacactaggataaattgccgggaaatgaaagagttatatttttataactccaaccacaataactgatgtaaatatagatccagctatgcaggatacaaagttgtatcttcaattatggaaagagatgacatagaacaattgagaatgactagaaatggcccacacatcagcatttacctccataatttctaaattttctacagcagtcaggggtgctttgattttcaggagtttgttgtatgctcagggataaggggctctgggtttttcctccacattgcccagatctgctgtaggctcttgggggccacatttttgtatctccgttcactgtgctggtccatgaagtcagatgttagatcacacagttctttgcgtctcagagggctgctgttagggactgactgaggtagcagatttgggagtattatgaaaagttaaaaggacttgtcaaagggacatcaatacttaatcccagtccagcctctggtgatggtggtggtgcctgtgcatttggacttaaagaggatgatgtgtggtgagcagctgttggaggaagaagcatttgcaggtggctggtcattggcattgctccggctgcctctgcctgtctggaagtgttgctgggaagattagaattaatctctaggaagggcctggctcttgtaggcacttaacaaatgtcagatttaacattggacgcgactgaaccctttaaacataagcctttctaaactggcctctctgtctttgactttagtcattaaagaagaaactaatcacagtgaaatggcagaagacctgtgcaagataggatcagagagatctctcgtgctggacagactagcaagtaacgtcgccaaacgtaagagctctatgcctcagaaatttcttggtaagagttaaatgtttgctgtctcttaaaaaaaaactatgtgggtgttttagatgcaagtagaaatgagttgagggtggaagaaagggaaaaaaatcttattttttcaaaaggaaaaattggtaagcttaacattccttaaatatcttagaattttttccaataagtatcttaaaaataacaaacctcccatcagtttttcctagatttgattttgcagcatctggggcctgccctgtgatctgcctgtggacatcgctcttaggggcggctgcaccagcgtgcacagggtggagagtttgggcctggctcgtccgggggacaccacactgcaggacactccaggcctggccggcttctcagagcttcagatcctcatttttcatatgaagctcctaatgctccccttatgggggactctgaagggttaatgggaggaatcatacagtgactgacccctgagaagtgtccagtgaagacagggcttagctaggattgctgttttgcctaatgctctgcgggattaaaaaaaaagaagaagaagaacaagaccattcgtctctctaggagcattgcccagagtaggtattagacacaccaacaccaccatccagccagacgctgcagggacagtgagccagggtccgagtggaaaggcgctaggcttgggaaccagctcagagtcaatacagagccaccgccactcaccaactctgtcagcttagtaaaatggctctgcccctagagccctggttccatcctttagtatctcacagggtgattgtgaatatcccatgactccaagattgagaaaacgtttagaatccctcggtgtgaaggttaactctgtccggaaagaggaccagtaaaagcttcatgaggctgagatgcactttggaagaggaatagagtttcagcacattctaggtgttggaggaatgggggaatctaggcagatgtttaaaatcaatgagaaaccagaatgctgaccatgagggttggagtgggggcctaaggacatgacggaggagcagggtgtgttcccagcttaactcaggtacccatggggaagcaggaaaagtgaaggtgtcctaggcagctctgccacaggatgaatggcttcagatgccaggtgagcgagggacccttcattcagtcagcaggaaagaagcactggcatattttttatgagaacaaaggctaggatagtaaagacagcaagtaccaaaaaatgactggaaaagggagactgtggaggcagtggcagcaggcatggaaagaagggcttgtgaaggggaaggggtggtgtcagaggaacatagggctgggggcagggattaggtgagggaaaccatgagtcacactgattctagagtagtgtgcccttgatgaaaaggataacaccaggttctaggaaaagatggggttctgtttttgacgtgttgattttcaaggacttctggtgtttgtgacacatggggaaattgtggtgggagagaggtggggccagaacaggggctggtgaggccaagggtcccagagggcacctgttgacctgcaggatgacatgaagggggaaggacagaggcaaggccaagtcctgggcaccagcctccctcttgcagcttcaaatagggctccattttgaccttttgattaattagaggtttgtcataggttgggggttgagaggagcaagggagagaaggattcagtgtacaaaaagaatgaaagccactggctgagccagtggggagttgtccacacacacatgagcctttggaccatgagaacgagggaggccttgccttcctgaacggagtaggagtgaggtcctgtgctgagcgtaagcagtgggattcccacagcactgggcacagagcccacgggctgcctcctgagcagccagcatctgcctggggtggacacagtgacagagagatgggtggtgactggggtatgggcagagataaggcagcaagtgtgtgcaaggggagtgaagggttactgaccttaagaagcagggatggcgtcctctgtcaggtgaggagcctggagaatgctttggtgaatgaacgtttgcagccccttttagcttttggagacttgaaaccaaaggagagattcatctgtgaaactctactggagccactccccaacccccacccttgtgagaccacaatgtgggcgttggcttgagatgcttctgtgttagtagaagaaataaacaacacagtgctctgatgaggcaaagcgaagatgaaaaaggagttcccaggggacatagtaggaacagtggacgagggtagcagaagaggagtttggagcaaaagactcacaagcagctgcataatctgttggtgcttggcagttcatttgtaaaaatgatgcctcttcctgccctaaaatacctaccttacccccgcttcaacttgatgagatttccatcagtcactcccaatgtgtcacagcttctgcagccctaaaattaaaaggtgagtgagtctctgaggcccctctccacttctcggatgctgagtttagccttcatgtgaatgtggaaagactaggaatacagctgttatcacacaagctggcccaatagtggttcagttgagagagccccatccttcagagtcagctccagctaggagtgactggtggccttgagcatggtgctgggcttagtgttgccatctgtggaatgggtgtgggtctgttgccctgcctcctcccagagctattctgaggctcagaaggggtgatggatgtgatggtgctcccaacactagaaagcatcttaagaatgtaagattttcatgatgactgttgctcagagtggctattatagttttgctttattgttctataacctatgattaaaatttttaccttaaactttgacgtgagtgtgaataagtatttgttttgccagcaacattcctcaccactggggccattaaagatctccccctctgagaccatcaaatacaggtcaacaggactgattaatctaattagaaaagggcttgtattaaatagcaatgataattgttgtttttagtctgtctggtgtttgacttgggaacgtttttaaaatagagaaaagcacaaagaggaaaacaacaattaccaatattcctgctacccattataattatctaggtatattttcttcttttgtaagaaaaagaaaccctgttatattgttaaaataacacaaagttaatataaagaattttaatgcaaagattaatgttttcaaatcaccacaaaacccaacatccagaaattaccaatattaaaagtagaaaagtatcattctaaatattttctgttgcatatgtatgtgagtggataggctgatgaattaggtggattgatggataggtaaatatgaaataaatactttcataaatattccaacttatcatacatgccttaaattcaagaggtgaaaaaagacccaaacaaaactagagaagcggcttattttaaatatcctctgacataaaggaatattatatttaaaggatcctctaagattaaaaatatgtactatgaaaaacattaagaaatttgaattttttttaatccatttgtttcaatttaagcagcatctactggctcactgctttgaaaaataaggacagtattccagttcacattcagtgttccagtgttcacattatcttattatttttacattgtccagctttgtaatattcacattctattctgtaatcataattcatagtagtttagttatttattactaactctatttaaatagattcaaggatcagaccctgcccttttcttcttatttatgtttattttgattaatctcttaattgattggactttacattcaagcaacttttttaaaaaaaagtttctatagatgttctatttctatcattgtattgtttttgaggatgttggcctgttgcctttgtatttgatgagcattttgacagagtctatggtcttgggccactctttctttttctcccttgagaactttttagattttgctgatggcattgcttgttgaatgttgctgtggaaacatcaagtctagtgtaactgtttcttcttcaaggtgatttgcattttattcctgaatgcctgagggttctttatttaaccttgaagttaaataccctaattaggatgtatcttggtctattcattcggaataaaaaattcctgccattttgtctagagagtcccttttttttctctttatttctgggaaattctcttttatataaatatgttttgttccatctattgtgatctctgttgagggataccagttgtccatatgttagataatttgtcttccatatctgttaacagttcttaaagttttttgtttatttctttgtctatttttacatttactcactgttctcttgtggttttcctctgtcagtaatttaatttttagtagttcctgttctattacttgctatttttaatccatgcattaattttataataatattattttgctccttattttgtctcctgagacccgaaatctcttttttcctcttactctgttgcttttgcattttattttgaatacttttaaaattgattccatgttatgaagcaattatgaggcatttcctctcttgttggaattaacgattttttcccctaggagggactctatggtctgtgttttacttcctttcttcccctgtatttctagaaaatattttcctagtaaccctgacatttcttttcatcttgcttattctagttggtctgatatagcttgattgacatttcagccttcttcccactatatttttttttcctgtgagagctattgggttttctaaatcctgaaagaatgccaaagatggggttggaggagtttggtgaggcaaagtgcagcctttgttaaaatacttttcctttgctctctctccctcatctgaaatttagttaaataccctaagccatcagcactgtacctagttggggaatgctttcatccccacaggagattctctggggctttgggccatcttccccttcagtgtagaccacagaggactttgcttctgtcccagggagcccgcaggggctcacttctccatgttcatctgattcttgtcagccaaggtttcaaatgcttttctgatcagaacagggaaaagatacctatctgaatcatgtctttatagatatgaggctatgagggaaaattctgaggttattcttgactcacacctaaagatttggaaatgagattagcagcaaagctttgccctacatctcatgtcagaattttctgtttctttctagtctttgagtgtatgtgtgttctcacacacgccataatgaaatgcatattatatataattatgtgtatatataatattctatgactatacatgacatgttcctttagctgattgctgttaagagaaatttataggtttttatttttcttgttttgttgggtattaaggaagagaaattctatggtaattttcatgtggcacagtaatctggcatatatgttgatttttttcctacacccatttgttgtgataccaagtttgaaaacaacagatttcagtggttgcttgggaaaccacagaaccatgacttggggagagacaggatgattaggtgggaaagcacccttttggtggggctgtaaagacttttatatttagcaaaattggctacaaagtccattcccctccttttcttgccttgatttggtagagggatagacttggatacaaactagaatggattcattcttctctggagttagtgtaacaagacatttagctgctcaacacaaaaacagaaacaaaaaaattgtgtggtttcagcagtgctatacaattactttttctgacctttaatggagagaaacaccacttctttggtccctaccatcagcttcatagggttttcatcctgttctgtttctgggagggcgtaactggccatgcacaagttttttttctctaatcagagtatgtgccacttctgaccaccagtagatgaaaacgaatggaaaccaggctattatatgatacatatccattacaaaataagacatgaaactcaaaggtactttatggtataatggggcatatattcctggacaattcttaatggtcacagattttataaaaggactattagtaaatgtatgaattacagagtaatttatccttctgttagtaagaaccagctgatgacctcagtgtcaggtgcatcgtggaaggtgttgggaccttcccttgccaccaccctcaccagccatcatcagccataacctgcacattggggaagttttgacttatccctcacttttgcccctcttcaagctgttctttccacagtgaatgagaaggccacttcttccttcaaacctttcagtggtttccattttcctttagacaaagtctctgcctagctggcctctgcctgcccctcctgcctacctctcgagcactgcccccacctagggctctggttccccaaccttcactcggtcctgccacacctcccagccccttctcccttcagaactttccttcttgttgtccccaacactgggacacaaaaccctccttatcaaccctccttatctggctgactcttacaagatcagaaacctgtgtaatgctctcatggcacgctccccttgtcttcgtggatttctcagatgggaaggaattatccatgcaatcacacataaacttctacctaccctcccctagtagctgtctgctgctaaggatggggaccattctcacttactcactgttctgtccctctgcccagtccagatgtgttgaaggatggaaatatacagagtagtggtaaaatataaaccgttcagacattccaaggatgggctcatgtgctttgactcattaatgtaccactgctgaaaacagaacacagccgcagtcttgccagtaagagtgcagttactgtaattaatgaatttgctaattaagccatgatttcatactgaacttatgaccaacatattgagaaggtgtgtcttcaagaaaatttattttttgtattaagatatttactccaaagctaattgaagaagccaaatctaggctctggtttcaccattgccagggaaatgagctcatggactcctatgaactgatgatgttagatcagaagtttctcaaggccagggcccaatcactgctgaggcgtcaacagtagttccttgtacatcaataattctcattacttttaaaaaataacagatgaatagcaactattttccctgtagctcccttgctgtgcctcctaccctccaccacatgtttctggggagccctgcttcgggcctgccaactacagagaattacttttgagtatcccttccactctcatctcaagacagagttcatctacctttgggttatttgtcaaaaatgtgtcattttattacaaaaaatatacaatcatcatgtattttgattaaattttacactagattattaaaattattaaatacaattattaaaattaataatttaacatatcacatattttaaatatattgtatataatgaataataatataattattgtctattttaattcaataaatgtatagtaagttagccagttgtaaattactgagaacactctactgaaaaagcatcatttcaaatacactatttaaaatattaaatgaaatacaataacataattaaactaatctttggttcccctatttatgtattcatttatccaacaaaatctccttaagtgcttataatgggtaggtcctggctcggtgtcccctagacagacgcatgggccttcccccagcccgtcagtatggtgcaggtgtgatgtgtccgcaggtgtgtgtgtatgtgtgcaggtgtggggtccgcaggcgtgctgggcccccaggccgtgttccccttcccctccccggttgtagatttcagctgttgctgccagacctgaccggttccggaggtggccgcgccccactcactgtcgcctgctttccacaggggacaagggcctgtccgacacgccctacgacagcagcgccagctacgagaaggagaacgaaatgatgaagtcccacgtgatggaccaagccatcaacaacgccatcaactacctgggggccgagtccctgcgcccgctggtgcagacgcccccgggcggttccgaggtggtcccggtcatcagcccgatgtaccagctgcacaagccgctcgcggagggcaccccgcgctccaaccactcggcccaggacagcgccgtggagaacctgctgctgctctccaaggccaagttggtgccctcggagcgcgaggcgtccccgagcaacagctgccaagactccacggacaccgagagcaacaacgaggagcagcgcagcggtctcatctacctgaccaaccacatcgccccgcacgcgcgcaacgggctgtcgctcaaggaggagcaccgcgcctacgacctgctgcgcgccgcctccgagaactcgcaggacgcgctccgcgtggtcagcaccagcggggagcagatgaaggtgtacaagtgcgaacactgccgggtgctcttcctggatcacgtcatgtacaccatccacatgggctgccacggcttccgtgatccttttgagtgcaacatgtgcggctaccacagccaggaccggtacgagttctcgtcgcacataacgcgaggggagcaccgcttccacatgagctaaagccctcccgcgcccccaccccagaccccgagccaccccaggaaaagcacaaggactgccgccttctcgctcccgccagcagcatagactggactggaccagacaatgttgtgtttggatttgtaactgttttttgttttttgtttgagttggttgattggggtttgatttgcttttgaaaagatttttatttttagaggcagggctgcattgggagcatccagaactgctaccttcctagatgtttccccagaccgctggctgagattccctcacctgtcgcttcctagaatccccttctccaaacgattagtctaaattttcagagagaaatagataaaacacgccacagcctgggaaggagcgtgctctaccctgtgctaagcacggggttcgcgcaccaggtgtctttttccagtccccagaagcagagagcacagcccctgctgtgtgggtctgcaggtgagcagacaggacaggtgtgccgccacccaagtgccaagacacagcagggccaacaacctgtgcccaggccagcttcgagctacatgcatctagggcggagaggctgcacttgtgagagaaaatactatttcaagtcatattctgcgtaggaaaatgaattggttggggaaagtcgtgtctgtcagactgccctgggtggagggagacgccgggctagagcctttgggatcgtcctggattcactggctttgcggaggctgctcagatggcctgagcctcccgaggcttgctgccccgtaggaggagactgtcttcccgtgggcatatctggggagccctgttccccgctttttcactcccatacctttaatggcccccaaaatctgtcactacaatttaaacaccagtcccgaaatttggatcttctttctttttgaatctctcaaacggcaacattcctcagaaaccaaagctttatttcaaatctcttccttccctggctggttccatctagtaccagaggcctcttttcctgaagaaatccaatcctagccctcattttaattatgtacatctgtttgtagccacaagcctgaatttctcagtgttggtaagtttctttacctaccctcactatatattattctcgttttaaaacccataaaggagtgatttagaacagtcattaattttcaactcaatgaaatatgtgaagcccagcatctctgttgctaacacacagagctcacctgtttgaaaccaagctttcaaacatgttgaagctctttactgtaaaggcaagccagcatgtgtgtccacacatacataggatggctggctctgcacctgtaggatattggaatgcacagggcaattgagggactgagccagaccttcggagagtaatgccaccagatcccctaggaaagaggaggcaaatggcactgcaggtgagaaccccgcccatccgtgctatgacatggaggcactgaagcccgaggaaggtgtgtggagattctaatcccaacaagcaagggtctccttcaagattaatgctatcaatcattaaggtcattactctcaaccacctaggcaatgaagaatataccatttcaaatatttacagtacttgtcttcaccaacactgtcccaaggtgaaatgaagcaacagagaggaaattgtacataagtacctcagcatttaatccaaacaggggttcttagtctcagcactatgacattttgggctgactacttatttgttaggcgggagctctcctgtgcattgtaggataattagcagtatccctggtggctacccaatagacgccagtagcaccccgaattgacaacccaaactctccagacatcaccaactgtcccctgcgaggagaaatcactcctgggggagaaccactgacccaaatgaattctaaaccaatcaaatgtctgggaagccctccaagaaaaaaaatagaaaagcacttgaagaatattcccaatattcccggtcagcagtatcaaggctgacttgtgttcatgtggagtcattataaattctataaatcaattattccccttcggtcttaaaaatatatttcctcataaacatttgagttttgttgaaaagatggagtttacaaagataccattcttgagtcatggatttctctgctcacagaagggtgtggcatttggaaacgggaataaacaaaattgctgcaccaatgcactgagtgaaggaagagagacagaggatcaagggctttagacagcactccttcaatatgcaatcacagagaaagatgcgccttatccaagttaatatctctaaggtgagagccttcttagagtcagtttgttgcaaatttcacctactctgttcttttccatccatccccctgagtcagttggttgaagggagttattttttcaagtggaattcaaacaaagctcaaaccagaactgtaaatagtgattgcaggaattcttttctaaactgctttgccctttcctctcactgccttttatagccaatataaatgtctctttgcacaccttttgttgtggttttatattgtaacaccatttttctttgaaactattgtatttaaagtaaggtttcatattatgtcagcaagtaattaacttatgtttaaaaggtggccatatcatgtaccaaaagttgctgaagtttctcttctagctggtaaagtaggagtttgcatgacttcacactttttttgcgtagtttcttctgttgtatgatggcgtgagtgtgtgtcttgggtaccgctgtgtactactgtgtgcctagattccatgcactctcgttgtgtttgaagtaaatattggagaccggagggtaacaggttggcctgttgattacagctagtaatcgctgtgtcttgttccgccccctccctgacaccccagcttcccaggatgtggaaagcctggatctcagctccttgccccatatcccttctgtaatttgtacctaaagagtgtgattatcctaattcaagagtcactaaaactcatcacattatcattgcatatcagcaaagggtaaagtcctagcaccaattgcttcacataccagcatgttccatttccaatttagaattagccacataataaaatcttagaatcttccttgagaaagagctgcctgagatgtagttttgttatatggttccccaccgaccatttttgtgcttttttcttgttttgttttgttttgactgcactgtgagttttgtagtgtcctcttcttgccaaaacaaacgcgagatgaactggacttatgtagacaaatcgtgatgccagtgtatccttcctttcttcagttccagcaataatgaatggtcaacttttttaaaatctagatctctctcattcatttcaatgtatttttactttaagatgaaccaaaattattagacttatttaagatgtacaggcatcagaaaaaagaagcacataatgcttttggtgcgatggcactcactgtgaacatgtgtaaccacatattaatatgcaatattgtttccaatactttctaatacagttttttataatgttgtgtgtggtgattgttcaggtcgaatctgttgtatccagtacagctttaggtcttcagctgcccttctggcgagtacatgcacaggattgtaaatgagaaatgcagtcatatttccagtctgcctctatgatgatgttaaattattgctgtttagctgtgaacaagggatgtaccactggaggaatagagtatccttttgtacacattttgaaatgcttcttctgtagtgatagaacaaataaatgcaacgaatactctgtctgccctatcccgtgaagtccacactggcgtaagagaaggcccagcagagcaggaatctgcctagactttctcccaatgagatcccaatatgagagggagaagagatgggcctcaggacagctgcaataccacttgggaacacatgtggtgtcttgatgtggccagcgcagcagttcagcacaacgtacctcccatctacaacagtgctggacgtgggaattctaagtcccagtcttgagggtgggtggagatggagggcaacaagagatacatttccagttctccactgcagcatgcttcagtcattctgtgagtggccgggcccagggccctcacaatttcactaccttgtcttttacatagtcataagaattatcctcaacatagccttttgacgctgtaaatcttgagtattcattt

SEQ ID NO: 2 - DNA, 30 bp, Homo sapiens
gtgacagagtcgtgggggacaagggcctgt

SEQ ID NO: 3 - DNA, 30 bp, Homo sapiens
tgtcccaagtttcaggggacaagggcctgt

SEQ ID NO: 4 - DNA, 30 bp, Homo sapiens
aagcgcgacgcacaaatccacgggacaagg

SEQ ID NO: 5 - DNA, 38 bp, Homo sapiens
ggaccggtacgagttctcgtcgcacataacgcgagggg

SEQ ID NO: 6 - DNA, 38 bp, Homo sapiens
ggaccggtacgagttctcgtagcacataacgcgagggg

SEQ ID NO: 7 - DNA, 30 bp, Homo sapiens
ccagggatctcagaaattattagtacatcc

SEQ ID NO: 8 - DNA, 27 bp, Homo sapiens
ccagggatctcagaaattattagtaca

SEQ ID NO: 9 - DNA, 28 bp, Homo sapiens
ccagggatctcagaaattattagtacat

SEQ ID NO: 10 - DNA, 15 bp, Homo sapiens
ccagggatctcagca

SEQ ID NO: 11 - DNA, 17 bp, Homo sapiens
ccagggatctcagcatc

SEQ ID NO: 12 - DNA, 2 bp, Homo sapiens
cc

SEQ ID NO: 13 - DNA, 18 bp, Homo sapiens
ccagggatctcagcatcc

SEQ ID NO: 14 - DNA, 14 bp, Homo sapiens
ccagggatctcagc

SEQ ID NO: 15 - DNA, 24 bp, Homo sapiens
ccagggatctcagaaattattagt

SEQ ID NO: 16 - DNA, 18 bp, Homo sapiens
ccagggatctcagcatcc

SEQ ID NO: 17 - DNA, 18 bp, Homo sapiens
ccagggatctcagcatcc

SEQ ID NO: 18 - DNA, 17 bp, Homo sapiens
ccagggatctcagcatc

SEQ ID NO: 19 - DNA, 14 bp, Homo sapiens
ccagggatctcagc

SEQ ID NO: 20 - DNA, 13 bp, Homo sapiens
ccagggatctcag

SEQ ID NO: 21 - DNA, 39 bp, Homo sapiens
ccagggatctcagaaattattagtacatcccacagtgaa

SEQ ID NO: 22 - DNA, 23 bp, Homo sapiens
aaacatcaagtctagtgtaactg

SEQ ID NO: 23 - DNA, 24 bp, Homo sapiens
gaaacatcaagtctagtgtaactg

SEQ ID NO: 24 - DNA, 21 bp, Homo sapiens
acatcaagtctagtgtaactg

SEQ ID NO: 25 - DNA, 25 bp, Homo sapiens
ggaaacatcaagtctagtgtaactg

SEQ ID NO: 26 - DNA, 22 bp, Homo sapiens
aacatcaagtctagtgtaactg

SEQ ID NO: 27 - DNA, 22 bp, Homo sapiens
aacatcaagtctagtgtaactg

SEQ ID NO: 28 - DNA, 16 bp, Homo sapiens
aagtctagtgtaactg

SEQ ID NO: 29 - DNA, 20 bp, Homo sapiens
catcaagtctagtgtaactg

SEQ ID NO: 30 - DNA, 23 bp, Homo sapiens
aaacatcaagtctagtgtaactg

SEQ ID NO: 31 - DNA, 20 bp, Homo sapiens
catcaagtctagtgtaactg

SEQ ID NO: 32 - DNA, 25 bp, Homo sapiens
ggaaacatcaagtctagtgtaactg

SEQ ID NO: 33 - DNA, 21 bp, Homo sapiens
acatcaagtctagtgtaactg

SEQ ID NO: 34 - DNA, 24 bp, Homo sapiens
gaaacatcaagtctagtgtaactg

SEQ ID NO: 35 - DNA, 21 bp, Homo sapiens
acatcaagtctagtgtaactg

SEQ ID NO: 36 - DNA, 34 bp, Homo sapiens
tgttgctgtggaaacatcaagtctagtgtaactg

SEQ ID NO: 37 - DNA, 52 bp, Homo sapiens
agggatctcagaaattattagtacagcgaaacatcaagtctagtgtaactgt

SEQ ID NO: 38 - DNA, 52 bp, Homo sapiens
ggcatccagggatctcagaaattattagtacatccgggcctaaacatcaagt

SEQ ID NO: 39 - DNA, 52 bp, Homo sapiens
ggcatccagggatctcagaaattattagtacatggggacatcaagtctagtg

SEQ ID NO: 40 - DNA, 33 bp, Homo sapiens
catccagggatctcagaaattattagtacatcc

SEQ ID NO: 41 - DNA, 30 bp, Homo sapiens
catccagggatctcagaaattattagtaca

SEQ ID NO: 42 - DNA, 31 bp, Homo sapiens
catccagggatctcagaaattattagtacat

SEQ ID NO: 43 - DNA, 18 bp, Homo sapiens
catccagggatctcagca

SEQ ID NO: 44 - DNA, 20 bp, Homo sapiens
catccagggatctcagcatc

SEQ ID NO: 45 - DNA, 5 bp, Homo sapiens
catcc

SEQ ID NO: 46 - DNA, 21 bp, Homo sapiens
catccagggatctcagcatcc

SEQ ID NO: 47 - DNA, 17 bp, Homo sapiens
catccagggatctcagc

SEQ ID NO: 48 - DNA, 27 bp, Homo sapiens
catccagggatctcagaaattattagt

SEQ ID NO: 49 - DNA, 21 bp, Homo sapiens
catccagggatctcagcatcc

SEQ ID NO: 50 - DNA, 21 bp, Homo sapiens
catccagggatctcagcatcc

SEQ ID NO: 51 - DNA, 20 bp, Homo sapiens
catccagggatctcagcatc

SEQ ID NO: 52 - DNA, 17 bp, Homo sapiens
catccagggatctcagc

SEQ ID NO: 53 - DNA, 16 bp, Homo sapiens
catccagggatctcag

SEQ ID NO: 54 - DNA, 31 bp, Homo sapiens; identical to SEQ ID NO: 42

SEQ ID NO: 55 - DNA, 33 bp, Homo sapiens; identical to SEQ ID NO: 40

SEQ ID NO: 56 - DNA, 33 bp, Homo sapiens; identical to SEQ ID NO: 40

SEQ ID NO: 57 - DNA, 30 bp, Homo sapiens; identical to SEQ ID NO: 41

SEQ ID NO: 58 - DNA, 32 bp, Homo sapiens
catccagggatctcagaaattattagtacatc

SEQ ID NO: 59 - DNA, 29 bp, Homo sapiens
catccagggatctcagaaattattagtac

SEQ ID NO: 60 - DNA, 33 bp, Homo sapiens; identical to SEQ ID NO: 40

SEQ ID NO: 61 - DNA, 17 bp, Homo sapiens
catccagggatctcaga

SEQ ID NO: 62 - DNA, 29 bp, Homo sapiens
catccagggatctcagaaattattagtac

SEQ ID NO: 63 - DNA, 30 bp, Homo sapiens; identical to SEQ ID NO: 41

SEQ ID NO: 64 - DNA, 33 bp, Homo sapiens; identical to SEQ ID NO: 40

SEQ ID NO: 65 - DNA, 19 bp, Homo sapiens
catccagggatctcagaaa

SEQ ID NO: 66 - DNA, 22 bp, Homo sapiens
catccagggatctcagcatccc

SEQ ID NO: 67 - DNA, 42 bp, Homo sapiens
catccagggatctcagaaattattagtacatcccacagtgaa

SEQ ID NO: 68 - DNA, 72 bp, Homo sapiens
aaacatcaagtctagtgtaactgtttcttcttcaaggtgatttgcattttattcctgaatgcctgagggttc

SEQ ID NO: 69 - DNA, 73 bp, Homo sapiens
gaaacatcaagtctagtgtaactgtttcttcttcaaggtgatttgcattttattcctgaatgcctgagggttc

SEQ ID NO: 70 - DNA, 70 bp, Homo sapiens
acatcaagtctagtgtaactgtttcttcttcaaggtgatttgcattttattcctgaatgcctgagggttc

SEQ ID NO: 71 - DNA, 74 bp, Homo sapiens
ggaaacatcaagtctagtgtaactgtttcttcttcaaggtgatttgcattttattcctgaatgcctgagggttc

SEQ ID NO: 72 - DNA, 71 bp, Homo sapiens
aacatcaagtctagtgtaactgtttcttcttcaaggtgatttgcattttattcctgaatgcctgagggttc

SEQ ID NO: 73 - DNA, 71 bp, Homo sapiens; identical to SEQ ID NO: 72

SEQ ID NO: 74 - DNA, 65 bp, Homo sapiens
aagtctagtgtaactgtttcttcttcaaggtgatttgcattttattcctgaatgcctgagggttc

SEQ ID NO: 75 - DNA, 69 bp, Homo sapiens
catcaagtctagtgtaactgtttcttcttcaaggtgatttgcattttattcctgaatgcctgagggttc

SEQ ID NO: 76 - DNA, 72 bp, Homo sapiens; identical to SEQ ID NO: 68

SEQ ID NO: 77 - DNA, 69 bp, Homo sapiens; identical to SEQ ID NO: 75

SEQ ID NO: 78 - DNA, 74 bp, Homo sapiens; identical to SEQ ID NO: 71

SEQ ID NO: 79 - DNA, 70 bp, Homo sapiens; identical to SEQ ID NO: 70

SEQ ID NO: 80 - DNA, 73 bp, Homo sapiens; identical to SEQ ID NO: 69

SEQ ID NO: 81 - DNA, 70 bp, Homo sapiens; identical to SEQ ID NO: 70

SEQ ID NO: 82 - DNA, 71 bp, Homo sapiens; identical to SEQ ID NO: 72

SEQ ID NO: 83 - DNA, 73 bp, Homo sapiens; identical to SEQ ID NO: 69

SEQ ID NO: 84 - DNA, 63 bp, Homo sapiens
gtctagtgtaactgtttcttcttcaaggtgatttgcattttattcctgaatgcctgagggttc

SEQ ID NO: 85 - DNA, 56 bp, Homo sapiens
gtaactgtttcttcttcaaggtgatttgcattttattcctgaatgcctgagggttc

SEQ ID NO: 86 - DNA, 72 bp, Homo sapiens; identical to SEQ ID NO: 68

SEQ ID NO: 87 - DNA, 72 bp, Homo sapiens; identical to SEQ ID NO: 68

SEQ ID NO: 88 - DNA, 73 bp, Homo sapiens; identical to SEQ ID NO: 69

SEQ ID NO: 89 - DNA, 52 bp, Homo sapiens
ctgtttcttcttcaaggtgatttgcattttattcctgaatgcctgagggttc

SEQ ID NO: 90 - DNA, 62 bp, Homo sapiens
tctagtgtaactgtttcttcttcaaggtgatttgcattttattcctgaatgcctgagggttc

SEQ ID NO: 91 - DNA, 67 bp, Homo sapiens
tcaagtctagtgtaactgtttcttcttcaaggtgatttgcattttattcctgaatgcctgagggttc

SEQ ID NO: 92 - DNA, 52 bp, Homo sapiens; identical to SEQ ID NO: 89

SEQ ID NO: 93 - DNA, 30 bp, Homo sapiens
tgcattttattcctgaatgcctgagggttc

SEQ ID NO: 94 - DNA, 58 bp, Homo sapiens
gtgtaactgtttcttcttcaaggtgatttgcattttattcctgaatgcctgagggttc

SEQ ID NO: 95 - DNA, 83 bp, Homo sapiens
tgttgctgtggaaacatcaagtctagtgtaactgtttcttcttcaaggtgatttgcattttattcctgaatgcctgagggttc

SEQ ID NO: 96 - DNA, 25 bp, Homo sapiens
ctcttcgcccccgaggatcagtctt

SEQ ID NO: 97 - DNA, 25 bp, Homo sapiens
gaaggcggcagtccttgtgcttttc

SEQ ID NO: 98 - DNA, 26 bp, Homo sapiens
agtgtgacgtggacatccgcaaagac

SEQ ID NO: 99 - DNA, 26 bp, Homo sapiens
gcttgctgatccacatctgctggaag

SEQ ID NO: 100 - DNA, 21 bp, Homo sapiens
ggatgctgatgagggtcaaga

SEQ ID NO: 101 - DNA, 24 bp, Homo sapiens
ttcccacacagctatctcataagg

SEQ ID NO: 102 - DNA, 19 bp, Homo sapiens
atgtcccaagtttcaggtg

SEQ ID NO: 103 - DNA, 20 bp, Homo sapiens
gaaggaaagcccccctgtaa

SEQ ID NO: 104 - DNA, 18 bp, Homo sapiens
gatcggcatgggctcatc

SEQ ID NO: 105 - DNA, 17 bp, Homo sapiens
cgatactccagatgagg

SEQ ID NO: 106 - DNA, 16 bp, Homo sapiens
tgcatcgggcccaatg

SEQ ID NO: 107 - DNA, 20 bp, Homo sapiens
aactgagccaggccttacca

SEQ ID NO: 108 - DNA, 17 bp, Homo sapiens
ctcatggttcacaaaag

SEQ ID NO: 109 - DNA, 18 bp, Homo sapiens
caacctgctccggcacat

SEQ ID NO: 110 - DNA, 20 bp, Homo sapiens
tgcagaggtggcatttgaag

SEQ ID NO: 111 - DNA, 15 bp, Homo sapiens
aagctgcattccggg

SEQ ID NO: 112 - DNA, 25 bp, Homo sapiens
gcgaagctctttagaggaacataaa

SEQ ID NO: 113 - DNA, 19 bp, Homo sapiens
aggcccatgctttccaagt

SEQ ID NO: 114 - DNA, 14 bp, Homo sapiens
agcgctgccacaac

SEQ ID NO: 115 - DNA, 24 bp, Homo sapiens
aatcacagtgaaatggcagaagac

SEQ ID NO: 116 - DNA, 22 bp, Homo sapiens
tctgtccagcacgagagatctc

SEQ ID NO: 117 - DNA, 18 bp, Homo sapiens
tgtgcaagataggatcag

SEQ ID NO: 118 - DNA, 24 bp, Homo sapiens
agacagaggatcaagggctttaga

SEQ ID NO: 119 - DNA, 21 bp, Homo sapiens
ggcgcatctttctctgtgatt

SEQ ID NO: 120 - DNA, 17 bp, Homo sapiens
agcactccttcaatatg

SEQ ID NO: 121 - DNA, 18 bp, Homo sapiens
tcgggaggacagcaaagc

SEQ ID NO: 122 - DNA, 18 bp, Homo sapiens
tgtcggacaggcccttgt

SEQ ID NO: 123 - DNA, 17 bp, Homo sapiens
ccaagagtgacagaggg

SEQ ID NO: 124 - DNA, 26 bp, Homo sapiens
ccacagggcaagtcatccacattttg

SEQ ID NO: 125 - DNA, 30 bp, Homo sapiens
cagaccatagagtccctcctaggggaaaaa

SEQ ID NO: 126 - DNA, 30 bp, Homo sapiens
ttcttagaagtctggagtctgtgaaggtca

SEQ ID NO: 127 - protein, 12 aa, Homo sapiens
DRYEFSSHITRG

SEQ ID NO: 128 - DNA, 12 bp, Homo sapiens
aaaagaaaaccc

SEQ ID NO: 129 - DNA, 10 bp, Homo sapiens
cccttgggag

SEQ ID NO: 130 - DNA, 24 bp, Homo sapiens
ggactttccgggggggtgtctttc

SEQ ID NO: 131 - protein, 6 aa, Homo sapiens
DRYEFS

(19) United States
(12) Patent Application Publication
(10) Pub. No.: US 2010/0240057 A1
(43) Pub. Date: Sep. 23, 2010

(54) METHODS AND COMPOSITIONS FOR THE DIAGNOSIS AND TREATMENT OF CHRONIC MYELOID LEUKEMIA AND ACUTE LYMPHOBLASTIC LEUKEMIA

(75) Inventors: James Downing, Memphis, TN (US); Charles Mullighan, Memphis, TN (US)

Correspondence Address: ALSTON AND BIRD LLP;ST. JUDE CHILDREN'S RESEARCH HOSPITAL, BANK OF AMERICA PLAZA, 101 SOUTH TRYON STREET, SUITE 4000, CHARLOTTE, NC, 28280-4000, US

(73) Assignee: St. Jude Children's Research Hospital, Memphis, TN (US)

(21) Appl. No.: 12/738,759

(22) PCT Filed: Nov. 6, 2008
(86) PCT No.: PCT/US08/82592

§ 371 Date: Apr. 19, 2010

Related U.S. Application Data
(60) Provisional application No. 61/002,351, filed on Nov. 8, 2007.
(60) Provisional application No. 60/986,530, filed on Nov. 8, 2007.
(60) Provisional application No. 61/012,554, filed on Dec. 10, 2007.

(51) Int. Cl. C12Q 1/68

(52) U.S. Cl. 435/6

(57) ABSTRACT

Compositions and methods for the identification, prognosis, classification, treatment, and diagnosis of leukemia or a genetic predisposition to leukemia are provided. The present invention is based on the discovery of various genomic abnormalities of the IKZFl gene which are shown herein to be associated with acute lymphoblastic leukemia (ALL), more particularly, associated with BCR-ABL1 positive ALL and/or shown to be associated with chronic myeloid leukemia (CML), more particularly, associated with blast crisis chronic myeloid leukemia (BC-CML) and/or the likelihood of progression into blastic transformation of CML. These various genomic abnormalities of the IKZFl gene can further be used as prognostic markers to identify a subgroup of ALL having very poor outcomes. Such genomic abnormalities of IKZFl find use in methods and compositions useful in the identification and/or prognosis and/or predisposition and/or treatment of ALL, more particularly, BCR-ABL1 positive ALL and/or in the identification and/or prognosis and/or predisposition and/or treatment of CML, more particularly, of BC-CML and/or the likelihood of progression into blastic transformation of CML and/or as prognostic markers to identify a subgroup of ALL having very poor outcomes.

### FIELD OF THE INVENTION

The present invention relates generally to the detection and/or prognosis and/or diagnosis and/or treatment of sub-types of acute lymphoblastic leukemia and/or chronic myeloid leukemia.

### BACKGROUND OF THE INVENTION

Leukemia's are classified into four multiple groups or types, including: acute myeloid leukemia (AML), acute lymphatic leukemia (ALL), chronic myeloid (CML) and chronic lymphocytic leukemia (CLL). Within these groups, several subcategories can be further identified using a panel of standard diagnostic techniques. These different subcategories of leukemia are associated with varying clinical outcomes and therefore are the basis for different treatment strategies.

The development of new specific drugs and treatment approaches requires the identification of specific subtypes that may benefit from a distinct therapeutic protocol and, thus, can improve outcome of distinct subsets of leukemia. As it is mandatory for the patients suffering from these specific leukemia subtypes to be identified as fast as possible so that the best therapy can be applied, diagnostics today must accomplish sub-classification with maximal precision. Thus, methods and compositions are needed in the art to provide means for additional leukemia diagnostic and prognostic markers.

### SUMMARY OF THE INVENTION

Compositions and methods for the identification, prognosis, classification, diagnosis and/or treatment of leukemia or a genetic predisposition to leukemia are provided. In one embodiment, the present invention is based on the discovery of multiple genomic abnormalities of the IKZF1 (Ikaros) gene which are shown herein to be associated with acute lymphoblastic leukemia (ALL), more particularly, with BCR-ABL1 positive ALL, and to be associated with chronic myeloid leukemia (CML), more particularly, a subtype of CML termed blast crisis chronic myeloid leukemia (BC-CML). In another embodiment, the present invention demonstrates that the genomic abnormalities of the IKZF1 gene can be used as prognostic markers to identify a subgroup of BCR-ABL1 negative ALL having very poor outcomes. The present invention therefore provides compositions comprising polynucleotides, including both genomic sequences of the various IKZF1 genomic abnormalities disclosed herein and any transcripts encoded thereby. Such polynucleotides comprising the genomic abnormalities of the IKZF1 gene find use, for example, as biomarkers for use in methods for detecting genomic abnormalities which are associated with ALL, more specifically, which are associated with BCR-ABL1 positive ALL, and/or for detecting genomic abnormalities which are associated with CML, more particularly, with BC-CML or the likelihood of progression into blastic transformation of CML. In another embodiment, the biomarkers can be used as a prognostic markers to identify a subgroup of ALL having very poor outcomes. Accordingly, the present invention encompasses methods and compositions useful in the identification and/or the prognosis and/or predisposition and/or treatment of a subject with ALL and/or a subject with CML, more particularly, with BC-CML or the likelihood of progression into blastic transformation of CML.

The compositions of the invention can further be employed in methods for selecting a therapy for a subject affect by leukemia. Including, for example, selecting an appropriate therapy for ALL and/or selecting a therapy for CML, more particularly, a therapy for a patient with BC-CML or for a patient with CML having a likelihood of progression into blastic transformation of CML. Further provided are methods for identifying agents that target a polypeptide expressed from the IKZF1 genomic abnormality. Thus, methods to screen for compounds that can serve as molecular targets for drugs useful in modulating the activity of the polypeptides expressed from the IKZF1 genomic abnormalities are provided. Such compounds can find use in treating ALL and/or treating a subject with CML, more particularly, treating a subject with BC-CML or a patient having CML with the likelihood of progression into blastic transformation of CML. Accordingly, the present invention encompasses methods and compositions useful in the identification and/or the prognosis and/or predisposition and/or treatment of ALL and/or CML, more specifically, BC-CML.

### DESCRIPTION OF THE FIGURES

FIG. 1A-C depicts IKZF1 deletions in BCR-ABL1 ALL. a, Domain structure of IKZF1. Coding exons 3-5 encode four N-terminal zinc fingers (black boxes) responsible for DNA binding. The C-terminal zinc fingers encoded by exon 7 are essential for homo- and heterodimerization. b. Genomic organization of IKZF1 and location of each of the 36 deletions observed in BCR-ABL1 B-progenitor ALL. Each line depicts the deletion(s) observed in each case. In five cases, two discontiguous deletions were observed. Hemizygous deletions are solid lines and homozygous deletions dashed. Arrows indicate deletions extending beyond the limits of the figure. The exact boundaries of the deletions were defined by genomic qPCR, and for IKZF1 Δ3-6, by long-range genomic PCR. c, dChipSNP raw log₂ratio copy number data depicting IKZF1 deletions for 29 BCR-ABL1 cases and 3 B-progenitor ALL cell lines.

FIG. 2 provides the structure of Ikaros isoforms. IKZF1 has 8 exons (0-7), of which exons 1-7 (gray boxes) are coding. Exons 3-5 encode four N-terminal zinc fingers (black boxes) responsible for DNA binding. The C-terminal zinc fingers encoded by exon 7 are essential for homo- and heterodimerization. Two novel Ikaros isoforms that arise from genomic deletions of exons 2-6 (Ik9) or 1-6 (Ik10) were identified. Neither is translated into a detectable protein in ALL blasts.

FIGS. 3A and B provides Ikaros isoforms in ALL blasts. a, Domain structure of the IKZF1 isoforms detected by RT-PCR, examples of which are shown in panel b. b, RT-PCR for IKZF1 transcripts (using exon 0 and 7 specific primers) in representative cases with various IKZF1 genomic abnormalities. Each case expressing an aberrant isoform had a corresponding IKZF1 genomic deletion. IKZF1 Δ3-6 was also detected in the BCR-ABL1 ALL cell lines SUP-B15 and OP1, and Δ1-6 in the ALL cell line 380. Western blotting for Ikaros using a C-terminus specific polyclonal antibody. Ik6 was only detectable in cases with IKZF1 Δ3-6. The Δ1-6 and Δ2-6 deletions do not produce a detectable protein. In three cases with multiple focal hemizygous deletions involving different regions of IKZF1 (BCR-ABL-SNP-#26, -#29, and -#31), no wild type Ikaros was detectable by RT-PCR or western blotting, indicating that the deletions involve both copies of IKZF1 in each case.

FIG. 4A-C demonstrates that sequencing of RT-PCR products confirms the expression of non-DNA binding Ikaros isoforms in IKZF1 deleted cases. The junction of BCR-ABL-SNP-#34 is set forth in SEQ ID NO:2. The junction of BCR-ABL-SNP-#19 is set forth in SEQ ID NO:3. The junction of BCR-ABL-SNP-#23 is set forth in SEQ ID NO:4.

FIG. 5 shows that quantitative RT-PCR for the Ik6 transcript confirms that expression of this isoform is restricted to cases with IKZF1 Δ3-6. Exact Wilcoxon-Mann-Whitney P value is shown.

FIG. 6A-D shows IKZF1 deletions in blast crisis CML. a, Examples of peripheral blood smears of chronic phase and (myeloid) blast crisis CML. b, dChipSNP log₂ratio copy number heatmaps of four CML cases showing acquisition of IKZF1 deletions at progression to blast crisis. c, Pherograms of IKZF1 exon 7 sequencing demonstrating acquisition of the c. 1520C>A, p.Ser507X mutation at blast crisis in case CML-#7. As this case has a concomitant hemizygous IKZF1 deletion involving exon 7, the mutation appears homozygous. The junction for CML-#7-CP is set forth in SEQ ID NO:5 and SEQ ID NO: 127 and the junction for CML-#7-BC is set forth in SEQ ID NO:6 and SEQ ID NO:131.

FIG. 7A-C presents pherograms of sequencing of IKZF1 Δ3-6 breakpoints. Regions matching the reference genomic IKZF1 sequence are shown by arrows, separated by additional nucleotides not matching the consensus sequence. The sequence for BCR-ABL-SNP-#4 is set forth in SEQ ID NO:37. The sequence for BCR-ABL-SNP-#1 is set forth in SEQ ID NO:38. The sequence for BCR-ABL-SNP-#7 is set forth in SEQ ID NO:39.

FIG. 8 shows genomic PCR of IKZF1 Δ3-6. Primers used were C814 and C814; products were then directly sequenced to characterize the sequence flanking deletion breakpoints.

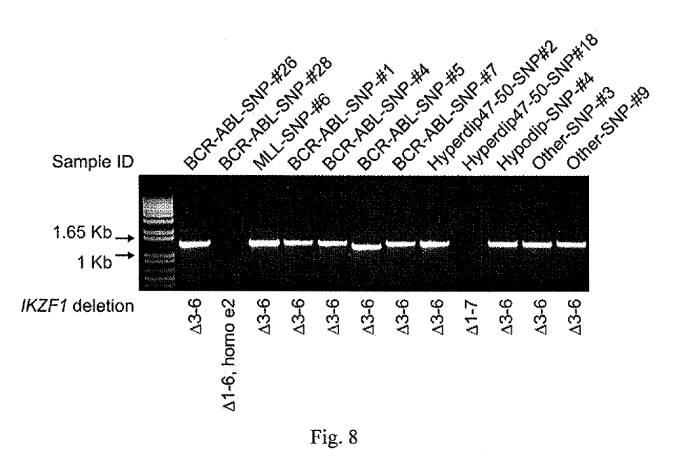

FIG. 9 shows the PAX5 deletions in P9906 ALL. Specifically, the Raw log ratio copy number at the PAX5 locus is shown for all cases with an IKZF1 copy number alterations (CAN). Blue is deletion, and red gain. HD, hyperdiploid.

FIG. 10 shows the IKZF1 deletions in P9906 ALL. Specifically, the Raw log₂ ratio copy number at the IKZF1 locus is shown for all cases with an IKZF1 CNA.

FIG. 11A-E shows the gene set enrichment analysis (GSEA) of poor outcome P9906 ALL, poor outcome St Jude ALL, and BCR-ABLJ positive St Jude ALL. A, Genes are ranked (bottom of panel, green) based on correlation between expression and class distinction (here SPC predicted poor outcome v non-poor outcome). GSEA then determines if the members of a gene set (here a gene set of the top 100 upregulated genes in St Jude poor outcome ALL) are randomly distributed in the ranked gene list, or primarily found at the top or bottom. Occurrences of members of the gene set in the ranked gene list are shown as vertical black lines above the ranked signature. An enrichment score ES is calculated that reflects the degree to which a gene set is overrepresented at the top or bottom of the entire ranked list. The ES is a running sum, Kolmogorov-Smirnov like statistic calculated by walking down list L and increasing the statistic when a gene in S is encountered, and decreasing it when it is not. The magnitude of the increment depends on the strength of association with phenotype, and the ES is the maximum deviation from zero encountered in the random walk, and is depicted as a red curve. The “leading edge” genes are those members of the gene set responsible for the observed enrichment, and are those hits occurring to the left of the vertical dotted red line. The significance level of ES is calculated by phenotype-based permutation testing, and when a database of gene sets are evaluated, as in this analysis, the significance level is adjusted for multiple hypothesis testing by calculation of a false discovery rate (FDR). Here there is highly significant enrichment of the St Jude poor outcome upregulated gene set in the P9906 poor outcome signature. B, enrichment of the P9906 poor outcome upregulated gene set in the St Jude poor outcome signature. These analyses demonstrate similarity between the signatures of P9906 and St Jude poor outcome ALL. C, enrichment of the P9906 poor outcome upregulated gene set in St Jude BCR-ABL1 positive ALL, demonstrating similarity of P9906 poor outcome (BCRABL1 negative) and St Jude BCR-ABL1 positive signatures. D, heatmap of St Jude ALL and P9906 poor outcome upregulated genes, corresponding to the GSEA plot in C. B-A, BCR-ABL1 positive; E-R, ETV6-RUNXJ positive; H50, high hyperdiploid; Hypo, hypodiploid; T-P, TCF3-PBX1. Increased expression genes of the P9906 poor outcome gene set is seen in BCR-ABL1 ALL; “leading edge” genes responsible for the enrichment are shown at the right of the panel. E, negative enrichment of B cell antigen receptor/signal transduction genes in P9906 poor outcome ALL.

FIG. 12 shows the primary structure of IKAROS, showing location of the six zinc fingers (green) and missense (▾), frameshift (♦), and nonsense (▴) mutations identified in the P9906 cohort.

FIG. 13A-D shows the associations between the supervised principal components derived CNA predictors and outcome in P9906 and St Jude cohorts. P9906 predictor and cumulative incidence of any adverse events (A) and any relapse (B) in the St Jude cohort. St Jude predictor and cumulative incidence of adverse events (C) and relapse (D) in the P9906 cohort. HR, SPC predicted poor outcome; LR, SPC predicted poor outcome.

FIG. 14A-I shows the association of IKZF1, EBF1 and BTLA/CD200 genetic alterations and incidence of any relapse in the P9906 cohort (A-C), the entire St Jude B-ALL cohort (D-F), and the St Jude cohort after exclusion of BCR-ABL1 positive cases (G-I). Only IKZF1 abnormalities were associated with outcome in both P9906 and St Jude cohorts.

FIG. 15 shows the clonal relationship of diagnosis and relapse samples in ALL. The majority of relapse cases have a clear relationship to the presenting diagnostic leukemic clone, either arising through the acquisition of additional genetic lesions, or more commonly, arising from a ancestral (pre-diagnosis) clone. In the latter scenario, the relapse clone retains some but not all of the lesions found in the diagnostic sample, while acquiring new lesions. Lesion specific backtracking studies revealed that in most cases the relapse clone exists as a minor sub-clone within the diagnostic sample prior to the initiation of therapy. In only a minority of ALL cases does the relapse clone represent the emergence of a genetically distinct and thus unrelated second leukemia.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I Genomic Abnormalities of IKZF1

In one embodiment, the present invention has identified various genomic abnormalities in the IKZF1 gene that are correlated with ALL, more particularly, with BCR-ABL1 positive ALL, and that are correlated with CML, more particularly, BC-CML or the likelihood of progression into blastic transformation of CML. In addition, the genomic abnormalities in the IKZF1 gene can further be used as prognostic markers of ALL, more particularly, prognostic markers for subtypes of ALL having very poor outcomes, including, the B-progenitor ALL subtypes, including BCR-ABL1(+) and BCR-ABL1(−) subtypes. Various methods and compositions that allow for the direct detection of such genomic abnormalities in IKZF1 are provided. Compositions of the invention include IKZF1 polynucleotides and variants and fragments thereof that can be used to detect the chromosomal abnormalities in the IKZF1 gene that are associated with ALL, more particularly, with BCR-ABL1 positive ALL, and that are associated with CML, more particularly, BC-CML and that are associated with the prognosis of subtype of ALL having very poor outcomes, including, B-progenitor ALL. “Acute lymphoblastic leukemia” or “ALL” comprises a heterogeneous group of leukemic disorders characterized by recurring chromosomal abnormalities including translocations, trisomies and deletions. As used herein “BCR-ABL1” comprises an ALL subtype that is characterized by the presence of the Philadelphia chromosome arising from the t(9; 22)(q34; q11.2) translocation, which encodes the constitutively activated BCR-ABL1 tyrosine kinase. See, for example, Riberio et al. (1987) Blood 70:948 and Gleissner et al. (2002) Blood 99:1536, both of which are herein incorporated by reference. Chronic myeloid leukemia is a myeloproliferative disorder characterized by the presence of the BCR-ABL1 transcript in most cases. CML typically presents as an indolent chronic phase, and subsequently progresses through a more aggressive accelerated phase, eventually terminating in an overt blastic phase (blast crisis), which may be of lymphoid or myeloid lineage.

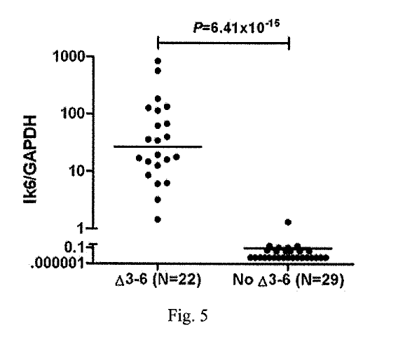

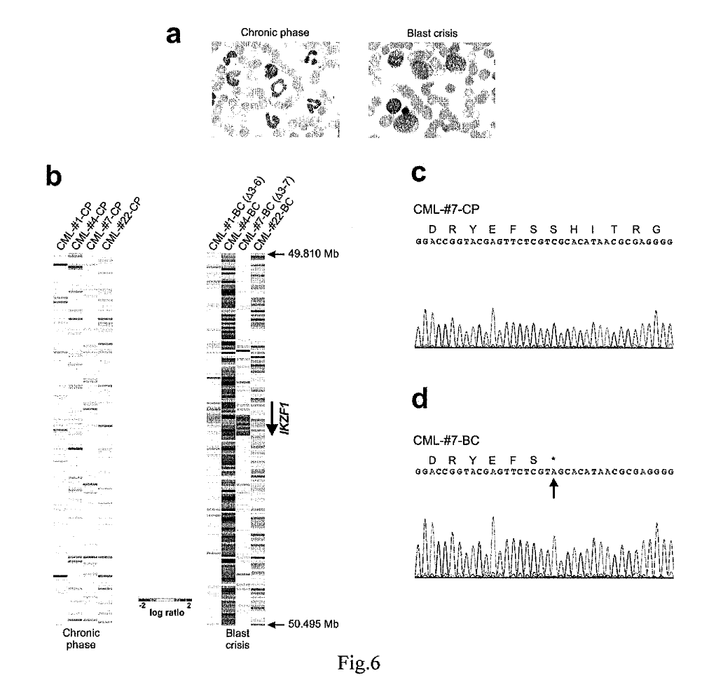

As used herein, the “IKZF1” gene or the “Ikaros” gene refers to a genomic polynucleotide that encodes an IKZF1 polypeptide, where the encoded polypeptide is a member of a family of zinc finger nuclear proteins that is required for normal lymphoid development. The IKZF1 polypeptide has a central DNA-binding domain consisting of four zinc fingers, and a homo- and heterodimerization domain consisting of the two C-terminal zinc fingers (FIGS. 5 and 6). See, for example, Hahm et al. (1994) Mol Cell Biol 14 (11): 7111; Molnar et al., (1994) Mol Cell Biol 14 (12):8292; Molnar et al., (1996) J Immunol 156 (2): 585; Rebollo et al. (2003) Immunol Cell Biol 81 (3): 171; Sun (1996) Embo J 15 (19):5358, each of which is herein incorporated by reference. The human genomic sequence of IKZF1 is set forth in SEQ ID NO:1. The various exons/introns of the IKZF1 genomic sequence are further illustrated in SEQ ID NO:1. It will be appreciated by those skilled in the art that DNA sequence polymorphisms may exist within a population (e.g., the human population). Such genetic polymorphisms in a polynucleotide comprising the IKZF1 gene as set forth in SEQ ID NO:1 may exist among individuals within a population due to natural allelic variation. The term IKZF1 gene encompasses such natural variations.

The term “gene” refers to a nucleic acid (e.g., DNA) sequence that comprises coding sequences necessary for the production of a polypeptide, precursor, or RNA (e.g., rRNA, tRNA). The term also encompasses the coding region of a structural gene and the sequences located adjacent to the coding region on both the 5′ and 3′ end which allow for the expression of the sequence. Sequences located 5′ of the coding region and present on the mRNA are referred to as 5′ non-translated sequences. Sequences located 3′ or downstream of the coding region and present on the mRNA are referred to as 3′ non-translated sequences. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, a “genomic abnormality” refers to any alteration in the genomic sequence. Such rearrangements include a point mutation, a deletion, a substitution, or amplification of the gene, including a complete or partial deletion or amplification of any one or any combination of the promoter, the 5′ regulatory region of the IKZF1 gene, the coding region of the IKZF1 gene, and/or the 3′ regulatory region of the IKZF1 gene. Substitutions and/or deletions and/or additions can range from 1, 2, 3, 5, 10, 30, 60, 100, 200, 300, 400, 500 nucleotides in length or higher. Rearrangements can further include an insertion into the genomic sequence in any one or any combination of the various regions outlined above. In specific embodiments, the genomic abnormality comprises a deletion of the entire IKZF1 gene. In other embodiments, the genomic abnormality comprises an intragenic deletion. In other embodiments, the genomic abnormality comprises sequence mutations (nucleotide substitutions) of the gene.

As used herein, a “genomic abnormality” of IKZF1 is characterized phenotypically by the association of the genomic abnormality with ALL and/or CML, more particularly, with BCR-ABL1 positive ALL and/or with a BC-CML; the likelihood of progression into blastic transformation of CML. In still other embodiments, the genomic abnormality of the IKZF1 gene is characterized phenotypically by the association of the genomic abnormality with a subgroup of ALL having very poor outcomes, including, BCR-ABL1 positive and BCR-ABL1 negative B-progenitor ALL subtypes.

The term “intragenic deletion” refers to any internal deletion in the genomic DNA of a gene. Thus, the term “intragenic deletion of IKZF1” refers to any internal deletion in the genomic DNA comprising the IKZF1 gene. As used herein, an intragenic deletion of an IKZF1 allele is characterized phenotypically by the association of the intragenic deletion with ALL and/or CML, more particularly, with BCR-ABL1 positive ALL and/or BC-CML or the likelihood of progression into BC-CML. At the genetic level, the intragenic deletion is part of the genetic make-up of the cell (contained within the genomic DNA). In specific embodiments, the intragenic deletion of IKZF1 comprises an internal deletion of various exons including, for example, a deletion of at least one of exon 0, exon 1, exon 2, exon 3, exon 4, exon 5, exon 6, and/or exon 7 of the IKZF1 gene or any combination thereof. It is recognized that as used herein, a deletion of an exon or intron can encompass both the complete absence of the recited exon or intron sequence, or the absence of at least a fragment of the full exon or full intron. In other words, the chromosomal break can occur anywhere within the recited exon or in the flanking intron. The exons of the human IKZF1 gene are designated in the genomic sequence of the human IKZF1 gene in SEQ ID NO: 1.

In specific embodiments, the genomic abnormality of the IKZF1 gene comprises a deletion of exon 3 through exon 6. In further embodiments, the genomic abnormality resulting in the deletion of exon 3 through exon 6 results from a proximal chromosomal break point occurring within intron 2 and a distal chromosomal break point occurring within intron 6. See, for example, Table 9. The specific genomic abnormality depicted in Table 9 is referred to herein as IKZF1Δexon3-6 or IK6.

Additional, non-limiting examples of genomic abnormalities of the IKZF1 gene are shown throughout the experimental section. For instance, the genomic abnormality of the IKZF1 gene can comprise a deletion of exon 2 through exon 6 (referred to here in as IKZF1Δexon2-6 or Ik9). In such rearrangements, the genomic abnormality could result from a proximal chromosomal break point occurring in intron 1 or in exon 2 and a distal chromosomal break point occurring in intron 6 or exon 6. In still other examples, the genomic abnormality of the IKZF1 gene can comprise a deletion of exon 1 through exon 6 (referred to herein as IKZF1Δexon1-6 or Ik6). In such rearrangements, the genomic abnormality could result from a proximal chromosomal break point occurring upstream of exon 1 or in exon 1 and a distal chromosomal break point occurring in intron 6 or exon 6.

The term “intragenic substitution” refers to any internal substitution in the genomic DNA of a gene. Thus, the term “intragenic substitution of IKZF1” refers to any internal substitution or point mutations in the genomic DNA comprising the IKZF1 gene. As used herein, an intragenic substitution of an IKZF1 allele is characterized phenotypically by the association of the intragenic deletion with ALL and/or CML, more particularly, with BCR-ABL1 positive ALL; and/or with BC-CML or progression into blastic transformation CML; and/or with a subgroup of ALL with very poor outcomes.

The term “intragenic addition” refers to any internal addition in the genomic DNA of a gene. Thus, the term “intragenic addition of IKZF1” refers to any internal addition in the genomic DNA comprising the IKZF1 gene. As used herein, an intragenic addition of an IKZF1 allele is characterized phenotypically by the association of the intragenic addition with ALL and/or CML, more particularly, with BCR-ABL1 positive ALL; and/or with BC-CML or progression into blastic transformation CML; and/or with a subgroup of ALL with very poor outcomes.

Further provided are a series of genetic abnormality which are shown to be associated with CML in blast crisis which, in specific embodiments, comprise point mutations in IKZF1.

In specific embodiments, the genomic abnormality in the IKZF1 gene results in the expression of a dominate negative isoform of the IKZF1 polypeptide. In specific embodiments, the dominant negative isoform of the IKZF1 polypeptide lacks the ability to bind DNA. In other embodiments, the genomic abnormality in the IKZF1 gene results in the complete loss of expression of the IKZF1 polypeptide. In still further embodiments, the genomic abnormality of the IKZF1 gene results from a recombinase activating gene (RAG) mediated recombination event. Representative methods to assay for such activities are disclosed herein in the experimental section.

The term “junction of a genomic abnormality” refers to the region of the polynucleotide which is joined following the occurrence of the genomic abnormality. In view of the characterization of the various chromosomal abnormalities of IKZF1 disclosed herein, novel polynucleotides are provided that comprise the novel polynucleotide junctions of IKZF1 that occur following the various genomic abnormalities.

In specific embodiments, the polynucleotides comprising the IKZF1 genomic abnormalities or active variants and fragments thereof, do not encode an IKZF1 polypeptide, but rather have the ability to specifically detect the IKZF1 genomic abnormality in the genomic DNA of a biological sample, and thereby allow for the identification/classification and/or the prognosis and/or predisposition of the biological sample to ALL, more particularly, BCR-ABL1 positive ALL and/or to CML, more particularly, to BC-CML or the likelihood of progression of blastic transformation of CML. In other embodiments, the polynucleotides comprising IKZF1 genomic abnormalities or active fragments or variants thereof allow for the detection of prognostic markers of a subtype of ALL having very poor outcomes. Various methods and compositions to carry out such methods are disclosed elsewhere herein.

In specific embodiments, detecting the IKZF1 genomic abnormalities find use in selecting a therapy for a subject affect by leukemia. Thus, upon the detection of the IKZF1 genomic abnormality, and in specific embodiments, the identification of the specific IKZF1 genomic abnormality, a therapy may be selected or customized for the subject in view of the IKZF1 genomic abnormalities.

In one embodiment, a method for making a prognosis of an acute lymphoblastic leukemia having a poor outcome in a patient is provided. Thus, the genomic abnormalities of the IKZF1 gene can be used as prognostic markers that allow for the prediction of the probable course and outcome of ALL and/or the likelihood of recovery from the disease. As demonstrated herein, the genomic abnormalities of IKZF1 identify a subgroup of ALL with very poor outcomes. Thus, the identification of genomic abnormalities can be used to improve the ability to accurately stratify patients for appropriate therapy. Such a prognosis can be used to improve outcome prediction, predict risk of relapse, predict risk of treatment failure, and/or design treatment regimes. Such methods comprise assaying the nucleic acid complement of a biological sample for a genomic abnormality in the IKZF1 gene. Such methods comprise detecting the genomic abnormality of the IKZF1 gene in the nucleic acid complement of the biological sample, where the presence of the genomic abnormality of the IKZF1 gene is indicative of a subgroup of ALL with poor outcomes. A prognosis of the patient's ALL based on the genomic abnormalities of IKZF1 gene is then provided.

As used herein, the “nucleic acid complement” of a sample comprises any polynucleotide contained in the sample. The nucleic acid complement that is employed in the methods and compositions of the invention can include all of the polynucleotides contained in the sample or any fraction thereof. For example, the nucleic acid complement could comprise the genomic DNA and/or the mRNA and/or cDNAs of the given biological sample. Thus, the genomic abnormalities in the IKZF1 gene can be detected in the genomic DNA or through the transcribed products thereof.

Methods are further provided that allow for determining the progression of chronic myeloid leukemia in a patient. In one embodiment, a method for classifying a cell sample as BC-CML or having a likelihood of progression into blastic transformation of CML is provided. Such methods can comprise determining if the biological sample comprises a genomic abnormality of the IKZF1 gene. The presence of the genomic abnormality of the IKZF1 gene is indicative of progression into blastic transformation of CML. Thus, the methods and compositions of the invention allow for one to distinguish patients having a likelihood of progression of blastic transformation of CML and/or to determine the general course of treatment for these patients.

### II. Methods of Detecting Genomic Abnormalities

Various methods and compositions for identifying a genomic abnormality in the IKZF1 gene are provided. Such methods find use in identifying and/or detecting such rearrangements in any biological material and thus allow for the identification, prognosis, classification, treatment, and/or diagnosis of leukemia or a genetic predisposition to ALL, more particularly, BCR-ABL1 positive ALL and/or to CML, more particularly, with BC-CML or the likelihood of progression into blastic transformation of CML. Such methods further find use to detect a subset of BCR-ABL1 positive and BCR-ABL1 negative B-progenitor ALL subtypes having very poor outcomes.

In one embodiment, a method is provided for assaying a biological sample for a genomic abnormality of the IKZF1 gene. The method comprises (a) providing a biological sample from a subject, wherein the biological sample comprises genomic DNA of the subject and (b) determining if the genomic DNA comprises a genomic abnormality in the IKZF1 gene. In one embodiment, the presence of the genomic abnormality of the IKZF1 gene is indicative of ALL, more particularly, BCR-ABL1 positive ALL. In another embodiment, the presence of the genomic abnormality of the IKZF1 gene is indicative of CML, more particularly, BC-CML or the likelihood of progression into blastic transformation of CML. In still another embodiment, the presence of the genomic abnormality of the IKZF1 gene is used as a prognostic marker to identify a subgroup of ALL with very poor outcomes, including the BCR-ABL1 positive and BCR-ABL1 negative B-progenitor ALL subtypes.

Such methods can be used to identify various IKZF1 genomic abnormalities including for example, a deletion of the entire IKZF1 gene, an intragenic deletion of the IKZF1 gene, or a deletion of at least one exon of the IKZF1 gene. In specific methods, the IKZF1 genomic abnormality that is detected comprises a deletion of exon 3 through exon 6 of the IKZF1 gene; a deletion of exon 2 through exon 6 of the IKZF1 gene; or a deletion of exon 1 through exon 6 of the IKZF1 gene. Alternatively, such methods can be employed to detect any of the additional IKZF1 genomic abnormalities disclosed herein.

It is further recognized that the diagnostic method used to detect the genomic abnormalities may be one which allows for the detection of the rearrangement without discriminating between the various IKZF1 genomic abnormalities disclosed herein.

Alternatively, the method employed may be such as to allow for a specific IKZF1 rearrangement to be distinguished. In other methods, an initial assay may be performed to confirm the presence of an IKZF1 genomic abnormality but not identify the specific genomic abnormality. If desired, a secondary assay can then be performed to determine the identity of the particular IKZF1 genomic abnormality. The second assay may use a different detection technology than the initial assay.

It is further recognized that the IKZF1 genomic abnormalities may be detected along with other markers in a multiplex or panel format. Markers are selected for their predictive value alone or in combination with the IKZF1 genomic abnormalities. Markers for other leukemias, diseases, infections, and metabolic conditions are also contemplated for inclusion in a multiplex of panel format. For example, when detecting IKZF1 genomic abnormalities to identify a subgroup of ALL with very poor outcomes, a test for the BCR-ABL1 translocation can also be performed. Such a test, however, is not required. Ultimately, the information provided by the methods of the present invention will assist a physician in choosing the best course of treatment for a particular patient.

As used herein, a “biological sample” can comprise any sample in which one desires to determine if the nucleic acid complement of the sample contains an IKZF1 genomic abnormality. For example, a biological sample can comprise a sample from any organism, including a mammal, such as a human, a primate, a rodent, a domestic animal (such as a feline or canine) or an agricultural animal (such as a ruminant, horse, swine or sheep). The biological sample can be derived from any cell, tissue or biological fluid from the organism of interest. The sample may comprises any clinically relevant tissue, such as, but not limited to, bone marrow samples, tumor biopsy, fine needle aspirate, or a sample of bodily fluid, such as, blood, plasma, serum, lymph, ascitic fluid, cystic fluid or urine. The sample used in the methods of the invention will vary based on the assay format, nature of the detection method, and the tissues, cells or extracts which are used as the sample. It is recognized that the sample typically requires preliminary processing designed to isolate or enrich the sample for the genomic DNA. A variety of techniques known to those of ordinary skill in the art may be used for this purpose.

As used herein, a “probe” is an isolated polynucleotide to which is attached a conventional detectable label or reporter molecule, e.g., a radioactive isotope, ligand, chemiluminescent agent, enzyme, etc. Such a probe is complementary to a strand of a target polynucleotide, which in specific embodiments of the invention comprise a polynucleotide comprising a junction of the IKZF1 genomic abnormality. Deoxyribonucleic acid probes may include those generated by PCR using IKZF1 specific primers, olignucleotide probes synthesized in vitro, or DNA obtained from bacterial artificial chromosome or cosmid libraries. Probes include not only deoxyribonucleic or ribonucleic acids but also polyamides and other probe materials that can specifically detect the presence of the target DNA sequence. For nucleic acid probes, examples of detection reagents include, but are not limited to radiolabeled probes, enzymatic labeled probes (horse radish peroxidase, alkaline phosphatase), affinity labeled probes (biotin, avidin, or steptavidin), and fluorescent labeled probes (6-FAM, VIC, TAMRA, MGB). One skilled in the art will readily recognize that the nucleic acid probes described in the present invention can readily be incorporated into one of the established kit formats which are well known in the art.

As used herein, “primers” are isolated polynucleotides that are annealed to a complementary target DNA strand by nucleic acid hybridization to form a hybrid between the primer and the target DNA strand., then extended along the target DNA strand by a polymerase, e.g., a DNA polymerase. Primer pairs of the invention refer to their use for amplification of a target polynucleotide, e.g., by the polymerase chain reaction (PCR) or other conventional nucleic-acid amplification methods. “PCR” or “polymerase chain reaction” is a technique used for the amplification of specific DNA segments (see, U.S. Pat. Nos. 4,683,195 and 4,800,159; herein incorporated by reference).

Probes and primers are of sufficient nucleotide length to bind to the target DNA sequence and specifically detect and/or identify a polynucleotide comprising an IKZF1 genomic abnormality or a junction of an IKZF1 genomic abnormality. It is recognized that the hybridization conditions or reaction conditions can be determined by the operator to achieve this result. This length may be of any length that is of sufficient length to be useful in a detection method of choice. Generally, 8, 11, 14, 16, 18, 20, 22, 24, 26, 28, 30, 40, 50, 75, 100, 200, 300, 400, 500, 600, 700 nucleotides or more, or between about 11-20, 20-30, 30-40, 40-50, 50-100, 100-200, 200-300, 300-400, 400-500, 500-600, 600-700, 700-800, or more nucleotides in length are used. Such probes and primers can hybridize specifically to a target sequence under high stringency hybridization conditions. Probes and primers according to embodiments of the present invention may have complete DNA sequence identity of contiguous nucleotides with the target sequence, although probes differing from the target DNA sequence and that retain the ability to specifically detect and/or identify a target DNA sequence may be designed by conventional methods. Accordingly, probes and primers can share about 80%, 85%, 90%, 91%, 92%, 93%, 94%, 95%, 96%, 97%, 98%, 99% or greater sequence identity or complementarity to the target polynucleotide (i.e., SEQ ID NO: 1 or to a fragment thereof). Probes can be used as primers, but are generally designed to bind to the target DNA or RNA and are not used in an amplification process.

Specific primers can be used to amplify the junction of an IKZF1 genomic abnormality to produce an amplicon that can be used as a “specific probe” or can itself be detected for identifying an IKZF1 genomic abnormality in a biological sample. When the probe is hybridized with the polynucleotides of a biological sample under conditions which allow for the binding of the probe to the sample, this binding can be detected and thus allow for an indication of the presence of the IKZF1 genomic abnormality in the biological sample. Such identification of a bound probe has been described in the art. The specific probe may comprise a sequence of at least 80%, between 80 and 85%, between 85 and 90%, between 90 and 95%, and between 95 and 100% identical (or complementary) to a specific region of the IKZF1 gene.

As used herein, “amplified DNA” or “amplicon” refers to the product of polynucleotide amplification of a target polynucleotide that is part of a nucleic acid template. For example, to determine whether the nucleic acid complement of a biological sample comprises an IKZF1 genomic abnormality, the nucleic acid complement of the biological sample may be subjected to a polynucleotide amplification method using a primer pair that includes a first primer derived from the 5′ flanking sequence adjacent to a junction of an IKZF1 genomic abnormality, and a second primer derived from the 3′ flanking sequence adjacent to the junction of the IKZF1 genomic abnormality to produce an amplicon that is diagnostic for the presence of the IKZF1 genomic abnormality. By “diagnostic” for an IKZF1 genomic abnormality is intended the use of any method or assay which discriminates between the present or the absence of an IKZF1 genomic abnormality in a biological sample. The amplicon is of a length and has a sequence that is also diagnostic for the IKZF1 genomic abnormality (i.e., has a junction sequence of the IKZF1 genomic abnormality). The amplicon may range in length from the combined length of the primer pairs plus one nucleotide base pair to any length of amplicon producible by a DNA amplification protocol. A member of a primer pair derived from the flanking sequence may be located a distance from the junction or breakpoint. This distance can range from one nucleotide base pair up to the limits of the amplification reaction, or about twenty thousand nucleotide base pairs. The use of the term “amplicon” specifically excludes primer dimers that may be formed in the DNA thermal amplification reaction.

Methods for preparing and using probes and primers are described, for example, in Molecular Cloning: A Laboratory Manual, 2.sup.nd ed, vol. 1-3, ed. Sambrook et al., Cold Spring Harbor Laboratory Press, Cold Spring Harbor, N.Y. 1989 (hereinafter, “Sambrook et al., 1989”); Current Protocols in Molecular Biology, ed. Ausubel et al., Greene Publishing and Wiley-Interscience, New York, 1992 (with periodic updates) (hereinafter, “Ausubel et al., 1992”); and Innis et al., PCR Protocols: A Guide to Methods and Applications, Academic Press: San Diego, 1990. PCR primer pairs can be derived from a known sequence, for example, by using computer programs intended for that purpose such as the PCR primer analysis tool in Vector NTI version 10 (Informax Inc., Bethesda Md.); PrimerSelect (DNASTAR Inc., Madison, Wis.); and Primer (Version 0.5.COPYRGT., 1991, Whitehead Institute for Biomedical Research, Cambridge, Mass.). Additionally, the sequence can be visually scanned and primers manually identified using guidelines known to one of skill in the art.

As outline in further detail below, any conventional nucleic acid hybridization or amplification or sequencing method can be used to specifically detect the presence of a polynucleotide arising due to an IKZF1 genomic abnormality. By “specifically detect” is intended that the polynucleotide can be used either as a primer to amplify the junction of an IKZF1 genomic abnormality or the polynucleotide can be used as a probe that hybridizes under stringent conditions to a polynucleotide having an IKZF1 genomic abnormality. The level or degree of hybridization which allows for the specific detection of the IKZF1 genomic abnormality is sufficient to distinguish the polynucleotide with the IKZF1 genomic abnormality from a polynucleotide that does not contain the rearrangement and thereby allow for discriminately identifying an IKZF1 genomic abnormality. By “shares sufficient sequence identity or complentarity to allow for the amplification of an IKZF1 chromosome rearrangement” is intended the sequence shares at least 80%, 85%, 90%, 91%, 92%, 93%, 94%, 95%, 96%, 97%, 98%, 99% or 100% identity or complementarity to a fragment or across the full length of the IKZF1 polynucleotide.

The IKZF1 genomic abnormalities may be detected using a variety of nucleic acid techniques known to those of ordinary skill in the art, including but not limited to: nucleic acid sequencing; nucleic acid hybridization; and, nucleic acid amplification. Nucleic acid hybridization includes methods using labeled probes directed against purified DNA, amplified DNA, and fixed leukemic cell preparations (fluorescence in situ hybridization).

Illustrative non-limiting examples of nucleic acid sequencing techniques include, but are not limited to, chain terminator (Sanger) sequencing and dye terminator sequencing. Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom. Dye terminator sequencing alternatively labels the terminators. Complete sequencing can be performed in a single reaction by labeling each of the di-deoxynucleotide chain-terminators with a separate fluorescent dye, which fluoresces at a different wavelength.

The present invention further provides methods for identifying nucleic acids containing an IKZF1 genomic abnormality which do not necessarily require sequence amplification and are based on, for example, the known methods of Southern (DNA:DNA) blot hybridizations, in situ hybridization and FISH of chromosomal material, using appropriate probes. Such nucleic acid probes can be used that comprise nucleotide sequences in proximity to the IKZF1 genomic abnormality junction, or breakpoint. By “in proximity to” is intended within about 100 kilobases (kb) of the IKZF1 genomic abnormality junction.

In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts. In some embodiments, the IKZF1 genomic abnormalities are detected using fluorescence in situ hybridization (FISH).

In specific embodiments, probes for detecting an IKZF1 genomic abnormality are labeled with appropriate fluorescent or other markers and then used in hybridizations. The Examples section provided herein sets forth various protocol that are effective for detecting the genomic abnormalities, but one of skill in the art will recognize that many variations of these assay can be used equally well. Specific protocols are well known in the art and can be readily adapted for the present invention. Guidance regarding methodology may be obtained from many references including: In situ Hybridization:

Medical Applications (eds. G. R. Coulton and J. de Belleroche), Kluwer Academic Publishers, Boston (1992); In situ Hybridization: hi Neurobiology; Advances in Methodology (eds. J. H. Eberwine, K. L. Valentino, and J. D. Barchas), Oxford University Press Inc., England (1994); In situ Hybridization: A Practical Approach (ed. D. G. Wilkinson), Oxford University Press Inc., England (1992)); Kuo et al. (1991) Am. J. Hum. Genet. 42:112-119; Klinger et al. (1992) Am. J. Hum. Genet. 51:55-65; and Ward et al. (1993) Am. J. Hum. Genet. 52:854-865). There are also kits that are commercially available and that provide protocols for performing FISH assays (available from e.g., Oncor, Inc., Gaithersburg, Md.). Patents providing guidance on methodology include U.S. Pat. Nos. 5,225,326; 5,545,524; 6,121,489 and 6,573,043. All of these references are hereby incorporated by reference in their entirety and may be used along with similar references in the art and with the information provided in the Examples section herein to establish procedural steps convenient for a particular laboratory.

Southern blotting can be used to detect specific DNA sequences. In such methods, DNA that is extracted from a sample is fragmented, electrophoretically separated on a matrix gel, and transferred to a membrane filter. The filter bound DNA is subject to hybridization with a labeled probe complementary to the sequence of interest. Hybridized probe bound to the filter is detected.

In hybridization techniques, all or part of a polynucleotide that selectively hybridizes to a target polynucleotide having an IKZF1 genomic abnormality is employed. By “stringent conditions” or “stringent hybridization conditions” when referring to a polynucleotide probe is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of identity are detected (heterologous probing). Generally, a probe is less than about 1000 nucleotides in length or less than 500 nucleotides in length.

As used herein, a substantially identical or complementary sequence is a polynucleotide that will specifically hybridize to the complement of the nucleic acid molecule to which it is being compared under high stringency conditions. Appropriate stringency conditions which promote DNA hybridization, for example, 6× sodium chloride/sodium citrate (SSC) at about 45° C., followed by a wash of 2×SSC at 50° C., are known to those skilled in the art or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In hybridization reactions, specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ, of less than 45° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.) and Haymes et al. (1985) In: Nucleic Acid Hybridization, a Practical Approach, IRL Press, Washington, D.C. A polynucleotide is said to be the “complement” of another polynucleotide if they exhibit complementarity. As used herein, molecules are said to exhibit “complete complementarity” when every nucleotide of one of the polynucleotide molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions.

Regarding the amplification of a target polynucleotide (e.g., by PCR) using a particular amplification primer pair, “stringent conditions” are conditions that permit the primer pair to hybridize to the target polynucleotide to which a primer having the corresponding sequence (or its complement) would bind and preferably to produce an identifiable amplification product (the amplicon) having a junction of an IKZF1 genomic abnormality in a DNA thermal amplification reaction. In a PCR approach, oligonucleotide primers can be designed for use in PCR reactions to amplify a junction of an IKZF1 genomic abnormality. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Methods of amplification are further described in U.S. Pat. Nos. 4,683,195, 4,683,202 and Chen et al. (1994) PNAS 91:5695-5699. These methods as well as other methods known in the art of DNA amplification may be used in the practice of the embodiments of the present invention. It is understood that a number of parameters in a specific PCR protocol may need to be adjusted to specific laboratory conditions and may be slightly modified and yet allow for the collection of similar results. These adjustments will be apparent to a person skilled in the art.

The amplified polynucleotide (amplicon) can be of any length that allows for the detection of the IKZF1 genomic abnormality. For example, the amplicon can be about 10, 50, 100, 200, 300, 500, 700, 100, 2000, 3000, 4000, 5000 nucleotides in length or longer.

Any primer can be employed in the methods of the invention that allows a junction of the IKZF1 genomic abnormality to be amplified and/or detected. For example, in specific embodiments, at least one of the primers employed in the method of detection or amplification comprises the sequence set forth in SEQ ID NO:74, 75, 76, 77, 78, 79, 80, 81, 82, 83, 84, 85, 86, 87, 88, 89, 90, 91, 92, 93, 94, 95, 96, 97, 98, 99, 100, 101, 102, 103, and/or 104. Methods for designing PCR primers are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Other known methods of PCR that can be used in the methods of the invention include, but are not limited to, methods using paired primers, nested primers, single specific primers, degenerate primers, gene-specific primers, mixed DNA/RNA primers, vector-specific primers, partially mismatched primers, and the like.

Thus, in specific embodiments, a method of detecting the presence of an IKZF1 genomic abnormality in a biological sample is provided. The method comprises (a) providing a sample comprising the genomic DNA of a subject; (b) providing a pair of DNA primer molecules that can amplify an amplicon having a junction of an IKZF1 genomic abnormality (c) providing DNA amplification reaction conditions; (d) performing the DNA amplification reaction, thereby producing a DNA amplicon molecule; and (e) detecting the DNA amplicon molecule. In order for a nucleic acid molecule to serve as a primer or probe it need only be sufficiently complementary in sequence to be able to form a stable double-stranded structure under the particular solvent and salt concentrations employed.

In still other embodiments, genomic abnormalities of genomic DNA may be amplified prior to or simultaneous with detection. Illustrative non-limiting examples of nucleic acid amplification techniques include, but are not limited to, polymerase chain reaction (PCR), ligase chain reaction (LCR), strand displacement amplification (SDA), and nucleic acid sequence based amplification (NASBA). The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. For other various permutations of PCR see, e.g., U.S. Pat. Nos. 4,683,195, 4,683,202 and 4,800,159; Mullis et al, (1987) Meth. Enzymol. 155: 335; and, Murakawa et al., (1988) DNA 7: 287, each of which is herein incorporated by reference in its entirety.

The ligase chain reaction (Weiss (1991) Science 254: 1292, herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker et al. (1992) Proc. Natl. Acad. Sci. USA 89: 392-396; U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTP[alpha]S to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Non-amplified or amplified IKZF1 genomic abnormalities can be detected by any conventional means. For example, the genomic abnormalities can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Nelson et al. (1995) Nonisotopic Probing, Blotting, and Sequencing, ch. 17 (Larry J. Kricka ed., 2d ed., each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products in the present invention. See, e.g., U.S. Pat. No. 5,814,447 (herein incorporated by reference in its entirety).

Various methods can be used to detect the IKZF1 genomic abnormality or amplicon having a junction of an IKZF1 genomic abnormality, including, but not limited to, Genetic Bit Analysis (Nikiforov et al. (1994) Nucleic Acid Res. 22: 4167-4175) where a DNA oligonucleotide is designed which overlaps both the adjacent flanking DNA sequence and the inserted DNA sequence. The oligonucleotide is immobilized in wells of a microwell plate. Following PCR of the region of interest (using one primer in the inserted sequence and one in the adjacent flanking sequence) a single-stranded PCR product can be hybridized to the immobilized oligonucleotide and serve as a template for a single base extension reaction using a DNA polymerase and labeled ddNTPs specific for the expected next base. Readout may be fluorescent or ELISA-based. A signal indicates presence of the insert/flanking sequence due to successful amplification, hybridization, and single base extension.

Another detection method is the Pyrosequencing technique as described by Winge ((2000) Innov. Pharma. Tech. 00: 18-24). In this method, an oligonucleotide is designed that overlaps the junction. The oligonucleotide is hybridized to a single-stranded PCR product from the region of interest (one primer in the inserted sequence and one in the flanking sequence) and incubated in the presence of a DNA polymerase, ATP, sulfurylase, luciferase, apyrase, adenosine 5′ phosphosulfate and luciferin. dNTPs are added individually and the incorporation results in a light signal which is measured. A light signal indicates the presence of the transgene insert/flanking sequence due to successful amplification, hybridization, and single or multi-base extension.

Fluorescence Polarization as described by Chen et al. ((1999) Genome Res. 9: 492-498, 1999) is also a method that can be used to detect an amplicon of the invention. Using this method, an oligonucleotide is designed which overlaps the inserted DNA junction. The oligonucleotide is hybridized to a single-stranded PCR product from the region of interest (one primer in the inserted DNA and one in the flanking DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the genomic abnormality sequence due to successful amplification, hybridization, and single base extension.

Taqman® (PE Applied Biosystems, Foster City, Calif.) is described as a method of detecting and quantifying the presence of a DNA sequence and is fully understood in the instructions provided by the manufacturer. Briefly, a FRET oligonucleotide probe is designed which overlaps the junction. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization.

In one embodiment, the method of detecting a genomic abnormality of IKZF1 comprises (a) contacting the biological sample with a polynucleotide probe that hybridizes under stringent hybridization conditions with a polynucleotide having an IKZF1 genomic abnormality and specifically detects the IKZF1 genomic abnormality; (b) subjecting the sample and probe to stringent hybridization conditions; and (c) detecting hybridization of the probe to the polynucleotide, wherein detection of hybridization indicates the presence of the IKZF1 genomic abnormality.

### III. Kits

The materials used in the above assay methods are ideally suited for the preparation of a kit. Various detection reagents can be developed and used to assay the presence of the IKZF1 genomic abnormality. The terms “kits” and “systems,” as used herein in the context of the IKZF1 genomic abnormality detection reagents, are intended to refer to such things as combinations of multiple IKZF1 genomic abnormality detection reagents, or one or more IKZF1 genomic abnormality detection reagents in combination with one or more other types of elements or components (e.g., other types of biochemical reagents, containers, packages, such as packaging intended for commercial sale, substrates to which SNP detection reagents are attached, electronic hardware components, and the like). Accordingly, the present invention further provides IKZF1 genomic abnormality detection kits and systems, including but not limited to, packaged probe and primer sets (e.g., TaqMan probe/primer sets), arrays/microarrays of nucleic acid molecules, and beads that contain one or more probes, primers, or other detection reagents for detecting one or more IKZF1 genomic abnormality. The kits/systems can optionally include various electronic hardware components. For example, arrays (e.g., DNA chips) and microfluidic systems (e.g., lab-on-a-chip systems) provided by various manufacturers typically include hardware components. Other kits/systems (e.g., probe/primer sets) may not include electronic hardware components, but can include, for example, one or more IKZF1 genomic abnormality detection reagents along with other biochemical reagents packaged in one or more containers.

In some embodiments, a IKZF1 genomic abnormality kit typically contains one or more detection reagents and other components (e.g., a buffer, enzymes, such as DNA polymerases or ligases, chain extension nucleotides, such as deoxynucleotide triphosphates, positive control sequences, negative control sequences, and the like) necessary to carry out an assay or reaction, such as amplification and/or detection of a polynucleotide comprising a junction of one of the IKZF1 genomic abnormalities. A kit can further contain means for determining the amount of the target polynucleotide and means for comparing with an appropriate standard, and can include instructions for using the kit to detect the IKZF1 genomic abnormality. In one embodiment, kits are provided which contain the necessary reagents to carry out one or more assays to detect one or more of the IKZF1 genomic abnormality as disclosed herein. The IKZF1 genomic abnormality detection kits/systems may contain, for example, one or more probes, or pairs of probes, that hybridize to a nucleic acid molecule at or near the junction region.

In specific embodiments, a kit for identifying an IKZF1 genomic abnormality in a biological sample is provided. The kit comprises a first and a second primer, wherein the first and second primer amplify a polynucleotide comprising an IKZF1 genomic abnormality junction and thereby detect an IKZF1 genomic abnormality.

Further provided are polynucleotide detection kits comprising at least one polynucleotide that can specifically detect an IKZF1 genomic abnormality. In specific embodiments, the polynucleotide comprises at least one polynucleotide molecule of a sufficient length of contiguous nucleotides homologous or complementary to SEQ ID NO: 1 or a variant thereof to allow for the detection of an IKZF1 genomic abnormality.

### III. Compounds Useful in Modulating the Activity of Polypeptides Expressed From the IKZF1 Genomic Abnormalities

Further provided are methods for identifying agents that target a polypeptide expressed from the IKZF1 genomic abnormalities. Thus, methods to screen for compounds that can serve as molecular targets for drugs useful in modulating the activity of the polypeptides expressed from the IKZF1 genomic abnormalities are provided. Such compounds can find use in treating All (i.e., BCR-ABL1 positive ALL, B-progenitor (+) ALL or B-progenitor (−) ALL, and/or in treating CML, more particularly, in treating BC-CML or treating, preventing or delaying progression into BC-CML. The invention provides a method (also referred to herein as a “screening assay”) for identifying modulators, i.e., candidate or test compounds or agents (e.g., peptides, peptidomimetics, small molecules, or other drugs) that modulate (e.g. inhibits) the activity of a polypeptide expressed from the IKZF1 gene having a genomic abnormality.

The test compounds of the present invention can be obtained using any of the numerous approaches in combinatorial library methods known in the art, including biological libraries, spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869), or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

The compounds screened in the above assay can be, but are not limited to, small molecules, peptides, carbohydrates, or vitamin derivatives. The agents can be selected and screened at random or rationally selected or designed using protein modeling techniques. For random screening, agents such as peptides or carbohydrates are selected at random and are assayed for their ability to bind to the polypeptide expressed from the IKZF1 gene having the genomic abnormality. Alternatively, agents may be rationally selected or designed. As used herein, an agent is said to be “rationally selected or designed” when the agent is chosen based on the configuration of the polypeptide expressed from the IKZF1 gene having the genomic abnormality. For example, one skilled in the art can readily adapt currently available procedures to generate peptides capable of binding to a specific peptide sequence in order to generate rationally designed antipeptide peptides, see, for example, Hurby et al., “Application of Synthetic Peptides: Antisense Peptides,” in Synthetic Peptides: A User's Guide, W.H. Freeman, New York (1992), pp. 289-307; and Kaspczak et al., Biochemistry 28:9230-2938 (1989).

Determining the ability of the test compound to specifically bind to the polypeptide expressed from the IKZF1 gene having the genomic abnormality can be accomplished, for example, by coupling the test compound with a radioisotope or enzymatic label such that binding of the test compound to the polypeptide expressed from the IKZF1 gene having the genomic abnormality can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In another embodiment, an assay of the present invention is a cell-free assay comprising contacting a polypeptide expressed from the IKZF1 gene having the genomic abnormality with a test compound and determining the ability of the test compound to specifically bind to the polypeptide expressed from the IKZF1 gene having the genomic abnormality. Binding of the test compound to the polypeptide expressed from the IKZF1 gene having the genomic abnormality can be determined either directly or indirectly as described above.

In another embodiment, an assay is a cell-free assay comprising contacting the polypeptide expressed from the IKZF1 gene having the genomic abnormality with a test compound and determining the ability of the test compound to specifically modulate (i.e., inhibit or activate) the activity of the polypeptide expressed from the IKZF1 gene having the genomic abnormality. Determining the ability of the test compound to inhibit the activity of a polypeptide expressed from the IKZF1 gene having the genomic abnormality using any method that can assay for IKZF1 activity. In addition, one could assay for the treatment of ALL (i.e., BCR-ABL1 positive ALL, B-progenitor (+) ALL or B-progenitor (−) ALL) and/or in the treatment of CML, more particularly, in the treatment of BC-CML or treating, preventing or delaying progression into BC-CML.

Such desired compounds may be further screened for selectivity by determining whether they suppress or eliminate phenotypic changes or activities associated with expression of the polypeptides expressed from IKZF1 genes having a genomic abnormality in the cells. The agents are screened by administering the agent to the cell or alternatively, the activity of the selective agent can be monitored in an in vitro assay. It is recognized that it is preferable that a range of dosages of a particular agent be administered to the cells to determine if the agent is useful for treating ALL, more particularly, BCR-ABL1 positive ALL and/or in the treatment of CML, more particularly, in the treatment of BC-CML and/or treating, preventing or delaying progression into BC-CML.

There are numerous variations of the above assays which can be used by a skilled artisan in order to isolate agonists. See, for example, Burch, R. M., in Medications Development. Drug Discovery, Databases, and Computer-Aided Drug Design, NIDA Research Monograph 134, NIH Publication No. 93-3638, Rapaka, R. S., and Hawks, R. L., eds., U.S. Dept. of Health and Human Services, Rockville, Md. (1993), pages 37-45.

Using the above procedures, the present invention provides compound capable of binding or modulating the activity of a polypeptide expressed from the IKZF1 gene having the genomic abnormality, produced by a method comprising the steps of (a) contacting said compound with the polypeptide expressed from the IKZF1 gene having the genomic abnormality, and (b) determining whether the agent specifically binds or modulates the activity of the polypeptide expressed from the IKZF1 gene having the genomic abnormality. Additional step(s) to determine whether such binding is selective for the IKZF1 polypeptide expressed from a IKZF1 gene lacking a genomic abnormality may also be employed.

### V. Sequence Identity

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

By “fragment” is intended a portion of the polynucleotide. Fragments of an IKZF1 polynucleotide or an exon or intron or promoter or 5′/3′ regulatory region thereof or fragments of a polynucleotide comprising an IKZF1 genomic abnormality are useful as, for example, probes and primers and need not encode the IKZF1 polypeptide. Instead, such fragments and variants are able to detect an IKZF1 genomic abnormality that is associated with ALL, more particularly with BCR-ABL1 positive ALL and/or associated with CML, more particularly, BC-CML or the likelihood of progression into blastic transformation of CML. Alternatively, such fragments and variants are able to detect an IKZF1 genomic abnormality that is predictive of a subtype of ALL having a very poor outcome. Thus, fragments of a nucleotide sequence may range from at least about 10, about 15, 20 nucleotides, about 50 nucleotides, about 75 nucleotides, about 100 nucleotides, 200 nucleotides, 300 nucleotides, 400 nucleotides, 500 nucleotides, 600 nucleotides, 700 nucleotides and up to the full-length polynucleotide employed in the invention. Methods to assay for the activity of a desired polynucleotide or polypeptide are described elsewhere herein.

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. Generally, variants of a particular polynucleotide of the invention having the desired activity will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

An “isolated” or “purified” polynucleotide or polypeptide or biologically active fragment or variant thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For purposes of the invention, “isolated” when used to refer to nucleic acid molecules excludes isolated chromosomes. For example, in various embodiments, the isolated nucleic acid molecules can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived.

As used herein, the use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The following examples are offered by way of illustration and not by way of limitation.

### EXPERIMENTAL

### Example 1

### Abstract

The Philadelphia chromosome, encoding BCR-ABL1, is the defining lesion of chronic myelogenous leukemia (CML) and a subset of acute lymphoblastic leukemia (ALL). To define oncogenic lesions that cooperate with BCR-ABL1 to induce ALL, we performed genome-wide analysis of diagnostic leukemic samples from 304 individuals with ALL, including 43 BCR-ABL1 B-progenitor ALLs, and 34 CML cases. IKZF1 (encoding the transcription factor Ikaros) was deleted in 83.7% of BCR-ABL1 ALL, but not in chromic phase CML. Deletion of IKZF1 was also identified as an acquired lesion in lymphoid blast crisis of CML. The IKZF1 deletions resulted in haploinsufficiency, expression of a dominant negative Ikaros isoform or the complete loss of Ikaros expression. Sequencing of IKZF1 deletion breakpoints suggested that aberrant RAG-mediated recombination is responsible for the deletions. These findings suggest that genetic lesions resulting in the loss of Ikaros function are a key event in the development of BCR-ABL1 ALL.

### Methods

Patients and samples. Two hundred eighty two patients with acute lymphoblastic leukemia (ALL) treated at St. Jude Children's Research Hospital, 22 adult BCR-ABL1 ALL patients treated at the University of Chicago, and 49 samples obtained from 23 adult patients with chronic myeloid leukemia (CML) treated at the Institute of Medical and Veterinary Science, Adelaide, and 36 AML and ALL cell lines were studied (Tables 1 and 2). The CML cohort included 24 chronic phase, 7 accelerated phase and 15 blast crisis samples, and three samples obtained at complete cytogenetic response. All blast crisis samples were flow sorted to at least 90% blast purity prior to DNA extraction using FACS Vantage SE (with DiVa option) flow cytometers (BD Biosciences, San Jose, Calif.) and fluorescein isothiocyanate labelled CD45, allophycocyanin labelled CD33 and phycoerythrin labelled CD19 and CD13 antibodies (BD Biosciences). Germline tissue was obtained by also sorting the non-blast population in 7 cases. Informed consent for the use of leukemic cells for research was obtained from patients, parents or guardians in accordance with the Declaration of Helsinki, and study approval was obtained from the SJCRH institutional review board.

Single nucleotide polymorphism microarray analysis. Collection and processing of diagnostic and remission bone marrow and peripheral blood samples for Affymetrix single nucleotide polymorphism microarray analysis has been previously reported in detail⁹. Affymetrix 250K Sty and Nsp arrays were performed on all samples. 50 k Hind 240 and 50 k Xba 240 arrays were performed for 252 ALL samples (Table 1).

Fluorescent in situ hybridization. Fluorescence in situ hybridization for IKZF1 deletion was performed using diagnostic bone marrow or peripheral blood leukemic cells in Carnoy's fixative as previously described⁹. BAC clones CTD-2382L6 and CTC-79 1O3 (for IKZF1, Open Biosystems, Huntsville, Ala.) were labelled with fluorescein isothiocyanate, and control 7q3 1 probes RP1 1-460K21 (Children's Hospital Oakland Research Institute, Oakland, Calif.) and CTB-133K23 (Open Biosystems), were labelled with rhodamine. At least 100 interphase nuclei were scored per case.

IKZF1 PCR, cloning, quantitative PCR and genomic sequencing. RNA was extracted and reverse transcribed using random hexamer primers and Superscript III (Invitrogen, Carlsbad, Calif.) as previously described⁹. IKZF1 transcripts were amplified from cDNA using the Advantage 2 PCR enzyme (Clontech, Mountain View) as previously described⁹ using primers that anneal in exon 0 and 7 of IKZF1. PCR products were purified, and sequenced directly and after cloning into pGEM-T-Easy (Promega, Madison, Wis.). Genomic quantitative PCR for exons 1-7 of IKZF1, and real-time PCR to quantify expression of Ik6 were performed as previously described⁹. All primers and probes are listed in Table 10. Genomic sequencing of IKZF1 exons 0-7 in all ALL and CML samples was performed as previously described⁹.

Western blotting. Whole cell lysates of 3-6×10⁶ leukemic cells were prepared and blotted as previously described⁹ using N- and C-terminus specific rabbit polyclonal Ikaros antibodies (Santa Cruz Biotechnology, Santa Cruz, Calif.).

Methylation analysis. Methylation status of the IKZF1 promoter CpG island (chr7:5012 1508-50121714) was performed using MALDI-TOF mass spectrometry of PCR-amplified, bisulfite modified genomic DNA extracted from leukemic cells as previously described^{8,29}. Statistical analysis. Associations between ALL subtype and IKZF1 deletion frequency were calculated using the exact likelihood ratio test. Differences in Ik6 expression between IKZF1 Δ3-6 and non-Δ3-6 cases was assessed using the exact Wilcoxon-Mann-Whitney test. All P values reported are two-sided. Analyses were performed using StatXact v8.0.0 (Cytel, Cambridge, Mass.).

Cell lines examined by SNP array. Thirty-six acute myeloid and lymphoid leukemia cell lines were genotyped using the Affymetrix Mapping 250 k Sty and Nsp arrays. These were the ALL cell lines 380 (MYC-IGH and BCL2-IGH B-precursor), 697 (TCF3-PBX1), AT 1 (ETV6-R UNX1), BV1 73 (CML in lymphoid blast crisis), CCRF-CEM (TAL-SIL), Jurkat (T-ALL), Kasumi-2 (TCF3-PBX1), MHH-CALL-2 (hyperdiploid B-precursor ALL), MHH-CALL-3 (TCF3-PBX1), MOLT3 (T-ALL), MOLT4 (T-ALL), NALM-6 (B-precursor ALL), OP1 (BCR-ABL1), Reh (ETV6-RUNX1), RS4; 11 (MLL-AF4), SD1 (BCR-ABL1), SUP-B15 (BCR-ABL1), TOM-1 (BCR-ABL1), U-937 (PICALM-AF10), UOCB1 (TCF3-HLF), YT (NK leukemia); and the AML cell lines CMK (FAB M7), HL-60 (FAB M2), K-562 (CML in myeloid blast crisis), Kasumi-1 (RUNX1-RUNX1T1), KG-1 (myelocytic leukemia), ME-1 (CBFB-MYH11), ML-2 (MLL-AF6), M-07e (FAB M7), Mono Mac 6 (MLL-AF9), MV4-1 1 (MLL-AF4), NB4 (PML-RARA), NOMO-1 (MLL-AF9), PL21 (FAB M3), SKNO-1 (RUNX1-RUNX1T1) and THP-1 (FAB M5). Cell lines were obtained from the Deutsche Sammlung von Mikroorganismen and Zellkulturen, Braunschweig, Germany; the American Type Culture Collection, Manassas, Va., from local institutional repositories, or were gifts from Olaf Heidenreich (SKNO-1) and Dario Campana (OP1). Cells were culture in accordance with previously published recommendations³⁰. The paediatric BCR-ABL1 B-precursor ALL cell line OP1³¹ was cultured in RPMI-1640 containing 100 units/ml penicillin, 100 μg/ml streptomycin, 2 mM glutamine and 10% fetal bovine serum. DNA was extracted from 5×10⁶ cells obtained during log phase growth after washing in PBS using the Qlamp DNA blood mini kit (Qiagen, Valencia, Calif.).

Obtaining primary SNP array data. SNP array CEL and SNP call TXT files (generated by Affymetrix GTYPE 4.0 using the DM algorithm) have been deposited in NCBIs Gene Expression Omnibus (GEO, www.ncbi.nlm.nih.gov/geo/) and are accessible through GEO Series accession numbers GSE9109-91 13. These accessions contain the following data: GSE9109: Sty and Nsp files for 304 ALL samples, and Hind and Xba files for 252 of these samples; GSE91 10: Sty and Nsp files for 56 CML samples; GSE9 111: Sty, Nsp, Hind and Xba files for 50 remission acute leukemia samples used as references for copy number analysis; GSE91 12: Sty and Nsp files for 36 acute leukemia cell lines; GSE9 11 3: A superseries containing all of the above data.

### Results

Acute lymphoblastic leukemia (ALL) comprises a heterogeneous group of disorders characterized by recurring chromosomal abnormalities including translocations, trisomies and deletions. An ALL subtype with especially poor prognosis is characterized by the presence of the Philadelphia chromosome arising from the t(9; 22)(q34; q1 1.2) translocation, which encodes the constitutively activated BCR-ABL1 tyrosine kinase. BCR-ABL1 positive ALL constitutes 5% of paediatric B-progenitor ALL and approximately 40% of adult ALL^{1,2}. Expression of BCR-ABL1 is also the pathologic lesion underlying chronic myelogenous leukemia (CML)³. Data from murine studies demonstrates that expression of BCR-ABL1 in haematopoietic stem cells can alone induce a CML-like myeloproliferative disease, but cooperating oncogenic lesions are required for the generation of a blastic leukemia^{4,5}. Although the p210 and p190 BCR-ABL1 fusions are most commonly found in CML and paediatric BCR-ABL1 ALL respectively, either fusion may be found in adult BCR-ABL1 ALL⁶. Importantly, a number of genetic lesions including additional cytogenetic aberrations and mutations in tumor suppressor genes have been described in CML cases progressing to blast crisis⁷. However, the specific lesions responsible for the generation of BCR-ABL1 acute lymphoid leukemia and blastic transformation of CML remain incompletely understood⁷. To identify cooperating oncogenic lesions in ALL, we recently performed a genome-wide analysis of paediatric ALL⁸. This analysis identified an average of 6.8 genomic copy number alterations (CNA) in 9 BCR-ABL1 ALL cases, including deletions in genes that play a regulatory role in normal B cell development.

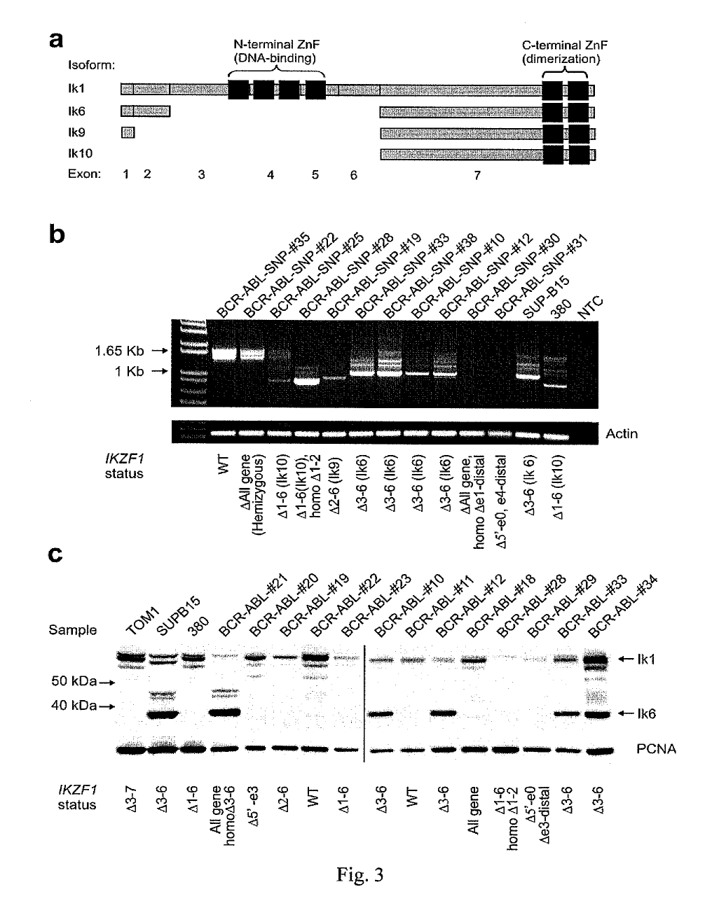

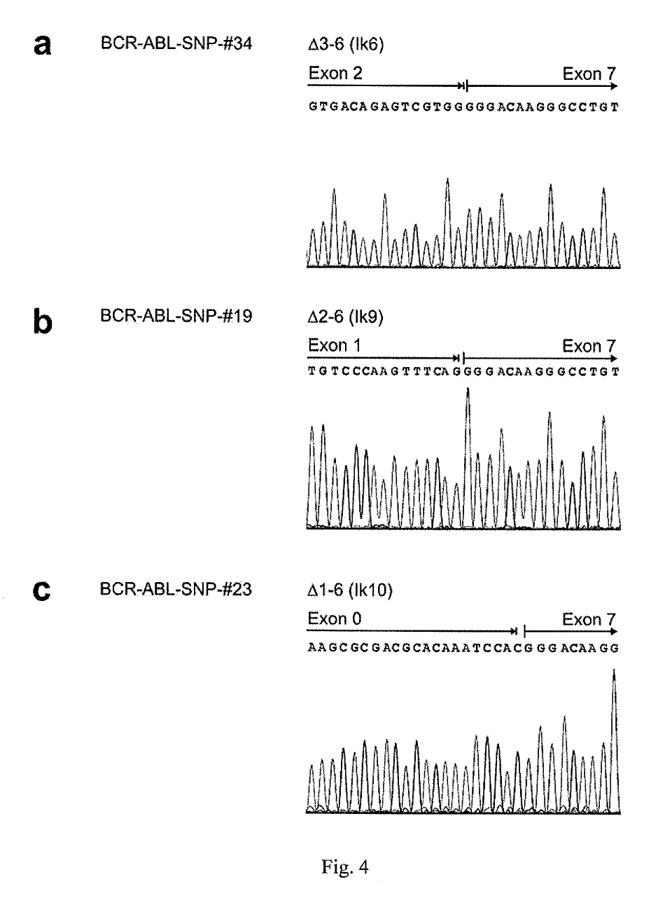

To extend this analysis and identify lesions that distinguish CML from BCR-ABL1 ALL, we have now examined DNA from leukemic samples from 304 paediatric and adult ALLs (254 B-progenitor, 50 T-lineage), including 21 paediatric and 22 adult BCR-ABL1 ALL, and 23 adult CML samples (Table 1). Samples were analyzed using the 250 k Sty and Nsp Affymetrix SNP arrays (and also the 100K arrays for most cases). This identified a mean of 8.79 somatic CNA per BCR-ABL1 ALL case (range 1-26), with 1.44 gains (range 0-13) and 7.33 losses (range 0-25) (Table 2). No significant differences were noted in the frequency of CNAs between paediatric and adult BCR-ABL1 ALL cases. The most frequent somatic CNA was deletion of IKZF1, which encodes the transcription factor Ikaros (Table 3). IKZF1 was deleted in 36 (83.7%) of 43 BCR-ABL1 ALL cases, including 76.2% of paediatric and 90.9% of adult BCR-ABL1 ALL cases. CDKN2A was deleted in 53.5% of BCR-ABL1 ALL cases, most of which (87.5%) also had deletions of IKZF1 (FIGS. 3 and 4). Conversely, of the BCR-ABL1 ALL cases with IKZF1 deletions, 41.6% lacked CDKN2A alterations. Deletion of PAX5 occurred in 51% of BCR-ABL ALL cases, again with the majority also having a deletion of IKZF1 (95%) (FIGS. 3 and 4). No other defining CNAs were identified in the rare BCR-ABL1 ALL cases that lacked a deletion of IKZF1.

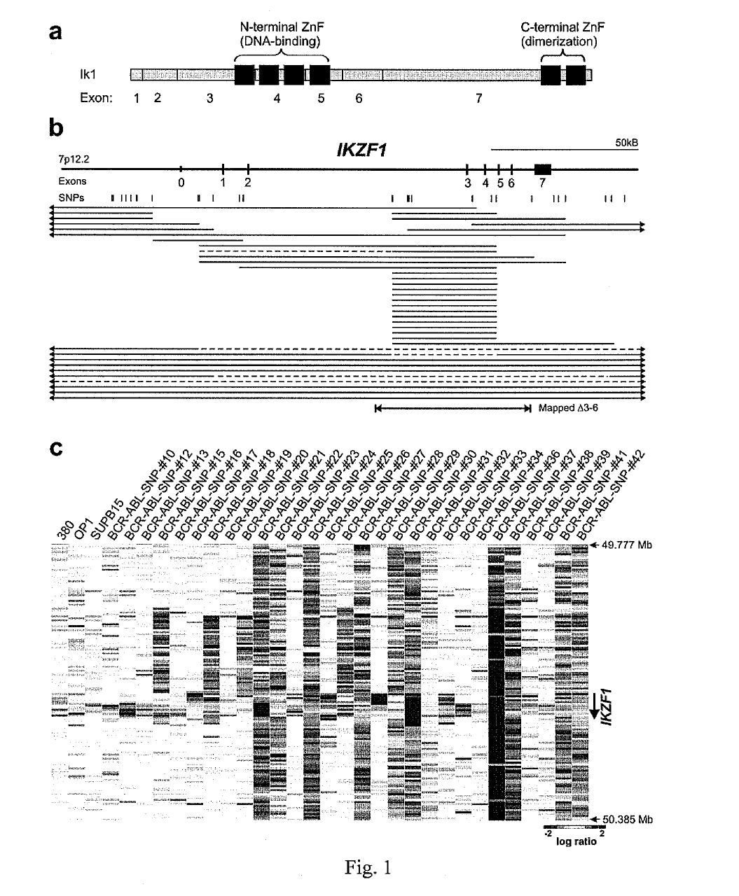

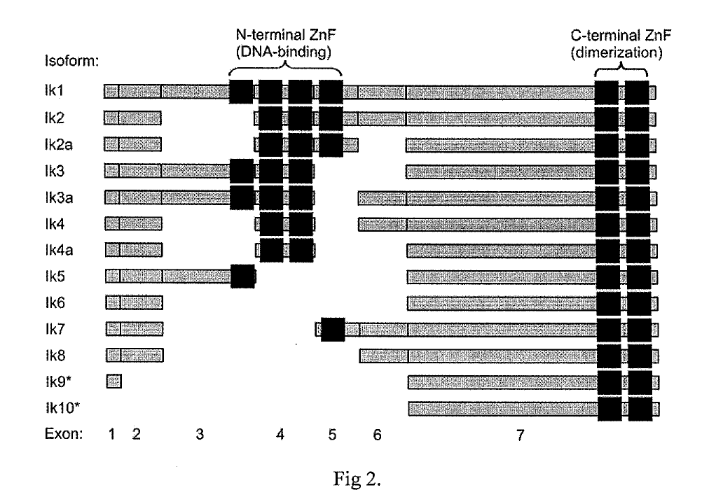

Ikaros is a member of a family of zinc finger nuclear proteins that is required for normal lymphoid development⁹⁻¹². Ikaros has a central DNA-binding domain consisting of four zinc fingers, and a homo- and heterodimerization domain consisting of the two C-terminal zinc fingers¹³ (FIGS. 5 and 6). Alternative splicing generates multiple Ikaros isoforms, several of which lack the N-terminal zinc fingers required for DNA binding; however, the physiological relevance of these isoforms in normal hematopoiesis remains unclear^{9-11,14} (FIG. 2). The IKZF1 deletions identified in BCR-ABL1 ALL were predominantly mono-allelic and were limited to the gene in 25 cases, conclusively identifying IKZF1 as the genetic target (FIG. 1). In 19 cases the deletions were confined to a subset of internal IKZF1 exons, most commonly exons 3-6 (Δ3-6; N=15). Importantly, the Δ3-6 deletion is predicted to encode an Ikaros isoform that lacks the DNA-binding domain but retains the C-terminal zinc fingers. The IKZF1 deletions were confirmed by FISH and genomic quantitative PCR, and were in the predominant leukemic clone (Table 5 and data not shown). Detailed analysis failed to reveal any evidence of either IKZF1 point mutations or inactivation of its promoter by CpG methylation in primary ALL samples (data not shown).

The expression of aberrant, dominant negative Ikaros isoforms in B- and T-lineage ALL has been previously reported by several groups¹⁵⁻²², although alternative splicing has been reported to be the underlying mechanism²³. Importantly, the Δ3-6 isoform of Ikaros has been shown to function as a dominant negative inhibitor of the transcriptional activity of Ikaros and related family members¹³. Moreover, mice heterozygous for a null IKZF1 allele develop clonal T cell expansions²⁴ and mice transgenic for the an IKZF1 A3-6 gene lack T, B, NK and dendritic cells, and develop a T cell lymphoproliferative diseases^{25,26}, demonstrating that alteration in the level of IKZF1 expression is oncogenic.

The high frequency of focal deletions in IKZF1 in BCR-ABL1 ALL suggests that expression of alternative IKZF1 transcripts may be the result of specific genetic lesions, and not alternative splicing of an intact gene. To further explore this possibility, we performed RT-PCR analysis for IKZF1 transcripts in 159 cases (FIG. 3). This demonstrated that expression of the Ik6 transcript, which lacks exons 3-6, was exclusively observed in cases harbouring the IKZF1 A3-6 deletion (FIG. 3b). Furthermore, we detected two novel Ikaros isoforms exclusively in cases with larger deletions; Ik9 in a case with deletion of exons 2-6, and Ik10 in three cases with deletion of exons 1-6 (FIGS. 3A and B, and FIG. 4). For each isoform, Ik6, 9 and 10, there was concordance between the transcripts detected by RT-PCR and the extent of deletion defined by SNP array and genomic PCR analysis (FIG. 3b). Moreover, analysis of 22 IKZF1 A3-6 and 29 non-A3-6 cases with a quantitative PCR assay specific for the Ik6 transcript confirmed that Ik6 expression was restricted to cases with the A3-6 deletion (P=6.41×10⁻¹⁵, FIG. 5). In addition, the Ik6 protein isoform was only detectable by western blotting in cases with a A3-6 IKZF1 deletion (data not shown). We also did not observe expression of Ik6 following the enforced expression of BCR-ABL1 in Arf null or wild type murine hematopoietic precursors (data not shown). Together, these data indicate that the expression of non-DNA binding Ikaros isoforms is due to IKZF1 genomic abnormalities, and not aberrant post-transcriptional splicing induced by BCR-ABL1, as has been suggested²³.

To identify CNAs in CML, we performed SNP array analysis on 23 CML cases. In addition to chronic phase CML (CP-CML), we also examined matched accelerated phase (APCML, N=7) and blast crisis (BC-CML, N=15, 12 myeloid and 3 lymphoid) samples (Table 6). This identified only 0.47 CNAs per CP-CML case (range 0-8) (Table 7), suggesting that BCR-ABL1 is sufficient to induce CML, but alone does not result in substantial genomic instability. Importantly, no recurrent lesions were identified. In contrast, there was a mean of 7.8 CNAs per BC-CML case (range 0-28) (Table 7), with IKZF1 deletions in four BC samples, including two of the three cases with lymphoid blast crisis (FIG. 6b). Two of the IKZF1 deletions involved the entire gene (CML-#4-BC and #22-BC), one Δ3-6 (CML-#1-BC, which was associated with Ik6 expression by RT-PCR) and one Δ3-7 (CML-#7-BC). CML-#7-BC also had an IKZF1 nonsense mutation in the C-terminal zinc finger domain of exon 7 in the non-deleted allele (c.1520C>A, p. Ser507X, FIG. 6c). One BC sample had a CDKN2A deletion, and four cases had CNAs involving PAX5 (two deletions, one internal amplification, one trisomy 9), with two of these also having IKZF1 deletion. CNAs were identified in two AP-CML samples. These data demonstrate an increased burden of genomic aberrations during progression of CML, with IKZF1 mutation a frequent event in the transformation of CML to lymphoid blast crisis.

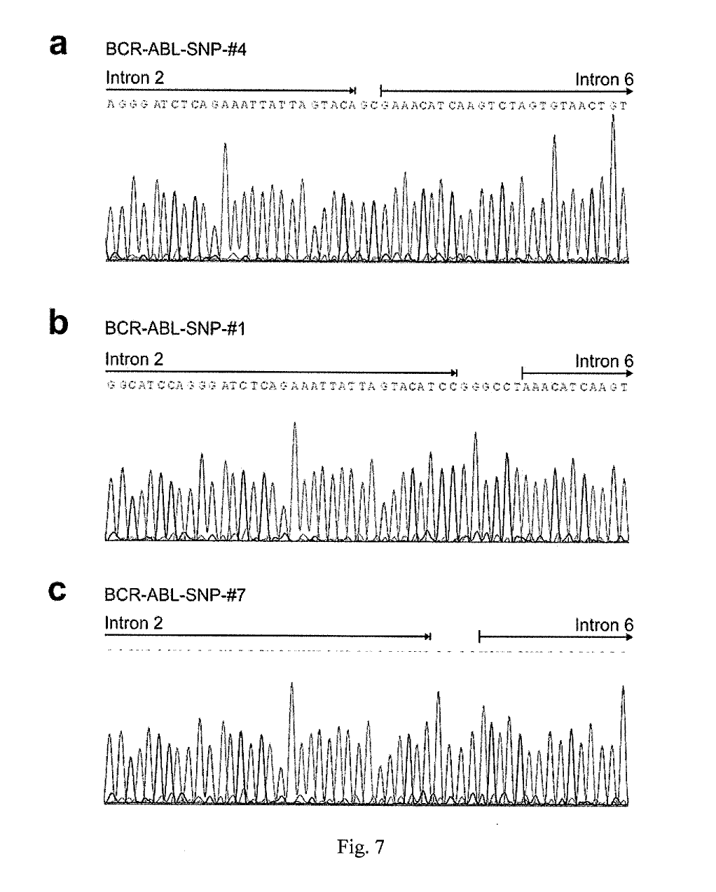

To explore the mechanism responsible for the identified IKZF1 deletion, we sequenced the IKZF1 Δ3-6 genomic breakpoints (Table 8 and FIG. 7). The deletions were restricted to highly localized sequences in introns 2 and 6 (Table 8 and FIG. 7). Moreover, heptamer recombination signal sequences (RSSs) recognized by the RAG enzymes during V(D)J recombination²⁷ were located immediately internal to the deletion breakpoints, and a variable number of additional nucleotides were present between the consensus intron 2 and 6 sequences, suggestive of the action of terminal deoxynucleotidyl transferase (TdT). Together, these data suggest that the IKZF1 Δ3-6 deletion arises due to aberrant RAG-mediated recombination.

In summary, we have identified a high frequency of CNAs in BCR-ABL1 ALL and BCCML, but not in CP-CML. The high frequency of recurring CNAs suggests that these lesions directly contribute to the generation of BCR-ABL1 ALL. Among the identified lesions, our analysis revealed a near obligate deletion of IKZF1 in BCR-ABL1 ALL, with 83.7% of paediatric and adult cases containing a deletion that leads to a reduction in dose and/or the expression of an altered Ikaros isoform. By contrast, deletion of IKZF1 was not detected in CP-CML, but was identified as an acquired lesion in 2 of 3 lymphoid BC-CML samples. Furthermore, our data suggest that the IKZF1 deletions result from aberrant RAG-mediated recombination. These data, and the low frequency of IKZF1 deletions in other paediatric B-progenitor ALL cases, suggests that alterations in Ikaros directly contribute to the pathogenesis of BCR-ABL1 ALL. How reduced activity of Ikaros, and possibly that of other family members through the expression of dominant negative Ikaros isoforms, collaborates with BCR-ABL1 to induce lymphoblastic leukemia remains to be determined. Importantly, mice with attenuated Ikaros expression exhibit a partial block of B lymphoid maturation at the pro-B cell stage²⁸, suggesting that Ikaros loss may contribute to the arrested B lymphoid maturation in BCR-ABL1 ALL. However, the high co-occurrence of PAX5 deletions in many cases suggests that IKZF1 deletion contributes to transformation in additional ways. The frequent co-deletion of CDKN2A (encoding INK4A/ARF) with IKZF1 in BCR-ABL1 ALL is a notable finding. This suggests that attenuated Ikaros activity may either collaborate with disruption of INK4A/ARF-mediated tumor suppression, or act through alternative uncharacterized tumor suppressor pathways in ALL. Dissecting the contribution of altered Ikaros activity to BCR-ABL1 leukaemogenesis should not only provide valuable mechanistic insights, but will also help to determine if the presence of this genetic lesion can be used to gain a therapeutic advantage against this aggressive leukemia.

Table 1. The acute lymphoblastic leukemia cases studied by Affymetrix SNP array. * Adult BCR-ABL1 B-ALL cases. ^{†}IKZF1 sequencing for these cases was not performed or failed due to limited DNA. ND, not done.

TABLE 1

| | 350K | | | | |
|---|---|---|---|---|---|
| | SNP | | | | Nsp |
| | data | Hind | Xba | Sty | SNP |
| ALL SNP identifier | reported³ | SNP call | SNP | SNP | call rate |
| Hyperdip>50-SNP-#1 | Yes | 91.7 | 98.5 | 93.2 | 93.4 |
| Hyperdip>50-SNP-#2 | Yes | 96.2 | 99.0 | 97.1 | 88.7 |
| Hyperdip>50-SNP-#3 | Yes | 95.2 | 97.0 | 94.8 | 92.2 |
| Hyperdip>50-SNP-#4 | Yes | 93.1 | 97.0 | 93.4 | 91.9 |
| Hyperdip>50-SNP-#5 | Yes | 88.6 | 94.7 | 92.3 | 93.8 |
| Hyperdip>50-SNP-#6 | Yes | 90.7 | 97.2 | 89.4 | 96.4 |
| Hyperdip>50-SNP-#7 | Yes | 90.9 | 98.0 | 91.2 | 90.6 |
| Hyperdip>50-SNP-#8 | Yes | 89.8 | 90.8 | 93.2 | 93.9 |
| Hyperdip>50-SNP-#9 | Yes | 90.3 | 94.5 | 91.5 | 93.5 |
| Hyperdip>50-SNP-#10 | Yes | 87.8 | 93.5 | 94.1 | 92.5 |
| Hyperdip>50-SNP-#11 | Yes | 85.8 | 97.1 | 90.9 | 85.5 |
| Hyperdip>50-SNP-#12 | Yes | 91.9 | 95.6 | 91.3 | 88.8 |
| Hyperdip>50-SNP-#13 | Yes | 95.8 | 92.6 | 97.4 | 90.9 |
| Hyperdip>50-SNP-#14 | Yes | 88.4 | 95.8 | 94.8 | 88.2 |
| Hyperdip>50-SNP-#15 | Yes | 89.3 | 95.9 | 97.3 | 92.3 |
| Hyperdip>50-SNP-#16 | Yes | 91.0 | 93.0 | 97.2 | 89.0 |
| Hyperdip>50-SNP-#17 | Yes | 94.7 | 94.0 | 95.1 | 89.0 |
| Hyperdip>50-SNP-#18 | Yes | 92.4 | 90.9 | 88.5 | 89.6 |
| Hyperdip>50-SNP-#19 | Yes | 94.1 | 94.1 | 86.8 | 88.5 |
| Hyperdip>50-SNP-#20 | Yes | 93.6 | 93.7 | 84.3 | 92.0 |
| Hyperdip>50-SNP-#21 | Yes | 85.1 | 95.7 | 92.8 | 86.4 |
| Hyperdip>50-SNP-#22 | Yes | 89.7 | 92.4 | 84.1 | 89.7 |
| Hyperdip>50-SNP-#23 | Yes | 92.0 | 96.5 | 91.8 | 96.7 |
| Hyperdip>50-SNP-#24 | Yes | 96.9 | 98.1 | 83.6 | 91.8 |
| Hyperdip>50-SNP-#25 | Yes | 97.4 | 97.5 | 95.3 | 92.2 |
| Hyperdip>50-SNP-#26 | Yes | 94.2 | 97.5 | 88.2 | 88.5 |
| Hyperdip>50-SNP-#27 | Yes | 96.4 | 97.8 | 90.5 | 94.4 |
| Hyperdip>50-SNP-#28 | Yes | 97.3 | 98.3 | 87.2 | 95.7 |
| Hyperdip>50-SNP-#29 | Yes | 94.7 | 97.4 | 84.8 | 92.4 |
| Hyperdip>50-SNP-#30 | Yes | 94.2 | 97.4 | 93.9 | 93.4 |
| Hyperdip>50-SNP-#31 | Yes | 89.0 | 98.1 | 94.8 | 93.4 |
| Hyperdip>50-SNP-#32 | Yes | 97.0 | 97.3 | 93.1 | 92.8 |
| Hyperdip>50-SNP-#33 | Yes | 96.1 | 97.7 | 92.4 | 92.1 |
| Hyperdip>50-SNP-#34 | Yes | 94.6 | 96.8 | 94.7 | 92.3 |
| Hyperdip>50-SNP-#35 | Yes | 95.1 | 97.6 | 91.8 | 91.1 |
| Hyperdip>50-SNP-#36 | Yes | 92.0 | 97.8 | 92.2 | 93.0 |
| Hyperdip>50-SNP-#37 | Yes | 93.5 | 96.6 | 93.1 | 94.5 |
| Hyperdip>50-SNP-#38 | Yes | 95.4 | 97.5 | 94.5 | 89.9 |
| Hyperdip>50-SNP-#39 | Yes | 91.1 | 98.2 | 94.7 | 89.8 |
| Hyperdip>50-SNP-#40 | No | 94.3 | 94.9 | 91.3 | 94.4 |
| E2A-PBX1-SNP-#1 | Yes | 94.6 | 98.6 | 95.2 | 90.8 |
| E2A-PBX1-SNP-#2 | Yes | 86.0 | 97.2 | 89.7 | 96.6 |
| E2A-PBX1-SNP-#3 | Yes | 88.8 | 95.8 | 91.8 | 92.8 |
| E2A-PBX1-SNP-#4 | Yes | 90.5 | 94.9 | 77.7 | 91.9 |
| E2A-PBX1-SNP-#5 | Yes | 93.9 | 96.4 | 80.4 | 82.9 |
| E2A-PBX1-SNP-#6 | Yes | 92.1 | 98.3 | 90.6 | 91.2 |
| E2A-PBX1-SNP-#7 | Yes | 93.0 | 96.5 | 96.4 | 86.7 |
| E2A-PBX1-SNP-#8 | Yes | 92.7 | 94.4 | 96.2 | 86.1 |
| E2A-PBX1-SNP-#9 | Yes | 94.6 | 99.0 | 86.6 | 87.7 |
| E2A-PBX1-SNP-#10 | Yes | 96.3 | 99.2 | 89.6 | 93.2 |
| E2A-PBX1-SNP-#11 | Yes | 93.9 | 99.1 | 90.3 | 94.8 |
| E2A-PBX1-SNP-#12 | Yes | 92.9 | 98.8 | 94.3 | 90.5 |
| E2A-PBX1-SNP-#13 | Yes | 96.0 | 98.4 | 94.5 | 86.3 |
| E2A-PBX1-SNP-#14 | Yes | 94.9 | 99.0 | 74.0 | 91.1 |
| E2A-PBX1-SNP-#15 | Yes | 94.4 | 98.8 | 96.0 | 87.6 |
| E2A-PBX1-SNP-#16 | Yes | 91.7 | 94.7 | 96.3 | 88.4 |
| E2A-PBX1-SNP-#17 | Yes | 95.2 | 99.2 | 96.1 | 85.4 |
| TEL-AML1-SNP-#1 | Yes | 97.3 | 99.2 | 97.8 | 84.1 |
| TEL-AML1-SNP-#2 | Yes | 96.9 | 99.0 | 96.6 | 93.2 |
| TEL-AML1-SNP-#3 | Yes | 95.8 | 98.3 | 96.1 | 92.8 |
| TEL-AML1-SNP-#4 | Yes | 97.0 | 99.2 | 97.0 | 95.9 |
| TEL-AML1-SNP-#5 | Yes | 95.4 | 98.0 | 95.0 | 94.6 |
| TEL-AML1-SNP-#6 | Yes | 94.8 | 98.8 | 95.4 | 91.7 |
| TEL-AML1-SNP-#7 | Yes | 97.2 | 98.9 | 94.8 | 93.3 |
| TEL-AML1-SNP-#8 | Yes | 95.0 | 98.7 | 96.0 | 94.7 |
| TEL-AML1-SNP-#9 | Yes | 91.6 | 95.0 | 94.1 | 90.0 |
| TEL-AML1-SNP-#10 | Yes | 93.3 | 94.3 | 93.6 | 95.1 |
| TEL-AML1-SNP-#11 | Yes | 92.8 | 92.9 | 93.7 | 93.9 |
| TEL-AML1-SNP-#12 | Yes | 96.0 | 87.2 | 89.8 | 94.1 |
| TEL-AML1-SNP-#13 | Yes | 85.5 | 91.1 | 92.9 | 94.0 |
| TEL-AML1-SNP-#14 | Yes | 90.8 | 95.9 | 90.4 | 93.2 |
| TEL-AML1-SNP-#15 | Yes | 83.7 | 93.8 | 87.6 | 89.6 |
| TEL-AML1-SNP-#16 | Yes | 85.5 | 89.9 | 95.1 | 93.7 |
| TEL-AML1-SNP-#17 | Yes | 87.0 | 94.2 | 95.7 | 92.2 |
| TEL-AML1-SNP-#18 | Yes | 94.0 | 86.7 | 96.0 | 90.7 |
| TEL-AML1-SNP-#19 | Yes | 90.1 | 95.3 | 97.1 | 89.9 |
| TEL-AML1-SNP-#20 | Yes | 94.9 | 94.1 | 98.2 | 92.6 |
| TEL-AML1-SNP-#21 | Yes | 94.2 | 96.3 | 97.2 | 93.1 |
| TEL-AML1-SNP-#22 | Yes | 93.1 | 88.9 | 87.8 | 91.5 |
| TEL-AML1-SNP-#23 | Yes | 93.0 | 95.3 | 83.6 | 89.4 |
| TEL-AML1-SNP-#24 | Yes | 89.2 | 90.0 | 89.6 | 89.9 |
| TEL-AML1-SNP-#25 | Yes | 90.1 | 92.7 | 89.1 | 92.7 |
| TEL-AML1-SNP-#26 | Yes | 93.3 | 93.5 | 94.7 | 90.8 |
| TEL-AML1-SNP-#27 | Yes | 91.8 | 94.0 | 82.8 | 90.0 |
| TEL-AML1-SNP-#28 | Yes | 90.0 | 94.0 | 92.4 | 86.8 |
| TEL-AML1-SNP-#29 | Yes | 96.2 | 96.7 | 94.5 | 94.4 |
| TEL-AML1-SNP-#30 | Yes | 97.4 | 98.2 | 90.4 | 94.7 |
| TEL-AML1-SNP-#31 | Yes | 97.6 | 98.9 | 89.0 | 94.1 |
| TEL-AML1-SNP-#32 | Yes | 97.8 | 99.1 | 88.1 | 90.2 |
| TEL-AML1-SNP-#33 | Yes | 96.5 | 98.8 | 89.4 | 91.3 |
| TEL-AML1-SNP-#34 | Yes | 88.4 | 98.7 | 89.9 | 93.1 |
| TEL-AML1-SNP-#35 | Yes | 97.2 | 98.8 | 89.0 | 92.5 |
| TEL-AML1-SNP-#36 | Yes | 98.3 | 98.2 | 88.7 | 93.6 |
| TEL-AML1-SNP-#37 | Yes | 97.2 | 97.5 | 85.5 | 92.2 |
| TEL-AML1-SNP-#38 | Yes | 97.6 | 97.8 | 82.4 | 93.5 |
| TEL-AML1-SNP-#39 | Yes | 97.3 | 98.9 | 84.5 | 94.9 |
| TEL-AML1-SNP-#40 | Yes | 94.8 | 98.9 | 95.5 | 93.2 |
| TEL-AML1-SNP-#41 | Yes | 98.0 | 97.9 | 93.5 | 94.5 |
| TEL-AML1-SNP-#42 | Yes | 95.2 | 97.9 | 92.6 | 92.2 |
| TEL-AML1-SNP-#43 | Yes | 96.7 | 98.9 | 93.0 | 91.9 |
| TEL-AML1-SNP-#44 | Yes | 92.6 | 99.1 | 94.6 | 95.2 |
| TEL-AML1-SNP-#45 | Yes | 97.1 | 98.6 | 96.2 | 93.6 |
| TEL-AML1-SNP-#46 | Yes | 94.8 | 97.5 | 94.7 | 91.5 |
| TEL-AML1-SNP-#47 | Yes | 94.5 | 98.1 | 96.3 | 90.7 |
| TEL-AML1-SNP-#48 | No | 93.9 | 98.1 | 95.9 | 90.4 |
| MLL-SNP-#1 | Yes | 89.3 | 96.2 | 91.0 | 95.0 |
| MLL-SNP-#2 | Yes | 95.4 | 96.6 | 93.0 | 93.0 |
| MLL-SNP-#3 | Yes | 92.3 | 96.7 | 97.0 | 92.8 |
| MLL-SNP-#4 | Yes | 94.1 | 97.6 | 97.0 | 92.0 |
| MLL-SNP-#5 | Yes | 94.9 | 99.5 | 96.0 | 95.0 |
| MLL-SNP-#6 | Yes | 92.9 | 99.0 | 89.9 | 95.0 |
| MLL-SNP-#7 | Yes | 97.1 | 98.7 | 96.0 | 95.0 |
| MLL-SNP-#8 | Yes | 93.7 | 99.4 | 94.0 | 98.0 |
| MLL-SNP-#9 | Yes | 96.9 | 98.7 | 95.9 | 95.6 |
| MLL-SNP-#10 | Yes | 96.4 | 99.2 | 94.0 | 92.0 |
| MLL-SNP-#11 | Yes | 93.7 | 99.1 | 95.7 | 72.8 |
| MLL-SNP-#12 | No | ND | ND | 95.4 | 92.8 |
| MLL-SNP-#13 | No | ND | ND | 92.9 | 90.2 |
| MLL-SNP-#15 | No | ND | ND | 94.9 | 80.9 |
| MLL-SNP-#16 | No | ND | ND | 94.1 | 96.0 |
| MLL-SNP-#17 | No | ND | ND | 93.9 | 89.3 |
| MLL-SNP-#18 | No | ND | ND | 92.3 | 93.5 |
| MLL-SNP-#19 | No | ND | ND | 91.0 | 83.2 |
| MLL-SNP-#20 | No | ND | ND | 92.5 | 90.1 |
| MLL-SNP-#21 | No | ND | ND | 90.8 | 94.3 |
| MLL-SNP-#22 | No | ND | ND | 95.2 | 94.3 |
| MLL-SNP-#23 | No | ND | ND | 94.9 | 93.5 |
| BCR-ABL-SNP-#1 | Yes | 95.4 | 97.2 | 94.8 | 94.7 |
| BCR-ABL-SNP-#2 | Yes | 90.0 | 95.8 | 91.5 | 96.5 |
| BCR-ABL-SNP-#3 | Yes | 92.1 | 95.4 | 94.7 | 93.4 |
| BCR-ABL-SNP-#4 | Yes | 94.0 | 96.3 | 96.0 | 84.9 |
| BCR-ABL-SNP-#5 | Yes | 90.9 | 97.6 | 92.3 | 92.7 |
| BCR-ABL-SNP-#6 | Yes | 87.9 | 94.5 | 92.1 | 86.1 |
| BCR-ABL-SNP-#7 | Yes | 92.9 | 93.4 | 93.8 | 86.8 |
| BCR-ABL-SNP-#8 | Yes | 90.2 | 98.6 | 87.7 | 91.9 |
| BCR-ABL-SNP-#9 | Yes | 97.0 | 99.1 | 95.3 | 93.6 |
| BCR-ABL-SNP-#10 | No | ND | ND | 90.3 | 83.1 |
| BCR-ABL-SNP-#11 | No | ND | ND | 94.6 | 89.6 |
| BCR-ABL-SNP-#12 | No | ND | ND | 96.8 | 92.2 |
| BCR-ABL-SNP-#13^{†} | No | ND | ND | 96.0 | 94.0 |
| BCR-ABL-SNP-#14 | No | ND | ND | 95.4 | 84.3 |
| BCR-ABL-SNP-#15 | No | ND | ND | 95.1 | 92.8 |
| BCR-ABL-SNP-#16 | No | ND | ND | 95.8 | 96.4 |
| BCR-ABL-SNP-#17 | No | ND | ND | 94.9 | 94.5 |
| BCR-ABL-SNP-#18 | No | ND | ND | 96.0 | 94.1 |
| BCR-ABL-SNP-#19 | No | ND | ND | 93.9 | 94.0 |
| BCR-ABL-SNP-#20 | No | ND | ND | 93.6 | 90.3 |
| BCR-ABL-SNP-#21 | No | ND | ND | 94.2 | 87.9 |
| BCR-ABL-SNP-#22* | No | ND | ND | 95.9 | 91.7 |
| BCR-ABL-SNP-#23* | No | ND | ND | 92.3 | 92.7 |
| BCR-ABL-SNP-#24* | No | ND | ND | 96.4 | 94.8 |
| BCR-ABL-SNP-#25* | No | ND | ND | 95.2 | 92.8 |
| BCR-ABL-SNP-#26* | No | ND | ND | 95.4 | 87.6 |
| BCR-ABL-SNP-#27* | No | ND | ND | 92.7 | 92.7 |
| BCR-ABL-SNP-#28* | No | ND | ND | 93.9 | 94.4 |
| BCR-ABL-SNP-#29* | No | ND | ND | 92.3 | 88.0 |
| BCR-ABL-SNP-#30* | No | ND | ND | 94.1 | 86.8 |
| BCR-ABL-SNP-#31* | No | ND | ND | 97.4 | 88.4 |
| BCR-ABL-SNP-#32* | No | ND | ND | 95.5 | 94.7 |
| BCR-ABL-SNP-#33* | No | ND | ND | 97.9 | 93.1 |
| BCR-ABL-SNP-#34* | No | ND | ND | 97.2 | 93.0 |
| BCR-ABL-SNP-#35* | No | ND | ND | 96.0 | 89.6 |
| BCR-ABL-SNP-#36* | No | ND | ND | 94.8 | 91.7 |
| BCR-ABL-SNP-#37* | No | ND | ND | 95.8 | 91.8 |
| BCR-ABL-SNP-#38* | No | ND | ND | 95.7 | 80.6 |
| BCR-ABL-SNP-#39* | No | ND | ND | 94.2 | 85.9 |
| BCR-ABL-SNP-#40* | No | ND | ND | 96.8 | 92.0 |
| BCR-ABL-SNP-#41* | No | ND | ND | 94.3 | 91.2 |
| BCR-ABL-SNP-#42* | No | ND | ND | 94.3 | 93.1 |
| BCR-ABL-SNP-#43* | No | ND | ND | 89.3 | 92.2 |
| Hyperdip47-50-SNP-#1 | Yes | 95.3 | 97.8 | 93.1 | 94.0 |
| Hyperdip47-50-SNP-#2 | Yes | 96.5 | 97.0 | 95.4 | 95.8 |
| Hyperdip47-50-SNP-#3 | Yes | 93.3 | 98.7 | 96.0 | 90.1 |
| Hyperdip47-50-SNP-#4 | Yes | 91.2 | 92.9 | 95.0 | 94.4 |
| Hyperdip47-50-SNP-#5 | Yes | 92.4 | 96.4 | 91.3 | 95.2 |
| Hyperdip47-50-SNP-#6 | Yes | 92.9 | 92.5 | 93.7 | 92.1 |
| Hyperdip47-50-SNP-#7 | Yes | 92.9 | 95.7 | 98.5 | 93.9 |
| Hyperdip47-50-SNP-#8 | Yes | 93.0 | 95.9 | 96.4 | 87.4 |
| Hyperdip47-50-SNP-#9 | Yes | 92.5 | 93.5 | 97.6 | 90.4 |
| Hyperdip47-50-SNP-#10 | Yes | 86.2 | 94.7 | 97.8 | 92.6 |
| Hyperdip47-50-SNP-#11 | Yes | 94.2 | 88.6 | 97.3 | 93.5 |
| Hyperdip47-50-SNP-#12 | Yes | 92.9 | 96.3 | 97.8 | 94.7 |
| Hyperdip47-50-SNP-#13 | Yes | 93.3 | 94.4 | 90.3 | 81.4 |
| Hyperdip47-50-SNP-#14 | Yes | 88.0 | 96.2 | 95.8 | 91.9 |
| Hyperdip47-50-SNP-#15 | Yes | 84.6 | 94.9 | 90.2 | 91.3 |
| Hyperdip47-50-SNP-#16 | Yes | 97.7 | 99.1 | 88.6 | 87.8 |
| Hyperdip47-50-SNP-#17 | Yes | 96.5 | 98.6 | 93.2 | 93.0 |
| Hyperdip47-50-SNP-#18 | Yes | 96.9 | 99.0 | 94.4 | 92.8 |
| Hyperdip47-50-SNP-#19 | Yes | 94.4 | 99.0 | 93.5 | 93.3 |
| Hyperdip47-50-SNP-#20 | Yes | 94.1 | 95.2 | 96.4 | 91.0 |
| Hyperdip47-50-SNP-#21 | Yes | 98.0 | 94.4 | 95.6 | 96.7 |
| Hyperdip47-50-SNP-#22 | Yes | 94.2 | 97.0 | 93.9 | 93.8 |
| Hyperdip47-50-SNP-#23 | Yes | 88.6 | 99.3 | 95.2 | 88.4 |
| Hyperdip47-50-SNP-#24 | No | 95.7 | 97.1 | 96.0 | 91.0 |
| Hypodip-SNP-#1 | Yes | 97.3 | 97.6 | 95.6 | 88.8 |
| Hypodip-SNP-#2 | Yes | 93.6 | 99.0 | 96.7 | 90.7 |
| Hypodip-SNP-#3 | Yes | 93.3 | 96.1 | 95.1 | 97.5 |
| Hypodip-SNP-#4 | Yes | 94.3 | 98.6 | 91.3 | 94.6 |
| Hypodip-SNP-#5 | Yes | 93.0 | 98.9 | 90.3 | 93.2 |
| Hypodip-SNP-#6 | Yes | 91.0 | 98.7 | 85.7 | 90.8 |
| Hypodip-SNP-#7 | Yes | 96.1 | 99.1 | 85.0 | 92.7 |
| Hypodip-SNP-#8 | Yes | 96.5 | 98.2 | 90.7 | 89.1 |
| Hypodip-SNP-#9 | Yes | 92.3 | 97.5 | 93.1 | 95.3 |
| Hypodip-SNP-#10 | Yes | 96.0 | 99.1 | 93.9 | 90.1 |
| Other-SNP-#1 | Yes | 93.6 | 96.3 | 93.2 | 85.9 |
| Other-SNP-#2 | Yes | 93.4 | 95.7 | 97.3 | 86.1 |
| Other-SNP-#3 | Yes | 93.3 | 98.2 | 98.9 | 92.5 |
| Other-SNP-#4 | Yes | 93.0 | 97.8 | 94.3 | 90.5 |
| Other-SNP-#5 | Yes | 91.1 | 92.1 | 87.1 | 85.3 |
| Other-SNP-#6 | Yes | 93.1 | 98.6 | 91.9 | 94.6 |
| Other-SNP-#7 | Yes | 93.5 | 92.2 | 92.7 | 92.1 |
| Other-SNP-#8 | Yes | 95.0 | 94.0 | 97.5 | 87.2 |
| Other-SNP-#9 | Yes | 93.6 | 97.4 | 97.6 | 88.3 |
| Other-SNP-#10 | Yes | 80.6 | 99.3 | 92.8 | 92.1 |
| Other-SNP-#11 | Yes | 95.3 | 98.8 | 95.7 | 87.0 |
| Other-SNP-#12 | Yes | 98.1 | 99.1 | 96.2 | 90.7 |
| Other-SNP-#13 | Yes | 98.1 | 95.2 | 92.7 | 91.3 |
| Other-SNP-#14 | Yes | 95.6 | 99.3 | 90.7 | 94.3 |
| Other-SNP-#15 | Yes | 91.1 | 98.9 | 92.4 | 77.4 |
| Other-SNP-#16 | Yes | 95.4 | 97.7 | 94.2 | 90.1 |
| Other-SNP-#17 | No | 95.7 | 87.5 | 93.0 | 90.0 |
| Other-SNP-#18 | No | 95.8 | 96.4 | 93.0 | 85.0 |
| Other-SNP-#19 | No | 96.9 | 95.8 | 93.2 | 95.1 |
| Other-SNP-#20 | No | 97.8 | 96.2 | 95.0 | 91.8 |
| Other-SNP-#21 | No | ND | ND | 93.9 | 86.8 |
| Other-SNP-#22 | No | ND | ND | 92.4 | 94.5 |
| Other-SNP-#23 | No | ND | ND | 91.8 | 87.9 |
| Other-SNP-#24 | No | ND | ND | 93.8 | 90.7 |
| Other-SNP-#25 | No | ND | ND | 92.9 | 89.1 |
| Other-SNP-#26 | No | ND | ND | 91.6 | 85.2 |
| Pseudodip-SNP-#1 | Yes | 96.4 | 99.2 | 95.0 | 75.2 |
| Pseudodip-SNP-#2 | Yes | 97.2 | 98.6 | 95.5 | 95.4 |
| Pseudodip-SNP-#3 | Yes | 94.0 | 94.1 | 97.1 | 93.4 |
| Pseudodip-SNP-#4 | Yes | 92.6 | 95.8 | 96.9 | 95.2 |
| Pseudodip-SNP-#5 | Yes | 92.5 | 88.7 | 95.1 | 95.3 |
| Pseudodip-SNP-#6 | Yes | 93.0 | 94.6 | 95.1 | 93.9 |
| Pseudodip-SNP-#7 | Yes | 92.7 | 93.8 | 88.0 | 90.9 |
| Pseudodip-SNP-#8 | Yes | 87.0 | 94.5 | 90.1 | 96.1 |
| Pseudodip-SNP-#9 | Yes | 88.9 | 95.5 | 85.6 | 86.4 |
| Pseudodip-SNP-#10 | Yes | 89.0 | 95.1 | 89.7 | 91.7 |
| Pseudodip-SNP-#11 | Yes | 88.2 | 95.5 | 94.5 | 87.8 |
| Pseudodip-SNP-#12 | Yes | 91.1 | 97.5 | 90.6 | 95.7 |
| Pseudodip-SNP-#13 | Yes | 91.3 | 99.2 | 84.5 | 90.5 |
| Pseudodip-SNP-#14 | Yes | 94.2 | 98.6 | 90.9 | 93.3 |
| Pseudodip-SNP-#15 | Yes | 96.5 | 98.8 | 88.7 | 96.4 |
| Pseudodip-SNP-#16 | Yes | 94.4 | 97.8 | 86.1 | 96.0 |
| Pseudodip-SNP-#17 | Yes | 94.6 | 99.4 | 93.8 | 94.4 |
| Pseudodip-SNP-#18 | Yes | 97.9 | 99.0 | 93.0 | 97.3 |
| Pseudodip-SNP-#19 | Yes | 96.7 | 99.3 | 95.0 | 90.0 |
| Pseudodip-SNP-#20 | Yes | 97.0 | 99.2 | 96.1 | 93.6 |
| Pseudodip-SNP-#21 | No | ND | ND | 90.7 | 90.5 |
| Pseudodip-SNP-#22 | No | 95.0 | 98.3 | 91.9 | 87.1 |
| Pseudodip-SNP-#23 | No | 95.6 | 96.5 | 92.0 | 92.0 |
| Pseudodip-SNP-#24 | No | 96.8 | 94.3 | 95.0 | 92.0 |
| T-ALL-SNP-#1 | Yes | 95.3 | 98.8 | 97.2 | 94.5 |
| T-ALL-SNP-#2 | Yes | 96.5 | 98.8 | 95.7 | 89.7 |
| T-ALL-SNP-#3 | Yes | 96.1 | 97.7 | 92.8 | 90.5 |
| T-ALL-SNP-#4 | Yes | 97.6 | 97.8 | 92.1 | 92.9 |
| T-ALL-SNP-#5 | Yes | 95.7 | 98.9 | 93.5 | 92.8 |
| T-ALL-SNP-#6 | Yes | 90.2 | 96.9 | 92.9 | 91.9 |
| T-ALL-SNP-#7 | Yes | 91.5 | 95.2 | 97.2 | 94.3 |
| T-ALL-SNP-#8 | Yes | 87.3 | 93.8 | 80.9 | 96.9 |
| T-ALL-SNP-#9 | Yes | 85.9 | 92.8 | 96.8 | 95.3 |
| T-ALL-SNP-#10 | Yes | 91.2 | 96.0 | 83.6 | 97.4 |
| T-ALL-SNP-#11 | Yes | 94.4 | 97.4 | 88.6 | 97.6 |
| T-ALL-SNP-#12 | Yes | 94.9 | 97.6 | 89.9 | 92.9 |
| T-ALL-SNP-#13 | Yes | 93.8 | 98.8 | 94.0 | 95.0 |
| T-ALL-SNP-#14 | Yes | 93.1 | 98.2 | 88.0 | 91.2 |
| T-ALL-SNP-#15 | Yes | 95.0 | 92.0 | 87.6 | 94.6 |
| T-ALL-SNP-#16 | Yes | 87.0 | 98.2 | 91.8 | 88.7 |
| T-ALL-SNP-#17 | Yes | 87.7 | 98.4 | 90.3 | 95.1 |
| T-ALL-SNP-#18 | Yes | 89.4 | 94.8 | 93.2 | 93.1 |
| T-ALL-SNP-#19 | Yes | 80.6 | 95.9 | 92.1 | 89.7 |
| T-ALL-SNP-#20 | Yes | 94.6 | 97.2 | 96.3 | 95.5 |
| T-ALL-SNP-#21 | Yes | 96.0 | 85.1 | 98.7 | 93.3 |
| T-ALL-SNP-#22 | Yes | 94.1 | 90.7 | 98.3 | 90.6 |
| T-ALL-SNP-#23 | Yes | 87.0 | 91.0 | 96.6 | 93.5 |
| T-ALL-SNP-#24 | Yes | 94.7 | 96.5 | 95.9 | 89.8 |
| T-ALL-SNP-#25 | Yes | 93.0 | 96.1 | 81.8 | 87.3 |
| T-ALL-SNP-#26 | Yes | 92.4 | 95.6 | 91.1 | 91.4 |
| T-ALL-SNP-#27 | Yes | 91.1 | 94.6 | 90.9 | 90.2 |
| T-ALL-SNP-#28 | Yes | 84.8 | 94.0 | 95.9 | 89.7 |
| T-ALL-SNP-#29 | Yes | 96.7 | 98.9 | 97.6 | 94.0 |
| T-ALL-SNP-#30 | Yes | 97.9 | 99.0 | 91.7 | 92.1 |
| T-ALL-SNP-#31 | Yes | 95.3 | 98.9 | 85.3 | 91.5 |
| T-ALL-SNP-#32 | Yes | 93.8 | 98.3 | 89.8 | 94.1 |
| T-ALL-SNP-#33 | Yes | 96.9 | 98.4 | 92.5 | 90.6 |
| T-ALL-SNP-#34 | Yes | 97.3 | 98.4 | 94.5 | 93.7 |
| T-ALL-SNP-#35 | Yes | 98.4 | 98.6 | 96.2 | 96.9 |
| T-ALL-SNP-#36 | Yes | 98.4 | 98.6 | 90.6 | 94.6 |
| T-ALL-SNP-#37 | Yes | 97.7 | 92.5 | 85.8 | 90.6 |
| T-ALL-SNP-#38 | Yes | 97.5 | 97.7 | 93.9 | 88.2 |
| T-ALL-SNP-#39 | Yes | 96.7 | 98.8 | 93.9 | 94.6 |
| T-ALL-SNP-#40 | Yes | 97.5 | 98.3 | 95.0 | 91.7 |
| T-ALL-SNP-#41 | Yes | 96.5 | 98.7 | 92.5 | 93.6 |
| T-ALL-SNP-#42 | Yes | 97.4 | 98.9 | 91.5 | 95.8 |
| T-ALL-SNP-#43 | Yes | 96.6 | 98.5 | 93.3 | 94.0 |
| T-ALL-SNP-#44 | Yes | 94.0 | 99.3 | 92.0 | 94.0 |
| T-ALL-SNP-#45 | Yes | 95.9 | 98.5 | 94.3 | 95.7 |
| T-ALL-SNP-#46 | Yes | 96.1 | 98.4 | 95.0 | 90.7 |
| T-ALL-SNP-#47 | Yes | 95.7 | 98.1 | 90.9 | 93.1 |
| T-ALL-SNP-#48 | Yes | 97.4 | 98.5 | 88.1 | 89.9 |
| T-ALL-SNP-#49 | Yes | 96.9 | 98.1 | 98.4 | 85.1 |
| T-ALL-SNP-#50 | Yes | 97.3 | 98.8 | 91.0 | 92.2 |

TABLE 2
The frequency of copy number abnormalities (CNAs) in pediatric
acute lymphoblastic leukemia. HD > 50, hyperdiploidy
with greater than 50 chromosomes.

| | | Deletions | All CNAs |
|---|---|---|---|
| | Gains (mean, range) | (mean, range) | (mean, range) |
| HD > 50 | 11.21 (6-21) | 1.92 (0-13) | 13.08 (6-30) |
| TCF3-PBX1 | 0.76 (0-3) | 1.24 (0-4) | 1.94 (0-5) |
| ETV6-RUNX1 | 1.25 (0-10) | 8.52 (0-33) | 9.69 (1-33) |
| MLL-rearranged | 0.26 (0-1) | 1.00 (0-5) | 1.26 (0-5) |
| BCR-ABL1 | 1.44 (0-13) | 7.33 (0-25) | 8.79 (1-26) |
| Hypodiploid | 1.10 (0-4) | 6.90 (3-20) | 8 (3-24) |
| Other | 1.57 (0-13) | 5.52 (0-22) | 7.09 (0-30) |
| T-ALL | 0.96 (0-10) | 6.08 (0-50) | 7.02 (0-50) |
| Total | 2.48 (0-21) | 5.34 (0-50) | 7.80 (0-50) |

TABLE 3
depicts the frequency of recurring DNA copy number abnormalities in ALL.

| ALL subtype (N) | IKZF1 | CDKN2A | PAX5 | C20orf94 | RB1 | MEF2C | EBF1 | BTG1 | DLEU | FHIT | ETV6 |
|---|---|---|---|---|---|---|---|---|---|---|---|
| B-progenitor | | | | | | | | | | | |
| (254) | | | | | | | | | | | |
| BCR-ABL1(43) | 36 | 23 | 22 | 10 | 8 | 6 | 6 | 6 | 4 | 4 | 3 |
| childhood (21) | 16 | 13 | 12 | 3 | 4 | 4 | 3 | 2 | 3 | 2 | 1 |
| adult (22) | 20 | 10 | 10 | 7 | 4 | 2 | 3 | 4 | 1 | 2 | 2 |
| Hypodiploid (10) | 5 | 10 | 10 | 0 | 0 | 0 | 1 | 1 | 0 | 1 | 2 |
| Other B-ALL | 15 | 25 | 22 | 4 | 1 | | 2 | 5 | 1 | 3 | 10 |
| (75) | | | | | | | | | | | |
| High hyperdiploid | 2 | 8 | 4 | 1 | 3 | 0 | 0 | 0 | 5 | 0 | 3 |
| (39) | | | | | | | | | | | |
| MLL-rearranged | 1 | 4 | 4 | 0 | 2 | 0 | 0 | 0 | 3 | 0 | 2 |
| (22) | | | | | | | | | | | |
| TCF3-PBX1 (17) | 0 | 6 | 7 | 0 | 2 | 0 | 0 | 0 | 2 | 0 | 0 |
| ETV6-RUNX1 | 0 | 14 | 16 | 6 | 2 | 0 | 5 | 7 | 4 | 6 | 33 |
| (48) | | | | | | | | | | | |
| T-lineage (50) | 2 | 36 | 5 | 1 | 6 | 1 | 3 | 0 | 3 | 0 | 4 |
| Total (304) | 61 | 126 | 90 | 22 | 24 | 7 | 17 | 19 | 22 | 14 | 57 |
| P | 6.6 × 10⁻²⁷ | 7.4 × 10⁻¹⁰ | 1.4 × 10⁻⁹ | 7.0 × 10⁻⁸ | 1.1 × 10⁻⁶ | 0.0004 | 0.0247 | 1.5 × 10⁻⁷ | 2.6 × 10⁻⁶ | 0.0076 | 9.1 × 10⁻¹⁵ |

The prevalence of recurring genomic abnormalities in BCR-ABL1 B-progenitor ALL identified by SNP array analysis is shown for each ALL subtype. The exact likelihood ratio P value for variation in the frequency of each lesion across ALL subtypes is shown. The DLEU region at 13q14 incorporates the miRNA genes MIRN16-1 and MIRN15A.

TABLE 4
The distribution of recurring CNAs observed in BCR-ABL1 ALL. e, exon.

| | IKZF1 | IKZF1 | | | | | | | | | | |
|---|---|---|---|---|---|---|---|---|---|---|---|---|
| ALL SNP paper code | deletion | deletion | FHIT | MEF2C | EBF | CDKNA | PAX5 | ETV6 | BTG1 | RB1 | DLEU2 | C20orf94 |
| Hyperdip>50-SNP-#1 | No | | No | No | No | No | No | No | No | No | No | No |
| Hyperdip>50-SNP-#2 | No | | No | No | No | No | No | No | No | No | No | No |
| Hyperdip>50-SNP-#3 | No | | No | No | No | No | No | No | No | Yes | Yes | No |
| Hyperdip>50-SNP-#4 | No | | No | No | No | Yes | No | No | No | No | No | No |
| Hyperdip>50-SNP-#5 | No | | No | No | No | No | No | No | No | No | No | No |
| Hyperdip>50-SNP-#6 | No | | No | No | No | No | No | No | No | No | No | No |
| Hyperdip>50-SNP-#7 | No | | No | No | No | Yes | No | No | No | No | No | No |
| Hyperdip>50-SNP-#8 | No | | No | No | No | No | No | No | No | No | No | No |
| Hyperdip>50-SNP-#9 | No | | No | No | No | No | No | No | No | No | No | No |
| Hyperdip>50-SNP-#10 | No | | No | No | No | No | No | No | No | Yes | Yes | No |
| Hyperdip>50-SNP-#11 | No | | No | No | No | No | No | No | No | No | No | No |
| Hyperdip>50-SNP-#12 | No | | No | No | No | Yes | No | No | No | No | No | No |
| Hyperdip>50-SNP-#13 | No | | No | No | No | No | No | No | No | No | No | No |
| Hyperdip>50-SNP-#14 | No | | No | No | No | No | No | No | No | No | No | No |
| Hyperdip>50-SNP-#15 | No | | No | No | No | No | No | No | No | No | No | No |
| Hyperdip>50-SNP-#16 | No | | No | No | No | No | No | No | No | No | No | No |
| Hyperdip>50-SNP-#17 | No | | No | No | No | Yes | Yes | Yes | No | No | No | No |
| Hyperdip>50-SNP-#18 | No | | No | No | No | No | No | No | No | No | No | No |
| Hyperdip>50-SNP-#19 | No | | No | No | No | No | No | No | No | No | No | No |
| Hyperdip>50-SNP-#20 | No | | No | No | No | No | No | No | No | No | No | No |
| Hyperdip>50-SNP-#21 | No | | No | No | No | No | No | No | No | No | Yes | No |
| Hyperdip>50-SNP-#22 | No | | No | No | No | No | No | No | No | No | No | No |
| Hyperdip>50-SNP-#23 | No | | No | No | No | No | No | No | No | No | No | No |
| Hyperdip>50-SNP-#24 | No | | No | No | No | Yes | Yes | Yes | No | No | No | No |
| Hyperdip>50-SNP-#25 | No | | No | No | No | Yes | No | No | No | No | No | No |
| Hyperdip>50-SNP-#26 | No | | No | No | No | No | No | No | No | No | No | No |
| Hyperdip>50-SNP-#27 | No | | No | No | No | No | No | No | No | No | No | No |
| Hyperdip>50-SNP-#28 | No | | No | No | No | No | Yes | No | No | Yes | Yes | Yes |
| Hyperdip>50-SNP-#29 | No | | No | No | No | No | Yes | No | No | No | No | No |
| Hyperdip>50-SNP-#30 | No | | No | No | No | No | No | No | No | No | No | No |
| Hyperdip>50-SNP-#31 | Yes | Promoter- | No | No | No | No | No | No | No | No | No | No |
| | | e2 | | | | | | | | | | |
| Hyperdip>50-SNP-#32 | No | | No | No | No | No | No | No | No | No | Yes | No |
| Hyperdip>50-SNP-#33 | No | | No | No | No | No | No | No | No | No | No | No |
| Hyperdip>50-SNP-#34 | Yes | All gene | No | No | No | No | No | Yes | No | No | No | No |
| Hyperdip>50-SNP-#35 | No | | No | No | No | No | No | No | No | No | No | No |
| Hyperdip>50-SNP-#36 | No | | No | No | No | No | No | No | No | No | No | No |
| Hyperdip>50-SNP-#37 | No | | No | No | No | Yes | No | No | No | No | No | No |
| Hyperdip>50-SNP-#38 | No | | No | No | No | No | No | No | No | No | No | No |
| Hyperdip>50-SNP-#39 | No | | No | No | No | Yes | No | No | No | No | No | No |
| E2A-PBX1-SNP-#1 | No | | No | No | No | No | No | No | No | No | No | No |
| E2A-PBX1-SNP-#2 | No | | No | No | No | No | No | No | No | No | No | No |
| E2A-PBX1-SNP-#3 | No | | No | No | No | No | No | No | No | No | No | No |
| E2A-PBX1-SNP-#4 | No | | No | No | No | No | No | No | No | No | No | No |
| E2A-PBX1-SNP-#5 | No | | No | No | No | Yes | Yes | No | No | No | No | No |
| E2A-PBX1-SNP-#6 | No | | No | No | No | Yes | Yes | No | No | No | No | No |
| E2A-PBX1-SNP-#7 | No | | No | No | No | No | No | No | No | No | No | No |
| E2A-PBX1-SNP-#8 | No | | No | No | No | No | Yes | No | No | Yes | Yes | No |
| E2A-PBX1-SNP-#9 | No | | No | No | No | No | No | No | No | No | No | No |
| E2A-PBX1-SNP-#10 | No | | No | No | No | Yes | Yes | No | No | No | No | No |
| E2A-PBX1-SNP-#11 | No | | No | No | No | Yes | Yes | No | No | Yes | Yes | No |
| E2A-PBX1-SNP-#12 | No | | No | No | No | No | No | No | No | No | No | No |
| E2A-PBX1-SNP-#13 | No | | No | No | No | Yes | Yes | No | No | No | No | No |
| E2A-PBX1-SNP-#14 | No | | No | No | No | No | No | No | No | No | No | No |
| E2A-PBX1-SNP-#15 | No | | No | No | No | No | No | No | No | No | No | No |
| E2A-PBX1-SNP-#16 | No | | No | No | No | Yes | Yes | No | No | No | No | No |
| E2A-PBX1-SNP-#17 | No | | No | No | No | No | No | No | No | No | No | No |
| TEL-AML1-SNP-#1 | No | | No | No | No | No | No | Yes | No | No | No | No |
| TEL-AML1-SNP-#2 | No | | No | No | No | No | No | No | No | No | Yes | No |
| TEL-AML1-SNP-#3 | No | | No | No | No | No | No | Yes | No | No | No | No |
| TEL-AML1-SNP-#4 | No | | No | No | No | Yes | No | Yes | No | No | No | No |
| TEL-AML1-SNP-#5 | No | | No | No | Yes | No | No | Yes | Yes | No | No | No |
| TEL-AML1-SNP-#6 | No | | No | No | No | No | No | Yes | No | No | No | No |
| TEL-AML1-SNP-#7 | No | | No | No | No | Yes | No | Yes | No | No | No | No |
| TEL-AML1-SNP-#8 | No | | No | No | No | No | No | Yes | Yes | No | No | No |
| TEL-AML1-SNP-#9 | No | | No | No | No | No | Yes | No | Yes | No | No | Yes |
| TEL-AML1-SNP-#10 | No | | Yes | No | No | Yes | No | No | No | No | No | No |
| TEL-AML1-SNP-#11 | No | | No | No | No | No | Yes | Yes | No | No | No | No |
| TEL-AML1-SNP-#12 | No | | No | No | Yes | Yes | No | Yes | No | No | No | Yes |
| TEL-AML1-SNP- | No | | No | No | No | No | No | No | No | No | No | No |
| TEL-AML1-SNP- | No | | No | No | No | Yes | No | Yes | No | No | No | No |
| TEL-AML1-SNP- | No | | No | No | No | No | Yes | No | No | No | No | No |
| TEL-AML1-SNP- | No | | No | No | No | No | No | Yes | No | No | No | No |
| TEL-AML1-SNP- | No | | No | No | No | No | No | Yes | No | No | No | No |
| TEL-AML1-SNP- | No | | No | No | No | No | Yes | Yes | No | No | No | No |
| TEL-AML1-SNP- | No | | No | No | No | No | Yes | Yes | No | No | No | No |
| TEL-AML1-SNP- | No | | No | No | No | No | Yes | Yes | No | No | No | No |
| TEL-AML1-SNP- | No | | No | No | No | No | Yes | Yes | No | No | No | No |
| TEL-AML1-SNP- | No | | No | No | No | Yes | No | Yes | No | No | No | Yes |
| TEL-AML1-SNP- | No | | Yes | No | No | No | Yes | Yes | No | No | No | No |
| TEL-AML1-SNP- | No | | No | No | No | No | No | Yes | Yes | No | No | No |
| TEL-AML1-SNP- | No | | No | No | No | No | No | No | No | No | No | No |
| TEL-AML1-SNP- | No | | No | No | Yes | No | No | Yes | No | No | No | No |
| TEL-AML1-SNP- | No | | No | No | No | No | Yes | No | No | No | No | No |
| TEL-AML1-SNP- | No | | Yes | No | No | No | Yes | No | Yes | No | No | Yes |
| TEL-AML1-SNP- | No | | No | No | No | Yes | No | No | No | No | No | No |
| TEL-AML1-SNP- | No | | Yes | No | No | Yes | Yes | No | Yes | No | No | No |
| TEL-AML1-SNP- | No | | No | No | No | No | No | Yes | No | No | No | No |
| TEL-AML1-SNP- | No | | No | No | No | No | Yes | Yes | No | Yes | Yes | No |
| TEL-AML1-SNP- | No | | No | No | Yes | No | Yes | Yes | No | No | No | No |
| TEL-AML1-SNP- | No | | No | No | No | No | No | Yes | No | Yes | Yes | No |
| TEL-AML1-SNP- | No | | No | No | No | Yes | No | No | No | No | No | No |
| TEL-AML1-SNP- | No | | Yes | No | No | Yes | No | Yes | No | No | No | No |
| TEL-AML1-SNP- | No | | No | No | No | No | No | No | No | No | No | No |
| TEL-AML1-SNP- | No | | No | No | No | No | Yes | Yes | No | No | No | No |
| TEL-AML1-SNP- | No | | No | No | No | Yes | No | Yes | No | No | No | Yes |
| TEL-AML1-SNP- | No | | No | No | No | No | No | Yes | No | No | No | No |
| TEL-AML1-SNP- | No | | No | No | No | No | No | Yes | No | No | No | No |
| TEL-AML1-SNP- | No | | No | No | Yes | No | Yes | Yes | No | No | No | No |
| TEL-AML1-SNP- | No | | No | No | No | Yes | No | No | No | No | No | No |
| TEL-AML1-SNP- | No | | Yes | No | No | Yes | No | No | Yes | No | No | Yes |
| TEL-AML1-SNP- | No | | No | No | No | No | No | Yes | No | No | Yes | No |
| TEL-AML1-SNP- | No | | No | No | No | No | No | Yes | No | No | No | No |
| TEL-AML1-SNP- | No | | No | No | No | No | Yes | Yes | No | No | No | No |
| TEL-AML1-SNP- | No | | No | No | No | Yes | No | No | No | No | No | No |
| MLL-SNP-#12 | No | | No | No | No | No | No | No | No | No | No | No |
| MLL-SNP-#13 | No | | No | No | No | No | No | No | No | No | No | No |
| MLL-SNP-#15 | No | | No | No | No | No | No | No | No | No | No | No |
| MLL-SNP-#1 | No | | No | No | No | No | No | No | No | No | No | No |
| MLL-SNP-#2 | No | | No | No | No | Yes | Yes | Yes | No | No | No | No |
| MLL-SNP-#3 | No | | No | No | No | No | No | No | No | No | No | No |
| MLL-SNP-#4 | No | | No | No | No | No | No | No | No | No | No | No |
| MLL-SNP-#16 | No | | No | No | No | Yes | Yes | Yes | No | Yes | Yes | No |
| MLL-SNP-#17 | No | | No | No | No | No | Yes | No | No | Yes | Yes | No |
| MLL-SNP-#18 | No | | No | No | No | No | No | No | No | No | No | No |
| MLL-SNP-#19 | No | | No | No | No | No | Yes, | No | No | No | No | No |
| | | | | | | | amplifie | | | | | |
| MLL-SNP-#5 | No | | No | No | No | No | No | No | No | No | No | No |
| MLL-SNP-#6 | Yes | A3-6 | No | No | No | No | No | No | No | No | No | No |
| MLL-SNP-#7 | No | | No | No | No | No | No | No | No | No | No | No |
| MLL-SNP-#20 | No | | No | No | No | Yes | No | No | No | No | No | No |
| MLL-SNP-#8 | No | | No | No | No | No | No | No | No | No | No | No |
| MLL-SNP-#9 | No | | No | No | No | No | No | No | No | No | No | No |
| MLL-SNP-#10 | No | | No | No | No | No | No | No | No | No | No | No |
| MLL-SNP-#21 | No | | No | No | No | Yes | No | No | No | No | Yes | No |
| MLL-SNP-#22 | No | | No | No | No | No | No | No | No | No | No | No |
| MLL-SNP-#11 | No | | No | No | No | No | No | No | No | No | No | No |
| MLL-SNP-#23 | No | | No | No | No | No | No | No | No | No | No | No |
| BCR-ABL-SNP-#1 | Yes | A3-6 | Yes | Yes | No | Yes | Yes | No | Yes | Yes | No | Yes |
| BCR-ABL-SNP-#2 | No | | No | No | No | No | No | No | No | No | No | No |
| BCR-ABL-SNP-#3 | Yes | e3-distal | No | No | No | No | No | No | No | No | No | No |
| BCR-ABL-SNP-#4 | Yes | A3-6 | No | No | No | Yes | Yes | No | No | No | No | No |
| BCR-ABL-SNP-#5 | Yes | A3-6 | No | No | Yes | No | No | No | Yes | No | No | No |
| BCR-ABL-SNP-#6 | No | | No | No | No | Yes | No | No | No | No | No | No |
| BCR-ABL-SNP-#7 | Yes | A3-6 | No | No | No | No | Yes | Yes | No | No | No | Yes |
| BCR-ABL-SNP-#10 | Yes | A3-6 | No | No | No | No | Yes | No | No | No | No | No |
| BCR-ABL-SNP-#11 | No | | No | No | No | Yes | No | No | No | No | No | Yes |
| BCR-ABL-SNP-#12 | Yes | A3-6 | No | No | No | No | Yes | No | No | No | Yes | No |
| BCR-ABL-SNP-#13 | Yes | A3-6 | No | No | No | Yes | Yes | No | No | Yes | No | Yes |
| BCR-ABL-SNP-#14 | No | | No | No | No | No | No | No | No | No | No | No |
| BCR-ABL-SNP-#15 | Yes | promoter | No | No | Yes | Yes | Yes | No | No | Yes | No | Yes |
| | | e3- | | | | | | | | | | |
| BCR-ABL-SNP-#16 | Yes | A3-6 | No | No | No | Yes | No | No | No | No | No | No |
| BCR-ABL-SNP-#17 | Yes | promoter-e2 | No | No | Yes | No | No | No | No | No | No | No |
| BCR-ABL-SNP-#18 | Yes | All gene | No | No | No | No | No | No | No | No | No | No |
| BCR-ABL-SNP-#8 | No | | No | No | No | No | No | No | No | No | No | No |
| BCR-ABL-SNP-#19 | Yes | A1-6 | No | Yes | No | Yes | Yes | No | Yes | Yes | No | Yes |
| BCR-ABL-SNP-#20 | Yes | All gene-e3 | No | No | No | Yes | Yes | No | No | No | No | No |
| BCR-ABL-SNP-#9 | Yes | All gene, | Yes | No | No | No | Yes | No | No | No | No | No |
| | | homozygous | | | | | | | | | | |
| | | A1-distal | | | | | | | | | | |
| BCR-ABL-SNP-#21 | Yes | All gene, | No | No | No | Yes | No | Yes | Yes | No | No | Yes |
| | | homo A3- | | | | | | | | | | |
| Hyperdip47-50-SNP-#1 | No | | No | No | No | No | No | Yes | No | Yes | Yes | No |
| Hyperdip47-50-SNP-#2 | Yes | A3-6 | No | No | No | Yes | No | No | No | No | No | No |
| Hyperdip47-50-SNP-#3 | No | | No | No | No | No | No | No | No | No | No | No |
| Hyperdip47-50-SNP-#4 | No | | No | No | No | Yes | No | No | No | No | No | No |
| Hyperdip47-50-SNP-#5 | No | | No | No | No | Yes | Yes | No | No | No | No | Yes |
| Hyperdip47-50-SNP-#6 | No | | No | No | No | Yes | Yes | No | No | No | No | No |
| Hyperdip47-50-SNP-#7 | No | | No | No | No | No | No | No | No | No | No | Yes |
| Hyperdip47-50-SNP-#8 | No | | No | No | No | No | Yes | No | No | No | No | No |
| Hyperdip47-50-SNP-#9 | No | | No | No | No | Yes | Yes | No | No | No | No | No |
| Hyperdip47-50-SNP-#10 | No | | No | No | No | Yes | Yes | Yes | No | No | No | No |
| Hyperdip47-50-SNP-#1 | Yes | All gene | Yes | No | No | No | No | No | Yes | No | No | No |
| Hyperdip47-50-SNP-#12 | No | | No | No | No | No | No | No | No | No | No | No |
| Hyperdip47-50-SNP-#13 | No | | No | No | No | No | No | No | No | No | No | No |
| Hyperdip47-50-SNP-#14 | No | | No | No | No | No | No | No | No | No | No | No |
| Hyperdip47-50-SNP-#15 | No | | No | No | No | No | No | No | No | No | No | No |
| Hyperdip47-50-SNP-#16 | No | | No | No | No | No | No | No | No | No | No | No |
| Hyperdip47-50-SNP-#17 | No | | No | No | No | No | No | No | No | No | No | No |
| Hyperdip47-50-SNP-#18 | Yes | A1-7 | No | No | No | Yes | No | No | No | No | No | No |
| Hyperdip47-50-SNP-#19 | No | | No | No | No | Yes | Yes | No | Yes | No | No | No |
| Hyperdip47-50-SNP-#20 | No | | No | No | Yes | No | Yes | No | No | No | No | No |
| Hyperdip47-50-SNP-#21 | No | | No | No | No | Yes | No | No | Yes | No | No | No |
| Hyperdip47-50-SNP-#22 | No | | No | No | No | Yes | No | No | No | No | No | Yes |
| Hyperdip47-50-SNP-#23 | No | | No | No | No | No | No | No | No | No | No | No |
| Hyperdip-SNP-#1 | Yes | All gene | No | No | No | Yes | Yes | Yes | No | No | No | No |
| Hyperdip-SNP-#26 | No | | No | No | No | Yes | Yes | No | No | No | No | No |
| Hyperdip-SNP-#3 | No | | No | No | No | Yes | Yes | Yes | No | No | No | No |
| Hyperdip-SNP-#4 | Yes | Δ3-6 | No | No | No | Yes | Yes | No | No | No | No | No |
| Hyperdip-SNP-#5 | Yes | All gene | No | No | Yes | Yes | Yes | No | No | No | No | No |
| Hyperdip-SNP-#6 | No | | No | No | No | Yes | Yes | No | No | No | No | No |
| Hyperdip-SNP-#7 | Yes | All gene | No | No | No | Yes | yes | No | Yes | No | No | No |
| Hyperdip-SNP-#8 | Yes | All gene | Yes | No | No | Yes | Yes | No | No | No | No | No |
| Hyperdip-SNP-#9 | No | | No | No | No | Yes | Yes | No | No | No | No | No |
| Hyperdip-SNP-#10 | No | | No | No | No | Yes | Yes | No | No | No | No | No |
| Pseudodip-SNP-#1 | No | | No | No | No | Yes | Yes | Yes | No | No | No | No |
| Other-SNP-#1 | No | | No | No | No | No | No | No | No | No | No | No |
| Pseudodip-SNP-#2 | No | | No | No | No | Yes | Yes | No | No | No | No | No |
| Pseudodip-SNP-#3 | No | | No | No | No | Yes | No | No | No | No | No | No |
| Pseudodip-SNP-#4 | No | | No | No | No | No | Yes | No | Yes | No | No | Yes |
| Pseudodip-SNP-#22 | No | | No | No | No | No | No | No | No | No | No | No |
| Pseudodip-SNP-#5 | No | | No | No | No | No | No | No | No | No | No | No |
| Pseudodip-SNP-#6 | Yes | All gene | No | No | No | Yes | Yes | No | No | No | No | No |
| Pseudodip-SNP-#7 | No | | No | No | No | No | No | Yes | No | No | No | No |
| Other-SNP-#2 | Yes | All gene | No | No | No | No | No | Yes | No | No | No | No |
| Other-SNP-#3 | Yes | Δ3-6 | No | No | Yes | No | Yes | No | No | No | No | No |
| Other-SNP-#4 | No | | No | No | No | No | Yes | Yes | No | No | No | No |
| Other-SNP-#5 | No | | No | No | No | No | Yes | No | No | No | No | No |
| Pseudodip-SNP-#8 | No | | No | No | No | No | No | No | No | No | No | No |
| Pseudodip-SNP-#9 | No | | No | No | No | Yes | Yes | No | No | No | No | No |
| Pseudodip-SNP-#10 | No | | No | No | No | No | No | No | No | No | No | No |
| Pseudodip-SNP-#11 | No | | No | No | No | No | Yes | No | No | No | No | No |
| Other-SNP-#6 | No | | No | No | No | No | No | No | No | No | No | No |
| Pseudodip-SNP-#12 | No | | No | No | No | Yes | Yes | No | No | No | No | No |
| Other-SNP-#7 | No | | No | No | No | No | No | No | No | No | No | No |
| Pseudodip-SNP-#23 | No | | No | No | No | Yes | No | No | No | No | No | No |
| Pseudodip-SNP-#24 | No | | No | No | No | No | No | No | No | No | No | No |
| Other-SNP-#17 | Yes | Δ3-6 | No | No | No | No | No | No | No | No | No | No |
| Hyperdip47-50-SNP-#24 | Yes | Δ3-6 | No | No | No | No | No | No | No | No | No | No |
| Other-SNP-#8 | No | | No | No | No | No | No | No | No | No | No | No |
| Other-SNP-#9 | Yes | Δ3-6 | No | No | No | No | No | No | No | No | No | No |
| Pseudodip-SNP-#13 | No | | No | No | No | No | Yes | No | No | No | No | No |
| Pseudodip-SNP-#14 | No | | No | No | No | No | No | No | No | No | No | No |
| Other-SNP-#10 | No | | No | No | No | Yes | No | No | No | No | No | No |
| Pseudodip-SNP-#15 | No | | No | No | No | Yes | No | No | No | No | No | No |
| Other-SNP-#18 | No | | No | No | No | No | No | No | No | No | No | No |
| Pseudodip-SNP-#16 | No | | No | No | No | Yes | No | No | No | No | No | No |
| Other-SNP-#11 | No | | No | No | No | No | No | No | No | No | No | No |
| Pseudodip-SNP-#21 | No | | No | No | No | No | No | Yes | No | No | No | No |
| Other-SNP-#12 | Yes | Δ3-6 | No | No | No | No | No | No | No | No | No | No |
| Hyperdip>50-SNP-#40 | No | | No | No | No | No | No | No | No | No | No | No |
| Other-SNP-#19 | Yes | Δ3-6 | No | No | No | No | No | No | No | No | No | No |
| Other-SNP-#13 | No | | No | No | No | No | No | No | No | No | No | No |
| Pseudodip-SNP-#17 | No | | Yes | No | No | Yes | No | No | No | No | No | No |
| Other-SNP-#14 | No | | No | No | No | Yes | Yes | No | No | No | No | No |
| Pseudodip-SNP-#18 | No | | No | No | No | No | No | Yes | No | No | No | No |
| Pseudodip-SNP-#19 | No | | No | No | No | No | No | Yes | No | No | No | No |
| Pseudodip-SNP-#20 | Yes | All gene | No | No | No | Yes | No | No | No | No | No | No |
| Other-SNP-#15 | No | | No | No | No | No | No | No | No | No | No | No |
| Other-SNP-#20 | No | | No | No | No | No | No | No | No | No | No | No |
| Other-SNP-#16 | No | | No | No | No | No | No | Yes | No | No | No | No |
| T-ALL-SNP-#1 | No | | No | No | No | Yes | No | No | No | Yes | No | No |
| T-ALL-SNP-#2 | No | | No | No | No | Yes | No | No | No | Yes | No | No |
| T-ALL-SNP-#3 | Yes | All gene | No | No | No | Yes | Yes | No | No | No | No | No |
| T-ALL-SNP-#4 | No | | No | No | No | No | No | Yes | No | Yes | Yes | No |
| T-ALL-SNP-#5 | No | | No | No | No | Yes | No | No | No | No | No | No |
| T-ALL-SNP-#6 | No | | No | No | No | Yes | Yes | No | No | No | No | Yes |
| T-ALL-SNP-#7 | No | | No | No | No | Yes | No | No | No | No | No | No |
| T-ALL-SNP-#8 | No | | No | No | No | No | No | No | No | No | No | No |
| T-ALL-SNP-#9 | No | | No | No | No | Yes | No | No | No | No | No | No |
| T-ALL-SNP-#10 | No | | No | No | No | No | No | No | No | No | No | No |
| T-ALL-SNP-#11 | No | | No | No | No | No | No | No | No | No | No | No |
| T-ALL-SNP-#12 | No | | No | No | Yes | No | No | No | No | No | No | No |
| T-ALL-SNP-#13 | No | | No | No | No | Yes | No | No | No | No | No | No |
| T-ALL-SNP-#14 | No | | No | No | No | Yes | No | No | No | No | No | No |
| T-ALL-SNP-#15 | Yes | Δ3-6 | No | No | No | Yes | No | No | No | No | No | No |
| T-ALL-SNP-#16 | No | | No | No | No | Yes | No | No | No | No | No | No |
| T-ALL-SNP-#17 | No | | No | No | No | No | No | Yes | No | No | No | No |
| T-ALL-SNP-#18 | No | | No | No | No | No | No | No | No | No | No | No |
| T-ALL-SNP-#19 | No | | No | No | No | Yes | Yes | No | No | No | No | No |
| T-ALL-SNP-#20 | No | | No | No | No | Yes | Yes | No | No | No | No | No |
| T-ALL-SNP-#21 | No | | No | No | No | Yes | No | Yes | No | No | No | No |
| T-ALL-SNP-#22 | No | | No | No | No | Yes | No | No | No | No | No | No |
| T-ALL-SNP-#23 | No | | No | No | No | Yes | No | No | No | No | No | No |
| T-ALL-SNP-#24 | No | | No | No | No | Yes | No | No | No | No | No | No |
| T-ALL-SNP-#25 | No | | No | No | No | Yes | No | No | No | No | No | No |
| T-ALL-SNP-#26 | No | | No | No | No | Yes | No | No | No | No | No | No |
| T-ALL-SNP-#27 | No | | No | No | No | Yes | No | No | No | No | No | No |
| T-ALL-SNP-#28 | No | | No | No | No | Yes | No | No | No | No | No | No |
| T-ALL-SNP-#29 | No | | No | No | No | Yes | No | No | No | No | No | No |
| T-ALL-SNP-#30 | No | | No | No | No | Yes | No | No | No | No | No | No |
| T-ALL-SNP-#31 | No | | No | No | No | Yes | No | No | No | No | No | No |
| T-ALL-SNP-#32 | No | | No | No | No | Yes | No | No | No | No | No | No |
| T-ALL-SNP-#33 | No | | No | No | No | Yes | No | No | No | No | No | No |
| T-ALL-SNP-#34 | No | | No | No | No | No | No | No | No | No | No | No |
| T-ALL-SNP-#35 | No | | No | No | No | No | No | No | No | No | No | No |
| T-ALL-SNP-#36 | No | | No | No | No | Yes | No | No | No | No | No | No |
| T-ALL-SNP-#37 | No | | No | No | No | Yes | No | No | No | No | No | No |
| T-ALL-SNP-#38 | No | | No | No | No | No | No | No | No | No | No | No |
| T-ALL-SNP-#39 | No | | No | No | No | No | No | No | No | Yes | Yes | No |
| T-ALL-SNP-#40 | No | | No | No | No | No | No | No | No | No | No | No |
| T-ALL-SNP-#41 | No | | No | No | Yes | No | No | Yes | No | Yes | Yes | No |
| T-ALL-SNP-#42 | No | | No | No | No | Yes | No | No | No | No | No | No |
| T-ALL-SNP-#43 | No | | No | Yes | Yes | Yes | No | No | No | No | No | No |
| T-ALL-SNP-#44 | No | | No | No | No | No | No | No | No | No | No | No |
| T-ALL-SNP-#45 | No | | No | No | No | Yes | No | No | No | No | No | No |
| T-ALL-SNP-#46 | No | | No | No | No | Yes | No | No | No | No | No | No |
| T-ALL-SNP-#47 | No | | No | No | No | Yes | No | No | No | No | No | No |
| T-ALL-SNP-#48 | No | | No | No | No | Yes | No | No | No | Yes | No | No |
| T-ALL-SNP-#49 | No | | No | No | No | Yes | Yes | No | No | No | No | No |
| T-ALL-SNP-#50 | No | | No | No | No | Yes | No | No | No | No | No | No |
| Other-SNP-#21 | No | | No | No | No | Yes | Yes | No | No | No | No | No |
| Other-SNP-#22 | Yes | A3-6 | No | No | No | No | Yes | No | No | No | No | No |
| Other-SNP-#23 | Yes | A3-6 | Yes | No | No | No | No | No | yes | No | No | No |
| Other-SNP-#24 | No | | No | No | No | Yes | Yes | No | No | No | No | No |
| Other-SNP-#25 | No | | No | No | No | No | No | No | No | No | No | No |
| Other-SNP-#26 | Yes | A3-6 | No | No | No | No | No | No | No | No | No | No |
| BCR-ABL-SNP-#22 | Yes | All gene | No | No | No | No | No | No | No | No | No | No |
| BCR-ABL-SNP-#23 | Yes | A1-6 | No | Yes | Yes | No | No | No | No | No | No | No |
| BCR-ABL-SNP-#24 | Yes | All gene | Yes | Yes | Yes | Yes | Yes | No | No | No | No | No |
| BCR-ABL-SNP-#25 | Yes | A1-7 | No | No | No | Yes | Yes | No | No | No | No | No |
| BCR-ABL-SNP-#26 | Yes | Promoter; | No | No | Yes | No | Yes | Yes | Yes | Yes | Yes | Yes |
| | | A3-6 | | | | | | | | | | |
| BCR-ABL-SNP-#27 | Yes | All gene | No | No | No | Yes | Yes | No | No | No | No | No |
| BCR-ABL-SNP-#28 | Yes | A1-6, | No | No | No | Yes | Yes | No | No | No | No | No |
| | | homozygous | | | | | | | | | | |
| | | A1-2 | | | | | | | | | | |
| BCR-ABL-SNP-#29 | Yes | Promoter- | No | No | No | No | No | No | No | No | No | No |
| | | e0; A3- | | | | | | | | | | |
| BCR-ABL-SNP-#30 | Yes | All gene, | No | Yes | No | Yes | Yes | No | No | No | No | No |
| | | homozygous | | | | | | | | | | |
| | | A1-distal | | | | | | | | | | |
| BCR-ABL-SNP-#31 | Yes | Promoter- | No | No | No | Yes | Yes | No | No | No | No | No |
| | | e0, A4- | | | | | | | | | | |
| BCR-ABL-SNP-#32 | Yes | A1-distal | No | No | No | Yes | No | No | No | No | No | No |
| BCR-ABL-SNP-#33 | Yes | A3-6 | Yes | No | No | No | No | No | No | Yes | No | No |
| BCR-ABL-SNP-#34 | Yes | A3-6 | No | No | No | Yes | Yes | No | No | No | Yes | No |
| BCR-ABL-SNP-#35 | No | | No | No | No | Yes | Yes | No | No | No | No | No |
| BCR-ABL-SNP-#36 | Yes | Homozygous | No | No | No | Yes | Yes | No | No | Yes | Yes | No |
| | | all gene | | | | | | | | | | |
| BCR-ABL-SNP-#37 | Yes | All gene | No | No | No | Yes | No | No | No | No | No | No |
| BCR-ABL-SNP-#38 | Yes | A3-6 | No | No | No | Yes | Yes | No | Yes | No | No | Yes |
| BCR-ABL-SNP-#39 | Yes | A3-6 | No | No | No | Yes | Yes | No | No | No | No | Yes |
| BCR-ABL-SNP-#40 | No | | No | Yes | No | No | No | No | No | No | No | No |
| BCR-ABL-SNP- | Yes | All gene | No | No | No | No | No | No | No | Yes | No | No |
| BCR-ABL-SNP- | Yes | All gene | No | No | No | No | No | No | No | No | No | No |
| BCR-ABL-SNP- | Yes | Δ3-6 | No | No | No | No | No | No | No | No | No | No |

TABLE 5

| | | | | | | | | | % cells |
|---|---|---|---|---|---|---|---|---|---|
| | Region of | Genomic qPCR IKZF1/RNAseP | | | | | | | with IKZF1 |
| | IKZF1 | ratio | | | | | | | deletion |
| ALL case | deletion | e1 | e2 | e3 | e4 | e5 | e6 | e7 | on FISH |
| Hyperdip > 50-SNP-#3 | Promoter - e2 | | 0.40 | 0.83 | | | | | |
| Hyperdip > 50-SNP- | All gene | | | | | | | | 95 |
| MLL-SNP-#6 | e3-e6 | | 1.09 | 0.54 | 0.52 | | | | |
| BCR-ABL-SNP-#1 | e3-e6 | | 0.98 | 0.43 | 0.57 | | | | |
| BCR-ABL-SNP-#2 | WT | 0.89 | 0.96 | 1.00 | 0.92 | 0.81 | 0.78 | 0.97 | |
| BCR-ABL-SNP-#3 | e3-distal | 0.96 | 1.02 | 0.56 | 0.82 | | | | |
| BCR-ABL-SNP-#4 | e3-e6 | 0.94 | 1.24 | 0.57 | 0.67 | 0.54 | 0.62 | 0.94 | |
| BCR-ABL-SNP-#5 | e3-e6 | | 1.17 | 0.54 | 0.52 | | | | |
| BCR-ABL-SNP-#6 | WT | 1.06 | 1.04 | 1.03 | 1.14 | 1.23 | 1.07 | 1.09 | |
| BCR-ABL-SNP-#7 | e3-e6 | 0.92 | 1.11 | 0.56 | 0.73 | | | | |
| BCR-ABL-SNP-#8 | WT | 0.96 | 1.04 | 1.18 | 1.16 | 1.10 | 1.05 | 1.27 | |
| BCR-ABL-SNP-#9 | All gene, homo | 0.03 | 0.03 | 0.04 | 0.03 | | | | 98 |
| | e1 - distal | | | | | | | | |
| BCR-ABL-SNP-#10 | e3-e6 | | 0.99 | 0.56 | 0.62 | | | | |
| BCR-ABL-SNP-#11 | WT | 0.95 | 1.11 | 1.01 | 1.25 | 1.13 | 1.09 | 1.00 | |
| BCR-ABL-SNP-#12 | e3-e6 | | 0.91 | 0.32 | 0.40 | | | | |
| BCR-ABL-SNP-#13 | e3-e6 | | 1.23 | 0.49 | 0.52 | | | | |
| BCR-ABL-SNP-#14 | WT | 1.08 | 1.10 | 1.21 | 1.23 | 1.16 | 1.12 | 1.21 | |
| BCR-ABL-SNP-#15 | promoter, e3- | | 1.03 | 0.48 | 0.58 | | | | 95 |
| BCR-ABL-SNP-#16 | e3-e6 | | 1.16 | 0.54 | 0.65 | | | | |
| BCR-ABL-SNP-#17 | promoter-e2 | 0.54 | 0.59 | 1.08 | 1.00 | | | | |
| BCR-ABL-SNP-#18 | All gene | | 0.48 | 0.46 | 0.45 | | | | 94 |
| BCR-ABL-SNP-#19 | e1-e6 | | 0.51 | 0.50 | 0.48 | | | | |
| BCR-ABL-SNP-#20 | All gene | | 0.52 | 0.51 | 0.52 | | | | 87 |
| BCR-ABL-SNP-#21 | All gene, homo | | 0.46 | 0.05 | 0.04 | | | | 95 |
| | e3-e6 | | | | | | | | |
| BCR-ABL-SNP-#22 | All gene | | 0.69 | 0.52 | 0.53 | | | | 90 |
| BCR-ABL-SNP-#23 | e1-e6 | | 0.55 | 0.45 | 0.56 | | | | |
| BCR-ABL-SNP-#24 | All gene | | 0.47 | 0.44 | 0.45 | | | | 78 |
| BCR-ABL-SNP-#25 | e1-e7 | | 0.51 | 0.50 | 0.58 | | | | |
| BCR-ABL-SNP-#26 | Promoter; e3-e6 | | 1.03 | 0.53 | 0.62 | | | | 75 |
| BCR-ABL-SNP-#27 | All gene | | 0.52 | 0.55 | 0.52 | | | | 100 |
| BCR-ABL-SNP-#28 | e1-e6, homo e1- | | 0.04 | 0.72 | 0.63 | | | | |
| BCR-ABL-SNP-#29 | Promoter-e0; | | 0.53 | 0.48 | 0.57 | | | | 90 |
| | e3 - distal | | | | | | | | |
| BCR-ABL-SNP-#30 | All gene, homo | | 0.08 | 0.09 | 0.08 | | | | |
| | e1 - distal | | | | | | | | |
| BCR-ABL-SNP-#31 | Promoter-e0, | | 1.02 | 0.81 | 0.98 | | | | |
| | e4- distal | | | | | | | | |
| BCR-ABL-SNP-#32 | e1-distal | | 0.62 | 0.60 | 0.57 | | | | |
| BCR-ABL-SNP-#33 | e3-e6 | | 1.34 | 0.69 | 0.53 | | | | |
| BCR-ABL-SNP-#34 | e3-e6 | | 1.33 | 0.54 | 0.65 | | | | |
| BCR-ABL-SNP-#35 | WT | 1.24 | 1.16 | 1.13 | 1.23 | 1.21 | 1.13 | 1.36 | |
| BCR-ABL-SNP-#36 | Homo all gene | | 0.06 | 0.07 | 0.07 | | | | 91 |
| BCR-ABL-SNP-#37 | All gene | | 0.54 | 0.46 | 0.46 | | | | 95 |
| BCR-ABL-SNP-#38 | e3-e6 | | 1.30 | 0.53 | 0.53 | | | | |
| BCR-ABL-SNP-#39 | e3-e6 | | 0.98 | 0.47 | 0.47 | | | | |
| BCR-ABL-SNP-#40 | WT | 1.29 | 1.04 | 1.15 | 1.18 | 1.14 | 1.21 | 1.31 | |
| BCR-ABL-SNP-#41 | All gene | | | | | | | | 94 |
| BCR-ABL-SNP-#42 | All gene | | 0.43 | 0.42 | 0.40 | | | | 84 |
| BCR-ABL-SNP-#43 | e3-e6 | | | 0.58 | 0.72 | 0.43 | 0.49 | | |
| Hyperdip47-50-SNP- | e3-e6 | 0.87 | 1.11 | 0.61 | 0.72 | | | | |
| Hyperdip47-50-SNP- | All gene | 0.50 | 0.59 | 0.57 | 0.61 | 0.48 | 0.56 | 0.46 | 86 |
| Hyperdip47-50-SNP- | e1-e7 | 0.56 | 0.59 | 0.60 | 0.64 | | | | |
| Hyperdip47-50-SNP- | e3-e6 | | 1.15 | 0.66 | 0.66 | | | | |
| Other-SNP-#2 | All gene | 0.58 | 0.59 | 0.58 | 0.37 | 0.55 | 0.57 | 0.66 | 100 |
| Other-SNP-#3 | e3-e6 | 0.94 | 1.26 | 0.54 | 0.66 | 0.54 | 0.59 | 1.11 | |
| Other-SNP-#9 | e3-e6 | 0.96 | 1.24 | 0.62 | 0.59 | 0.43 | 0.52 | 0.87 | |
| Other-SNP-#12 | e3-e6 | | 1.20 | 0.72 | 0.75 | | | | |
| Other-SNP-#17 | e3-e6 | | 1.31 | 0.64 | 0.53 | | | | |
| Other-SNP-#19 | e3-e6 | | 1.01 | 0.54 | 0.72 | | | | |
| Other-SNP-#22 | e3-e6 | | 1.11 | 0.48 | 0.51 | | | | |
| Other-SNP-#23 | e3-e6 | | 1.33 | 0.29 | 0.41 | | | | |
| Other-SNP-#26 | e3-e6 | | 1.36 | 0.68 | 0.61 | | | | |
| Pseudodip-SNP-#18 | WT | 0.94 | 1.14 | 1.09 | 0.78 | 0.86 | 0.90 | 1.24 | |
| Pseudodip-SNP-#20 | All gene | 0.47 | 0.48 | 0.56 | 0.48 | | | | 96 |
| Pseudodip-SNP-#6 | All gene | 0.47 | 0.63 | 0.63 | 0.71 | 0.46 | 0.54 | 0.44 | 97 |
| Hypodip-SNP-#1 | All gene | | | | | | | | 98 |
| Hypodip-SNP-#4 | e3-e6 | | 1.25 | 0.49 | 0.58 | | | | |
| Hypodip-SNP-#5 | All gene | 0.55 | 0.53 | 0.55 | 0.55 | 0.49 | 0.55 | 0.50 | 99 |
| Hypodip-SNP-#7 | All gene | | 0.54 | 0.56 | 0.44 | | | | |
| Hypodip-SNP-#8 | All gene | | | | | | | | 67 |
| T-ALL-SNP-#15 | 2-6 | 0.81 | 1.17 | 0.61 | 0.72 | 0.41 | 0.54 | 0.83 | |

Table 5 shows IKZF1 genomic quantitative PCR and fluorescent in situ hybridization (FISH) results. Genomic qPCR of all 7 coding IKZF1 exons was performed for 8 cases to verify the extent of IKZF1 deletions. In the remaining cases, a subset of exons was tested to confirm the focal IKZF1 deletions. IKZF1/RNAseP qPCR ratios of less than 0.75 indicate deletion, and ratios of less than 0.3 indicate homozygous deletion. e, exon; homo, homozygous (deletion); WT, wild type.

TABLE 6
Chronic myeloid leukemia (CML) cases examined by SNP
array.

| | | 250k Sty SNP | 250k Nsp |
|---|---|---|---|
| Sample ID | Sample status | call rate | SNP call rate |
| CML-#1-CP | Chronic phase | 90.5 | 94.6 |
| CML-#1-BC | Myeloid blast crisis | 88.8 | 94.1 |
| CML-#2-CP^{†} | Chronic phase | 89.9 | 94.1 |
| CML-#2-CP2 | Chronic phase | 94.0 | 88.8 |
| CML-#3-AP | Accelerated phase | 93.8 | 92.1 |
| CML-#3-BC | Myeloid blast crisis | 92.1 | 92.7 |
| CML-#4-CP | Chronic phase | 92.8 | 92.6 |
| CML-#4-Rem | Germline | 94.6 | 95.3 |
| CML-#4-BC | Lymphoid blast crisis | 92.3 | 94.5 |
| CML-#5-Rem^{†} | Germline | 94.5 | 93.6 |
| CML-#5-BC-GL | Germline | 92.3 | 90.0 |
| CML-#5-BC | Myeloid blast crisis | 94.7 | 92.2 |
| CML-#6-CP^{†} | Chronic phase | 91.2 | 86.4 |
| CML-#6-BC-GL | Germline | 90.5 | 95.1 |
| CML-#6-BC | Myeloid blast crisis | 88.9 | 89.8 |
| CML-#7-CP | Chronic phase | 92.5 | 92.4 |
| CML-#7-BC | Lymphoid blast crisis | 95.9 | 89.7 |
| CML-#8-CP | Chronic phase | 94.7 | 92.9 |
| CML-#8-AP | Accelerated phase | 92.8 | 91.3 |
| CML-#9-BC | Myeloid blast crisis | 92.7 | 90.9 |
| CML-#9-BC-GL | Germline | 93.1 | 92.2 |
| CML-#10-AP | Accelerated phase | 94.9 | 94.5 |
| CML-#10-CP | Chronic phase | 91.4 | 90.8 |
| CML-#11-CP | Chronic phase | 92.0 | 92.5 |
| CML-#11-AP | Accelerated phase | 95.7 | 91.6 |
| CML-#12-CP | Chronic phase | 92.3 | 87.5 |
| CML-#12-AP | Accelerated phase | 95.7 | 90.0 |
| CML-#12-CP | Chronic phase | 92.7 | 91.9 |
| CML-#13-CP | Chronic phase | 93.0 | 91.8 |
| CML-#13-CP2 | Chronic phase | 91.9 | 94.0 |
| CML-#14-BC | Myeloid blast crisis | 92.9 | 89.8 |
| CML-#14-Rem | Germline | 91.0 | 94.0 |
| CML-#15-CP | Chronic phase | 93.1 | 81.3 |
| CML-#15-CP2 | Chronic phase | 92.9 | 93.3 |
| CML-#15-BC | Myeloid blast crisis | 91.0 | 93.0 |
| CML-#16-CP | Chronic phase | 91.0 | 92.5 |
| CML-#16-CP2 | Chronic phase | 92.8 | 93.3 |
| CML-#16-BC | Myeloid blast crisis | 92.5 | 90.6 |
| CML-#16-BC-GL | Germline | 93.4 | 87.2 |
| CML-#17-CP | Chronic phase | 92.7 | 92.1 |
| CML-#17-AP | Accelerated phase | 91.5 | 90.0 |
| CML-#18-BC | Myeloid blast crisis | 95.5 | 91.4 |
| CML-#19-CP | Chronic phase | 92.7 | 90.1 |
| CML-#19-BC | Myeloid blast crisis | 94.2 | 89.7 |
| CML-#19-BC-GL | Germline | 88.5 | 93.5 |
| CML-#20-CP | Chronic phase | 89.5 | 86.1 |
| CML-#20-AP | Accelerated phase | 92.6 | 88.2 |
| CML-#20-BC | Myeloid blast crisis | 92.5 | 82.2 |
| CML-#21-CP^{†} | Chronic phase | 90.8 | 92.0 |
| CML-#21-CP2 | Chronic phase | 94.4 | 90.7 |
| CML-#22-CP | Chronic phase | 92.4 | 86.8 |
| CML-#22-BC | Myeloid blast crisis | 91.1 | 90.8 |
| CML-#22-BC-GL | Germline | 92.6 | 90.8 |
| CML-#23-CP | Chronic phase | 86.6 | 88.3 |
| CML-#23-BC | Lymphoid blast crisis | 93.4 | 85.8 |
| CML-#23-BC-GL | Germline | 91.2 | 93.1 |

^{†}IKZF1 sequencing for these cases was not performed or failed due to limited DNA.

TABLE 7
The frequency of copy number abnormalities in CML.

| | Deletions | Gains | |
|---|---|---|---|
| Stage | (Mean, range) | (Mean, range) | All lesions |
| Chronic Phase (N = 19) | 0.37 (0-6) | 0.11 (0-2) | 0.47 (0-8) |
| Accelerated Phase | 0.14 (0-1) | 1 (0-5) | 1.14 (0-6) |
| (N = 7) | | | |
| Blast Crisis (N = 15) | 4.93 (0-22) | 2.93 (0-10) | 7.8 (0-28) |

TABLE 8
Intron 2

| | | Additional |
|---|---|---|
| Case | Sequence | nucleotides |
| BCR-ABL-SNP-#1: | ccagggatctcagaaattattagtacatcc | gggcct |
| BCR-ABL-SNP-#4: | ccagggatctcagaaattattagtaca | gc |
| BCR-ABL-SNP-#7: | ccagggatctcagaaattattagtacat | gggg |
| BCR-ABL-SNP-#10: | ccagggatctcagca | cc |
| BCR-ABL-SNP-#12: | ccagggatctcagcatc | ggtt |
| BCR-ABL-SNP-#13: | cc | ggggg |
| BCR-ABL-SNP-#16: | ccagggatctcagcatcc | gagg |
| BCR-ABL-SNP-#21: | ccagggatctcagc | cgggt |
| BCR-ABL-SNP-#26: | ccagggatctcagaaattattagt | gcctt |
| BCR-ABL-SNP-#33: | ccagggatctcagcatcc | |
| BCR-ABL-SNP-#34: | ccagggatctcagcatcc | g |
| BCR-ABL-SNP-#38: | ccagggatctcagcatc | acccc |
| BCR-ABL-SNP-#39: | ccagggatctcagc | ttaa |
| BCR-ABL-SNP-#42: | ccagggatctcag | ggcg |
| Normal: | ccagggatctcagaaattattagtacatcccacagtgaa | |

Intron 6

| Case | Sequence |
|---|---|
| BCR-ABL-SNP-#1: | aaacatcaagtctagtgtaactg |
| BCR-ABL-SNP-#4: | gaaacatcaagtctagtgtaactg |
| BCR-ABL-SNP-#7: | acatcaagtctagtgtaactg |
| BCR-ABL-SNP-#10: | ggaaacatcaagtctagtgtaactg |
| BCR-ABL-SNP-#12: | aacatcaagtctagtgtaactg |
| BCR-ABL-SNP-#13: | aacatcaagtctagtgtaactg |
| BCR-ABL-SNP-#16: | aagtctagtgtaactg |
| BCR-ABL-SNP-#21: | catcaagtctagtgtaactg |
| BCR-ABL-SNP-#26: | aaacatcaagtctagtgtaactg |
| BCR-ABL-SNP-#38: | catcaagtctagtgtaactg |
| BCR-ABL-SNP-#39: | ggaaacatcaagtctagtgtaactg |
| BCR-ABL-SNP-#42: | acatcaagtctagtgtaactg |
| [symbol] | |
| BCR-ABL-SNP-#33: | gaaacatcaagtctagtgtaactg |
| BCR-ABL-SNP-#34: | acatcaagtctagtgtaactg |
| [symbol] | |
| Normal: | tgttgctgtggaaacatcaagtctactgtaactg |

[symbol]

indicates data missing or illegible when filed

heptamer RSSs located immediately within the deleted segment. Representative BCR-ABL1 cases are shown. The heptamer RSSs are shown underlined and in bold, and nucleotides matching the RSS exactly are shown in red. The additional nucleotides between the consensus genomic sequence suggests the action of TdT. The intron 2 junction sequence for the BCR-ABL-SNP clone #1, 4, 7, 10, 12, 13, 16, 21, 26, 33, 34, 38, 39, and 42 are set forth in SEQ ID NOS: 7, 8, 9, 10, 11, 12, 13, 14, 15, 16, 17, 18, 19, and 20, respectively. The normal sequence of intron 2 is set forth in SEQ ID NO:21. The intron 6 junction sequence for the BCR-ABL-SNP clone #1, 4, 7, 10, 12, 13, 16, 21, 26, 38, 39, 42, 33 and 34 are set forth in SEQ ID NOS:22, 23, 24, 25, 26, 27, 28, 29, 30, 31, 32, 33, 34, and 35, respectively. The normal sequence of intron 6 is set forth in SEQ ID NO:36.

TABLE 9

| Proximal (intron 2) | | Additional nucleotides | SEQ ID |
|---|---|---|---|
| BCR-ABL-SNP-#1: | catccagggatctcagaaattattagtacatcc | gggcct | 40 |
| BCR-ABL-SNP-#4: | catccagggatctcagaaattattagtaca | gc | 41 |
| BCR-ABL-SNP-#7: | catccagggatctcagaaattattagtacat | gggg | 42 |
| BCR-ABL-SNP-#10: | catccagggatctcagca | cc | 43 |
| BCR-ABL-SNP-#12: | catccagggatctcagcatc | ggtt | 44 |
| BCR-ABL-SNP-#13: | catcc | ggggg | 45 |
| BCR-ABL-SNP-#16: | catccagggatctcagcatcc | gagg | 46 |
| BCR-ABL-SNP-#21: | catccagggatctcagc | cgggt | 47 |
| BCR-ABL-SNP-#26: | catccagggatctcagaaattattagt | gcctt | 48 |
| BCR-ABL-SNP-#33: | catccagggatctcagcatcc | | 49 |
| BCR-ABL-SNP-#34: | catccagggatctcagcatcc | g | 50 |
| BCR-ABL-SNP-#3 8 | catccagggatctcagcatc | acccc | 51 |
| BCR-ABL-SNP-#39: | catccagggatctcagc | ttaa | 52 |
| BCR-ABL-SNP-#42: | catccagggatctcag | ggcg | 53 |
| Hyperdip47-50-SNP-#2: | catccagggatctcagaaattattagtacat | gggg | 54 |
| Hyperdip47-50-SNP-#24: | catccagggatctcagaaattattagtacatcc | | 55 |
| Hypodip-SNP-#4: | catccagggatctcagaaattattagtacatcc | ac | 56 |
| Other-SNP-#3: | catccagggatctcagaaattattagtaca | aa | 57 |
| Other-SNP-#9: | catccagggatctcagaaattattagtacatc | agat | 58 |
| Other-SNP-#17: | catccagggatctcagaaattattagtac | cc | 59 |
| Other-SNP-#22: | catccagggatctcagaaattattagtacatcc | aaaagaaaaccc | 60, 128 |
| Other-SNP-#23: | catccagggatctcaga | cccttgggag | 61, 129 |
| Other-SNP-#26: | catccagggatctcagaaattattagtac | cctatcaga | 62 |
| MLL-SNP-#6: | catccagggatctcagaaattattagtaca | cccttgtcc | 63 |
| CML-# 1-BC: | catccagggatctcagaaattattagtacatcc | ggactttccgggggggtgtctttc | 64, 130 |
| BV173: | catccagggatctcagaaa | cttgaggg | 65 |
| SUPB15: | catccagggatctcagcatccc | g | 66 |
| Normal: | catccagggatctcagaaattattagtacatcccacagtgaa | | 67 |

| Distal | SEQ | |
|---|---|---|
| (intron 6) | ID | |
| BCR-ABL-SNP-#1: | 68 | aaacatcaagtctagtgtaactgtttcttcttcaaggtgatttgcattttattcctgaatgcctgagggttc |
| BCR-ABL-SNP-#4: | 69 | gaaacatcaagtctagtgtaactgtttcttcttcaaggtgatttgcattttattcctgaatgcctgagggttc |
| BCR-ABL-SNP-#7: | 70 | acatcaagtctagtgtaactgtttcttcttcaaggtgatttgcattttattcctgaatgcctgagggttc |
| BCR-ABL-SNP-#10: | 71 | ggaaacatcaagtctagtgtaactgtttcttcttcaaggtgatttgcattttattcctgaatgcctgagggttc |
| BCR-ABL-SNP-#12: | 72 | aacatcaagtctagtgtaactgtttcttcttcaaggtgatttgcattttattcctgaatgcctgagggttc |
| BCR-ABL-SNP-#13: | 73 | aacatcaagtctagtgtaactgtttcttcttcaaggtgatttgcattttattcctgaatgcctgagggttc |
| BCR-ABL-SNP-#16: | 74 | aagtctagtgtaactgtttcttcttcaaggtgatttgcattttattcctgaatgcctgagggttc |
| BCR-ABL-SNP-#21: | 75 | catcaagtctagtgtaactgtttcttcttcaaggtgatttgcattttattcctgaatgcctgagggttc |
| BCR-ABL-SNP-#26: | 76 | aaacatcaagtctagtgtaactgtttcttcttcaaggtgatttgcattttattcctgaatgcctgagggttc |
| BCR-ABL-SNP-#38: | 77 | catcaagtctagtgtaactgtttcttcttcaaggtgatttgcattttattcctgaatgcctgagggttc |
| BCR-ABL-SNP-#39: | 78 | ggaaacatcaagtctagtgtaactgtttcttcttcaaggtgatttgcattttattcctgaatgcctgagggttc |
| BCR-ABL-SNP-#42: | 79 | acatcaagtctagtgtaactgtttcttcttcaaggtgatttgcattttattcctgaatgcctgagggttc |
| BCR-ABL-SNP-#33: | 80 | gaaacatcaagtctagtgtaactgtttcttcttcaaggtgatttgcattttattcctgaatgcctgagggttc |
| BCR-ABL-SNP-#34: | 81 | acatcaagtctagtgtaactgtttcttcttcaaggtgatttgcattttattcctgaatgcctgagggttc |
| Hyperdip47-50- | 82 | aacatcaagtctagtgtaactgtttcttcttcaaggtgatttgcattttattcctgaatgcctgagggttc |
| SNP-#2: | | |
| Hyperdip47-50- | 83 | gaaacatcaagtctagtgtaactgtttcttcttcaaggtgatttgcattttattcctgaatgcctgagggttc |
| SNP-#24: | | |
| Hypodip-SNP-#4: | 84 | gtctagtgtaactgtttcttcttcaaggtgatttgcattttattcctgaatgcctgagggttc |
| Other-SNP-#3: | 85 | gtaactgtttcttcttcaaggtgatttgcattttattcctgaatgcctgagggttc |
| Other-SNP-#9: | 86 | aaacatcaagtctagtgtaactgtttcttcttcaaggtgatttgcattttattcctgaatgcctgagggttc |
| Other-SNP-#17: | 87 | aaacatcaagtctagtgtaactgtttcttcttcaaggtgatttgcattttattcctgaatgcctgagggttc |
| Other-SNP-#22: | 88 | gaaacatcaagtctagtgtaactgtttcttcttcaaggtgatttgcattttattcctgaatgcctgagggttc |
| Other-SNP-#23: | 89 | ctgtttcttcttcaaggtgatttgcattttattcctgaatgcctgagggttc |
| Other-SNP-#26: | 90 | tctagtgtaactgtttcttcttcaaggtgatttgcattttattcctgaatgcctgagggttc |
| MLL-SNP-#6: | 91 | tcaagtctagtgtaactgtttcttcttcaaggtgatttgcattttattcctgaatgcctgagggttc |
| CML-# 1-BC: | 92 | ctgtttcttcttcaaggtgatttgcattttattcctgaatgcctgagggttc |
| BV173: | 93 | tgcattttattcctgaatgcctgagggttc |
| SUPB15: | 94 | gtgtaactgtttcttcttcaaggtgatttgcattttattcctgaatgcctgagggttc |
| Normal: | 95 | tgttgctgtggaaacatcaagtctagtgtaactgtttcttcttcaaggtgatttgcattttattcctgaatgcctgagggttc |

Table 9 shows the sequencing of IKZF1 Δ3-6 deletions that demonstrates the restricted location of the breakpoints in both introns 2 and 6, and the heptamer RSSs located immediately within the deleted segment. The heptamer RSSs are shown underlined and in bold, and nucleotides matching the RSS exactly are shown in red. The additional nucleotides between the consensus genomic sequence suggests the action of Terminal deoxynucleotidyl transferase (TdT).

TABLE 10

Primers used for IKZF1 PCR. Quantitative PCR primers were designed using
Primer Express 3.0 (Applied Biosystems, Foster City, CA). AS, antisense;
FAM, 6-carboxyfluorescein; MGB, minor groove binder; P, probe; S, sense.

| | | | |
|---|---|---|---|
| Primer | Description | Sequence (5′→3′) | SEQ ID NO |

| | | | |
|---|---|---|---|
| C506 | IKZF1 RT-PCR, exon 0, S | ctcttcgcccccgaggatcagtctt | 96 |
| C507 | IKZF1 RT-PCR, exon 7, AS | gaaggcggcagtccttgtgcttttc | 97 |
| C7 16 | Actin (RT-PCR control), S | agtgtgacgtggacatccgcaaagac | 98 |
| C7 17 | Actin (RT-PCR control), AS | gcttgctgatccacatctgctggaag | 99 |
| C567 | IKZF1 genomic qPCR, exon 1, S | ggatgctgatgagggtcaaga | 100 |
| C568 | IKZF1 genomic qPCR, exon 1, AS | ttcccacacagctatctcataagg | 101 |
| C569 | IKZF1 genomic qPCR, exon 1, P | FAM-atgtcccaagtttcaggtg-MGB | 102 |
| C570 | IKZF1 genomic qPCR, exon 2, S | gaaggaaagcccccctgtaa | 103 |
| C57 1 | IKZF1 genomic qPCR, exon 2, AS | gatcggcatgggctcatc | 104 |
| C572 | IKZF1 genomic qPCR, exon 2, P | FAM-cgatactccagatgagg-MGB | 105 |
| C5 12 | IKZF1 genomic qPCR, exon 3, S | tgcatcgggcccaatg | 106 |
| C5 13 | IKZF1 genomic qPCR, exon 3, AS | aactgagccaggccttacca | 107 |
| C514 | IKZF1 genomic qPCR, exon 3, P | FAM-ctcatggttcacaaaag-MGB | 108 |
| C558 | IKZF1 genomic qPCR, exon 4, S | caacctgctccggcacat | 109 |
| C559 | IKZF1 genomic qPCR, exon 4, AS | tgcagaggtggcatttgaag | 110 |
| C560 | IKZF1 genomic qPCR, exon 4, P | FAM-aagctgcattccggg-MGB | 111 |
| C561 | IKZF1 genomic qPCR, exon 5, S | gcgaagctctttagaggaacataaa | 112 |
| C562 | IKZF1 genomic qPCR, exon 5, AS | aggcccatgctttccaagt | 113 |
| C563 | IKZF1 genomic qPCR, exon 5, P | FAM-agcgctgccacaac-MGB | 114 |
| C564 | IKZF1 genomic qPCR, exon 6, S | aatcacagtgaaatggcagaagac | 115 |
| C565 | IKZF1 genomic qPCR, exon 6, AS | tctgtccagcacgagagatctc | 116 |
| C566 | IKZF1 genomic qPCR, exon 6, P | FAM-tgtgcaagataggatcag-MGB | 117 |
| C5 15 | IKZF1 genomic qPCR, exon 7, S | agacagaggatcaagggctttaga | 118 |
| C516 | IKZF1 genomic qPCR, exon 7, AS | ggcgcatctttctctgtgatt | 119 |
| C517 | IKZF1 genomic qPCR, exon 7, P | FAM-agcactccttcaatatg-MGB | 120 |
| C538 | Ik6 RNA qPCR, S | tcgggaggacagcaaagc | 121 |
| C539 | Ik6 RNA qPCR, AS | tgtcggacaggcccttgt | 122 |
| C540 | Ik6 RNA qPCR, P | FAM-ccaagagtgacagaggg-MGB | 123 |
| C8 13 | Δ3-6 genomic deletion mapping S | ccacagggcaagtcatccacattttg | 124 |
| C8 14 | Δ3-6 genomic deletion mapping | cagaccatagagtccctcctaggggaaaaa | 125 |
| C8 15 | Δ3-6 genomic deletion | ttcttagaagtctggagtctgtgaaggtca | 126 |
| | mapping sequencing | | |

### REFERENCES

1. Ribeiro, R. C. et al., Clinical and biologic hallmarks of the Philadelphia chromosome in childhood acute lymphoblastic leukemia. Blood 70 (4), 948 (1987).
2. Gleissner, B. et al., Leading prognostic relevance of the BCR-ABL translocation in adult acute B-lineage lymphoblastic leukemia: a prospective study of the German Multicenter Trial Group and confirmed polymerase chain reaction analysis. Blood 99 (5), 1536 (2002).
3. Goldman, J. M. and Melo, J. V., Chronic myeloid leukemia—advances in biology and new approaches to treatment. N Engl J Med 349 (15), 1451 (2003).
4. Daley, G. Q., Van Etten, R. A., and Baltimore, D., Blast crisis in a murine model of chronic myelogenous leukemia. Proc Natl Acad Sci USA 88 (24), 11335 (1991).
5. Williams, R. T., Roussel, M. F., and Sherr, C. J., Arf gene loss enhances oncogenicity and limits imatinib response in mouse models of Bcr-Abl-induced acute lymphoblastic leukemia. Proc Natl Acad Sci USA 103 (17), 6688 (2006).
6. Melo, J. V., The diversity of BCR-ABL fusion proteins and their relationship to leukemia phenotype. Blood 88 (7), 2375 (1996).
7. Melo, J. V. and Barnes, D. J., Chronic myeloid leukaemia as a model of disease evolution in human cancer. Nat Rev Cancer 7 (6), 441 (2007).
8. Mullighan, C. G. et al., Genome-wide analysis of genetic alterations in acute lymphoblastic leukaemia. Nature 446 (7137), 758 (2007).
9. Hahm, K. et al., The lymphoid transcription factor LyF-1 is encoded by specific, alternatively spliced mRNAs derived from the Ikaros gene. Mol Cell Biol 14 (11), 7111 (1994).
10. Molnar, A. and Georgopoulos, K., The Ikaros gene encodes a family of functionally diverse zinc finger DNA-binding proteins. Mol Cell Biol 14 (12), 8292 (1994).
11. Molnar, A. et al., The Ikaros gene encodes a family of lymphocyte-restricted zinc finger DNA binding proteins, highly conserved in human and mouse. J Immunol 156 (2), 585 (1996).
12. Rebollo, A. and Schmitt, C., Ikaros, Aiolos and Helios: transcription regulators and lymphoid malignancies. Immunol Cell Biol 81 (3), 171 (2003).
13. Sun, L., Liu, A., and Georgopoulos, K., Zinc finger-mediated protein interactions modulate Ikaros activity, a molecular control of lymphocyte development. Embo J 15 (19), 5358 (1996).
14. Klug, C. A. et al., Hematopoietic stem cells and lymphoid progenitors express different Ikaros isoforms, and Ikaros is localized to heterochromatin in immature lymphocytes. Proc Natl Acad Sci USA 95 (2), 657 (1998).
15. Sun, L. et al., Expression of dominant-negative Ikaros isoforms in T-cell acute lymphoblastic leukemia. Clin Cancer Res 5 (8), 2112 (1999).
16. Sun, L. et al., Expression of aberrantly spliced oncogenic ikaros isoforms in childhood acute lymphoblastic leukemia. J Clin Oncol 17 (12), 3753 (1999).
17. Sun, L. et al., Expression of dominant-negative and mutant isoforms of the antileukemic transcription factor Ikaros in infant acute lymphoblastic leukemia. Proc Natl Acad Sci USA 96 (2), 680 (1999).
18. Nakase, K. et al., Dominant negative isoform of the Ikaros gene in patients with adult B-cell acute lymphoblastic leukemia. Cancer Res 60 (15), 4062 (2000).
19. Olivero, S. et al., Detection of different Ikaros isoforms in human leukaemias using real-time quantitative polymerase chain reaction. Br J Haematol 110 (4), 826 (2000).
20. Nishii, K. et al., Expression of B cell-associated transcription factors in B-cell precursor acute lymphoblastic leukemia cells: association with PU. 1 expression, phenotype, and immunogenotype. Int J Hematol 71 (4), 372 (2000).
21. Takanashi, M. et al., Expression of the Ikaros gene family in childhood acute lymphoblastic leukaemia. Br J Haematol 117 (3), 525 (2002).
22. Tonnelle, C. et al., Overexpression of dominant-negative Ikaros 6 protein is restricted to a subset of B common adult acute lymphoblastic leukemias that express high levels of the CD34 antigen. Hematol J 4 (2), 104 (2003).
23. Klein, F. et al., BCR-ABL1 induces aberrant splicing of IKAROS and lineage infidelity in pre-B lymphoblastic leukemia cells. Oncogene 25 (7), 1118 (2006).
24. Wang, J. H. et al., Selective defects in the development of the fetal and adult lymphoid system in mice with an Ikaros null mutation. Immunity 5 (6), 537 (1996).
25. Winandy, S., Wu, P., and Georgopoulos, K., A dominant mutation in the Ikaros gene leads to rapid development of leukemia and lymphoma. Cell 83 (2), 289 (1995).
26. Nichogiannopoulou, A. et al., Defects in hemopoietic stem cell activity in Ikaros mutant mice. J Exp Med 190 (9), 1201 (1999).
27. Fugmann, S. D. et al., The RAG proteins and V(D)J recombination: complexes, ends, and transposition. Annu Rev Immunol 18, 495 (2000).
28. Kirstetter, P. et al., Ikaros is critical for B cell differentiation and function. Eur J Immunol 32 (3), 720 (2002).
29. Ehrich, M. et al., Quantitative high-throughput analysis of DNA methylation patterns by base-specific cleavage and mass spectrometry. Proc Natl Acad Sci USA 102 (44), 15785 (2005)
30. Drexler, H. G. The Leukemia-Lymphoma Cell Line Facts Book (Academic Press, London, 2001).
31. Manabe, A. et al. Interleukin-4 induces programmed cell death (apoptosis) in cases of high-risk acute lymphoblastic leukemia. Blood 83, 173 1-7 (1994).
32. Mullighan, C. G. et al. Genome-wide analysis of genetic alterations in acute lymphoblastic leukaemia. Nature 446, 75 8-64 (2007).

### Example 2

### Deletion of IKZF1 (Ikaros) is associated with Poor Prognosis in Acute Lymphoblastic Leukemia

Despite best current therapy, up to 20% of pediatric acute lymphoblastic leukemia (ALL) cases relapse. Recent genome-wide analyses have identified a high frequency of recurring DNA copy number abnormalities (CNA) in ALL, but the prognostic impact of these abnormalities has not been defined. We studied a cohort of 221 children with high-risk B-progenitor ALL that excluded known very high risk ALL subtypes (BCR-ABL1, hypodiploid and infant ALL), using single nucleotide polymorphism microarrays, transcriptional profiling and resequencing. A CNA poor outcome predictor was identified and tested in an independent validation cohort of 258 B-progenitor ALL cases.

Over 50 recurring CNA were identified, most commonly targeting genes encoding regulators of B-lymphoid development (66.8% of cases), with PAX5 targeted in 31.7% and IKZF1 in 28.6%. We identified a CNA predictor of very poor outcome in an independent validation cohort (P<0.0001), that was strongly associated with deletion or mutation of IKZF1, a gene that encodes the lymphoid transcription factor IKAROS. The gene expression signature of the poor outcome group was characterized by reduced expression of B-lineage specific genes, and was highly similar to the signature of BCR-ABL1 ALL, another high-risk ALL subtype also characterized by a high frequency of IKZF1 deletion. Genetic alterations of IKZF1 identify a subgroup of ALL with very poor outcome. Incorporation of molecular tests to identify IKZF1 alterations in diagnostic leukemic blasts should improve the ability to accurately stratify patients for appropriate therapy.

### Introduction

Cure rates for children with acute lymphoblastic leukemia (ALL) now exceed 80%', but current therapies result in substantial toxicities, and up to 20% of ALL cases relapse². In B-progenitor ALL, a number of recurring chromosomal abnormalities are used in risk stratification, including hyperdiploidy, hypodiploidy, translocations t(1 2; 2 1) [ETV6-R UNX1], t(9; 22)[BCR-ABL1], t(1; 19)[TCF3-PBX1] and rearrangement of MLL. Although treatment failure is common in BCRABL1 and MLL-rearranged ALL, relapse occurs in all subtypes, and the biological basis of resistance to therapy is poorly understood.

Recent genome-wide analyses of DNA copy number abnormalities (CNA) have identified numerous recurring genetic alterations in ALL³⁻⁶. Genes encoding transcriptional regulators of B lymphoid development, including PAX5, EBF1 and IKZF1 are mutated in over 40% of B-progenitor ALL³. Notably, deletion of IKZF1, encoding the early lymphoid transcription factor IKAROS, is a near obligate event in BCR-ABL1 positive ALL, and at the progression of chronic myeloid leukemia to lymphoid blast crisis. Other CNAs involve tumor suppressors and cell cycle regulators (CDKN2A/B, RB1, PTEN, ETV6), regulators of apoptosis (BTG1), drug receptor genes (NR3C1 and NR3C2), and lymphoid signaling molecules (BTLA, CD200)³.

A systematic analysis of associations between CNA and outcome in ALL has not been performed. Here we report a study examining CNAs in a cohort of 221 children with high-risk ALL. We identified a CNA outcome predictor driven by deletion of IKZF1 that predicts a high risk of relapse. Association of this CNA predictor with poor outcome was validated in an independent cohort of 258 B-progenitor ALL cases. This CNA predictor was associated with gene expression signature characterized by down regulation of B-lymphoid developmental genes and was also highly related to the expression signature of BCR-ABL1 pediatric ALL.

### Methods

### Patients and Samples

Two patient cohorts were examined, the first comprising 221 non-infant B-progenitor ALL cases treated on the Children's Oncology Group (COG) P9906 study that incorporated an augmented intensive regimen of post-induction chemotherapy (Table 11)^{7,8}. All patients were at high risk of treatment failure based on the presence of central nervous system or testicular disease, MLL gene rearrangement, or age, gender and presentation leukocyte count⁹. BCR-ABL1 and hypodiploid ALL, and patients with induction failure were excluded. One hundred seventy cases (76.9%) lacked a recurring chromosomal abnormality. The validation cohort comprised 258 children with B-progenitor ALL treated at St Jude Children's Research Hospital^{3,5}, and included both standard and high risk patients, common aneuploidies and recurring trans locations (including 21 BCR-ABL1 positive cases; Table 12). Informed consent and Institutional Review Board approval was obtained for both cohorts. Minimal residual disease (MRD) was measured at days 8 (peripheral blood) and 29 (bone marrow) of initial induction chemotherapy for 197 cases in the P9 906 cohort, and at days 19 and 46 for 160 cases in the St Jude cohort using multiparameter immunophenotyping as previously described^{8,10,11}.

The P9906 cohort comprised 221 B-progenitor ALL cases treated on the Children's Oncology Group P9 906 study with an augmented intensive regimen of post-induction chemotherapy⁷ (Table 11). All patients were high risk based on the presence of central nervous system or testicular disease, MLL rearrangement, or based on age, gender and presentation leukocyte count²⁸. BCR-ABL1 and hypodiploid ALL, and cases of primary induction failure were excluded. Hyperdiploid (as defined by trisomy of chromosomes 4 and 10 on cytogenetic analysis) and ETV6-RUNX1 cases were excluded unless CNS or testicular involvement was present at diagnosis. Of 276 cases enrolled, 271 were eligible, and 221 had suitable material for genomic analysis. Twenty-five (11.3%) cases were TCF3-PBX1 positive, 19 harbored MLL-rearrangements, four were hyperdiploid, and three were ETV6-RUNX1 positive.

One hundred seventy (7 6.9%) lacked a recurring chromosomal abnormality. The validation cohort comprised 258 B-progenitor ALL cases treated at St Jude Children's Research Hospital^{3,29}, and included 44 high hyperdiploid (greater than 50 chromosomes), 10 hypodiploid, 17 TCF3-PBX1 positive, 50 ETV6-RUNX1 positive, 21 BCR-ABL1 positive and 24 MLL rearranged B-progenitor ALL cases, and 92 cases with low hyperdiploid, pseudodiploid, normal or miscellaneous karyotypes. These cases were treated on St Jude Total XI (N=8), XII (N=13), XIII (N=105), XIV (N=4), XV (N=1 14) and Interfant-99 (infant; N=5) protocols³⁰⁻³⁴. Nine cases were treated off protocol. The clinical protocol was approved by the National Cancer Institute and by the Institutional Review Board at each of the Children's Research institutions. Patients and/or a parent/guardian provided informed consent to participate in the clinical trial and for future research using clinical specimens.

### Genomic Analyses

Leukemic and remission samples from all P9906 cases were genotyped using 250 k Sty and Nsp SNP arrays (Affymetrix, Santa Clara, Calif.). St Jude samples were genotyped with SNP 6.0 arrays (N=36), 250K Sty and Nsp arrays (N=37), and 250 k and 50 k arrays (N=1 85). SNP array analyses, gene expression profiling, and the use of Gene Set Enrichment Analysis³⁶ and Gene Set Analysis'³ to compare gene expression signatures and examine associations between gene sets and outcome are described herein.

### Single Nucleotide Polymorphism Microarray Analyses

All cases in the P9906 cohort were genotyped using 250K Sty and Nsp arrays, which together examine over 500,000 genomic loci. Thirty-six cases from the St Jude cohort were genotyped using SNP 6.0 arrays which examine over 1.87 million loci; 37 with 250K Sty and Nsp arrays, and 185 with both 250K and two 50K arrays that together examine over 615,000 markers were used in the remainder. SNP array data preprocessing and inference of DNA copy number abnormalities (CNAs) and loss-of-heterozygosity (LOH) was performed as previously described^{3,5}. Briefly, SNP calls were generated using the DM or Birdseed algorithms in GTYPE 4.0 or Genotyping Console (Affymetrix) Summarization of probe level data was performed using the PM/MM (50K and 250K arrays) or PM-only (SNP 6.0 arrays) model-based expression algorithms in dChip (www.dchip.org)¹¹. Normalization of array signals was performed using a reference normalization algorithm that utilizes only those SNP probes from diploid regions of each array to guide normalization³. To identify all tumor-acquired regions of CNA for each sample, circular binary segmentation³⁶ (implemented as the DNAcopy package in R) was performed by directly comparing each tumor sample to the corresponding remission sample.

### Genomic Resequencing of PAX5, EBF1 and IKZF1 and Mutation Detection.

Genomic resequencing of all the coding exons of PAX5, EBF1 and IKZF1 was performed for all P9906 samples. Genomic resequencing of all the coding exons of PAX5, IKZF1 and EBF1 was performed for all P9906 samples by Agencourt Biosciences (Beverley, Mass.). Genomic DNA was amplified in 384 well plates, with each PCR reaction containing 10 ng DNA, 1× HotStar buffer, 0.8 mM dNTPs, 1 mM MgCl2, 0.2 U HotStar enzyme (Qiagen) and 0.2 M forward and reverse primers in 10 l reaction volumes. PCR cycling parameters were: one cycle of 95° C. for 15 min, 35 cycles of 95° C. for 20 s, 60° C. for 30 s and 72° C. for 1 min, followed by one cycle of 72° C. for 3 min. PCR products were purified using proprietary large scale automated template purification systems using solid-phase reversible immobilization, and then sequenced using dye-terminator chemistry and ABI 3700/3730 machines (Applied Biosystems, Foster City, Calif.). Base calls and quality scores were determined using the program PHRED^{37,38}.

Sequence variations including substitutions and insertion/deletions (indel) were analyzed using the SNPdetector³⁹ and the IndelDetector⁴⁰ software. A useable read was required to have at least one 30-bp window in which 90% of the bases have PHRED quality score of at least 30. Poor quality reads were filtered prior to variation detection. The minimum threshold of secondary to primary peak ratio for substitution and indel detection was set to be 20% and 10%, respectively. All sequence variations were annotated using a previously developed variation annotation pipeline⁴¹. Any variation that did not match a known polymorphism (defined as a dbSNP record that does not belong to OMIM SNP nor COSMIC somatic variation database^{42,43}) and resulted in a non-silent amino acid change was considered a putative mutation.

All putative sequence mutations were confirmed by repeat genomic PCR and sequencing of both tumor and remission DNA. Where possible, expression of mutated PAX5 and IKZF1 alleles was confirmed by amplification and direct sequencing of full length PAX5 and IKZF1 cDNA as previously described^{3,29}. Transcripts were then cloned into pGEM-T-Easy (Promega, Madison, Wis.) and multiple colonies sequenced. Confirmation of CNAs involving PAX5 and IKZF1 by genomic quantitative PCR was performed as previously described^{3,29}.

### Structural Modeling of PAX5 Mutations

Missense substitutions were generated in the PAX5 (residues 1-1 49)/ETS-1 (residues 331-440)/DNA structure⁴⁴ and subjected to local refinement using the program O²¹. Structural representation was performed with the program PyMOL (Delano Scientific)⁴⁶.

### Analysis of Associations Between DNA Copy Number Abnormalities and Outcome

Supervised principal components (SPC) analysis^{46,47} was used to examine associations between CNAs and outcome of therapy in a genome-wide fashion. This method has previously been used to examine associations between transcriptional profiling data and outcome in cancer⁴⁷. In this approach, regions of somatic DNA deletion for each sample were transformed into a matrix in which each column represented an individual case, each row represented an individual gene, and each cell represented copy number status for each gene targeted by CNAs in at least one case. Using the P9906 cohort as the training set, a modified univariate Cox score was calculated for the association between copy number status of each gene and event-free survival, and genes whose Cox score exceeded a threshold that best predicted survival were used to carry out supervised principal components analysis. To determine the Cox threshold, the training set was split and principal components were derived from one half of the samples, and then used in a Cox model to predict survival in the other half. By varying the threshold of Cox scores and using twofold cross-validation, this process was repeated ten times, and a threshold of ±1.8 (averaged over ten separate repeats of this procedure) was used to generate the principal components subsequently used to predict outcome.

For each case, we used the first principal component in a regression model to calculate a SPC risk score that represents the sum of the weighted copy number levels for each gene found to be significantly associated with prognosis. To validate the SPC predictor, we computed risk scores for each of the 258 cases in the St Jude validation cohort using the model developed in the P9906 training set, and tested whether these scores were correlated with survival. To illustrate the performance of the SPC risk score in predicting survival, cases in the validation cohort were classified as being high or low risk according to the calculated SPC risk score, and cumulative incidence of hematologic relapse and any relapse in each SPC risk group analyzed using Gray's estimator⁴⁷. To examine the role of individual genes in determining outcome, we computed importance scores for genes with Cox scores exceeding the threshold defined by cross validation. The importance score is equivalent to the correlation between each gene and the first supervised principal component. Associations between genes with the top importance scores and hematologic and any relapse were then calcula ed using Gray's estimator. Event-free survival (EFS) was defined as the time from diagnosis until the date of failure (relapse, death, or second malignancy) or until the last follow-up date for all event-free survivors. Associations between genetic variables (deletions±sequence mutations of individual genes, presence and number B-cell pathway lesions) and EFS were estimated by the methods of Kaplan and Meier. Standard errors were calculated by the methods of Peto et al⁴⁸. The Mantel-Haenszel test was used to compare EFS estimates for patients with and without lesion at each locus⁴⁹. The proportional hazards model of Fine and Gray was used to adjust for age, presentation leukocyte count, cytogenetic subtype and levels of minimal residual disease (MRD)⁵⁰. Analyses were performed using R (www.r-project.org)⁵¹, SAS (SAS v9. 1.2, SAS Institute, Cary, N.C.) and SPLUS (SPLUS 7.0, Insightful Corp., Palo Alto, Calif.)

To evaluate associations between genetic alterations and MRD, MRD data was converted into an ordinal variable (<0.01% 0.01≦MRD<1% and ≧1%) and association analyses performed using the Chi-Square test (FREQ procedure, SAS) with estimation of false discovery rate (MULTTEST, SAS). Significantly associated variables were then adjusted for age, presentation leukocyte count and genetic subtype using logistic regression.

### Gene Expression Profiling of High Risk all

Gene expression profiling was performed using U133 Plus 2 microarrays (Affymetrix) for 198 P9906 samples, and using U133A microarrays (Affymetrix) for 175 St Jude samples. Probe intensities were generated using the MAS 5.0 algorithm, probe sets called absent in all samples in each cohort were excluded, and expression data log-transformed. To define the gene expression signature of poor outcome ALL in each cohort, we used limma (Linear Models for Microarray Analysis)⁵², the empirical Bayes t-test implemented in Bioconductor⁵³ and the Benjamini-Hochberg method of false discovery rate (FDR) estimation⁵⁴ to identify probe sets differentially expressed between cases defined as high or low risk according to their SPC risk score. This approach was also used to define the gene expression signature of BCR-ABL1 positive de novo pediatric ALL in the St Jude cohort.

To assess similarity between the high-risk gene expression signatures of the P9906 and St Jude cohorts, and between the high-risk signatures and the signature of BCR-ABL1 positive ALL, gene set enrichment analysis (GSEA)⁵⁵ and direct comparison of the signatures was performed. Gene sets of the top up- and down-regulated genes in the signatures of high risk P9906 and St Jude ALL, and BCR-ABL1 positive ALL were created and added to the collection of curated gene sets available at the Molecular Signatures Database (www.broad.mit.edu/gsea/msigdb/). GSEA of high risk ALL was then performed for each cohort using this expanded collection of gene sets. In a complementary approach, we determined the fraction of the top 100 differentially expressed probe sets in P9906 high-risk ALL that were also differentially expressed in St Jude BCR-ABL1 positive ALL (at an FDR threshold of 5%). The Gene Set Analysis (GSA) algorithm, a modification of GSEA that allows testing of associations between gene sets and time-dependent variables such as survival time⁵⁶, was used to examine associations between gene sets and EFS in the P9906 cohort.

### Genomic Data Access

P9906 SNP array data are available to academic researchers upon request at caArray at CaBIG (the National Cancer Institute Cancer Biomedical Informatics Grid) (www.array.nci.nih.gov/caarray/project/mulli-001 12), and St Jude SNP array data at the National Center for Biotechnology Information (NCBI) Gene Expression Omnibus (GEO)^{57,58} (www.ncbi.nlm.nih.gov/geo, accession GSE1 1445). Primary gene expression data are available at GEO (P9906 data accession GSE11877, St Jude data accession GSE 12995) and (for P9906 data), caArray. All P9906 SNP array, gene expression, and sequence analysis data are available at http://target.cancer.gov/data/. All sequencing traces and sequencing primer Information have been deposited with NCBIs trace archive.

### Results

### Copy Number Alterations in High Risk All

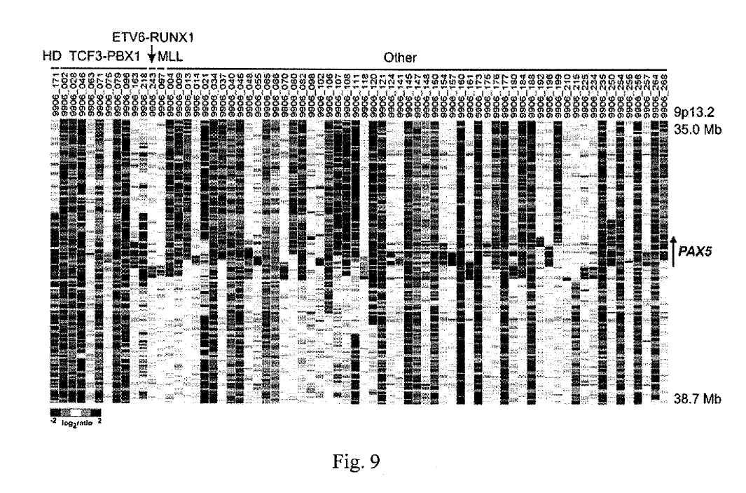

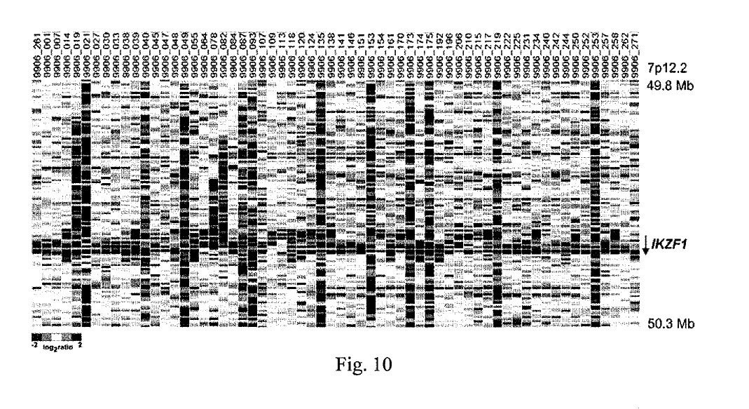

We identified a mean of 8.36 CNAs per case in the P9906 cohort (Table 13), and over 50 recurring CNA where the minimal common region of change involved one or few genes (Table 14). The most common alterations were deletions of CDKN2A/B (45.7%), the lymphoid transcription factor genes PAX5 (31.7%, FIG. 9 and Table 16) and IKZF1 (28.6%, FIG. 10), ETV6 (TEL, 12.7%), RB1 (11.3%), and BTG1 (10.4%).

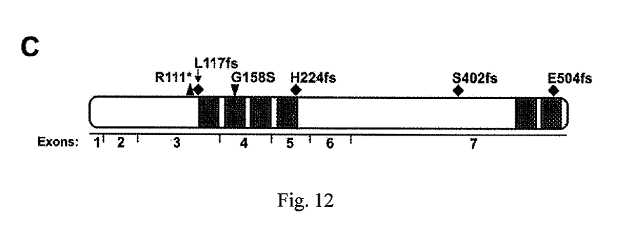

Twenty-two cases harbored 27 PAX5 sequence mutations (Table 17). The most frequent was the previously identified P80R mutation in the paired domain of PAX5 that results in marked attenuation of the DNA-binding and transactivating activity of PAX5³ (FIG. 12A). Several novel paired domain missense (R59G, T75R), and transactivating domain splice site and frameshift mutations were identified. Each of the paired domain mutations is predicted to result in impaired binding of PAX5 to DNA, or disruption of the interaction of PAX5 with ETS 1 that is required for high affinity binding of PAX5 to target DNA sequences¹⁶ (FIG. 12B).

Sixty-three (28.6%) cases had deletion of IKZF1 (Tables 14 and 18, FIG. 10), which involved the entire IKZF1 locus in 16 cases. In the remainder, a subset of exons or the genomic region immediately upstream of IKZF1 was deleted. In 20 cases, coding exons 3-6 were deleted, which results in the expression of a dominant negative form of IKAROS, Ik6, that lacks all N-terminal, DNA-binding zinc fingers⁵. We also identified six novel missense, frameshift and nonsense IKZF1 mutations (FIG. 12C), each of which is predicted to impair IKAROS function. Mutation of G1 58 is known to attenuate the DNA binding activity of IKAROS¹⁷, and thus the G158S mutation we identified would likely act as a dominant negative IKAROS allele. Overall, 66.8% of the high-risk ALL cases harbored at least one mutation of genes regulating B lymphoid development (Tables 14 and 19), with significant variation in the frequency of lesions between ALL subtypes (Table 20).

Associations with Outcome

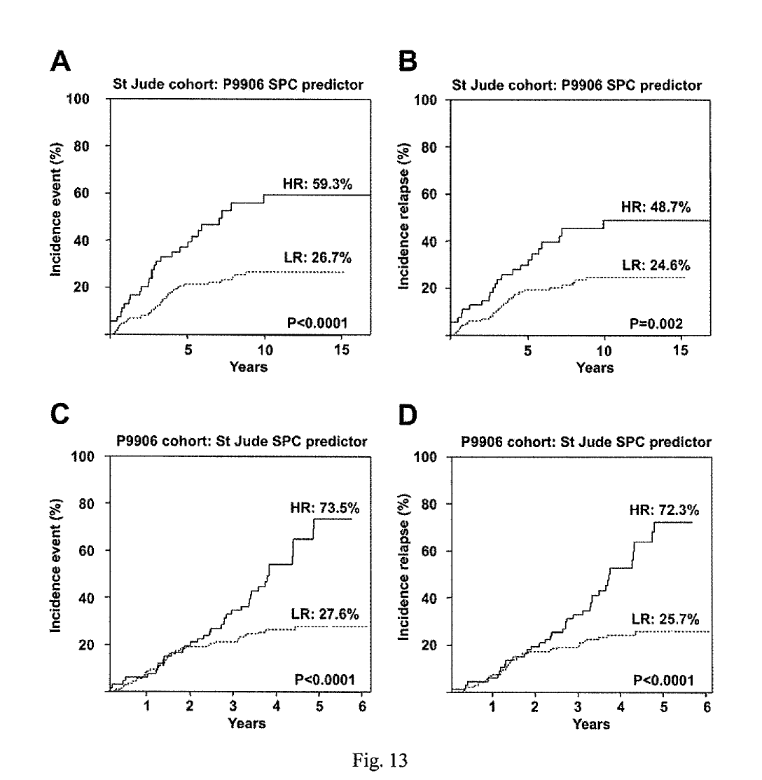

Supervised principal components (SPC) analysis of the P9906 cohort identified associations between copy number status of 23 genes and treatment outcome (Table 21). The resulting SPC risk score was associated with the risk of experiencing any adverse event in the St Jude validation cohort. The 10 year incidence of events in SPC-predicted high risk cases was 59.3% (95% confidence interval (CI) 43.6%-75.1%), compared to 26.7% (CI 19.5%-33.9%) for predicted low risk cases (P<0.0001; FIG. 10A); the 10 year incidence of relapse was 48.7% (CI 33.1%-64.3%) and 24.6% (CI 17.5%-3 1.6%) for high v. low risk cases (P=0.002; FIG. 13B). Conversely, using the St Jude cohort as the training set, a SPC predictor was identified that was associated with outcome in the P9906 cohort (high risk five year adverse events incidence 73.5% (CI 57.4%-89.6%) v. low risk 27.6% (CI 20.0%-35.2%), P<0.0001; relapse 72.3% (CI 56.1%-88.5%) v. 25.7% (CI 18.2% v 33.1%), P<0.0001, FIG. 13D).

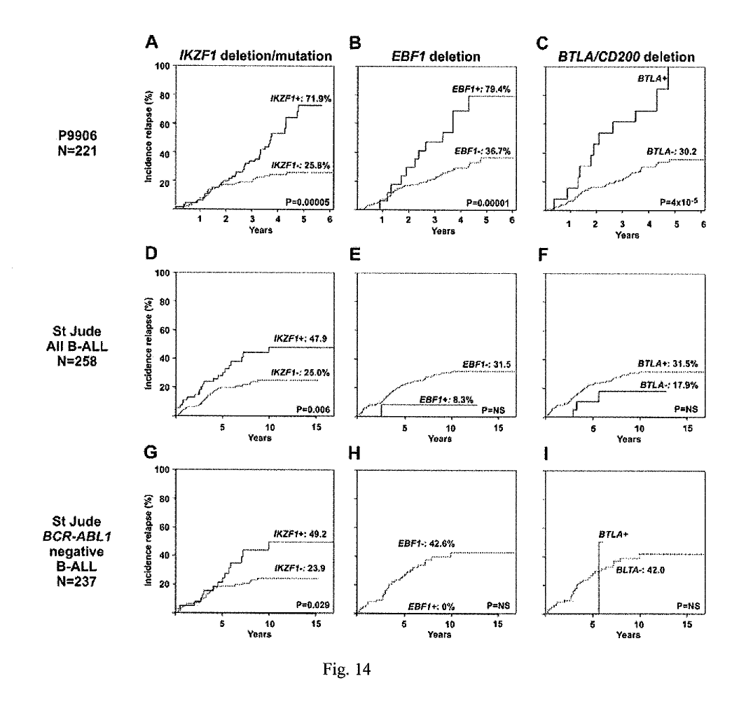

Alterations of IKZF1, BTLA/CD200 and EBF1 were most significantly associated with the P9906 SPC predictor (Table 21). Of these, only IKZF1 was significantly associated with the predictor defined in the St Jude cohort (Table 22). Deletion or mutation of IKZF1 was significantly associated with increased risk of relapse and adverse events in both cohorts (Table 37, FIG. 14A,D, Tables 23-25). IKZF1 deletions were also associated with inferior outcome in St Jude BCR-ABL1 negative ALL (Table 37, FIG. 14G).

Furthermore, alteration of IKZF1 had independent prognostic significance after adjusting for age, presenting leukocyte count and cytogenetic subtype (Table 25). Deletions of EBF1 and BTLA/CD200 were only associated with inferior outcome in the P9906 cohort. Whilst an increasing number of genetic alterations targeting B cell development was also associated with inferior outcome (Supplementary Tables 23-25), no independent association between PAX5 lesions and outcome were observed in either cohort.

Associations with Minimal Residual Disease During Remission Induction Therapy

Consistent with previous data^{8,10,11}, elevated MRD levels were strongly associated with increased risk of relapse in both cohorts (COG day 8 P<0.0001, day 29 P<0.0001; St Jude day 19 P<0.0001, day 46 P<0.0001). IKZF1 and EBF1 alterations were strongly associated with elevated day 29 MRD levels in the P9906 cohort. Sixteen of 66 (24.2%) IKZF1 deleted/mutated cases had high-level (>1%) MRD at day 29, compared to 6.5% of those without this abnormality (P=0.0002, Table 38 and Table 27). These associations remained significant in multivariable analysis adjusting for age, presentation leukocyte count and genetic subtype (EBF1 odds ratio (OR) 5.5, P=0.001; IKZF1 OR 2.7, P=0.002; Table 28). Importantly, the associations of IKZF1 with relapse and adverse events remained significant after adjusting for age, leukocyte count, subtype and MRD in this cohort (Table 29).

IKZF1 alterations were also associated with outcome in the subgroup of St Jude cases with MRD data (N=160; Tables 30 and 31). Deletion or mutation of IKZF1 was strongly associated with elevated MRD levels in this subset of patients. Thirteen (61.9%) IKZF1 deleted/mutated cases had high (≧1.0%) levels of residual disease at day 19, compared to 9.3% of cases without deletion (P<0.0001, Table 38 and Table 32). This association was also observed for day 46 MRD (33.3% v 0.7%, P<0.0001, Table 38 and Table 33). IKZF1 status was also associated with both day 19 (P=0.0001) and day 46 MRD (P=0.0001) in the BCR-ABL1 negative St Jude cohort (Tables 34 and 35).

### Gene Expression Profiling of High-Risk ALL

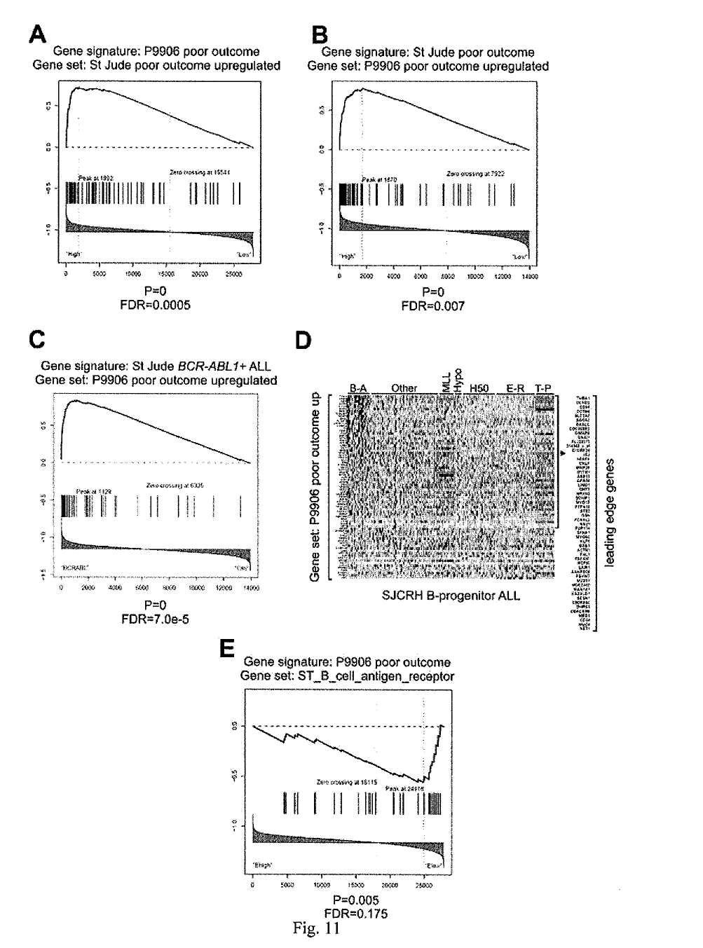

The association between IKZF1 alterations and outcome in both cohorts, as well as prior data showing that deletion of IKZF1 is a frequent in BCR-ABL1 positive ALL⁵, suggest that IKAROS abnormalities are important in the pathogenesis of both poor outcome, BCR-ABL1 negative ALL and BCR-ABL1 positive ALL. To explore this, we used gene set enrichment analysis to compare the gene expression signatures of P9906 and St Jude poor outcome ALL, and BCR-ABL1 positive and P9906 poor outcome (BCR-ABL1 negative) ALL. This analysis revealed significant similarity of signatures of the poor outcome P9906 and St Jude ALL groups (Supplementary FIG. 3A-B). We also observed highly significant enrichment of the P9906 high risk signature in BCR-ABL1 positive St Jude ALL (Supplementary FIG. 3C-D). Moreover, 60% of the top 100 differentially expressed genes in P9906 poor outcome ALL were present in the St Jude BCRABL1 signature (at a false discovery level of 5%), indicating substantial similarity between the two signatures. These findings indicate that mutation of IKZF1 influences the transcriptome of both BCR-ABL1 positive and poor outcome BCR-ABL1 negative ALL. We also observed negative enrichment of a gene set comprising genes mediating B lymphocyte receptor signaling and development¹⁸ in the P9906 poor outcome group (FIG. 11E, Table 36), suggesting that IKZF1 mutation results in impaired B lymphoid development. Finally, gene set analysis¹³ using time to first event as phenotype demonstrated that the BCR-ABL1 signature was the gene set most strongly predictive of poor outcome in the P9906 (BCR-ABL1 negative) cohort (P<0.0001).

TABLE 11
Samples studied frGL tIG CIildPen's
KHcoloKyLGrGUSHPJJV6 cohort

| Sample ID | Group | U133 Plus 2 data |
|---|---|---|
| 9906_001 | | Yes |
| 9906_002 | TCF3-PBX1 | Yes |
| 9906_003 | TCF3-PBX1 | Yes |
| 9906_004 | | Yes |
| 9906_005 | | Yes |
| 9906_006 | MLL | Yes |
| 9906_007 | | Yes |
| 9906_008 | | Yes |
| 9906_009 | | Yes |
| 9906_010 | | |
| 9906_011 | | |
| 9906_012 | | Yes |
| 9906_013 | | Yes |
| 9906_014 | | |
| 9906_016 | | |
| 9906_017 | TCF3-PBX1 | Yes |
| 9906_018 | | Yes |
| 9906_019 | | Yes |
| 9906_020 | | Yes |
| 9906_021 | | Yes |
| 9906_022 | | Yes |
| 9906_023 | | |
| 9906_024 | | Yes |
| 9906_027 | | Yes |
| 9906_028 | TCF3-PBX1 | Yes |
| 9906_030 | | Yes |
| 9906_031 | | Yes |
| 9906_032 | MLL | Yes |
| 9906_033 | | Yes |
| 9906_034 | | Yes |
| 9906_036 | | Yes |
| 9906_037 | | Yes |
| 9906_038 | | Yes |
| 9906_039 | | Yes |
| 9906_040 | | |
| 9906_041 | MLL | Yes |
| 9906_042 | | Yes |
| 9906_043 | TCF3-PBX1 | Yes |
| 9906_045 | | Yes |
| 9906_046 | TCF3-PBX1 | Yes |
| 9906_047 | | Yes |
| 9906_048 | | Yes |
| 9906_049 | | Yes |
| 9906_050 | | Yes |
| 9906_051 | MLL | Yes |
| 9906_052 | | Yes |
| 9906_055 | | Yes |
| 9906_057 | | |
| 9906_058 | TCF3-PBX1 | Yes |
| 9906_060 | | Yes |
| 9906_061 | | Yes |
| 9906_062 | | Yes |
| 9906_063 | TCF3-PBX1 | Yes |
| 9906_064 | | Yes |
| 9906_065 | | Yes |
| 9906_066 | | Yes |
| 9906_069 | | Yes |
| 9906_070 | | |
| 9906_071 | TCF3-PBX1 | Yes |
| 9906_073 | | Yes |
| 9906_074 | MLL | Yes |
| 9906_075 | TCF3-PBX1 | |
| 9906_076 | | Yes |
| 9906_078 | | |
| 9906_079 | TCF3-PBX1 | Yes |
| 9906_080 | | Yes |
| 9906_082 | | Yes |
| 9906_083 | ETV6-RUNX1 | Yes |
| 9906_084 | | Yes |
| 9906_085 | | Yes |
| 9906_086 | | Yes |
| 9906_087 | | |
| 9906_090 | | Yes |
| 9906_092 | | Yes |
| 9906_093 | | Yes |
| 9906_094 | | Yes |
| 9906_095 | MLL | Yes |
| 9906_096 | TCF3-PBX1 | Yes |
| 9906_097 | MLL | Yes |
| 9906_098 | | Yes |
| 9906_099 | | Yes |
| 9906_100 | TCF3-PBX1 | |
| 9906_101 | | Yes |
| 9906_102 | | Yes |
| 9906_106 | | Yes |
| 9906_107 | | Yes |
| 9906_108 | | Yes |
| 9906_109 | | |
| 9906_110 | | Yes |
| 9906_111 | | Yes |
| 9906_113 | | Yes |
| 9906_114 | | Yes |
| 9906_115 | MLL | Yes |
| 9906_116 | MLL | Yes |
| 9906_117 | | Yes |
| 9906_118 | | Yes |
| 9906_119 | | Yes |
| 9906_120 | | Yes |
| 9906_121 | | Yes |
| 9906_122 | Hyperdiploid | Yes |
| 9906_123 | MLL | Yes |
| 9906_124 | | Yes |
| 9906_126 | | Yes |
| 9906_128 | MLL | |
| 9906_129 | | Yes |
| 9906_132 | | Yes |
| 9906_133 | | Yes |
| 9906_135 | | |
| 9906_136 | | Yes |
| 9906_137 | MLL | Yes |
| 9906_138 | | Yes |
| 9906_139 | MLL | Yes |
| 9906_141 | | Yes |
| 9906_142 | MLL | Yes |
| 9906_143 | | Yes |
| 9906_144 | | Yes |
| 9906_145 | | Yes |
| 9906_146 | | Yes |
| 9906_147 | | Yes |
| 9906_148 | | Yes |
| 9906_149 | | |
| 9906_150 | | Yes |
| 9906_151 | | Yes |
| 9906_152 | TCF3-PBX1 | Yes |
| 9906_153 | | Yes |
| 9906_154 | | |
| 9906_155 | | Yes |
| 9906_156 | TCF3-PBX1 | Yes |
| 9906_157 | | Yes |
| 9906_159 | TCF3-PBX1 | Yes |
| 9906_160 | | Yes |
| 9906_161 | | Yes |
| 9906_163 | TCF3-PBX1 | Yes |
| 9906_165 | | |
| 9906_166 | TCF3-PBX1 | Yes |
| 9906_167 | | Yes |
| 9906_168 | | Yes |
| 9906_170 | | Yes |
| 9906_171 | Hyperdiploid | Yes |
| 9906_173 | | Yes |
| 9906_174 | | Yes |
| 9906_175 | | Yes |
| 9906_176 | | Yes |
| 9906_177 | | Yes |
| 9906_179 | | Yes |
| 9906_180 | | Yes |
| 9906_182 | | |
| 9906_183 | | |
| 9906_184 | | Yes |
| 9906_185 | | Yes |
| 9906_186 | | Yes |
| 9906_187 | TCF3-PBX1 | Yes |
| 9906_188 | | Yes |
| 9906_189 | | Yes |
| 9906_190 | | Yes |
| 9906_192 | | Yes |
| 9906_193 | | |
| 9906_195 | | Yes |
| 9906_196 | | Yes |
| 9906_198 | TCF3-PBX1 | Yes |
| 9906_199 | | Yes |
| 9906_202 | TCF3-PBX1 | Yes |
| 9906_203 | TCF3-PBX1 | |
| 9906_206 | | Yes |
| 9906_207 | | Yes |
| 9906_209 | Hyperdiploid | Yes |
| 9906_210 | | Yes |
| 9906_211 | | |
| 9906_214 | | Yes |
| 9906_215 | | Yes |
| 9906_216 | | Yes |
| 9906_217 | | Yes |
| 9906_218 | TCF3-PBX1 | Yes |
| 9906_219 | | Yes |
| 9906_220 | MLL | Yes |
| 9906_221 | | Yes |
| 9906_222 | | Yes |
| 9906_224 | ETV6-RUNX1 | Yes |
| 9906_225 | | Yes |
| 9906_227 | MLL | Yes |
| 9906_228 | | Yes |
| 9906_229 | MLL | Yes |
| 9906_230 | | Yes |
| 9906_231 | | |
| 9906_233 | | Yes |
| 9906_234 | | Yes |
| 9906_235 | | Yes |
| 9906_236 | TCF3-PBX1 | Yes |
| 9906_238 | | Yes |
| 9906_239 | | Yes |
| 9906_240 | | Yes |
| 9906_241 | | Yes |
| 9906_242 | | Yes |
| 9906_243 | ETV6-RUNX1 | Yes |
| 9906_244 | | Yes |
| 9906_245 | Hyperdiploid | Yes |
| 9906_246 | | Yes |
| 9906_247 | MLL | Yes |
| 9906_248 | | Yes |
| 9906_249 | | Yes |
| 9906_250 | | |
| 9906_251 | | Yes |
| 9906_252 | | |
| 9906_253 | | Yes |
| 9906_254 | | Yes |
| 9906_255 | | Yes |
| 9906_256 | | Yes |
| 9906_257 | | Yes |
| 9906_258 | | Yes |
| 9906_259 | | Yes |
| 9906_260 | | Yes |
| 9906_261 | MLL | Yes |
| 9906_262 | | Yes |
| 9906_263 | | Yes |
| 9906_264 | | Yes |
| 9906_265 | | Yes |
| 9906_267 | | Yes |
| 9906_268 | | |
| 9906_269 | | |
| 9906_271 | | Yes |
| 9906_272 | TCF3-PBX1 | Yes |

TABLE 12
258 St Jude B-progenitor ALL cases examined.

| Sample ID | SNP platform | U133A expression chip |
|---|---|---|
| Hyperdip>50-SNP-#01 | 250K/50K | JD-ALD485-v5-U133A |
| Hyperdip>50-SNP-#02 | 250K/50K | JD0070-ALL-v5-U133A |
| Hyperdip>50-SNP-#03 | 250K/50K | JD-ALD510-v5-U133A |
| Hyperdip>50-SNP-#04 | 250K/50K | JD0017-ALL-v5-U133A |
| Hyperdip>50-SNP-#05 | 250K/50K | JD0020-ALL-v5-U133A |
| Hyperdip>50-SNP-#06 | 250K/50K | JD0023-ALL-v5-U133A |
| Hyperdip>50-SNP-#07 | 250K/50K | JD-ALD611-v5-U133A |
| Hyperdip>50-SNP-#08 | 250K/50K | JD-ALD612-v5-U133A |
| Hyperdip>50-SNP-#09 | 250K/50K | JD0041-ALL-v5-U133A |
| Hyperdip>50-SNP-#10 | 250K/50K | JD0077-ALL-v5-U133A |
| Hyperdip>50-SNP-#11 | 250K/50K | JD0111-ALL-v5-U133A |
| Hyperdip>50-SNP-#12 | 250K/50K | JD0097-ALL-v5-U133A |
| Hyperdip>50-SNP-#13 | 250K/50K | JD0117-ALL-v5-U133A |
| Hyperdip>50-SNP-#14 | 250K/50K | JD0120-ALL-v5-U133A |
| Hyperdip>50-SNP-#15 | 250K/50K | JD0121-ALL-v5-U133A |
| Hyperdip>50-SNP-#16 | 250K/50K | JD0127-ALL-v5-U133A |
| Hyperdip>50-SNP-#17 | 250K/50K | JD0151-ALL-v5-U133A |
| Hyperdip>50-SNP-#18 | 250K/50K | JD0168-B-ALL-v5-U133A |
| Hyperdip>50-SNP-#19 | 250K/50K | JD0178-ALL-v5-U133A |
| Hyperdip>50-SNP-#20 | 250K/50K | JD0191-ALL-v5-U133A |
| Hyperdip>50-SNP-#21 | 250K/50K | JD0196-ALL-v5-U133A |
| Hyperdip>50-SNP-#22 | 250K/50K | JD0219-ALL-v5-U133A |
| Hyperdip>50-SNP-#23 | 250K/50K | JD0222-ALL-v5-U133A |
| Hyperdip>50-SNP-#24 | 250K/50K | JD-ALD085-v5-U133A |
| Hyperdip>50-SNP-#25 | 250K/50K | |
| Hyperdip>50-SNP-#26 | 250K/50K | |
| Hyperdip>50-SNP-#27 | SNP 6.0 | JD-ALD013-v5-U133A |
| Hyperdip>50-SNP-#28 | 250K/50K | |
| Hyperdip>50-SNP-#29 | 250K/50K | JD-ALD112-v5-U133A |
| Hyperdip>50-SNP-#30 | 250K/50K | JD-ALD163-v5-U133A |
| Hyperdip>50-SNP-#31 | 250K/50K | |
| Hyperdip>50-SNP-#32 | 250K/50K | |
| Hyperdip>50-SNP-#33 | 250K/50K | |
| Hyperdip>50-SNP-#34 | 250K/50K | |
| Hyperdip>50-SNP-#35 | 250K/50K | |
| Hyperdip>50-SNP-#36 | 250K/50K | |
| Hyperdip>50-SNP-#37 | 250K/50K | |
| Hyperdip>50-SNP-#38 | 250K/50K | |
| Hyperdip>50-SNP-#39 | 250K/50K | |
| Hyperdip50-SNP-#51 | SNP 6.0 | |
| Hyperdip50-SNP-#52 | SNP 6.0 | |
| Hyperdip50-SNP-#53 | SNP 6.0 | |
| Hyperdip50-SNP-#54 | SNP 6.0 | |
| Hyperdip50-SNP-#55 | SNP 6.0 | |
| E2A-PBX1-SNP-#01 | 250K/50K | JD0004-ALL-v5-U133A |
| E2A-PBX1-SNP-#02 | 250K/50K | JD0015-ALL-v5-U133A |
| E2A-PBX1-SNP-#03 | 250K/50K | JD0036-ALL-v5-U133A |
| E2A-PBX1-SNP-#04 | 250K/50K | JD0042-ALL-v5-U133A |
| E2A-PBX1-SNP-#05 | 250K/50K | JD0083-ALL-v5-U133A |
| E2A-PBX1-SNP-#06 | 250K/50K | JD0099-ALL-v5-U133A |
| E2A-PBX1-SNP-#07 | 250K/50K | JD0104-ALL-v5-U133A |
| E2A-PBX1-SNP-#08 | 250K/50K | Failed Sample |
| E2A-PBX1-SNP-#09 | 250K/50K | JD0203-ALL-v5-U133A |
| E2A-PBX1-SNP-#10 | 250K/50K | JD-ALD019-v5-U133A |
| E2A-PBX1-SNP-#11 | 250K/50K | JD-ALD025-v5-U133A |
| E2A-PBX1-SNP-#12 | SNP 6.0 | JD-ALD437-v5-U133A |
| E2A-PBX1-SNP-#13 | 250K/50K | JD-ALD034-v5-U133A |
| E2A-PBX1-SNP-#14 | 250K/50K | JD-ALD041-v5-U133A |
| E2A-PBX1-SNP-#15 | 250K/50K | JD-ALD071-v5-U133A |
| E2A-PBX1-SNP-#16 | 250K/50K | JD-ALD073-v5-U133A |
| E2A-PBX1-SNP-#17 | 250K/50K | JD-ALD079-v5-U133A |
| TEL-AML1-SNP-#01 | 250K/50K | JD0002-ALL-v5-U133A |
| TEL-AML1-SNP-#02 | 250K/50K | JD0066-ALL-v5-U133A |
| TEL-AML1-SNP-#03 | 250K/50K | JD0056-ALL-v5-U133A |
| TEL-AML1-SNP-#04 | 250K/50K | JD-ALD493-v5-U133A |
| TEL-AML1-SNP-#05 | 250K/50K | JD0058-ALL-v5-U133A |
| TEL-AML1-SNP-#06 | 250K/50K | JD0059-ALL-v5-U133A |
| TEL-AML1-SNP-#07 | 250K/50K | JD0005-ALL-v5-U133A |
| TEL-AML1-SNP-#08 | 250K/50K | JD0009-ALL-v5-U133A |
| TEL-AML1-SNP-#09 | 250K/50K | JD0033-ALL-v5-U133A |
| TEL-AML1-SNP-#10 | 250K/50K | JD0014-ALL-v5-U133A |
| TEL-AML1-SNP-#11 | 250K/50K | JD0016-ALL-v5-U133A |
| TEL-AML1-SNP-#12 | 250K/50K | JD0018-ALL-v5-U133A |
| TEL-AML1-SNP-#13 | 250K/50K | JD0048-ALL-v5-U133A |
| TEL-AML1-SNP-#14 | 250K/50K | JD0085-ALL-v5-U133A |
| TEL-AML1-SNP-#15 | 250K/50K | JD0101-ALL-v5-U133A |
| TEL-AML1-SNP-#16 | 250K/50K | JD0118-ALL-v5-U133A |
| TEL-AML1-SNP-#17 | 250K/50K | JD0107-ALL-v5-U133A |
| TEL-AML1-SNP-#18 | 250K/50K | JD0109-ALL-v5-U133A |
| TEL-AML1-SNP-#19 | 250K/50K | JD0123-ALL-v5-U133A |
| TEL-AML1-SNP-#20 | 250K/50K | JD0139-ALL-v5-U133A |
| TEL-AML1-SNP-#21 | 250K/50K | JD0149-ALL-v5-U133A |
| TEL-AML1-SNP-#22 | 250K/50K | JD0170-ALL-v5-U133A |
| TEL-AML1-SNP-#23 | 250K/50K | |
| TEL-AML1-SNP-#24 | 250K/50K | JD0175-ALL-v5-U133A |
| TEL-AML1-SNP-#25 | 250K/50K | JD0193-ALL-v5-U133A |
| TEL-AML1-SNP-#26 | 250K/50K | JD0201-ALL-v5-U133A |
| TEL-AML1-SNP-#27 | 250K/50K | |
| TEL-AML1-SNP-#28 | 250K/50K | JD0212-ALL-v5-U133A |
| TEL-AML1-SNP-#29 | 250K/50K | JD0221-ALL-v5-U133A |
| TEL-AML1-SNP-#30 | 250K/50K | |
| TEL-AML1-SNP-#31 | 250K/50K | JD-ALD004-v5-U133A |
| TEL-AML1-SNP-#32 | 250K/50K | JD-ALD005-v5-U133A |
| TEL-AML1-SNP-#33 | 250K/50K | JD-ALD006-v5-U133A |
| TEL-AML1-SNP-#34 | 250K/50K | JD-ALD096-v5-U133A |
| TEL-AML1-SNP-#35 | 250K/50K | |
| TEL-AML1-SNP-#36 | 250K/50K | JD-ALD108-v5-U133A |
| TEL-AML1-SNP-#37 | 250K/50K | JD-ALD109-v5-U133A |
| TEL-AML1-SNP-#38 | 250K/50K | |
| TEL-AML1-SNP-#39 | 250K/50K | |
| TEL-AML1-SNP-#40 | 250K/50K | |
| TEL-AML1-SNP-#41 | 250K/50K | |
| TEL-AML1-SNP-#42 | 250K/50K | |
| TEL-AML1-SNP-#43 | 250K/50K | |
| TEL-AML1-SNP-#44 | 250K/50K | JD-ALD054-v5-U133A |
| TEL-AML1-SNP-#45 | 250K/50K | |
| TEL-AML1-SNP-#46 | 250K/50K | |
| TEL-AML1-SNP-#47 | 250K/50K | |
| TEL-AML1-SNP-#48 | SNP 6.0 | |
| TEL-AML1-SNP-#49 | SNP 6.0 | |
| TEL-AML1-SNP-#50 | SNP 6.0 | |
| MLL-SNP-#01 | 250K/50K | JD0080-ALL-v5-U133A |
| MLL-SNP-#02 | 250K/50K | JD0084-ALL-v5-U133A |
| MLL-SNP-#03 | 250K/50K | |
| MLL-SNP-#04 | 250K/50K | JD0124-ALL-v5-U133A |
| MLL-SNP-#05 | 250K/50K | JD-ALD009-v5-U133A |
| MLL-SNP-#06 | 250K/50K | JD-ALD433-v5-U133A |
| MLL-SNP-#07 | 250K/50K | JD-ALD180-v5-U133A |
| MLL-SNP-#08 | 250K/50K | JD-ALD057-v5-U133A |
| MLL-SNP-#09 | 250K/50K | JD-ALD052-v5-U133A |
| MLL-SNP-#10 | 250K/50K | JD-ALD294-v5-U133A |
| MLL-SNP-#11 | 250K/50K | JD-ALD078-v5-U133A |
| MLL-SNP-#12 | 250K/50K | |
| MLL-SNP-#13 | 250K/50K | |
| MLL-SNP-#15 | 250K/50K | |
| MLL-SNP-#16 | 250K/50K | |
| MLL-SNP-#17 | 250K/50K | JD0284-ALL-v5-U133A |
| MLL-SNP-#18 | 250K/50K | JD-ALD232-v5-U133A |
| MLL-SNP-#19 | 250K/50K | |
| MLL-SNP-#20 | 250K/50K | |
| MLL-SNP-#21 | 250K/50K | |
| MLL-SNP-#22 | 250K/50K | |
| MLL-SNP-#23 | 250K/50K | JD-ALD385-v5-U133A |
| MLL-SNP-#24 | SNP 6.0 | |
| MLL-SNP-#25 | SNP 6.0 | |
| BCR-ABL-SNP-#01 | 250K/50K | JD-ALD494-v5-U133A |
| BCR-ABL-SNP-#02 | 250K/50K | JD-ALD613-v5-U133A |
| BCR-ABL-SNP-#03 | 250K/50K | JD0102-ALL-v5-U133A |
| BCR-ABL-SNP-#04 | 250K/50K | JD0129-ALL-v5-U133A |
| BCR-ABL-SNP-#05 | 250K/50K | JD0154-ALL-v5-U133A |
| BCR-ABL-SNP-#06 | 250K/50K | JD0192-ALL-v5-U133A |
| BCR-ABL-SNP-#07 | 250K/50K | JD0206-ALL-v5-U133A |
| BCR-ABL-SNP-#08 | 250K/50K | JD-ALD008-v5-U133A |
| BCR-ABL-SNP-#09 | 250K/50K | JD-ALD035-v5-U133A |
| BCR-ABL-SNP-#10 | 250K/50K | JD-ALD386-v5-U133A |
| BCR-ABL-SNP-#11 | SNP 6.0 | JD-ALD387-v5-U133A |
| BCR-ABL-SNP-#12 | 250K/50K | JD-ALD388-v5-U133A |
| BCR-ABL-SNP-#13 | 250K/50K | JD-ALD389-v5-U133A |
| BCR-ABL-SNP-#14 | 250K/50K | JD-ALD390-v5-U133A |
| BCR-ABL-SNP-#15 | SNP 6.0 | JD-ALD233-v5-U133A |
| BCR-ABL-SNP-#16 | SNP 6.0 | |
| BCR-ABL-SNP-#17 | 250K/50K | JD-ALD428-v5-U133A |
| BCR-ABL-SNP-#18 | SNP 6.0 | JD-ALD264-v5-U133A |
| BCR-ABL-SNP-#19 | 250K/50K | JD-ALD171-v5-U133A |
| BCR-ABL-SNP-#20 | 250K/50K | JD-ALD039-v5-U133A |
| BCR-ABL-SNP-#21 | 250K/50K | JD-ALD391-v5-U133A |
| Hypodip-SNP-#01 | 250K/50K | JD0057-ALL-v5-U133A |
| Hypodip-SNP-#02 | 250K/50K | JD-ALD536-v5-U133A |
| Hypodip-SNP-#03 | 250K/50K | JD0025-ALL-v5-U133A |
| Hypodip-SNP-#04 | 250K/50K | JD0037-ALL-v5-U133A |
| Hypodip-SNP-#05 | 250K/50K | JD0087-ALL-v5-U133A |
| Hypodip-SNP-#06 | 250K/50K | JD0095-ALL-v5-U133A |
| Hypodip-SNP-#07 | 250K/50K | |
| Hypodip-SNP-#08 | 250K/50K | |
| Hypodip-SNP-#09 | 250K/50K | JD-ALD196-v5-U133A |
| Hypodip-SNP-#10 | 250K/50K | |
| Hyperdip>50-SNP-#40 | 250K/50K | JD-ALD280-v5-U133A |
| Hyperdip47-50-SNP- | 250K/50K | JD0064-ALL-v5-U133A |
| Hyperdip47-50-SNP- | 250K/50K | JD-ALD509-v5-U133A |
| Hyperdip47-50-SNP- | 250K/50K | JD0062-ALL-v5-U133A |
| Hyperdip47-50-SNP- | SNP 6.0 | JD-ALD554-v5-U133A |
| Hyperdip47-50-SNP- | 250K/50K | JD0098-ALL-v5-U133A |
| Hyperdip47-50-SNP- | 250K/50K | JD0112-ALL-v5-U133A |
| Hyperdip47-50-SNP- | 250K/50K | JD0108-ALL-v5-U133A |
| Hyperdip47-50-SNP- | 250K/50K | JD0132-ALL-v5-U133A |
| Hyperdip47-50-SNP- | 250K/50K | JD0133-ALL-v5-U133A |
| Hyperdip47-50-SNP-#1 | 250K/50K | JD0137-ALL-v5-U133A |
| Hyperdip47-50-SNP-#1 | 250K/50K | JD0138-ALL-v5-U133A |
| Hyperdip47-50-SNP- | 250K/50K | JD0150-ALL-v5-U133A |
| Hyperdip47-50-SNP-#1 | 250K/50K | JD0157-ALL-v5-U133A |
| Hyperdip47-50-SNP- | 250K/50K | JD0181-ALL-v5-U133A |
| Hyperdip47-50-SNP-#1 | 250K/50K | JD0186B-ALL-v5-U133A |
| Hyperdip47-50-SNP-#1 | 250K/50K | |
| Hyperdip47-50-SNP-#1 | 250K/50K | |
| Hyperdip47-50-SNP-#1 | 250K/50K | |
| Hyperdip47-50-SNP-#1 | 250K/50K | |
| Hyperdip47-50-SNP- | 250K/50K | |
| Hyperdip47-50-SNP-#2 | 250K/50K | |
| Hyperdip47-50-SNP- | 250K/50K | |
| Hyperdip47-50-SNP- | 250K/50K | |
| Hyperdip47-50-SNP- | 250K/50K | JD-ALD242-v5-U133A |
| Other-SNP-#01 | 250K/50K | JD0065-ALL-v5-U133A |
| Other-SNP-#02 | 250K/50K | JD0116-ALL-v5-U133A |
| Other-SNP-#03 | 250K/50K | JD0122-ALL-v5-U133A |
| Other-SNP-#04 | 250K/50K | JD0131-ALL-v5-U133A |
| Other-SNP-#05 | 250K/50K | JD0166-ALL-v5-U133A |
| Other-SNP-#06 | 250K/50K | JD0202-ALL-v5-U133A |
| Other-SNP-#07 | 250K/50K | JD0226-ALL-v5-U133A |
| Other-SNP-#08 | 250K/50K | JD-ALD340-v5-U133A |
| Other-SNP-#09 | 250K/50K | JD-ALD363-v5-U133A |
| Other-SNP-#10 | 250K/50K | |
| Other-SNP-#11 | 250K/50K | |
| Other-SNP-#12 | 250K/50K | JD-ALD279-v5-U133A |
| Other-SNP-#13 | 250K/50K | |
| Other-SNP-#14 | 250K/50K | JD-ALD194-v5-U133A |
| Other-SNP-#15 | 250K/50K | JD-ALD066-v5-U133A |
| Other-SNP-#16 | 250K/50K | |
| Other-SNP-#17 | 250K/50K | JD-ALD329-v5-U133A |
| Other-SNP-#18 | 250K/50K | JD-ALD115-v5-U133A |
| Other-SNP-#19 | 250K/50K | JD-ALD185-v5-U133A |
| Other-SNP-#20 | 250K/50K | JD-ALD297-v5-U133A |
| Other-SNP-#21 | SNP 6.0 | JD0021-ARD-v5-U133A |
| Other-SNP-#22 | 250K/50K | JD0031-ARD-v5-U133A |
| Other-SNP-#23 | 250K/50K | JD0025-ARD-v5-U133A |
| Other-SNP-#24 | 250K/50K | JD0003-ARD-v5-U133A |
| Other-SNP-#25 | 250K/50K | JD0018-ARD-v5-U133A |
| Other-SNP-#26 | 250K/50K | JD0014-ARD-v5-U133A |
| Other-SNP-#27 | SNP 6.0 | |
| Other-SNP-#28 | SNP 6.0 | |
| Other-SNP-#29 | SNP 6.0 | |
| Other-SNP-#30 | SNP 6.0 | |
| Other-SNP-#31 | SNP 6.0 | |
| Other-SNP-#32 | SNP 6.0 | |
| Other-SNP-#33 | SNP 6.0 | |
| Other-SNP-#34 | SNP 6.0 | |
| Other-SNP-#35 | SNP 6.0 | |
| Other-SNP-#36 | SNP 6.0 | |
| Other-SNP-#37 | SNP 6.0 | JD-ALD146-v5-U133A |
| Other-SNP-#38 | SNP 6.0 | JD-ALD420-v5-U133A |
| Other-SNP-#39 | SNP 6.0 | |
| Other-SNP-#40 | SNP 6.0 | |
| Other-SNP-#41 | SNP 6.0 | |
| Other-SNP-#42 | SNP 6.0 | JD0019-ALL-v5-U133A |
| Other-SNP-#43 | SNP 6.0 | |
| Pseudodip-SNP-#01 | 250K/50K | JD0001-ALL-v5-U133A |
| Pseudodip-SNP-#02 | 250K/50K | JD0071-ALL-v5-U133A |
| Pseudodip-SNP-#03 | 250K/50K | JD0012-ALL-v5-U133A |
| Pseudodip-SNP-#04 | 250K/50K | JD0032-ALL-v5-U133A |
| Pseudodip-SNP-#05 | 250K/50K | JD0021-ALL-v5-U133A |
| Pseudodip-SNP-#06 | 250K/50K | JD-ALD610-v5-U133A |
| Pseudodip-SNP-#07 | 250K/50K | JD0103-ALL-v5-U133A |
| Pseudodip-SNP-#08 | 250K/50K | Failed Sample |
| Pseudodip-SNP-#09 | 250K/50K | JD0173-ALL-v5-U133A |
| Pseudodip-SNP-#10 | 250K/50K | JD0185B-ALL-v5-U133A |
| Pseudodip-SNP-#11 | 250K/50K | JD0188-ALL-v5-U133A |
| Pseudodip-SNP-#12 | 250K/50K | JD0225-ALL-v5-U133A |
| Pseudodip-SNP-#13 | 250K/50K | |
| Pseudodip-SNP-#14 | 250K/50K | |
| Pseudodip-SNP-#15 | 250K/50K | |
| Pseudodip-SNP-#16 | 250K/50K | JD-ALD164-v5-U133A |
| Pseudodip-SNP-#17 | 250K/50K | |
| Pseudodip-SNP-#18 | SNP 6.0 | |
| Pseudodip-SNP-#19 | 250K/50K | |
| Pseudodip-SNP-#20 | 250K/50K | |
| Pseudodip-SNP-#21 | 250K/50K | JD-ALD176-v5-U133A |
| Pseudodip-SNP-#22 | 250K/50K | JD0088-ALL-v5-U133A |
| Pseudodip-SNP-#23 | 250K/50K | JD-ALD136-v5-U133A |
| Pseudodip-SNP-#24 | 250K/50K | JD-ALD325-v5-U133A |

TABLE 13
DNA copy number abnormality frequency in high-risk pediatric ALL

| | All lesions | | | Deletions | | | Gains | | |
|---|---|---|---|---|---|---|---|---|---|
| Group | Mean | Median | Range | Mean | Median | Range | Mean | Median | Range |
| ETV6-RUNX1 | 9.00 | 10 | 1-16 | 8.67 | 9 | 1-16 | .67 | 0 | 0-2 |
| N = 3 | | | | | | | | | |
| TCF3-PBX1 | 3.52 | 4 | 0-9 | 2.44 | 2 | 0-8 | 1.08 | 1 | 0-4 |
| N = 25 | | | | | | | | | |
| MLL-rearranged | 1.84 | 1 | 0-11 | 1.26 | 1 | 0-10 | .58 | 0 | 0-2 |
| N = 19 | | | | | | | | | |
| High hyperdiploid | 16.5 | 16.5 | 6-27 | 2.0 | 2 | 0-4 | 14.5 | 14.5 | 0-23 |
| N = 4 | | | | | | | | | |
| Other | 9.59 | 7 | 0-86 | 5.84 | 5 | 0-33 | 3.78 | 1 | 0-75 |
| N = 170 | | | | | | | | | |
| Total | 8.36 | 6 | 0-86 | 5.03 | 4 | 0-33 | 3.35 | 1 | 0-75 |
| N = 221 | | | | | | | | | |

| | | | |
|---|---|---|---|
| P | <0.0001 | <0.0001 | <0.0001 |

TABLE 14
Regions of recurring copy number alteration in the P9906 cohort.

| | | | | ETV6- | | TCF3- | | | | | | | |
|---|---|---|---|---|---|---|---|---|---|---|---|---|---|
| Lesion | Location | All | % | RUNX1 | % | PBX1 | % | MLL | % | Hyperdiploid | % | Other | % |
| | | 221 | | 3 | | 25 | | 19 | | 4 | | 170 | |
| PDE4B | 1p31.2 | 3 | 1.4 | 0 | 0 | 0 | 0 | 0 | 0 | 0 | 0 | 3 | 1.8 |
| NRAS | 1p13.1 | 4 | 1.8 | 0 | 0 | 0 | 0 | 0 | 0 | 0 | 0 | 4 | 2.4 |
| ADAR | 1q22 | 4 | 1.8 | 0 | 0 | 0 | 0 | 0 | 0 | 0 | 0 | 4 | 2.4 |
| LOC440742* | 1q44 | 6 | 2.7 | 0 | 0 | 0 | 0 | 0 | 0 | 0 | 0 | 6 | 3.5 |
| 1q gain | 1q23.3 [symbol] 1 | 23 | 10.4 | 0 | 0 | 16 | 64 | 0 | 0 | 1 | 25 | 6 | 3.5 |
| ARPP-21 | 3p22.3 | 7 | 3.2 | 0 | 0 | 0 | 0 | 0 | 0 | 0 | 0 | 7 | 4.1 |
| FHIT | 3p14.2 | 2 | .9 | 0 | 0 | 0 | 0 | 0 | 0 | 0 | 0 | 2 | 1.2 |
| FLNB | 3p14.3 | 5 | 2.3 | 0 | 0 | 0 | 0 | 0 | 0 | 0 | 0 | 5 | 2.9 |
| BTLA/CD200 | 3q13.2 | 13 | 5.9 | 0 | 0 | 0 | 0 | 0 | 0 | 0 | 0 | 13 | 7.6 |
| MBNL1 | 3q25.1 | 8 | 3.6 | 1 | 33.3 | 0 | 0 | 0 | 0 | 0 | 0 | 7 | 4.1 |
| TBL1XR1 | 3q26.32 | 7 | 3.2 | 0 | 0 | 0 | 0 | 0 | 0 | 0 | 0 | 7 | 4.1 |
| IL1RAP | 3q28 | 3 | 1.4 | 0 | 0 | 0 | 0 | 0 | 0 | 0 | 0 | 3 | 1.8 |
| ARHGAP24 | 4q21.23 | 4 | 1.8 | 0 | 0 | 0 | 0 | 0 | 0 | 0 | 0 | 4 | 2.4 |
| NR3C2 | 4q31.23 | 5 | 2.3 | 2 | 66.7 | 0 | 0 | 0 | 0 | 0 | 0 | 3 | 1.8 |
| FBXW7 | 4q31.3 | 3 | 1.4 | 0 | 0 | 0 | 0 | 0 | 0 | 0 | 0 | 3 | 1.8 |
| EBF1 | 5q33.3 | 17 | 7.7 | 1 | 33.3 | 0 | 0 | 0 | 0 | 0 | 0 | 16 | 9.4 |
| Histone cluster | 6p22.2 | 9 | 4.0 | 1 | 33.3 | 0 | 0 | 0 | 0 | 0 | 0 | 8 | 4.7 |
| GRIK2 | 6q16 | 14 | 6.3 | 1 | 33.3 | 2 | 8 | 0 | 0 | 0 | 0 | 11 | 6.5 |
| ARMC2/SESN1 | 6q21 | 15 | 6.8 | 1 | 33.3 | 2 | 8 | 0 | 0 | 0 | 0 | 12 | 7.1 |
| LOC389437 | 6q25.3 | 7 | 3.2 | 1 | 33.3 | 1 | 4 | 0 | 0 | 0 | 0 | 5 | 2.9 |
| IKZF1 | 7p13 | 63 | 28.6 | 0 | 0 | 0 | 0 | 1 | 5.3 | 0 | 0 | 61 | 35.9 |
| IKZF1 CNA or | 7p13 | 67 | 30.3 | 0 | 0 | 0 | 0 | 2 | 10.5 | 0 | 0 | 65 | 38.2 |
| sequence | | | | | | | | | | | | | |
| MSRA | 8p23 | 4 | 1.8 | 0 | 0 | 0 | 0 | 0 | 0 | 0 | 0 | 4 | 2.4 |
| TOX | 8q12.1 | 8 | 3.6 | 1 | 33.3 | 0 | 0 | 0 | 0 | 0 | 0 | 7 | 4.1 |
| CCDC26 | 8q24.21 | 23 | 10.4 | 0 | 0 | 3 | 12 | 2 | 10.5 | 2 | 50 | 16 | 9.4 |
| CDKN2A/B | 9p21.3 | 101 | 45.7 | 1 | 33.3 | 9 | 38 | 4 | 21.1 | 2 | 50 | 85 | 50 |
| PAX5 CNA | 9p13.2 | 70 | 31.7 | 1 | 33.3 | 10 | 40 | 1 | 5.3 | 1 | 25 | 57 | 33.5 |
| PAX5 CNA or | 9p13.2 | 81 | 36.7 | 1 | 33.3 | 11 | 44 | 1 | 5.3 | 1 | 25 | 67 | 39.4 |
| sequence | | | | | | | | | | | | | |
| ABL1 | 9q34.13 | 3 | 1.4 | 0 | 0 | 0 | 0 | 0 | 0 | 0 | 0 | 3 | 1.8 |
| ADARB2 | 10p15.2 | 4 | 1.8 | 0 | 0 | 0 | 0 | 0 | 0 | 0 | 0 | 4 | 2.4 |
| COPEB/KLF6 | 10p15 | 2 | 0.9 | 0 | 0 | 0 | 0 | 0 | 0 | 0 | 0 | 2 | 1.17 |
| ADD3 | 10q25.2 | 18 | 8.1 | 1 | 33.3 | 1 | 4 | 0 | 0 | 0 | 0 | 16 | 9.4 |
| RAG1/2 | 11p12 | 8 | 3.6 | 0 | 0 | 0 | 0 | 1 | 5.3 | 0 | 0 | 7 | 4.1 |
| NUP160/PTPRJ | 11p11.2 | 4 | 1.8 | 0 | 0 | 0 | 0 | 0 | 0 | 0 | 0 | 4 | 2.4 |
| ETV6 | 12p13.2 | 28 | 12.7 | 1 | 33.3 | 0 | 0 | 0 | 0 | 0 | 0 | 27 | 15.8 |
| KRAS | 12p12.1 | 14 | 6.3 | 0 | 0 | 2 | 8 | 1 | 5.3 | 0 | 0 | 11 | 6.5 |
| BTG1 | 12q21.3 | 23 | 10.4 | 0 | 0 | 0 | 0 | 0 | 0 | 0 | 0 | 23 | 13.5 |
| ZMYM5 | 13q12.1 | 3 | 1.4 | 0 | 0 | 0 | 0 | 0 | 0 | 0 | 0 | 3 | 1.8 |
| ELF1 | 13q14.1 | | | | | | | | | | | | |
| C13orf21/TSC22D1 | 13q14 | 20 | 9.1 | 0 | 0 | 5 | 20 | 0 | 0 | 0 | 0 | 15 | 8.8 |
| RB1 | 13q14.2 | 25 | 11.3 | 0 | 0 | 5 | 20 | 0 | 0 | 0 | 0 | 20 | 11.8 |
| DLEU2/7/mir15/ | 13q14 | 21 | 9.5 | 0 | 0 | 5 | 20 | 0 | 0 | 0 | 0 | 16 | 9.4 |
| [symbol] 16a) | | | | | | | | | | | | | |
| ATP10A | 15q12 | 6 | 2.7 | 0 | 0 | 0 | 0 | 0 | 0 | 0 | 0 | 6 | 3.5 |
| SPRED1 (5′) | 15q14 | 0 | 0 | 0 | 0 | 0 | 0 | 0 | 0 | 0 | 0 | 0 | 0 |
| LTK | 15q15.1 | 0 | 0 | 0 | 0 | 0 | 0 | 0 | 0 | 0 | 0 | 0 | 0 |
| NF1 | 17q11.2 | 6 | 2.7 | 0 | 0 | 1 | 4 | 1 | 5.3 | 0 | 0 | 4 | 2.3 |
| TCF3 | 19p13.3 | 21 | 9.5 | 0 | 0 | 15 | 60 | 0 | 0 | 0 | 0 | 6 | 3.5 |
| C20orf94 | 20p12.2 | 19 | 8.6 | 0 | 0 | 1 | 4 | 0 | 0 | 0 | 0 | 18 | 10.6 |
| ERG | 21q22 | 11 | 5 | 0 | 0 | 0 | 0 | 0 | 0 | 0 | 0 | 11 | 6.5 |
| iAmp21* | 21, | 10 | 4.5 | 0 | 0 | 0 | 0 | 0 | 0 | 0 | 0 | 10 | 5.8 |
| | varies | | | | | | | | | | | | |
| VPREB1 | 22q11.2 | 57 | 25.8 | 2 | 66.7 | 0 | 0 | 0 | 0 | 0 | 0 | 55 | 32.4 |
| IL3RA | Xp22.33 | 15 | 6.8 | 0 | 0 | 0 | 0 | 0 | 0 | 0 | 0 | 15 | 8.8 |
| DMD | Xp21.1 | 15 | 6.8 | 0 | 0 | 5 | 20 | 0 | 0 | 0 | 0 | 10 | 5.8 |
| B cell pathway | | 147 | 66.5 | 121 | 71.2 | 2 | 66.7 | 18 | 72 | 1 | 25 | 5 | 26.3 |
| B cell pathway | | 154 | 69.7 | 128 | 75.3 | 2 | 66.7 | 18 | 72 | 1 | 25 | 5 | 26.3 |
| including | | | | | | | | | | | | | |
| | | 221 | | 3 | | 25 | | 19 | | 4 | | 170 | |

| | | | | | | |
|---|---|---|---|---|---|---|
| B cell pathway | 1.26 (0-5) | 1.5 (0-5) | 1.7 (0-3) | 1.0 (0-2) | 0.3 (0-1) | 0.3 (0-1) |
| lesion per case | | | | | | |
| [symbol] | | | | | | |

Abnormalities are deletions unless otherwise indicated.
*B cell pathway lesions include deletions or sequence mutations involving BCL11A (N = 1), BLNK (N = 2), EBF1 (N = 17), IKZF1 (N = 67), IKZF2 (N = 1), LEF1 (N = 1), MEF2C (N = 1), PAX5 (N = 81), RAG1/2 (N = 8), SOX4 (N = 1), SPI1 (N = 1) and TCF3 (N = 21); no lesions were found in CD79A, GABPA, IKZF3, IL7RA, IRF4, IRF8, STAT3, STAT5A, or STAT5B. iAmp21, Intrachromosomal amplification of chromosome 21.
*Adjacent ZNF238 [symbol]
indicates data missing or illegible when filed

TABLE 15
Regions of recurring copy number alteration in the St Jude cohort.

| | | | | | | TCF3- | | ETV6- | | | | | | | | | |
|---|---|---|---|---|---|---|---|---|---|---|---|---|---|---|---|---|---|
| Lesion | Location | All | % | H50 | % | PBX1 | % | RUNX | % | MLL | % | Ph | % | Hypo | % | Other | % |
| | | 258 | | 44 | | 17 | | 50 | | 24 | | 21 | | 10 | | 92 | |
| PDE4B | 1p31.2 | 2 | 8 | 0 | 0 | 0 | 0 | 2 | 4 | 0 | 0 | 0 | 0 | 0 | 0 | 0 | 0 |
| NRAS | 1p13.1 | 1 | 4 | 0 | 0 | 0 | 0 | 0 | 0 | 0 | 0 | 0 | 0 | 0 | 0 | 1 | 1.1 |
| ADAR | 1q22 | 2 | 8 | 0 | 0 | 0 | 0 | 0 | 0 | 0 | 0 | 0 | 0 | 0 | 0 | 2 | 2.2 |
| LOC440742* | 1q44 | 2 | 8 | 0 | 0 | 0 | 0 | 0 | 0 | 0 | 0 | 0 | 0 | 0 | 0 | 2 | 2.2 |
| 1q gain | 1q23.3 [symbol] 1 | 30 | 11.6 | 13 | 29.5 | 16 | 94.1 | 0 | 0 | 0 | 0 | 0 | 0 | 0 | 0 | 1 | 1.1 |
| ARPP-21 | 3p22.3 | 8 | 3.1 | 1 | 2.3 | 0 | 0 | 2 | 4 | 0 | 0 | 1 | 4.8 | 2 | 20 | 2 | 2.2 |
| FHIT | 3p14.2 | 12 | 4.7 | 0 | 0 | 0 | 0 | 6 | 12 | 0 | 0 | 2 | 9.5 | 1 | 10 | 3 | 3.3 |
| FLNB | 3p14.3 | 7 | 2.7 | 1 | 2.3 | 0 | 0 | 1 | 2 | 0 | 0 | 1 | 4.8 | 1 | 10 | 3 | 3.3 |
| BTLA/CD200 | 3q13.2 | 16 | 6.2 | 0 | 0 | 0 | 0 | 8 | 16 | 0 | 0 | 5 | 23.8 | 1 | 10 | 2 | 2.2 |
| MBNL1 | 3q25.1 | 9 | 3.5 | 2 | 4.5 | 0 | 0 | 3 | 6 | 0 | 0 | 2 | 9.5 | 1 | 10 | 1 | 1.1 |
| TBL1XR1 | 3q26.32 | 15 | 5.8 | 1 | 2.3 | 0 | 0 | 8 | 16 | 1 | 4.2 | 1 | 4.8 | 0 | 0 | 4. | 4.3 |
| IL1RAP | 3q28 | 3 | 1.2 | 0 | 0 | 0 | 0 | 1 | 2 | 0 | 0 | 1 | 4.8 | 1 | 10 | 0 | 0 |
| ARHGAP24 | 4q21.23 | 2 | 8 | 0 | 0 | 0 | 0 | 0 | 0 | 0 | 0 | 0 | 0 | 1 | 10 | 1 | 1.1 |
| NR3C2 | 4q31.23 | 10 | 3.9 | 0 | 0 | 0 | 0 | 6 | 12 | 0 | 0 | 0 | 0 | 1 | 10 | 3 | 3.3 |
| LEF1 | 4q25 | 5 | 1.9 | 0 | 0 | 0 | 0 | 2 | 4.0 | 0 | 0 | 0 | 0 | 1 | 10 | 2 | 2.2 |
| FBXW7 | 4q31.3 | 5 | 1.9 | 0 | 0 | 0 | 0 | 1 | 2 | 0 | 0 | 1 | 4.8 | 1 | 10 | 2 | 2.2 |
| EBF1 | 5q33.3 | 12 | 4.7 | 1 | 2.3 | 0 | 0 | 5 | 10 | 0 | 0 | 3 | 14.3 | 1 | 10 | 2 | 2.2 |
| Histone cluster | 6p22.2 | 21 | 8.1 | 1 | 2.3 | 0 | 0 | 3 | 6 | 0 | 0 | 3 | 14.3 | 3 | 30 | 11 | 12 |
| GRIK2 | 6q16 | 11 | 4.3 | 1 | 2.3 | 1 | 5.9 | 7 | 14 | 0 | 0 | 0 | 0 | 0 | 0 | 2 | 2.2 |
| ARMC2/SESN1 | 6q21 | 13 | 5 | 0 | 0 | 0 | 0 | 8 | 16 | 0 | 0 | 0 | 0 | 0 | 0 | 5 | 5.4 |
| 4LOC389437 | 6q25.3 | 7 | 2.7 | 0 | 0 | 0 | 0 | 4 | 8 | 0 | 0 | 0 | 0 | 1 | 10 | 2 | 2/2 |
| IKZF1 | 7p13 | 48 | 18.6 | 4 | 9.1 | 0 | 0 | 0 | 0 | 1 | 4.2 | 16 | 76.2 | 5 | 50 | 22 | 22.8 |
| CDK6 | 7q21.2 | 8 | 3.1 | 1 | 2.3 | 0 | 0 | 0 | 0 | 0 | 0 | 2 | 9.5 | 3 | 30 | 2 | 2.2 |
| MSRA | 8p23 | 6 | 2.3 | 0 | 0 | 0 | 0 | 2 | 4.0 | 0 | 0 | 1 | 4.8 | 2 | 20 | 1 | 1.1 |
| TOX | 8q12.1 | 11 | 4.3 | 0 | 0 | 0 | 0 | 5 | 10 | 0 | 0 | 1 | 4.8 | 0 | 0 | 5 | 5.4 |
| CCDC26 | 8q24.21 | 5 | 1.9 | 1 | 2.3 | 0 | 0 | 0 | 0 | 0 | 0 | 0 | 0 | 0 | 0 | 4 | 4.3 |
| CDKN2A/B | 9p21.3 | 87 | 33.7 | 9 | 20.5 | 6 | 35.3 | 15 | 30 | 4 | 16.7 | 11 | 52.4 | 10 | 100 | 32 | 34.8 |
| PAX5 CNA | 9p13.2 | 79 | 30.6 | 4 | 9.1 | 7 | 41.2 | 17 | 34 | 4 | 16.7 | 11 | 52.4 | 10 | 100 | 26 | 28.3 |
| PAX5 CNA or | 9p13.2 | | | | | | | | | | | | | | | | |
| sequence | | | | | | | | | | | | | | | | | |
| ABL1 | 9q34.13 | 5 | 1.9 | 0 | 0 | 0 | 0 | 0 | 0 | 0 | 0 | 4 | 19 | 1 | 10 | 0 | 0 |
| ADARB2 | 10p15.2 | 1 | 4 | 0 | 0 | 0 | 0 | 0 | 0 | 0 | 0 | 1 | 4.8 | 0 | 0 | 0 | 0 |
| COPEB/KLF6 | 10p15 | 0 | 0 | 0 | 0 | 0 | 0 | 0 | 0 | 0 | 0 | 0 | 0 | 0 | 0 | 0 | 0 |
| PTEN | 10q23.31 | | | | | | | | | | | | | | | | |
| BLNK | 10q24.1 | 3 | 1.2 | 0 | 0 | 0 | 0 | 2 | 4 | 0 | 0 | 0 | 0 | 0 | 0 | 1 | 1.1 |
| ADD3 | 10q25.2 | 14 | 5.4 | 1 | 2.3 | 0 | 0 | 4 | 8 | 0 | 0 | 5 | 23.8 | 0 | 0 | 4 | 4.3 |
| RAG1/2 | 11p12 | 15 | 5.8 | 0 | 0 | 0 | 0 | 8 | 16 | 1 | 4.2 | 0 | 0 | 0 | 0 | 6 | 6.5 |
| NUP160/PTPRJ | 11p11.2 | 1 | 4 | 0 | 0 | 0 | 0 | 0 | 0 | 0 | 0 | 0 | 0 | 0 | 0 | 1 | 1.1 |
| ATM | 11q22.3 | 7 | 2.7 | 0 | 0 | 0 | 0 | 2 | 4 | 0 | 0 | 1 | 4.8 | 0 | 0 | 4 | 4.3 |
| ETV6 | 12p13.2 | 63 | 24.4 | 5 | 11.5 | 0 | 0 | 34 | 68 | 2 | 8.3 | 2 | 9.5 | 2 | 20 | 18 | 19.6 |
| KRAS | 12p12.1 | 20 | 7.8 | 2 | 4.5 | 0 | 0 | 8 | 16 | 1 | 4.2 | 0 | 0 | 1 | 10 | 8 | 8.7 |
| BTG1 | 12q21.33 | 18 | 7.0 | 0 | 0 | 0 | 0 | 7 | 14 | 0 | 0 | 4 | 19 | 1 | 10 | 6 | 6.5 |
| ZMYM5 | 13q12.11 | 5 | 1.9 | 1 | 2.3 | 0 | 0 | 2 | 4 | 0 | 0 | 0 | 0 | 0 | 0 | 2 | 2.2 |
| ELF1 | 13q14.11 | 12 | 4.7 | 2 | 4.5 | 2 | 11.8 | 4 | 8 | 1 | 4.2 | 0 | 0 | 1 | 10 | 2 | 2.2 |
| C13orf21/TSC22 | 13q14 | 15 | 5.8 | 2 | 4.5 | 2 | 11.8 | 4 | 8 | 1 | 4.2 | 2 | 9.5 | 1 | 10 | 3 | 3.3 |
| RB1 | 13q14.2 | 15 | 5.8 | 3 | 6.8 | 2 | 11.8 | 2 | 4 | 2 | 8.3 | 4 | 19 | 0 | 0 | 2 | 2.2 |
| DLEU2/7/mir15 [symbol] | 13q14 | 16 | 6.2 | 5 | 11.4 | 2 | 11.8 | 3 | 6 | 3 | 12.5 | 1 | 4.8 | 0 | 0 | 2 | 2.2 |
| 16a) | | | | | | | | | | | | | | | | | |
| ATP10A | 15q12 | 5 | 1.9 | 0 | 0 | 0 | 0 | 1 | 2 | 0 | 0 | 1 | 4.8 | 1 | 10 | 2 | 2.2 |
| SPRED1 (5′) | 15q14 | 6 | 2.3 | 0 | 0 | 0 | 0 | 0 | 0 | 0 | 0 | 1 | 4.8 | 1 | 10 | 4 | 4.3 |
| LTK | 15q15.1 | 6 | 2.3 | 0 | 0 | 0 | 0 | 3 | 6 | 0 | 0 | 0 | 0 | 1 | 10 | 2 | 2.2 |
| NF1 | 17q11.2 | 8 | 3.1 | 1 | 2.3 | 0 | 0 | 2 | 4 | 0 | 0 | 0 | 0 | 1 | 10 | 4 | 4.3 |
| IKZF3 (AIOLOS) | 17q21.1 | 3 | 1.2 | 0 | 0 | 0 | 0 | 0 | 0 | 0 | 0 | 0 | 0 | 2 | 20 | 1 | 1.1 |
| TCF3 | 19p13.3 | 17 | 6.6 | 1 | 2.3 | 16 | 94.1 | 0 | 0 | 0 | 0 | 0 | 0 | 0 | 0 | 0 | 0 |
| C20orf94 | 20p12.2 | 20 | 7.8 | 2 | 4.5 | 0 | 0 | 7 | 14 | 0 | 0 | 7 | 33.3 | 0 | 0 | 4 | 4.3 |
| ERG | 21q22 | 14 | 5.4 | 0 | 0 | 0 | 0 | 0 | 0 | 0 | 0 | 0 | 0 | 0 | 0 | 14 | 15.2 |
| iAmp21* | 21, | 11 | 4.3 | 0 | 0 | 0 | 0 | 5 | 10 | 0 | 0 | 0 | 0 | 0 | 0 | 6 | 6.5 |
| | varies | | | | | | | | | | | | | | | | |
| VPREB1 | 22q11.22 | 80 | 31 | 7 | 15.9 | 1 | 5.9 | 35 | 70 | 1 | 4.2 | 7 | 33.3 | 3 | 30 | 26 | 28.3 |
| IL3RA | Xp22.33 | 18 | 7.0 | 1 | 2.3 | 0 | 0 | 6 | 12 | 0 | 0 | 0 | 0 | 1 | 10 | 10 | 10.9 |
| DMD | Xp21.1 | 11 | 4.3 | 1 | 2.3 | 0 | 0 | 4 | 8 | 0 | 0 | 0 | 0 | 0 | 0 | 6 | 6.5 |
| B pathway | | 135 | 52.3 | 11 | 25 | 17 | 100 | 27 | 54 | 6 | 25 | 16 | 76.2 | 10 | 100 | 48 | 52.2 |
| B pathway | | 166 | 64.3 | 16 | 36.4 | 17 | 100 | 42 | 64 | 6 | 25 | 16 | 76.2 | 10 | 100 | 59 | 64.1 |
| with VPREB | | | | | | | | | | | | | | | | | |

H50, high hyperdiploid,
iAmp21, Intrachromosomal amplification of chromosome 21.
*Adjacent to ZNF238. [symbol]
indicates data missing or illegible when filed

TABLE 16
Results of PAX5 genomic quantitative PCR.

| Sample | PAX5 | | | | |
|---|---|---|---|---|---|
| ID | Group | deletion region | Exon 3 | Exon 6 | Exon 8 |
| 9906_002 | TCF3-PBX1 | All gene | 0.60 | 0.58 | 0.61 |
| 9906_004 | Other | 5′ to distal | 0.13 | 0.16 | 0.20 |
| 9906_009 | Other | 5′ to distal | 0.47 | 0.53 | 0.58 |
| 9906_013 | Other | e3 - distal | 0.54 | 0.31 | 0.33 |
| 9906_014 | Other | e2-e5 | 0.57 | 1.05 | 1.05 |
| 9906_028 | TCF3-PBX1 | All gene | 0.44 | 0.50 | 0.44 |
| 9906_034 | Other | All gene | 0.31 | 0.30 | 0.29 |
| 9906_037 | Other | e6 - distal | 0.70 | 0.38 | 0.38 |
| 9906_040 | Other | All gene | 0.31 | 0.35 | 0.37 |
| 9906_045 | Other | All gene | 0.60 | 0.60 | 0.61 |
| 9906_046 | TCF3-PBX1 | All gene | 0.42 | 0.41 | 0.39 |
| 9906_048 | Other | 5′ - e7 | 0.39 | 0.12 | 0.65 |
| 9906_055 | Other | e2-e5 | 0.51 | 0.92 | 1.02 |
| 9906_063 | TCF3-PBX1 | e6 | 0.48 | 0.32 | 0.55 |
| 9906_065 | Other | All gene | 0.50 | 0.52 | 0.47 |
| 9906_070 | Other | Promoter - e3 | 0.59 | 0.76 | 0.96 |
| 9906_080 | Other | e7-distal | 0.80 | 0.84 | 0.50 |
| 9906_098 | Other | e9 | 0.95 | 0.95 | 0.94 |
| 9906_102 | Other | Amplification | 2.77 | 1.21 | 0.90 |
| | | e2-e5 | | | |
| 9906_107 | Other | e7-distal | 1.15 | 1.16 | 0.63 |
| 9906_111 | Other | e6-distal | 1.09 | 0.60 | 0.57 |
| 9906_118 | Other | Promoter - e5 | 0.72 | 1.16 | 0.97 |
| 9906_124 | Other | e2-e4, e6 | 0.54 | 0.53 | 1.00 |
| 9906_141 | Other | e2-e5 | 0.61 | 1.06 | 0.98 |
| 9906_154 | Other | e2-e8 | 0.49 | 0.48 | 0.43 |
| 9906_157 | Other | e2-e6 | 0.42 | 0.44 | 0.86 |
| 9906_160 | Other | All gene | 0.57 | 0.63 | 0.56 |
| 9906_161 | Other | 5′ - e3 | 0.33 | 0.59 | 0.65 |
| 9906_163 | TCF3-PBX1 | 5′ - e7 | 0.03 | 0.03 | 0.04 |
| 9906_175 | Other | e6-8 | 0.97 | 0.54 | 0.55 |
| 9906_180 | Other | 5′ - e7 | 0.71 | 0.35 | 0.81 |
| 9906_192 | Other | e8-9 | 0.78 | 0.83 | 0.43 |
| 9906_196 | Other | e2-e7, | 0.49 | 0.06 | 0.99 |
| | | homozygous | | | |
| | | e6-7 | | | |
| 9906_218 | TCF3-PBX1 | All gene | 0.44 | 0.46 | 0.50 |
| 9906_268 | Other | e6-distal | 1.13 | 0.64 | 0.76 |

Results represent means of duplicate measurements, and are ratios of PAX5 to control (RNAse P).
Values <0.75 represent hemizygous deletion, and <0.3 homozygous deletion.

TABLE 17
PAX5 sequence mutations in the P9906 cohort.

| | | | | PAX5 |
|---|---|---|---|---|
| | | PAX5 | PAX5 deletion | mutation |
| Sample ID | Group | deletion | region | description |
| 9906_034 | Other | Yes | All gene | P80R |
| 9906_060 | Other | No | | V151I |
| 9906_065 | Other | Yes | All gene | G24R |
| 9906_086 | Other | No | | G24R |
| 9906_106 | Other | Yes | All gene | P80R |
| 9906_110 | Other | No | | Exon 3 splice; |
| | | | | D53V |
| 9906_113 | Other | No | | I139T |
| 9906_121 | Other | Yes | All gene | P80R |
| 9906_156 | TCF3-PBX1 | No | | I301T |
| 9906_173 | Other | Yes | All gene | T333fs |
| 9906_179 | Other | No | | P80R; E201fs |
| 9906_180 | Other | Yes | Promoter-e7 | S213L |
| 9906_188 | Other | Yes | All gene | P80R |
| 9906_192 | Other | Yes | e8-9 | R59G |
| 9906_195 | Other | No | | T75R; V336fs |
| 9906_228 | Other | No | | P80R; E201fs |
| 9906_233 | Other | No | | P80R; E7 splice |
| 9906_234 | Other | Yes | Focal promoter | E9 splice |
| 9906_235 | Other | Yes | All gene | E9 splice |
| 9906_239 | Other | No | | V319FS |
| 9906_256 | Other | Yes | All gene | P80R |
| 9906_258 | Other | No | | V319FS |

Five cases had two point mutations in trans, and 11 cases had deletions of one PAX5 allele and point mutation of the second allele.
e, exon;
fs, frameshift

TABLE 18
Description all P9906 cases harboring IKZF1 deletions and/or sequence
mutations, and results of genomic quantitative PCR results for IKZF1 deletions.

| Sample | | IKZF1 | | gqPCR | gqPCR | gqPCR | gqPCR | IKZF1 |
|---|---|---|---|---|---|---|---|---|
| ID | Group | deletion | Region | e1 | e3 | e5 | e6 | mutation |
| 9906_261 | MLL | Yes | e3-e6 | 0.96 | | 0.58 | 0.59 | |
| 9906_001 | Other | Yes | e1-e6 | | | | | |
| 9906_007 | Other | Yes | e3-6 | 1.57 | 0.56 | | 0.53 | |
| 9906_014 | Other | Yes | e1-7 | 0.56 | 0.61 | | 0.56 | |
| 9906_019 | Other | Yes | All gene | 0.67 | 0.66 | | 0.59 | G158S |
| 9906_021 | Other | Yes | All gene | | | | | |
| 9906_024 | Other | No | | | | | | H224fs |
| 9906_027 | Other | Yes | e3-e6 | 1.09 | 0.63 | | 0.55 | |
| 9906_030 | Other | Yes | e3-e6 | 1.23 | | | 0.59 | |
| 9906_033 | Other | Yes | e3-e6 | 1.17 | 0.57 | | 0.54 | |
| 9906_038 | Other | Yes | e3 - distal | | | | | |
| 9906_039 | Other | Yes | All gene | 0.54 | | | 0.53 | |
| 9906_040 | Other | Yes | All gene | 0.73 | | | 0.59 | |
| 9906_045 | Other | Yes | e3-e6 | 0.97 | | 0.56 | 0.71 | |
| 9906_047 | Other | Yes | e3-e6 | 1.17 | | 0.71 | 0.68 | |
| 9906_048 | Other | Yes | e1-e6 | 0.65 | 0.72 | | 0.64 | |
| 9906_049 | Other | Yes | All gene | 0.71 | 0.75 | | 0.71 | |
| 9906_055 | Other | Yes | All gene | | | | | L117fs |
| 9906_064 | Other | Yes | 5′-e1 | 0.59 | | 0.99 | 1.02 | |
| 9906_065 | Other | No | | | | | | S402fs |
| 9906_078 | Other | Yes | 5′-e1 | 0.52 | 1.23 | | 1.08 | |
| 9906_082 | Other | Yes | 5′-e1 | 0.56 | | 1.02 | 1.09 | |
| 9906_084 | Other | Yes | e3-e6 | 1.22 | | 0.53 | 0.57 | |
| 9906_087 | Other | Yes | All gene | 0.64 | 0.70 | | 0.68 | |
| 9906_090 | Other | No | | | | | | R111* |
| 9906_093 | Other | Yes | All gene | | | | | |
| 9906_107 | Other | Yes | e3-e6 | 1.11 | | 0.57 | 0.65 | |
| 9906_109 | Other | Yes | 5′-e1 | 0.57 | | 1.05 | 1.20 | |
| 9906_113 | Other | Yes | 5′-e1 | 0.54 | | 1.05 | 1.47 | |
| 9906_118 | Other | Yes | All gene | 0.59 | 0.61 | | 0.61 | |
| 9906_120 | Other | Yes | All gene | | | | | |
| 9906_124 | Other | Yes | e1-e5 | 0.69 | | 0.57 | 0.81 | |
| 9906_135 | Other | Yes | All gene | 0.57 | 0.57 | | 0.57 | |
| 9906_138 | Other | Yes | e1-e5 | | | | | |
| 9906_141 | Other | Yes | e3-e6 | | | | | |
| 9906_146 | Other | Yes | e3-e6 | | | | | |
| 9906_151 | Other | Yes | e3-e6 | | | | | |
| 9906_153 | Other | Yes | All gene | | | | | |
| 9906_154 | Other | Yes | e1-e3 | 0.53 | | 1.00 | 1.40 | |
| 9906_161 | Other | Yes | e3 - distal | 1.29 | 0.97 | | 0.71 | |
| 9906_168 | Other | Yes | 5′ - e1 | 0.67 | 1.07 | | 1.05 | |
| 9906_170 | Other | Yes | e1-e4 | 0.49 | | 0.91 | 1.23 | |
| 9906_173 | Other | Yes | All gene | 0.74 | | 0.66 | 0.65 | |
| 9906_174 | Other | Yes | e3 - distal | 1.63 | | 0.57 | 0.63 | |
| 9906_175 | Other | Yes | All gene | 0.61 | | | 0.62 | |
| 9906_179 | Other | No | | | | | | E504fs |
| 9906_192 | Other | Yes | e3 - distal | 1.10 | 0.64 | | 0.58 | |
| 9906_196 | Other | Yes | e2-e5 | 1.09 | | 0.54 | 0.79 | |
| 9906_206 | Other | Yes | e1-e4 | 0.62 | | 0.92 | 0.98 | |
| 9906_210 | Other | Yes | e1-e6 | 0.58 | 0.52 | | 0.52 | |
| 9906_215 | Other | Yes | e1-e4 | 0.63 | | 0.95 | 0.95 | |
| 9906_217 | Other | Yes | 5′ - e6, | 0.11 | | 0.55 | 0.82 | |
| | | | homozygous | | | | | |
| | | | 5′ - e1 | | | | | |
| 9906_219 | Other | Yes | All gene | 0.63 | 0.75 | | 0.52 | |
| 9906_222 | Other | Yes | e3-e6 | 0.99 | 0.65 | | 0.63 | |
| 9906_225 | Other | Yes | e3-e6 | 1.21 | | 0.43 | 0.58 | |
| 9906_231 | Other | Yes | e3-e6 | | | | | |
| 9906_234 | Other | Yes | e1-e6 | 0.62 | | 0.66 | 0.58 | |
| 9906_240 | Other | Yes | e1-e6 | 0.59 | | 0.63 | 0.69 | |
| 9906_242 | Other | Yes | e3-e6 | 0.99 | | 0.49 | 0.48 | |
| 9906_244 | Other | Yes | e3-e6 | 1.06 | | 0.60 | 0.54 | |
| 9906_250 | Other | Yes | e1-e6 | 0.45 | | 0.50 | 0.49 | |
| 9906_252 | Other | Yes | e3-e6 | 1.04 | | 0.60 | 0.57 | |
| 9906_253 | Other | Yes | All gene | 0.45 | | 0.46 | 0.45 | |
| 9906_257 | Other | Yes | e3-e6 | 0.97 | 0.91 | | 0.65 | |
| 9906_258 | Other | Yes | e1-e6 | 0.61 | | 0.62 | 0.62 | |
| 9906_262 | Other | Yes | e3-e6 | 0.96 | | 0.53 | 0.52 | |
| 9906_271 | Other | Yes | e3-distal | | | | | |

Results represent means of duplicate measurements, and are ratios of IKZF1 to control (RNAse P).
Values <0.75 represent hemizygous deletion, and <0.3 homozygous deletion.
e, exon;
fs, frameshift (mutation);
*nonsense mutation

TABLE 19
Description of B-cell pathway lesions observed in the P9906 cohort.

| Sample | | Number | |
|---|---|---|---|
| ID | Group | of | B cell pathway lesions |
| 9906_001 | Other | 2 | IKZF1 deln; VPREB1 deln |
| 9906_002 | E2A | 2 | PAX5 deln; TCF3 deln |
| 9906_003 | E2A | 1 | TCF3 deln |
| 9906_004 | Other | 1 | PAX5 deln |
| 9906_007 | Other | 3 | IKZF1 deln; VPREB1 deln; MEF2C deln |
| 9906_009 | Other | 2 | PAX5 deln; VPREB1 deln |
| 9906_010 | Other | 2 | EBF1 deln; VPREB1 deln |
| 9906_011 | Other | 1 | IKZF2 deln |
| 9906_012 | Other | 1 | EBF1 deln |
| 9906_013 | Other | 1 | PAX5 deln; |
| 9906_014 | Other | 4 | EBF1 deln; IKZF1 deln; PAX5 deln; VPREB1 deln |
| 9906_016 | Other | 1 | EBF1 deln |
| 9906_017 | E2A | 1 | TCF3 deln |
| 9906_019 | Other | 3 | IKZF1 deln; G158S IKZF1 mutation; VPREB1 deln |
| 9906_020 | Other | 2 | RAG1/2 deln; VPREB1 deln |
| 9906_021 | Other | 3 | IKZF1 deln; PAX5 deln; VPREB1 deln |
| 9906_024 | Other | 1 | H224FS IKZF1 mutation |
| 9906_027 | Other | 3 | EBF1 deln; IKZF1 deln; VPREB1 deln |
| 9906_028 | E2A | 1 | PAX5 deln |
| 9906_030 | Other | 1 | IKZF1 deln |
| 9906_031 | Other | 1 | VPREB1 deln |
| 9906_033 | Other | 1 | IKZF1 deln |
| 9906_034 | Other | 4 | PAX5 deln; P80R PAX5 mutation; RAG1/2 deln; LEF1 deln |
| 9906_037 | Other | 2 | PAX5 deln; RAG1/2 deln |
| 9906_038 | Other | 1 | IKZF1 deln |
| 9906_039 | Other | 1 | IKZF1 deln |
| 9906_040 | Other | 2 | IKZF1 deln; PAX5 deln |
| 9906_045 | Other | 2 | IKZF1 deln; PAX5 deln |
| 9906_046 | E2A | 2 | PAX5 deln; TCF3 deln |
| 9906_047 | Other | 2 | IKZF1 deln; VPREB1 deln |
| 9906_048 | Other | 3 | IKZF1 deln; PAX5 deln, homozygous |
| 9906_049 | Other | 1 | IKZF1 deln |
| 9906_052 | Other | 1 | VPREB1 deln |
| 9906_055 | Other | 3 | IKZF1 deln; L1 17FS IKZF1 mutation; PAX5 deln |
| 9906_057 | Other | 1 | RAG1/2 deln |
| 9906_058 | E2A | 1 | TCF3 deln |
| 9906_060 | Other | 2 | V1 51I PAX5 mutation; VPREB1 deln |
| 9906_063 | E2A | 2 | PAX5 deln; TCF3 deln |
| 9906_064 | Other | 2 | IKZF1 deln; VPREB1 deln |
| 9906_065 | Other | 4 | S402FS IKZF1 mutation; PAX5 deln; G24R PAX5 |
| | | | mutation; VPREB1 deln |
| 9906_066 | Other | 1 | PAX5 deln |
| 9906_070 | Other | 1 | PAX5 deln |
| 9906_071 | E2A | 1 | PAX5 deln |
| 9906_073 | Other | 2 | RAG1/2 deln; TCF3 deln |
| 9906_075 | E2A | 2 | PAX5 deln; TCF3 deln |
| 9906_078 | Other | 2 | IKZF1 deln; VPREB1 deln |
| 9906_079 | E2A | 2 | PAX5 deln; TCF3 deln |
| 9906_080 | Other | 1 | PAX5 deln |
| 9906_082 | Other | 3 | IKZF1 deln; PAX5 deln; VPREB1 deln |
| 9906_083 | TEL | 3 | EBF1 deln; VPREB1 deln; SOX4 deln |
| 9906_084 | Other | 3 | EBF1 deln; IKZF1 deln; VPREB1 deln |
| 9906_085 | Other | 1 | RAG1/2 deln |
| 9906_086 | Other | 1 | G24R PAX5 mutation |
| 9906_087 | Other | 1 | IKZF1 deln |
| 9906_090 | Other | 4 | EBF1 deln; R1 11 * IKZF1 mutation;RAG1/2 deln; VPREB1 |
| 9906_092 | Other | 1 | VPREB1 deln |
| 9906_093 | Other | 1 | IKZF1 deln |
| 9906_094 | Other | 1 | BLNK deln |
| 9906_096 | E2A | 2 | PAX5 deln; TCF3 deln |
| 9906_097 | MLL | 1 | PAX5 deln |
| 9906_098 | Other | 1 | PAX5 deln |
| 9906_1 | Other | 2 | focal internal PAX5 amplification; VPREB1 deln |
| 9906_1 | Other | 2 | PAX5 deln; P80R PAX5 mutation |
| 9906_107 | Other | 2 | IKZF1 deln; PAX5 deln |
| 9906_108 | Other | 1 | PAX5 deln |
| 9906_1 | Other | 3 | EBF1 deln; IKZF1 deln; TCF3 deln |
| 9906_1 | Other | 3 | D53V and E3 splice PAX5 mutation; VPREB1 deln |
| 9906_111 | Other | 1 | PAX5 deln |
| 9906_1 | Other | 3 | IKZF1 deln; I139T PAX5 mutation; VPREB1 deln |
| 9906_114 | Other | 2 | VPREB1 deln; BCL11A deln |
| 9906_117 | Other | 2 | EBF1 deln; VPREB1 deln |
| 9906_118 | Other | 2 | IKZF1 deln; PAX5 deln |
| 9906_120 | Other | 3 | IKZF1 deln; PAX5 deln; VPREB1 deln |
| 9906_121 | Other | 2 | PAX5 deln; P80R PAX5 mutation |
| 9906_124 | Other | 3 | IKZF1 deln; PAX5 deln; VPREB1 deln |
| 9906_128 | MLL | 1 | RAG1/2 deln |
| 9906_135 | Other | 1 | IKZF1 deln |
| 9906_137 | MLL | 1 | RAG1/2 deln |
| 9906_138 | Other | 1 | IKZF1 deln |
| 9906_141 | Other | 3 | IKZF1 deln; PAX5 deln; VPREB1 deln |
| 9906_144 | Other | 1 | VPREB1 deln |
| 9906_145 | Other | 2 | PAX5 deln; VPREB1 deln |
| 9906_146 | Other | 1 | IKZF1 deln |
| 9906_147 | Other | 2 | PAX5 deln; VPREB1 deln |
| 9906_148 | Other | 1 | PAX5 deln |
| 9906_150 | Other | 2 | PAX5 deln; VPREB1 deln |
| 9906_151 | Other | 2 | IKZF1 deln; VPREB1 deln |
| 9906_153 | Other | 2 | IKZF1 deln; VPREB1 deln |
| 9906_154 | Other | 2 | IKZF1 deln; PAX5 deln |
| 9906_155 | Other | 1 | VPREB1 deln |
| 9906_156 | E2A | 1 | I301T PAX5 mutation |
| 9906_157 | Other | 2 | PAX5 deln; VPREB1 deln |
| 9906_159 | E2A | 1 | TCF3 deln |
| 9906_1 | Other | 2 | PAX5 deln; VPREB1 deln |
| 9906_161 | Other | 3 | IKZF1 deln; PAX5 deln; VPREB1 deln |
| 9906_1 | E2A | 2 | PAX5 deln; TCF3 deln |
| 9906_166 | E2A | 1 | TCF3 deln |
| 9906_168 | Other | 3 | EBF1 deln; IKZF1 deln; RAG1/2 deln |
| 9906_170 | Other | 1 | IKZF1 deln |
| 9906_171 | T4 10 | 1 | PAX5 deln |
| 9906_1 | Other | 3 | IKZF1 deln; PAX5 deln; T333FS PAX5 mutation |
| 9906_1 | Other | 1 | IKZF1 deln |
| 9906_175 | Other | 3 | IKZF1 deln; PAX5 deln; VPREB1 deln |
| 9906 | Other | 1 | PAX5 deln |
| 9906 | Other | 1 | PAX5 deln |
| 9906_179 | Other | 4 | E504FS IKZF1 mutation; P80R and E201FS PAX5 |
| | | | mutation; VPREB1 deln |
| 9906_1 | Other | 2 | PAX5 deln; S213L PAX5 mutation |
| 9906_1 | Other | 2 | RAG1/2 deln; SPI1 deln |
| 9906 | Other | 1 | PAX5 deln |
| 9906_1 | Other | 2 | VPREB1 deln; BLNK deln |
| 9906_1 | Other | 2 | PAX5 deln; P80R PAX5 mutation |
| 9906_1 | Other | 3 | IKZF1 deln; PAX5 deln; R59G PAX5 mutation |
| 9906 | Other | 1 | VPREB1 deln |
| 9906 | Other | 2 | T75R and V336FS PAX5 mutation |
| 9906_1 | Other | 4 | IKZF1 deln; PAX5 deln, homozygous; VPREB1 deln |
| 9906 | Other | 1 | PAX5 deln |
| 9906_202 | E2A | 1 | TCF3 deln |
| 9906_206 | Other | 2 | EBF1 deln; IKZF1 deln |
| 9906 | Other | 3 | IKZF1 deln; PAX5 deln; VPREB1 deln |
| 9906_2 | Other | 3 | PAX5 deln; IKZF1 deln; VPREB 1 deln |
| 9906_21 | Other | 3 | EBF1 deln; IKZF1 deln, homozygous |
| 9906_218 | E2A | 2 | PAX5 deln; TCF3 deln |
| 9906_21 | Other | 2 | IKZF1 deln; TCF3 deln |
| 9906_222 | Other | 2 | EBF1 deln; IKZF1 deln |
| 9906_225 | Other | 4 | IKZF1 deln; PAX5 deln; RAG1/2 deln; VPREB1 deln |
| 9906_227 | MLL | 1 | M3 1V IKZF1 mutation |
| 9906_228 | Other | 2 | P80R and E201FS PAX5 mutation |
| 9906_231 | Other | 2 | IKZF1 deln; VPREB1 deln |
| 9906_233 | Other | 2 | P80R and E7 splice PAX5 mutation |
| 9906_234 | Other | 3 | IKZF1 deln; PAX5 deln; E9 splice PAX5 mutation |
| 9906_235 | Other | 2 | PAX5 deln; E9 splice PAX5 mutation |
| 9906_236 | E2A | 1 | TCF3 deln |
| 9906_239 | Other | 2 | V3 1 9FS PAX5 mutation; VPREB1 deln |
| 9906_240 | Other | 1 | IKZF1 deln |
| 9906_242 | Other | 1 | IKZF1 deln |
| 9906_243 | TEL | 2 | PAX5 deln; VPREB1 deln |
| 9906_244 | Other | 1 | IKZF1 deln |
| 9906_250 | Other | 4 | EBF1 deln; IKZF1 deln; PAX5 deln; VPREB1 deln |
| 9906_252 | Other | 1 | IKZF1 deln |
| 9906_253 | Other | 1 | IKZF1 deln |
| 9906_254 | Other | 1 | PAX5 deln |
| 9906_255 | Other | 1 | focal internal PAX5 amplification |
| 9906_256 | Other | 2 | PAX5 deln; P80R PAX5 mutation |
| 9906_257 | Other | 3 | IKZF1 deln; PAX5 deln; VPREB1 deln |
| 9906_258 | Other | 5 | EBF1 deln; IKZF1 deln; V319FS PAX5 mutation; RAG1/2 |
| | | | VPREB1 deln |
| 9906_259 | Other | 1 | VPREB1 deln |
| 9906_260 | Other | 1 | RAG1/2 deln |
| 9906_261 | MLL | 1 | IKZF1 deln |
| 9906_262 | Other | 2 | IKZF1 deln; VPREB1 deln |
| 9906_263 | Other | 2 | TCF3 deln; VPREB1 deln |
| 9906_264 | Other | 1 | PAX5 deln; TCF3 deln |
| 9906_265 | Other | 1 | TCF3 deln |
| 9906_268 | Other | 1 | PAX5 deln |
| 9906_271 | Other | 3 | EBF1 deln; IKZF1 deln; VPREB1 deln |

In addition to PAX5 and IKZF1 abnormalities, lesions were also identified in CNAs were also observed in TCF3 (N = 21), EBF1 (N = 17), RAG1/2 (N = 8), BLNK (N = 2), BCL11A, IKZF2 (encoding the IKAROS family member HELIOS), LEF1, MEF2C, SOX4 and SPI1 (PU.1) (1 each).
deln, deletion.

TABLE 20
Variation in number of B cell pathway lesions between P9906
ALL subtypes

| | Mean number of B cell | |
|---|---|---|
| Group | pathway lesions | Range |
| All | 1.3 (0-5) | 0-5 |
| TCF3-PBX1 | 1.0 (0-2) | 0-2 |
| MLL-rearranged | 0.3 (0-1) | 0-1 |
| Other | 1.5 (0-5) | 0-5 |
| Hyperdiploid | 0.3 (0-1) | 0-1 |
| ETV6-RUNX1 | 1.7 (0-1) | 0-3 |
| ANOVA P = 0.0001 | | |

Results of ANOVA post hoc Fister's PLSD test

| | Mean Diff. | P-Value |
|---|---|---|
| Hyperdiploid, TCF3- | −0.79 | 0.1829 |
| PBX1 | | |
| Hyperdiploid, ETV6- | −1.417 | 0.0926 |
| RUNX1 | | |
| Hyperdiploid, MLL | −0.013 | 0.9826 |
| Hyperdiploid, Other | −1.215 | 0.0298 |
| TCF3-PBX1, ETV6- | −0.627 | 0.3512 |
| RUNX1 | | |
| TCF3-PBX1, MLL | 0.777 | 0.0210 |
| TCF3-PBX1, Other | −0.425 | 0.0723 |
| ETV6-RUNX1, MLL | 1.404 | 0.0408 |
| ETV6-RUNX1, Other | 0.202 | 0.7524 |
| MLL, Other | −1.202 | <0.0001 |

TABLE 21
Genes with univariate Cox score exceeding threshold of ±1.8 in SPC
analysis, P9906 cohort.

| Name | Raw score | Importance score |
|---|---|---|
| IKZF1 | −3.588 | −27.988 |
| BTLA | −2.178 | −5.401 |
| EBF1 | −1.858 | −5.171 |
| P2RY5 | 1.818 | 1.581 |
| FLNB | −1.96 | −1.499 |
| ZNF238 | −2.024 | −1.195 |
| RAG1 | −1.931 | −1.037 |
| CALM2 | −1.873 | 0.999 |
| HAAO | −1.937 | 0.958 |
| SRBD1 | −1.999 | 0.832 |
| MSH6 | −1.92 | 0.824 |
| SUSD3 | −1.953 | −0.743 |
| PRKCE | −1.808 | 0.72 |
| PPM1B | −2.07 | 0.611 |
| FAM82A | −1.986 | 0.525 |
| HEATR5B | −1.869 | 0.514 |
| C9orf71 | −1.888 | 0.511 |
| ZFP36L2 | −1.807 | 0.415 |
| FXN | −2.341 | 0.385 |
| SLC46A2 | −1.836 | −0.318 |
| SPAST | −2.068 | 0.304 |
| COX7A2L | −1.803 | 0.296 |
| PRKACG | −2.735 | −0.1 |

Raw score refers to the modified univariate Cox score calculated for each gene.
Importance score is a measure of correlation between each gene and the first principal component derived from the SPC analysis.

TABLE 22
Genes with univariate Cox score exceeding threshold of ±1.9 in SPC
analysis, St Jude cohort.

| Name | Raw Score | Importance Score |
|---|---|---|
| IKZF1 | −3.164 | −18.607 |
| TAS2R5 | −2.056 | −8.751 |
| LOC136242 | −2.034 | −8.674 |
| SVOPL | −1.951 | −8.584 |
| C7orf34 | −1.901 | −8.577 |
| FLJ36031 | −1.944 | −8.177 |
| GPR37 | −1.931 | −8.107 |

Raw score refers to the modified univariate Cox score calculated for each gene.
Importance score is a measure of correlation between each gene and the first principal component derived from the SPC analysis.

TABLE 23
Associations between of B cell pathway lesions, IKZF1 alterations and
hematologic relapse, P9906 cohort

| | | | Cumulative Incidence (SE)% | | |
|---|---|---|---|---|---|
| | | Relapse | Competing | | |
| Lesion Loci | N | N | Risks N | 5 year | P |
| Hematologic relapse | | | | | |
| B cell pathway | | | | | |
| No | 74 | 9 | 8 | 15.3 (5.6) | |
| Yes | 147 | 35 | 26 | 30.4 (4.9) | 0.084 |
| Number of B | | | | | |
| pathway lesions | | | | | |
| N = 0 | 67 | 9 | 6 | 17.1 (6.2) | |
| N = 1 | 67 | 12 | 8 | 23.6 (7.1) | |
| N = 2 | 52 | 9 | 9 | 25.4 (8.8) | |
| N >= 3 | 35 | 14 | 11 | 43.1 (9.4) | 0.02012 |
| IKZF1 deletion | | | | | |
| No | 158 | 20 | 22 | 14.4 (3.2) | |
| Yes | 63 | 24 | 12 | 52.7 (8.9) | 0.00004 |
| IKZF1 deletion or | | | | | |
| mutation | | | | | |
| No | 153 | 19 | 21 | 13.9 (3.1) | |
| Yes | 68 | 25 | 13 | 52.7 (8.8) | 0.00005 |
| Any relapse | | | | | |
| B cell pathway | | | | | |
| lesions | | | | | |
| No | 74 | 16 | 1 | 25.1 (6.3) | |
| Yes | 147 | 58 | 3 | 46.6 (5.2) | 0.021 |
| Number of B | | | | | |
| pathway lesions | | | | | |
| N = 0 | 67 | 14 | 1 | 24.9 (6.8) | |
| N = 1 | 67 | 19 | 1 | 34.1 (7.5) | |
| N = 2 | 52 | 17 | 1 | 41.3 (9.4) | |
| N >= 3 | 35 | 24 | 1 | 72.1 (8.7) | 0.00003 |
| IKZF1 deletion | | | | | |
| No | 158 | 39 | 3 | 26.7 (3.8) | |
| Yes | 63 | 35 | 1 | 71.8 (8.4) | 0.00006 |
| IKZF1 deletion or | | | | | |
| mutation | | | | | |
| No | 153 | 37 | 3 | 25.8 (3.8) | |
| Yes | 68 | 37 | 1 | 71.9 (8.4) | 0.00005 |

TABLE 24
Kaplan-Meier estimates of EFS by B cell pathway or IKZF1 lesions,
P9906 cohort

| | | Event-free survival (SE)% | | |
|---|---|---|---|---|
| | | Any Relapse | | |
| Lesion | N | or Death N | 5-Year | P-Value |
| B cell pathway lesions | | | | |
| No | 74 | 17 | 73.5 (12.6) | |
| Yes | 147 | 61 | 51.3 (8.7) | 0.019 |
| Number of B cell | | | | |
| pathway lesions | | | | |
| N = 0 | 67 | 15 | 73.6 (13.4) | |
| N = 1 | 67 | 20 | 64.4 (11.1) | |
| N = 2 | 52 | 18 | 56.7 (16.7) | |
| N >= 3 | 35 | 25 | 25 (12.5) | <0.00001 |
| IKZF1 deletion | | | | |
| No | 158 | 42 | 71.4 (8.3) | |
| Yes | 63 | 36 | 26.7 (10.2) | 0.00007 |
| IKZF1 deletion or | | | | |
| mutation | | | | |
| No | 153 | 40 | 72.2 (8.3) | |
| Yes | 68 | 38 | 26.7 (10.2) | 0.00008 |

TABLE 25
Hazard ratio estimates of B-cell pathway and IKZF1 lesions
on relapse and event free survival, P9906 cohort.

| | Hazard Ratio (95% CI) | P-value |
|---|---|---|
| Hematologic relapse | | |
| B cell pathway lesions | 2.10 (1.05-4.2) | 0.037 |
| Number of B cell pathway | 1.49 (1.11-2.02) | 0.0087 |
| lesions | | |
| IKZF1 deletion | 3.52 (1.85-6.7) | 0.00013 |
| IKZF1 deletion or mutation | 3.38 (1.78-6.4) | 0.00019 |
| Any relapse | | |
| B cell pathway lesion | 2.07 (1.18-3.65) | 0.011 |
| Number of B cell pathway | 1.72 (1.36-2.17) | 0.000005 |
| lesions | | |
| IKZF1 deletion | 2.89 (1.76-4.74) | 0.00003 |
| IKZF1 deletion or mutation | 2.87 (1.75-4.72 | 0.00003 |
| Event free survival | | |
| B cell pathway lesion | 2.08 (1.19-3.65) | 0.10 |
| Number of B cell pathway | 1.70 (1.34-2.15) | 0.00001 |
| lesions | | |
| IKZF1 deletion | 2.71 (1.64-4.45) | 0.00009 |
| IKZF1 deletion or mutation | 2.65 (1.61-4.33) | 0.00011 |

Fine and Gray test, after adjustment for age, presentation leukocyte count and cytogenetic subtype.

TABLE 26
Associations between genomic abnormalities and day 8 MRD, P9906 cohort.

| | Day 8 MRD N (%) | | | P- |
|---|---|---|---|---|
| Lesion Loci | MRD <= 0.01% | 0.01% < MRD <= 1.0% | MRD > 1.0% | Value |
| RB1 | | | | |
| No | 24 (14.20) | 59 (34.91) | 86 (50.89) | |
| Yes | 9 (33.33) | 9 (33.33) | 9 (33.33) | 0.038 |
| EBF1 | | | | |
| No | 32 (17.88) | 66 (36.87) | 81 (45.25) | |
| Yes | 1 (5.88) | 2 (11.76) | 14 (82.35) | 0.014 |
| IKZF1 deletion | | | | |
| No | 26 (18.06) | 51 (35.42) | 67 (46.53) | |
| Yes | 7 (13.46) | 17 (32.69) | 28 (53.85) | 0.61 |
| IKZF1 deletion or mutation | | | | |
| No | 26 (18.31) | 51 (35.92) | 65 (45.77) | |
| Yes | 7 (12.96) | 17 (31.48) | 30 (55.56) | 0.44 |
| PAX5 deletion or mutation | | | | |
| No | 14 (11.20) | 42 (33.60) | 69 (55.20) | |
| Yes | 19 (26.76) | 26 (36.62) | 26 (36.62) | 0.007 |
| Age group | | | | |
| 1LagJH10 years | 14 (21.21) | 21 (31.82) | 31 (46.97) | |
| Age > 10 years | 19 (14.62) | 47 (36.15) | 64 (49.23) | 0.49 |
| WBC group | | | | |
| WBC < 50K | 17 (17.35) | 39 (39.80) | 42 (42.86) | |
| WBC ≧ 50k | 16 (16.33) | 29 (29.59) | 53 (54.08) | 0.25 |
| Subtype | | | | |
| Hyperdiploid or ETV6- | 2 (40.00) | 2 (40.00) | 1 (20.00) | |
| RUNX1 | | | | |
| TCF3-PBX1 | 2 (9.52) | 12 (57.14) | 7 (33.33) | |
| MLL-rearranged | 4 (23.53) | 8 (47.06) | 5 (29.41) | |
| Others | 25 (16.34) | 46 (30.07) | 82 (53.59) | 0.075 |

TABLE 27
Associations between genetic lesions and day 29 MRD, P9906 cohort

| | Day 29 MRD | | | | | | |
|---|---|---|---|---|---|---|---|
| | N (%) | | | | | | |
| Lesion Loci | MRD <= 0.01% | | 0.01% < MRD <= 1.0 | | MRD > 1.0 | | P-Value |
| 1q gain | | | | | | | |
| No | 113 | | 46 | | 25 | | |
| Yes | (61.41) 18 | | (25.00) 2 | | (13.59) | | 0.034 |
| | [symbol] | | [symbol] | | [symbol] | | |
| ABL1 | | | | | | | |
| No | 131 | (65.17) | 48 | (23.88) | 22 | (10.95) | |
| Yes | 0 | (0.00) | 0 | (0.00) | 3 | (100.0) | <0.001 |
| ADD3 | | | | | | | |
| No | 124 | (66.67) | 44 | (23.66) | 18 | (9.68) | |
| Yes | 7 | (38.89) | 4 | (22.22) | 7 | (38.89) | 0.001 |
| BTLA/CD200 | | | | | | | |
| No | 127 | (66.49) | 44 | (23.04) | 20 | (10.47) | |
| Yes | 4 | (30.77) | 4 | (30.77) | 5 | (38.46) | 0.005 |
| C20orf94 | | | | | | | |
| No | 124 | (65.96) | 44 | (23.40) | 20 | (10.64) | |
| Yes | 7 | (43.75) | 4 | (25.00) | 5 | (31.25) | 0.044 |
| EBF1 | | | | | | | |
| No | 128 | (68.09) | 40 | (21.28) | 20 | (10.64) | |
| Yes | 3 | (18.75) | 8 | (50.00) | 5 | (31.25) | <0.001 |
| IKZF1 deletion | | | | | | | |
| No | 102 | (71.33) | 30 | (20.98) | 11 | (7.69) | |
| Yes | 29 | (47.54) | 18 | (29.51) | 14 | (22.95) | 0.00135 |
| IKZF1 deletion or mutation | | | | | | | |
| No | 100 | (72.46) | 29 | (21.01) | 9 | (6.52) | |
| Yes | 31 | (46.97) | 19 | (28.79) | 16 | (24.24) | 0.00019 |
| PAX5 | | | | | | | |
| No | 80 | (58.39) | 39 | (28.47) | 18 | (13.14) | |
| Yes | 51 | (76.12) | 9 | (13.43) | 7 | (10.45) | 0.034 |
| PAX5 deletion or mutation | | | | | | | |
| No | 72 | (55.81) | 39 | (30.23) | 18 | (13.95) | |
| Yes | 59 | (78.67) | 9 | (12.00) | 7 | (9.33) | 0.003 |
| RAG1/2 | | | | | | | |
| No | 130 | (66.33) | 42 | (21.43) | 24 | (12.24) | |
| Yes | 1 | (12.50) | 6 | (75.00) | 1 | (12.50) | 0.002 |
| Age | | | | | | | |
| 1 < LagJIH ≧ 10 | 49 | (74.24) | 14 | (21.21) | 3 | (4.55) | |
| [symbol] | | | | | | | |
| Age > 10 years | 82 | (59.42) | 34 | (24.64) | 22 | (15.94) | 0.039 |
| WCC | | | | | | | |
| WBC < 50K | 67 | (64.42) | 27 | (25.96) | 10 | (9.62) | |
| WBC ≧ 50K | 64 | (64.00) | 21 | (21.00) | 15 | (15.00) | 0.42 |
| Subtype | | | | | | | |
| Hyperdiploid or ETV6-RUNX1 | 5 | (83.33) | 0 | (0.00) | 1 | (16.67) | |
| TCF3-PBX1 | 22 | (100.0) | 0 | (0.00) | 0 | (0.00) | |
| MLL-rearranged | 7 | (43.75) | 8 | (50.00) | 1 | (6.25) | |
| Others | 97 | (60.63) | 40 | (25.00) | 23 | (14.38) | 0.002 |

[symbol]

indicates data missing or illegible when filed

TABLE 28
Association of genetic lesions with day 29 MRD adjusted by age,
presentation leukocyte count and genetic subtype in the
P9906 cohort

| | Odds Ratio (95% CI) | Adjusted |
|---|---|---|
| | Lesion vs. No Lesion | P-Value |
| 1q gain | 0.56 (0.09-3.29) | 0.513 |
| ABL1 | N/A | 0.968 |
| ADD3 | 4.57 (1.75-11.94) | 0.002 |
| BTLA/CD200 | 4.27 (1.46-12.49) | 0.008 |
| C20orf94 | 2.66 (0.98-7.23) | 0.055 |
| EBF1 | 5.54 (2.05-15.01) | 0.001 |
| IKZF1 | 2.38 (1.27-4.43) | 0.0065 |
| IKZF1 deletion or mutation | 2.66 (1.43-4.92) | 0.0019 |
| PAX5 | 0.55 (0.28-1.08) | 0.084 |
| PAX5 deletion or mutation | 0.39 (0.20-0.77) | 0.007 |
| RAG1/2 | 3.15 (0.84-11.80) | 0.088 |

TABLE 29
Multivariable analysis of associations between IKZF1 deletion or
mutation in the P9906 cohort adjusting for age, presentation
leukocyte count, leukemia subtype, and MRD level.

| | Hazard Ratio (95% CI) | P-Value |
|---|---|---|
| Hematologic relapse* | | |
| Day 29 MRD | 3.27 (1.80-5.94) | 0.0001 |
| Any relapse* | | |
| Day 29 MRD | 2.53 (1.62-3.98) | <0.0001 |
| Any event | | |
| Day 8 MRD | 2.69 (1.57-4.62) | 0.0003 |
| Day 29 MRD | 1.97 (1.14-3.38) | 0.014 |

*As there was no event for day 8 MRD < 0.01%, this analysis could not be performed for day 8 MRD and relapse

TABLE 30
Cumulative incidence of isolated or combined hematologic relapse by
genetic lesions, St Jude cohort with MRD data (N = 160).

| | | | | Cumulative Incidence (SE)% | | |
|---|---|---|---|---|---|---|
| | | | | | P-Value | P-Value |
| | | Hematologic | Competing | | Unstratified | Stratified Gray's |
| Lesion | Sub N | relapse N | Risks N | 5-Year | Gray's Test | Test* |
| RAG1/2 | | | | | | |
| No | 150 | 17 | 6 | 10.4 (2.7) | | |
| Yes | 10 | 2 | 0 | 25.0 (17.2) | 0.32 | 0.050 |
| ATM1 | | | | | | |
| No | 158 | 18 | 6 | 10.7 (2.7) | | |
| Yes | 2 | 1 | 0 | NA | 0.022 | 0.008 |
| KRAS | | | | | | |
| No | 147 | 15 | 6 | 9.8 (2.6) | | |
| Yes | 13 | 4 | 0 | 30.8 (16.9) | 0.015 | 0.013 |
| IKZF1 | | | | | | |
| No | 139 | 12 | 4 | 8.4 (2.6) | | |
| Yes | 21 | 7 | 2 | 29.4 (10.5) | 0.001 | 0.039 |

*Stratified according to treatment protocol: Total XIII intermediate to high risk N = 23, Total XIII low risk N = 28, Total XIV and XV standard and high risk N = 50, total XV low risk N = 59.

TABLE 31
Cumulative incidence of hematologic relapse by genetic lesions, St Jude
cohort with MRD data (N = 160).

| | | | | Cumulative Incidence (SE)% | | |
|---|---|---|---|---|---|---|
| | | | | | P-Value | P-Value |
| | | Hematologic | competing Risks | | Unstratified | Stratified |
| Lesion | N | relapse N | N | 5-Year | Gray's Test | Gray's Test |
| RAG1/2 | | | | | | |
| No | 141 | 12 | 5 | 7.1 (2.4) | | |
| Yes | 10 | 2 | 0 | 25.0 (17.2) | 0.157 | 0.032 |
| KRAS | | | | | | |
| No | 138 | 10 | 5 | 6.5 (2.3) | | |
| Yes | 13 | 4 | 0 | 30.8 (16.9) | 0.002 | 0.002 |
| IKZF1 | | | | | | |
| No | 136 | 10 | 4 | 6.8 (2.4) | | |
| Yes | 15 | 4 | 1 | 20.6 (11.1) | 0.022 | 0.114 |

*Stratified according to treatment protocol: Total XIII intermediate to high risk N = 20, Total XIII low risk N = 28, Total XIV and XV standard and high risk N = 44, total XV low risk N = 59.

TABLE 32
Associations of genetic lesions and day 19 MRD, St Jude cohort (all B-
progenitor ALL cases)

| | Day 19 MRD N (%) | | | |
|---|---|---|---|---|
| | MRD < 0.01% | 0.01% ≦ MRD < 1.0% | MRD ≧ 1.0% | |
| Lesion | 71 (44.1%) | 64 (39.8%) | 26 (16.2%) | exact P-Value |
| ATP10A | | | | |
| No | 71 (44.94) | 63 (39.87) | 24 (15.19) | |
| Yes | 0 (0.00) | 1 (33.33) | 2 (66.67) | 0.0347 |
| ARPP-21 | | | | |
| No | 70 (45.45) | 62 (40.26) | 22 (14.29) | |
| Yes | 1 (14.29) | 2 (28.57) | 4 (57.14) | 0.0101 |
| GAB1 | | | | |
| No | 71 (45.22) | 63 (40.13) | 23 (14.65) | |
| Yes | 0 (0.00) | 1 (25.00) | 3 (75.00) | 0.0064 |
| HIS T1H2BE | | | | |
| No | 68 (46.26) | 59 (40.14) | 20 (13.61) | |
| Yes | 3 (21.43) | 5 (35.71) | 6 (42.86) | 0.0134 |
| IKZF1 | | | | |
| No | 69 (49.29) | 58 (41.43) | 13 (9.29) | |
| Yes | 2 (9.52) | 6 (28.57) | 13 (61.90) | 0.0000 |
| CDK6 | | | | |
| No | 71 (45.81) | 63 (40.65) | 21 (13.55) | |
| Yes | 0 (0.00) | 1 (16.67) | 5 (83.33) | 0.0003 |
| ABL1 | | | | |
| No | 71 (44.94) | 64 (40.51) | 23 (14.56) | |
| Yes | 0 (0.00) | 0 (0.00) | 3 (100.0) | 0.0040 |

TABLE 33
Associations of genetic lesions and day 46 MRD, St Jude cohort (all B-
progenitor ALL cases)

| | Day 46 MRD N (Column %) | | | |
|---|---|---|---|---|
| | MRD < 0.01% | 0.1% ≦ MRD < 1% | MRD ≧ 1.0% | exact |
| Lesion Loci | 126 (78.8%) | 26 (16.3%) | 8 (5%) | P-value |
| NF1 | | | | |
| No | 123 (80.39) | 22 (14.38) | 8 (5.23) | |
| Yes | 3 (42.86) | 4 (57.14) | 0 (0.00) | 0.0429 |
| EBF1 | | | | |
| No | 122 (80.79) | 23 (15.23) | 6 (3.97) | |
| Yes | 4 (44.44) | 3 (33.33) | 2 (22.22) | 0.0210 |
| 6p22 Histone cluster | | | | |
| No | 120 (82.19) | 21 (14.38) | 5 (3.42) | |
| Yes | 6 (42.86) | 5 (35.71) | 3 (21.43) | 0.0030 |
| HBS1L (5′ of MYB) | | | | |
| No | 119 (78.81) | 26 (17.22) | 6 (3.97) | |
| Yes | 7 (77.78) | 0 (0.00) | 2 (22.22) | 0.0383 |
| IKZF1 | | | | |
| No | 119 (85.61) | 19 (13.67) | 1 (0.72) | |
| Yes | 7 (33.33) | 7 (33.33) | 7 (33.33) | 0.0000 |
| CDKN6 | | | | |
| No | 125 (81.17) | 23 (14.94) | 6 (3.90) | |
| Yes | 1 (16.67) | 3 (50.00) | 2 (33.33) | 0.0034 |
| ABL | | | | |
| No | 126 (80.25) | 25 (15.92) | 6 (3.82) | |
| Yes | 0 (0.00) | 1 (33.33) | 2 (66.67) | 0.0015 |

TABLE 34
Associations of genetic lesions and day 19 MRD, St Jude cohort (B-
progenitor ALL cases, excluding BCR-ABL1 ALL)

| | D19 MRD N (Column %) | | | |
|---|---|---|---|---|
| | MRD < 0.01% | 0.01% ≦ MRD < 1.0% | MRD ≧ 1.0% | |
| Lesion Loci | 71 (46.4%) | 61 (39.9%) | 8 (38.1%) | exact P-Value |
| ATP10A | | | | |
| No | 71 (47.33) | 60 (40.00) | 19 (12.67) | |
| Yes | 0 (0.00) | 1 (33.33) | 2 (66.67) | 0.0233 |
| ARPP-21 | | | | |
| No | 70 (47.62) | 59 (40.14) | 18 (12.24) | |
| Yes | 1 (16.67) | 2 (33.33) | 3 (50.00) | 0.0270 |
| GAB1 | | | | |
| No | 71 (47.65) | 60 (40.27) | 18 (12.08) | |
| Yes | 0 (0.00) | 1 (25.00) | 3 (75.00) | 0.0041 |
| IKZF1 | | | | |
| No | 69 (50.00) | 56 (40.58) | 13 (9.42) | |
| Yes | 2 (13.33) | 5 (33.33) | 8 (53.33) | 0.0001 |
| CDK6 | | | | |
| No | 71 (47.97) | 60 (40.54) | 17 (11.49) | |
| Yes | 0 (0.00) | 1 (20.00) | 4 (80.00) | 0.0007 |

TABLE 35
Associations of genetic lesions and day 46 MRD, St Jude cohort (B-
progenitor ALL cases).

| | D46MRD N (Column %) | | | |
|---|---|---|---|---|
| | MRD < 0.01% | 0.01% ≦ MRD < 1.0% | MRD ≧ 1.0% | |
| Lesion | 124 (82.2%) | 23 (15.2%) | 4 (2.6%) | exact P-Value |
| NF1 | | | | |
| No | 121 (84.03) | 19 (13.19) | 4 (2.78) | |
| Yes | 3 (42.86) | 4 (57.14) | 0 (0.00) | 0.0235 |
| IKZF1 | | | | |
| No | 117 (86.03) | 18 (13.24) | 1 (0.74) | |
| Yes | 7 (46.67) | 5 (33.33) | 3 (20.00) | 0.0001 |
| CDK6 | | | | |
| No | 123 (84.25) | 20 (13.70) | 3 (2.05) | |
| Yes | 1 (20.00) | 3 (60.00) | 1 (20.00) | 0.0096 |
| CCDC26 | | | | |
| No | 124 (82.67) | 23 (15.33) | 3 (2.00) | |
| Yes | 0 (0.00) | 0 (0.00) | 1 (100.0) | 0.0296 |

TABLE 36
Genes driving enrichment of the B-cell signal transduction gene set
negatively enriched in P9906 high-risk ALL.

| Gene | Running enrichment score | Core enrichment |
|---|---|---|
| LYN | 0.0174 | YES |
| AKT1 | −0.0397 | YES |
| SHC1 | −0.0897 | YES |
| AKT2 | −0.141 | YES |
| PIK3R1 | −0.183 | YES |
| SYK | −0.23 | YES |
| GRB2 | −0.27 | YES |
| ITPKB | −0.313 | YES |
| CD19 | −0.348 | YES |
| RAF1 | −0.381 | YES |
| NFKB2 | −0.418 | YES |
| BTK | −0.451 | YES |
| NFKB1 | −0.488 | YES |
| NFKBIB | −0.5 | YES |
| PLCG2 | −0.527 | YES |
| PIK3CD | −0.528 | NO |
| SOS2 | −0.475 | NO |
| MAPK1 | −0.487 | NO |
| DAG1 | −0.486 | NO |
| PPP1R13B | −0.466 | NO |
| SOS1 | −0.473 | NO |
| AKT3 | −0.389 | NO |
| BCR | −0.388 | NO |
| VAV1 | −0.386 | NO |
| NFKBIE | −0.372 | NO |
| PIK3CA | −0.362 | NO |
| MAP2K1 | −0.359 | NO |
| NFAT5 | −0.345 | NO |
| BAD | −0.346 | NO |
| NFKBIL2 | −0.314 | NO |
| MAP2K2 | −0.233 | NO |
| EPHB2 | −0.208 | NO |
| NFKBIL1 | −0.128 | NO |
| SERPINA4 | −0.144 | NO |
| CSK | −0.0774 | NO |
| NFKBIA | −0.0896 | NO |
| PI3 | −0.0744 | NO |
| ITPKA | −0.101 | NO |
| BLNK | −0.129 | NO |

TABLE 37
Associations between IKZF1, EBF1, and BTLA alterations and outcome
*4 year estimate

| | P9906 | | | | St Jude | | | |
|---|---|---|---|---|---|---|---|---|
| IKZF1 deletion or | | Hematologic | 5 year incidence | | | Hematologic | 10 year incidence | |
| mutation | N | relapse | (SE) % | P | N | relapse | (SE) % | P |
| No | 153 | 19 | 13.9 (3.1) | | 203 | 36 | 21.9 (3.5) | |
| Yes | 68 | 25 | 52.7 (8.8) | <0.0001 | 55 | 21 | 46.1 (8.2) | 0.002 |
| | | Any Relapse | | | | Any Relapse | | |
| No | 153 | 37 | 25.8 (3.8) | | 203 | 42 | 25.0 (3.6) | |
| Yes | 68 | 37 | 71.9 (8.4) | <0.0001 | 55 | 22 | 47.9 (8.2) | 0.006 |
| | | Any event | | | | Any event | | |
| No | 153 | 40 | 27.5 (3.9) | | 203 | 46 | 27.2 (3.9) | |
| Yes | 68 | 38 | 73.7 (8.2) | <0.0001 | 55 | 27 | 58.7 (8.3) | 0.0002 |
| | | Hematologic | 5 year incidence | | | Hematologic | 10 year incidence | |
| EBF1 deletion | N | relapse | (SE) % | P | N | relapse | (SE) % | P |
| No | 204 | 35 | 23.2 (4.1) | | 246 | 56 | 28.5 (3.5) | |
| Yes | 17 | 9 | 57.5 (14.9) | 0.0001 | 12 | 1 | 8.3 (8.3) | 0.32 |
| | | Any Relapse | | | | Any Relapse | | |
| No | 204 | 62 | 36.7 (4.4) | | 246 | 63 | 31.5 (3.6) | |
| Yes | 17 | 12 | 79.4 (13.5) | 0.001 | 12 | 1 | 8.3 (8.3) | 0.25 |
| | | Any event | | | | Any event | | |
| No | 204 | 66 | 38.7 (4.5) | | 246 | 72 | 35.7 (3.7) | |
| Yes | 17 | 12 | 79.4 (13.5) | 0.002 | 12 | 1 | 8.3 (8.3) | 0.17 |
| | | Hematologic | 5 year incidence | | | Hematologic | 10 year incidence | |
| BTLA deletion | N | relapse | (SE) % | P | N | relapse | (SE) % | P |
| No | 208 | 38 | 23.1 (3.8) | | 238 | 54 | 28.4 (3.6) | |
| Yes | 13 | 6 | 61.5 (21.1) | 0.018 | 20 | 3 | 17.9 (9.9) | 0.47 |
| | | Any Relapse | | | | Any Relapse | | |
| No | 208 | 63 | 30.2 (3.4) | | 238 | 61 | 31.5 (3.7) | |
| Yes | 13 | 11 | 69.2 (13.9)* | <0.0001 | 20 | 3 | 17.9 (9.9) | 0.32 |
| | | Any event | | | | Any event | | |
| No | 208 | 67 | 32.1 (3.4) | | 238 | 68 | 35.0 (3.8) | |
| Yes | 13 | 11 | 69.2 (13.9) | <0.0001 | 20 | 5 | 27.9 (11.3) | 0.84 |

TABLE 38
Associations between IKZF1 deletions or mutations and the presence of elevated levels of
minimal residual disease in P9906 and St Jude cohorts.

| | IKZF1 | | | | | | | |
|---|---|---|---|---|---|---|---|---|
| | deletion or | ≦0.01% | | 0.01% < MRD ≦ 1.0% | | >1.0% | | |
| Cohort | mutation | N (%) | | N (%) | | N (%) | | P-Value |
| P9906, day 8 | No | 26 | (18.31) | 51 | (35.92) | 65 | (45.77) | 0.44 |
| | Yes | 7 | (12.96) | 17 | (31.48) | 30 | (55.56) | |
| P9906, day 28 | No | 100 | (72.46) | 29 | (21.01) | 9 | (6.52) | |
| | Yes | 31 | (46.97) | 19 | (28.79) | 16 | (24.24) | 0.0002 |
| | | <0.01% | | 0.01% ≦ MRD < 1.0% | | ≧1.0% | | P-Value |
| St Jude, day 19 | No | 69 | (49.39) | 58 | (41.42) | 13 | (9.29) | |
| | Yes | 2 | (9.52) | 6 | (28.57) | 13 | (61.9) | <0.0001 |
| St Jude, day 46 | No | 119 | (85.61) | 19 | (13.67) | 1 | (0.72) | |
| | Yes | 7 | (33.33) | 7 | (33.33) | 7 | (33.33) | <0.0001 |

### Discussion

Accurate risk stratification is critical to ensure that patients with high-risk ALL receive treatment of appropriate intensity, while low-risk cases are spared unnecessary toxicity. Current risk stratification is primarily based upon clinical variables, immunophenotype, detection of sentinel cytogenetic/molecular lesions data and early response to therapy'. However, a substantial proportion of patients relapse but have no known risk factors at diagnosis. It is thus critical to identify new markers that improve outcome prediction and identify new treatment targets. Here we have used high-resolution genome-wide copy number analysis to identify genetic lesions associated with outcome.

The most striking finding was a strong association between deletions or mutation of IKZF1 (IKAROS) and poor outcome in two independent cohorts notable for markedly different sample composition and treatment schedules. Importantly, the association of IKZF1 status and outcome was independent of age, presenting leukocyte count, cytogenetic subtype and MRD levels, indicating that IZKF1 profiling at diagnosis will be useful in identifying individuals at high risk of treatment failure. Moreover, the gene expression signatures of poor outcome (IKZF1-deleted) P9906 and St Jude ALL were highly similar, and also similar to the signature of BCR-ABL1 positive ALL, where IKZF1 deletion is extremely common As BCR-ABL1 ALL also has a poor prognosis, these findings suggest that IKZF1 mutation may be a key determinant of the poor outcome of both BCR-ABL1 positive and negative disease. The similarity of the gene expression signatures of IKZF1-mutated, BCR-ABL1 negative ALL and BCR-ABL1 positive ALL raises the possibility that the poor outcome, IKZF1-deleted cases may harbor hitherto unidentified activating mutations in tyrosine kinases.

IKAROS is a transcription factor with well-established roles in lymphopoiesis and cancer¹⁹. Normal IKAROS contains four N-terminal zinc fingers required for normal DNA binding, and two C-terminal zinc fingers that mediate dimerization. IKAROS is required for the development of all lymphoid lineages¹⁹, and mice heterozygous for a dominant negative IKAROS mutation develop aggressive T-lineage hematopoietic disease²⁰. Ikzf1 is also a common target of integration in murine retroviral mutagenesis studies²¹.

Alternate IKAROS transcripts have been widely described in normal hematopoietic cells and leukemic blasts²². Isoforms lacking most or all of the N-terminal zinc fingers have attenuated DNA binding capacity but retain their ability to homo- and heterodimerize, and thus act as dominant negative inhibitors of IKAROS²³. These isoforms have been reported at variable frequency in ALL²². Recently, we reported a near obligate deletion of IKZF1 in BCR-ABL1 positive ALL and lymphoid blast crisis CML, suggesting that perturbation of IKAROS is a key event in the pathogenesis and progression of BCR-ABL1 ALL⁵. Importantly, there was complete correlation between the extent of genomic deletion and the expression of aberrant IKAROS isoforms⁵. For example, all cases expressing the dominant negative Ik6 isoform, that lacks exons 3-6 and all N-terminal zinc fingers, had genomic deletions of exons 3-6⁵.

The present study demonstrates that IKZF1 alterations are present in a substantial proportion of BCR-ABL1 negative B-progenitor ALL cases, predominantly in cases that lack other common recurrent cytogenetic abnormalities (3 8.8% of P9906 and 22.8% St Jude cases with normal or miscellaneous cytogenetic abnormalities). As in BCR-ABL1 positive ALL, IKZF1 deletions involved either the entire locus or subsets of exons, and are predicted to result in either haploinsufficiency or the expression of dominant negative IKAROS isoforms. Moreover, we have identified sequence mutations of IKZF1 in ALL that are predicted to result in loss of normal IKAROS function or expression of a novel dominant negative isoform, G158S.

Using GSEA, we found negative enrichment of genes involved in normal B lymphoid signaling and development in poor outcome ALL. This is compatible with the known requirement for IKAROS in lymphoid development¹⁹, and previous studies showing that expression of dominant negative IKAROS isoforms impairs B lymphoid differentiation²⁴. Together, these data suggest that attenuation of normal IKAROS activity and the resulting block in lymphoid maturation renders leukemic cells less susceptible to eradication by chemotherapeutic agents. Whether this relates to enrichment for properties that are characteristic of leukemia initiating or stem cells, including their inherent drug resistant mechanisms, remains to be determined²⁵.

Notably, we did not find outcome to be associated with extensively studied loci such as CDKN2A/B^{26,27}, or with PAX5 status, despite PAX5 alterations being the most common B-cell pathway lesions observed in both cohorts. This suggests that PAX5 is important in establishing the leukemic clone, whereas deletions of IKZF1 may also directly contribute to treatment resistance. Experimental studies addressing the relative contribution of these two lesions to leukemogenesis and treatment resistance will provide valuable insights into how these genetic alterations contribute to the molecular pathology of ALL.

In summary, we have identified alterations of IKZF1 as a new prognostic marker in childhood B-progenitor ALL, and integrated genomic analysis suggests that IKZF1 directly contributes to treatment resistance in ALL. These results provide a strong rationale for the integration of IKZF1 status analysis in the diagnostic evaluation of patients with ALL.

### REFERENCES

1. Pui C H, Robison L L, Look A T. Acute lymphoblastic leukaemia. Lancet 2008; 371:1030-43.
2. Rivera G K, Zhou Y, Hancock M L, et al. Bone marrow recurrence after initial intensive treatment for childhood acute lymphoblastic leukemia. Cancer 2005; 103:368-76.
3. Mullighan C G, Goorha S, Radtke I, et al. Genome-wide analysis of genetic alterations in acute lymphoblastic leukaemia. Nature 2007; 446:758-64.
4. Kuiper R P, Schoenmakers E F, van Reijmersdal S V, et al. High-resolution genomic profiling of childhood ALL reveals novel recurrent genetic lesions affecting pathways involved in lymphocyte differentiation and cell cycle progression. Leukemia 2007; 21:1258-66.
5. Mullighan C G, Miller C B, Phillips L A, et al. BCR-ABL1 lymphoblastic leukaemia is characterized by the deletion of Ikaros. Nature 2008; 453:110-4.
6. Kawamata N, Ogawa S, Zimmermann M, et al. Molecular allelokaryotyping of pediatric acute lymphoblastic leukemias by high-resolution single nucleotide polymorphism oligonucleotide genomic microarray. Blood 2008; 111:776-84.
7. Nachman J B, Sather H N, Sensel M G, et al. Augmented post-induction therapy for children with high-risk acute lymphoblastic leukemia and a slow response to initial therapy. N Engl J Med 1998; 338:1663-71.
8. Borowitz M J, Devidas M, Hunger S P, et al. Clinical significance of minimal residual disease in childhood acute lymphoblastic leukemia and its relationship to other prognostic factors: a Children's Oncology Group study. Blood 2008; 1 11:5477-85.
9. Shuster J J, Camitta B M, Pullen J, et al. Identification of newly diagnosed children with acute lymphocytic leukemia at high risk for relapse. Cancer Res Ther and Control 1999; 9:101-7.
10. Coustan-Smith E, Behm F G, Sanchez J, et al Immunological detection of minimal residual disease in children with acute lymphoblastic leukaemia. Lancet 1 998; 35 1:550-4.
11. Coustan-Smith E, Sancho J, Hancock M L, et al. Clinical importance of minimal residual disease in childhood acute lymphoblastic leukemia. Blood 2000; 96:2691-6.
12. Subramanian A, Tamayo P, Mootha V K, et al. Gene set enrichment analysis: a knowledge-based approach for interpreting genome-wide expression profiles. Proc Natl Acad Sci USA 2005; 102:15545-50.
13. Efron B, Tibshirani R. On testing the significance of sets of genes. Ann Appl Stat 2007; 1:107-29.
14. Bair E, Hasle H, Debashis P, Tibshirani R. Prediction by Supervised Principal Components. J Am Stat Assoc 2006; 101:119-37.
15. Bair E, Tibshirani R. Semi-supervised methods to predict patient survival from gene expression data. PLoS Biol 2004; 2:E108.
16. Maier H, Ostraat R, Parenti S, et al. Requirements for selective recruitment of Ets proteins and activation of mb-1/Ig-alpha gene transcription by Pax-5 (BSAP). Nucleic Acids Res 2003; 31:5483-9.
17. Cobb B S, Morales-Alcelay S, Kleiger G, Brown K E, Fisher A G, Smale S T. Targeting of Ikaros to pericentromeric heterochromatin by direct DNA binding. Genes Dev 2000; 14:2146-60.
18. Buhl A M, Nemazee D, Cambier J C, Rickert R, Hertz M. B-cell antigen receptor competence regulates B-lymphocyte selection and survival. Immunol Rev 2000; 176:141-53.
19. Georgopoulos K, Bigby M, Wang J H, et al. The Ikaros gene is required for the development of all lymphoid lineages. Cell 1 994; 79:143-56.
20. Winandy S, Wu P, Georgopoulos K. A dominant mutation in the Ikaros gene leads to rapid development of leukemia and lymphoma. Cell 1995; 83:289-99.
21. Uren A G, Kool J, Matentzoglu K, et al. Large-scale mutagenesis in p1 9(ARF)- and p53-deficient mice identifies cancer genes and their collaborative networks. Cell 2008; 133:727-41.
22. Rebollo A, Schmitt C. Ikaros, Aiolos and Helios: transcription regulators and lymphoid malignancies. Immunol Cell Biol 2003; 81:171-5.
23. Sun L, Liu A, Georgopoulos K. Zinc finger-mediated protein interactions modulate Ikaros activity, a molecular control of lymphocyte development. Embo J 1996; 15:5358-69.
24. Tonnelle C, Bardin F, Maroc C, et al. Forced expression of the Ikaros 6 isoform in human placental blood CD34(+) cells impairs their ability to differentiate toward the B-lymphoid lineage. Blood 2001; 98:2673-80.
25. le Viseur C, Hotfilder M, Bomken S, et al. In childhood acute lymphoblastic leukemia, blasts at different stages of immunophenotypic maturation have stem cell properties. Cancer Cell 2008; 14:47-58.
26. Calero Moreno T M, Gustafsson G, Garwicz S, et al. Deletion of the Ink4-locus (the p161nk4a, p14ARF and p151nk4b genes) predicts relapse in children with ALL treated according to the Nordic protocols NOPHO-86 and NOPHO-92. Leukemia 2002; 16:2037-45.
27. Mirebeau D, Acquaviva C, Suciu S, et al. The prognostic significance of CDKN2A, CDKN2B and MTAP inactivation in B-lineage acute lymphoblastic leukemia of childhood. Results of the EORTC studies 58881 and 58951. Haematologica 2006; 9 1:881-5.
28. Shuster J J, Camitta B M, Pullen J, et al. Identification of newly diagnosed children with acute lymphocytic leukemia at high risk for relapse. Cancer Res Ther and Control 1999; 9:101-7.
29. Mullighan C, Downing J. Ikaros and acute leukemia. Leuk Lymphoma 2008; 49:847-9.
30. Pui C H, Boyett J M, Rivera G K, et al. Long-term results of Total Therapy studies 11, 12 and 13A for childhood acute lymphoblastic leukemia at St Jude Children's Research Hospital. Leukemia 2000; 14:2286-94.
31. Pui C H, Sandlund J T, Pei D, et al. Improved outcome for children with acute lymphoblastic leukemia: results of Total Therapy Study XIIIB at St Jude Children's Research Hospital. Blood 2004; 104:2690-6.
32. Kishi S, Griener J, Cheng C, et al. Homocysteine, pharmacogenetics, and neurotoxicity in children with leukemia. J Clin Oncol 2003; 21:3084-91.
33. Pui C H, Relling M V, Sandlund J T, Downing J R, Campana D, Evans W E. Total Therapy study XV for newly diagnosed childhood acute lymphoblastic leukemia: study design and preliminary results. Ann Hematol 2006; 85 Suppl 1:88-91.
34. Pieters R, Schrappe M, De Lorenzo P, et al. A treatment protocol for infants younger than 1 year with acute lymphoblastic leukaemia (Interfant-99): an observational study and a multicentre randomised trial. Lancet 2007; 370:240-50.
35. Lin M, Wei U, Sellers W R, Lieberfarb M, Wong W H, Li C. dChipSNP: significance curve and clustering of SNP-array-based loss-of-heterozygosity data. Bioinformatics 2004; 20: 123 3-40.
36. Venkatraman E S, Olshen A B. A faster circular binary segmentation algorithm for the analysis of array CGH data. Bioinformatics 2007; 23:657-63.
37. Ewing B, Hillier L, Wendl M C, Green P. Base-calling of automated sequencer traces using phred. I. Accuracy assessment. Genome Res 1998; 8:175-85.
38. Ewing B, Green P. Base-calling of automated sequencer traces using phred. II. Error probabilities. Genome Res 1998; 8:186-94.
39. Zhang J, Wheeler D A, Yakub I, et al. SNPdetector: a software tool for sensitive and accurate SNP detection. PLoS Comput Biol 2005; 1:e53.
40. Zhang J, Finney R P, Rowe W, et al. Systematic analysis of genetic alterations in tumors using Cancer Genome WorkBench (CGWB). Genome Res 2007; 17:1 111-7.
41. Zhang J, Rowe W L, Struewing J P, Buetow K H. HapScope: a software system for automated and visual analysis of functionally annotated haplotypes. Nucleic Acids Res 2002; 30:5213-21.
42. Bamford S, Dawson E, Forbes S, et al. The COSMIC (Catalogue of Somatic Mutations in Cancer) database and website. Br J Cancer 2004; 91:355-8.
43. Sherry S T, Ward M H, Kholodov M, et al. dbSNP: the NCBI database of genetic variation. Nucleic Acids Res 2001; 29:308-1 1.
44. Garvie C W, Hagman J, Wolberger C. Structural studies of Ets-1/Pax5 complex formation on DNA. Mol Cell 2001; 8:1267-76.
45. Jones T A, Zou J Y, Cowan S W, Kjeldgaard M. Improved methods for building protein models in electron density maps and the location of errors in these models. Acta Crystallogr A 1991; 47 (Pt 2):110-9.
46. DeLano WL. The PyMOL Molecular Graphics System. In. San Carlos, Calif.; 2002.
47. Gray R J. A class of K-sample tests for comparing the cumulative incidence of a competing risk. Annals Statistics 1988; 16:1 141-54.
48. Peto R, Pike M C, Armitage P, et al. Design and analysis of randomized clinical trials requiring prolonged observation of each patient. II. analysis and examples. Br J Cancer 1977; 35:1-39.
49. Mantel N. Evaluation of survival data and two new rank order statistics arising in its consideration. Cancer Chemother Rep 1966; 50:163-70.
50. Fine J P, Gray R J. A Proportional Hazards Model for the Subdistribution of a Competing Risk. J Am Stat Assoc 1999; 94:496-509.
51. R Development Core Team. R: A language and environment for statistical computing. R Foundation for Statistical Computing 2006; Vienna:Austria.
52. Smyth G K. Linear models and empirical bayes methods for assessing differential expression in microarray experiments. Stat Appl Genet Mol Biol 2004; 3:Article3.
53. Gentleman R C, Carey V J, Bates D M, et al. Bioconductor: open software development for computational biology and bioinformatics. Genome Biol 2004; 5:R80.
54. Benjamini Y, Hochberg Y. Controlling the false discovery rate: a practical and powerful approach to multiple testing. J R Stat Soc B 1995; 57:289-300.
55. Subramanian A, Tamayo P, Mootha V K, et al. Gene set enrichment analysis: a knowledge-based approach for interpreting genome-wide expression profiles. Proc Natl Acad Sci USA 2005; 102:15545-50.
56. Efron B, Tibshirani R. On testing the significance of sets of genes. Ann Appl Stat 2007; 1:107-29.
57. Edgar R, Domrachev M, Lash A E. Gene Expression Omnibus: NCBI gene expression and hybridization array data repository. Nucleic Acids Res 2002; 30:207-10.
58. Barrett T, Troup D B, Wilhite S E, et al. NCBI GEO: mining tens of millions of expression profiles—database and tools update. Nucleic Acids Res 2007; 35:D760-5.

### Example 3

### Genomic Analysis of the Clonal Origins of Relapsed Acute Lymphoblastic Leukemia

Most children with acute lymphoblastic leukemia (ALL) can be cured, but for the subset of patients who undergo relapse prognosis is dismal. To explore the genetic basis of relapse, we performed genome-wide DNA copy number analyses on matched diagnosis and relapse samples from 61 patients with ALL. In the majority of cases, the diagnosis and relapse samples showed different patterns of genomic copy number abnormalities (CNAs), with the abnormalities acquired at relapse preferentially affecting genes involved in cell cycle regulation and B cell development. Although the diagnosis and relapse samples were genetically related, most relapse samples lacked some of the CNAs present at diagnosis, suggesting that the cells responsible for relapse are ancestral to the primary leukemia cells. Backtracking studies demonstrated that cells corresponding to relapse clone were often present as minor sub-populations at diagnosis. These data suggest that genomic abnormalities contributing to ALL relapse are selected for during treatment and that the signaling pathways affected by these acquired alterations may be rational targets for therapeutic intervention.

Despite cure rates for pediatric acute lymphoblastic leukemia (ALL) exceeding 80% (58), treatment failure remains a significant problem. Relapsed ALL ranks as the fourth most common childhood malignancy and has an overall survival rate of only 30% (59, 60). Important biological and clinical differences have been identified between diagnostic and relapsed leukemic cells including the acquisition of new chromosomal abnormalities, gene mutations, and reduced responsiveness to chemotherapeutic agents (61-64). However, many questions remain about the molecular abnormalities responsible for relapse, as well as the relationship between the cells giving rise to the primary and recurrent leukemias in individual patients.

Genome-wide analyses of DNA copy number abnormalities (CNAs) and loss-of-heterozygosity (LOH) using single nucleotide polymorphism (SNP) arrays have provided important insights into the pathogenesis of newly diagnosed ALL. We have previously reported multiple recurring somatic CNAs in genes encoding transcription factors, cell cycle regulators, apoptosis mediators, lymphoid signaling molecules and drug receptors in B-progenitor and T-lineage ALL (65, 66). To gain insights into the molecular lesions responsible for ALL relapse, we have now performed genome-wide CNA and LOH analyses on matched diagnostic and relapse bone marrow samples from 61 pediatric ALL patients (data not shown). These samples included 47 B-progenitor and 14 T-lineage ALL (T-ALL) cases (67). Samples were flow sorted to ensure at least 80% tumor cell purity prior to DNA extraction (data not shown). DNA copy number and LOH data were obtained using Affymetrix SNP 6.0 (47 diagnosis-relapse pairs) or 500K arrays (14 pairs). Remission bone marrow samples were also analyzed for 48 patients (data not shown).

These analyses identified a mean of 10.8 somatic CNAs per B-ALL case at diagnosis, and 7.1 CNAs per T-ALL case (data not shown). 48.9% of B-ALL cases at diagnosis had CNAs in genes known to regulate B-lymphoid development, including PAX5 (N=12), IKZF1 (N=12), EBF1 (N=2), and RAG1/2 (N=2) (tables S5, S6 and S9). Deletion of CDKN2A/B was present in 36.2% of B-ALL and 71.4% T-ALL cases, and deletion of ETV6 in 11 B-ALL cases. We also identified novel CNAs involving ARID2, which encodes a member of a chromatin remodeling complex (68), the cyclic AMP regulated phosphoprotein ARPP-21, the IL3RA and CSF2RA cytokine receptor genes (data not shown), and the Wnt/β-catenin pathway genes CTNNB1, WNT9B and CREBBP (data not shown).

Although evidence for clonal evolution and/or selection at relapse has been previously reported (61, 63, 64, 69-78), we observed a striking degree of change in the number, extent, and nature of CNAs between diagnosis and relapse in paired samples of ALL. A significant increase in the mean number of CNAs per case were observed in relapse B-ALL samples (10.8 at diagnosis versus 14.0 at relapse, P=0.0005) with the majority being additional regions of deletion (6.8 deletions/case at diagnosis versus 9.2/case at relapse, P=0.0006; and 4.0 gains/case at diagnosis versus 4.8 gains/case at relapse, P=0.03; data not shown). By contrast, no significant changes in lesion frequency were observed in T-ALL (data not shown).

The majority (88.5%) of relapse samples harbored at least some of the CNAs present in the matched diagnosis sample, indicating a common clonal origin (data not shown); however, 91.8% exhibited a change in the pattern of CNAs from diagnosis to relapse (data not shown). 34% acquired new CNAs, 12% showed loss of lesions present at diagnosis, and 46% both acquired new lesions and lost lesions present at diagnosis. In 11% of relapsed samples (three B-ALL and four T-ALL cases) all CNAs present at diagnosis were lost at relapse, raising the possibility that the relapse represents the emergence of a second unrelated leukemia. One case (BCR-ABL-SNP-#15) retained the same translocation at relapse, indicating a common clonal origin. In the remaining three cases, lack of similarity of the patterns of deletion at immunoglobulin (Ig) and T-cell antigen receptor (TcR) gene loci suggested that relapse represented emergence of a distinct leukemia (data not shown). For all other relapse cases (86%), analysis of 1 g/TCR deletions demonstrated a clonal relationship between diagnostic and relapse samples (data not shown).

The genes most frequently affected by CNAs acquired at relapse were CDKN2A/B, ETV6, and regulators of B-cell development (Table 39, and data not shown). Sixteen B- and two T-ALL cases acquired new CNAs of CDKN2A/B, 10 of which lacked CDKN2A/B deletions at diagnosis (data not shown). The CDKN2A/B deletions acquired at relapse were bi-allelic in 70% of cases, resulting in a complete loss of expression of all three encoded proteins: INK4A (p16), ARF (p14), and INK4B (p15). Deletion of ETV6, a frequent abnormality at diagnosis in ETV6-RUNX1 B-ALL (65, 76), was also common in relapsed ALL, being identified in 11 cases (10 B-ALLs and one T-ALL), with only one case ETV6-RUNX1 positive (data not shown). Mutations of genes regulating B cell development are common at diagnosis in B-ALL (65), and additional lesions in this pathway were observed at relapse, with a number of cases acquiring multiple hits within the pathway (data not shown). Four cases lacked CNAs in this pathway at diagnosis but acquired deletions in PAX5 (N=1), IKZF1 (N=2), or TCF3 (N=1) at relapse. Eleven cases with CNAs in this pathway at diagnosis acquired additional lesions at relapse, most commonly IKZF1 (5 cases), IKZF2 (two cases) and IKZF3 (one case) (data not shown). New CNAs were also observed in PAX5 (N=3), TCF3 (N=3), RAG1/2 (N=2; data not shown) and EBF1 (N=1, data not shown). CNAs involving genes encoding regulators of lymphoid development were also observed in four T-ALL relapse samples but involved the early lymphoid regulators IKZF1 (N=2), IKZF2 (N=1) and LEF1 (N=2; data not shown), rather than B lineage specific genes such as PAX5 and EBF1.

TABLE 39
Targets of relapse-acquired CNA in ALL, ranked in order of frequency

| | | B-progenitor | T-lineage |
|---|---|---|---|
| Lesion | | ALL | ALL |
| Deletion | CDKN2A | 16 | 2 |
| | ETV6 | 10 | 1 |
| | IKZF1 | 5 | 2 |
| | NR3C1 | 4 | 0 |
| | TCF3 | 3 | 0 |
| | DMD | 2 | 0 |
| | ARPP-21 | 2 | 0 |
| | CD200 | 2 | 0 |
| | RAG1/2 | 2 | 0 |
| | IKZF2 | 1 | 1 |
| | BTLA | 1 | 1 |
| | ADD3 | 1 | 0 |
| | C20orf94 | 1 | 0 |
| | TBL1XR1 | 1 | 0 |
| | IKZF3 | 1 | 0 |
| Gain | MYB | 0 | 2 |
| | DMD | 1 | 0 |

A number of other less frequent CNAs previously detected in diagnostic ALL samples (65) were also observed as new lesions at relapse, including CNAs of ADDS, ARPP-21, ATM, BTG1, CD200/BTLA, FHIT, KRAS, IL3RA/CSF2RA, NF1, PTCH, TBL1XR1, TOX, WT1, NR3C1 and DMD (data not shown); and progression of intrachromosomal amplification of chromosome 21, a poor prognostic marker in childhood ALL (79) (data not shown). In addition, relapsed T-ALL was remarkable for the loss and acquisition of sentinel lesions in T-ALL, including the loss of NUP214-ABL1 in one case, and the acquisition of NUP214-ABL1, LMO2, and MYB amplification at relapse (65, 80-82) (data not shown).

In addition to defining CNAs, we also performed an analysis of regions of copy-neutral LOH(CN-LOH) that can signify mutated, reduplicated genes. CN-LOH was only identified in 15 B- and 3 T-ALL cases (data not shown). The most common region involved was chromosome 9p (N=8), which in each case contained homozygous CDKN2A/B deletion, consistent with reduplication of a hemizygous CDKN2A/B deletion.

To determine which biologic pathways were most frequently targeted by relapse-acquired CNAs, we categorized each gene contained within altered genomic regions into one or more of 148 biologic pathways. The pathways were then assessed for their frequency of involvement by CNAs across the dataset using Fisher's exact test (66). This analysis identified cell cycle regulation and B-cell development as the most common pathways targeted at relapse (data not shown).

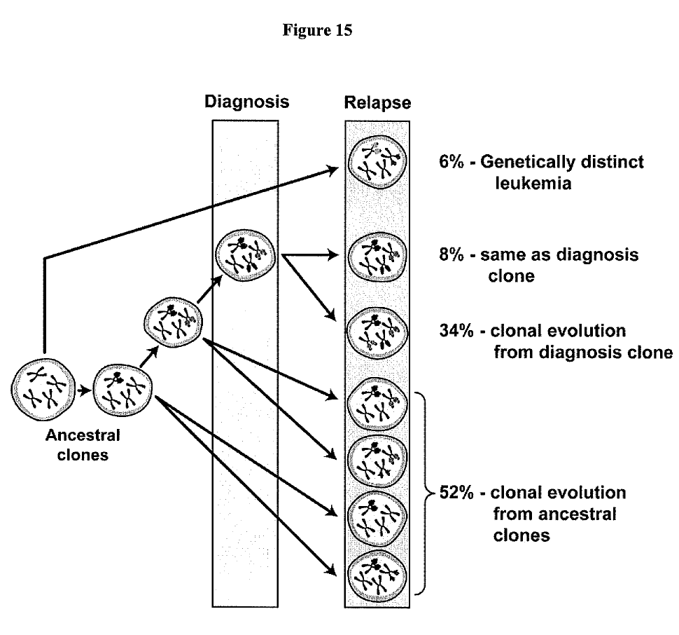

There was a clear clonal relationship between the diagnosis and relapse ALL samples in most cases (93.6% B-ALL and 71.4% T-ALL cases). This suggests that the relapse-associated CNAs were either present at low levels at diagnosis and selected for at relapse, or acquired as new genomic alterations after initial therapy. To explore these possibilities, we mapped the genomic breakpoints of several CNAs acquired at relapse (ADDS, C20orf94, DMD, ETV6, IKZF2, and IKZF3) and developed lesion-specific PCR assays. Evidence of the relapse clone was detected in 7 of 10 diagnostic samples analyzed (FIGS. 15C-H). Thus, the relapse clone is frequently present as a minor sub-population at diagnosis.

By carefully analyzing the changes in CNAs between matched diagnostic and relapse samples, we were able to map their evolutionary relationship (FIG. 15). In a minority of cases, “relapse” is a misnomer, as no CNAs were shared by the diagnostic and relapse clones. The recurrent disease in these cases either represents a secondary leukemia, or a leukemia arising from an ancestral clone that lacks any of the CNAs present in the diagnosis leukemia. In 8% of cases there were no differences in CNAs between the diagnostic and relapse clones, whereas in 34% of cases relapse represented clonal evolution of the diagnosis leukemic populations. Remarkably, however, in almost half of the cases the relapse clone was derived from an ancestral, pre-diagnosis leukemic precursor cell and not from the clone predominating at diagnosis. One illustrative case (Other-SNP-#29) had two relapse-acquired deletions (ETV6 and DMD), only one of which was present in the diagnostic sample as a minor clone (ETV6, data not shown), indicating that these lesions were acquired at different stages of evolution of the relapse clone. This case provides unequivocal evidence of a common ancestral clone that give rise to the major clone at diagnosis, and to a second clone that was present as a minor population at diagnosis but acquired different genetic alterations before emerging as the relapse clone.

These results extend previous studies examining individual genetic loci in relapsed ALL (71, 73, 77, 78, 83-85), and provide important insights into the spectrum of genetic lesions that underlie this process. Although our data are limited to a single class of mutations (CNAs), they demonstrate that no single genetic lesion or alteration of a single pathway is responsible for relapse. Moreover, global genomic instability does not appear to be a prevalent mechanism. Instead, a diversity of mutations appear to contribute to relapse with the most common alterations targeting key regulators of tumor suppression, cell cycle control, and lymphoid/B cell development. Notably, few lesions involved genes with roles in drug import, metabolism, export and/or response, (an exception being the glucocorticoid receptor gene NR3C1) suggesting that the mechanism of relapse is more complex than simple “drug resistance”.

The diversity of genes that are targeted by relapse-associated CNAs coupled with the presence of the relapse clone as a minor sub-population at diagnosis that escapes drug-induced killing represent formidable challenges to the development of effective therapy for relapsed ALL. Nevertheless, our study has identified several common pathways that may contain rational targets against which novel therapeutic agents can be developed.

### REFERENCES

58. C. H. Pui, L. L. Robison, A. T. Look, Lancet 371, 1030 (2008).
59. H. G. Einsiedel et al., J Clin Oncol 23, 7942 (2005).
60. G. K. Rivera et al., Cancer 103, 368 (2005).
61. S. C. Raimondi, C. H. Pui, D. R. Head, G. K. Rivera, F. G. Behm, Blood 82, 576 (1993).
62. E. Klumper et al., Blood 86, 3861 (1995).
63. K. W. Maloney, L. McGavran, L. F. Odom, S. P. Hunger, Blood 93, 2380 (1999).
64. J. A. Irving et al., Cancer Res 65, 3053 (2005).
65. C. G. Mullighan et al., Nature 446, 758 (2007).
66. C. Mullighan, J. Downing, Leuk Lymphoma 49, 847 (2008).
67. Materials and methods are available as supporting material on Science Online.
68. Z. Yan et al., Genes Dev 19, 1662 (2005).
69. J. J. Taylor et al., Leukemia 8, 60 (1994).
70. G. M. Marshall et al., Leukemia 9, 1847 (1995).
71. F. Davi, C. Gocke, S. Smith, J. Sklar, Blood 88, 609 (1996).
72. R. Rosenquist et al., Eur J Haematol 63, 171 (1999).
73. A. M. Ford et al., Blood 98, 558 (2001).
74. G. Germano et al., Leukemia 17, 1573 (2003).
75. S. Takeuchi et al., Oncogene 22, 6970 (2003).
76. J. Zuna et al., Clin Cancer Res 10, 5355 (2004).
77. E. R. Panzer-Grumayer et al., Clin Cancer Res 11, 7720 (2005).
78. S. Choi et al., Blood 110, 632 (2007).
79. A. V. Moorman et al., Blood 109, 2327 (2007).
80. C. Graux et al., Nat Genet. 36, 1084 (2004).
81. I. Lahortiga et al., Nat Genet. 39, 593 (2007).
82. E. Clappier et al., Blood 110, 1251 (2007).
83. A. Beishuizen et al., Blood 83, 2238 (1994).
84. M. Peham et al., Genes Chromosomes Cancer 39, 156 (2004).
85. M. Konrad et al., Blood 101, 3635 (2003).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method for making a prognosis of acute lymphoblastic leukemia (ALL) in a patient comprising
a) assaying the nucleic acid complement of a biological sample from said patient for a genomic abnormality in the IKZF1 gene comprising detecting the genomic abnormality of the IKZF1 gene in the nucleic acid complement of said biological sample, wherein the presence of the genomic abnormality of said IKZF1 gene is indicative of a subgroup of ALL having poor outcomes; and,
b) providing a prognosis of the patient's ALL based on the assay of step (a).

2. A method for determining the progression of chronic myeloid leukemia (CML) in a patient comprising
a) providing a biological sample from said patient, wherein said biological sample comprises genomic DNA of said sample,
b) determining if said genomic DNA comprises a genomic abnormality in the IKZF1 gene,
wherein the presence of the genomic abnormality of said IKZF1 gene is indicative of progression into blastic transformation of CML.

3. A method for classifying a cell as BCR-ABL1 positive ALL or as blast crisis chronic myeloid leukemia (BC-CML) comprising
a) providing a biological sample from a patient, wherein said biological sample comprises genomic DNA of said sample,
b) determining if said genomic DNA comprises a genomic abnormality in the IKZF1 gene,
wherein the presence of the genomic abnormality of said IKZF1 gene is indicative of BCR-ABL1 positive ALL or is indicative of progression into blastic transformation of CML.

4. A method for diagnosing BCR-ABL1 positive ALL in a leukemia patient comprising
a) providing a biological sample from said patient, wherein said biological sample comprises genomic DNA of said sample,
b) determining if said genomic DNA comprises a genomic abnormality in the IKZF1 gene,
wherein the presence of the genomic abnormality of said IKZF1 gene is indicative of BCR-ABL1 positive ALL.

5. The method of claim 1, further comprising selecting a therapy for said patient.

6. The method of claim 1, wherein the genomic abnormality in the IKZF1 gene comprises a deletion of the IKZF1 gene.

7. The method of claim 1, wherein the genomic abnormality in the IKZF1 gene comprises an intragenic deletion of the IKZF1 gene.

8. The method of claim 1, wherein said genomic abnormality in the IKZF1 gene comprises a deletion of at least one exon of the IKZF1 gene.

9. The method of claim 7, wherein said genomic abnormality of the IKZF1 gene comprises a deletion of exon 3 through exon 6 of the IKZF1 gene.

10. The method of claim 7, wherein said genomic abnormality of the IKZF1 gene comprises a deletion of exon 2 through exon 6 of the IKZF1 gene.

11. The method of claim 7, wherein said genomic abnormality of the IKZF1 gene comprises a deletion of exon 1 through exon 6 of the IKZF1 gene.

12. The method of claim 7, wherein said genomic abnormality of the IKZF1 gene comprises a deletion of the promoter or a deletion in the 5′ region of the IKZF1 gene.

13. The method claim 1, wherein said genomic abnormality of the IKZF1 gene results in the expression of a dominant negative isoform of a IKZF1 polypeptide, wherein said isoform does not bind DNA.

14. The method of claim 1, wherein said genomic abnormality of the IKZF1 gene results in the complete loss of expression of the IKZF1 polypeptide.

15. The method of claim 1, wherein said genomic abnormality of the IKZF1 gene results from a recombinase activating gene (RAG) mediated-recombination event.

16. The method of claim 1, wherein determining if said biological sample comprises the genomic abnormality in the IKZF1 gene comprises detecting genomic abnormalities of genomic DNA using a nucleic acid sequencing technique.

17. The method of claim 1, wherein determining if said biological sample comprises the genomic abnormalities in the IKZF1 gene comprises detecting said genomic abnormalities in a nucleic acid hybridization technique.

18. The method of claim 17, wherein said nucleic acid hybridization technique is selected from the group consisting of in situ hybridization (ISH) and Southern blot.

19. The method of claim 1, wherein determining if said biological sample comprises the genomic abnormality in the IKZF1 gene comprises detecting said genomic abnormalities in a nucleic acid amplification method.

20. The method of claim 19, wherein said nucleic acid amplification method is selected from the group consisting of polymerase chain reaction (PCR), transcription-mediated amplification (TMA), ligase chain reaction (LCR), strand displacement amplification (SDA), and nucleic acid sequence based amplification (NASBA).

21. The method of claim 1, wherein determining if said genomic DNA comprises a genomic abnormality in the IKZF1 gene employs at least one primer comprising a nucleotide sequence as set forth in SEQ ID NO: 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, or 104.

22. The method of claim 1, wherein said biological sample is selected from the group consisting of peripheral blood, bone marrow, apheresis samples, cerebrospinal fluid, saliva, urine, gonadal tissue, tissue (e.g. chloroma) biopsies, or any other human tissue sample potentially involved by leukemic infiltration.

23. The method of claim 1, wherein said biological sample is from a human.

24. The method of claim 4, wherein said genomic abnormality in the IKZF1 gene is detected by directly assaying the genomic DNA sequence.

25. The method of claim 4, wherein said genomic abnormality in the IKZF1 gene is detected by directly assaying the transcript produced from the genomic DNA.

26. A kit for classifying a cell in a biological sample as BCR-ABL1 positive ALL or the likelihood of progression into blast crisis chronic myeloid leukemia (BC-CML) or prognosing ALL with poor outcomes comprising a primer consisting of a polynucleotide sequence as set forth in SEQ ID NO: 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, and/or 104.

27. A method of screening for a compound capable of selectively binding a dominant negative isoform of a IKZF1 polypeptide, wherein said isoform does not bind DNA comprising
a) contacting said compound with the dominant negative isoform of the IKZF1 polypeptide, and
b) determining whether said compound specifically binds said dominant negative isoform of the IKZF1 polypeptide.

28. A method of screening for a compound capable of modulating the activity of a dominant negative isoform of a IKZF1 polypeptide wherein said isoform does not bind DNA Comprising
a) contacting said compound with the dominant negative isoform of the IKZF1 polypeptide, and
b) determining whether said compound modulates the activity of said dominant negative isoform of the IKZF1 polypeptide.
